(19) United States
(12) Patent Application Publication
(10) Pub. No.: US 2026/0077054 A1
(43) Pub. Date: Mar. 19, 2026

(54) COMBINATIONS OF ANTI-GIP ANTIBODIES AND INCRETINS AND USES THEREOF

(71) Applicant: HELICORE BIOPHARMA, INC., San Mateo, CA (US)

(72) Inventors: James Patterson, San Diego, CA (US); Jing Liang, Fishkill, NY (US); Paul Kierstead, Lafayette, CA (US)

(21) Appl. No.: 19/333,060

(22) Filed: Sep. 18, 2025

Related U.S. Application Data
(60) Provisional application No. 63/696,174, filed on Sep. 18, 2024.
(60) Provisional application No. 63/796,859, filed on Apr. 29, 2025.

(51) Int. Cl. A61K 47/68; A61P 3/04; C07K 16/26

(52) U.S. Cl. CPC A61K 47/6811; A61K 47/6847; A61P 3/04; C07K 16/26

(57) ABSTRACT

Provided for herein are anti-GIP antibodies, or antigen-binding fragments thereof. Conjugates of the anti-GIP antibodies, or antigen-binding fragments thereof, to biologically active peptides are also provided. Pharmaceutical compositions of the, anti-GIP antibodies, or antigen-binding fragments thereof, biologically active peptides, combinations thereof, or conjugates thereof are also provided. Methods of using the embodiments and compositions provided for herein are also provided.

### RELATED APPLICATIONS

This application claims the benefit of U.S. Provisional Application Ser. No. 63/696,174 filed Sep. 18, 2024, and U.S. Provisional Application Ser. No. 63/796,859 filed Apr. 29, 2025, each of which are hereby incorporated by reference in their entirety.

### REFERENCE TO SEQUENCE LISTING SUBMITTED ELECTRONICALLY

The instant application contains a Sequence Listing which has been submitted electronically in XML format and is hereby incorporated by reference in its entirety. Said XML copy, created on Sep. 17, 2025, is named “HEB-006US_SL.xml” and is 163,224 bytes in size.

### FIELD

The present disclosure relates generally to anti-GIP antibodies and combinations with biologically active peptides for the treatment of diseases.

### BACKGROUND

Incretins are a group of metabolic hormones that stimulate a decrease in blood glucose levels. Incretins are released after eating and augment the secretion of insulin released from pancreatic beta cells of the islets of Langerhans by a blood-glucose-dependent mechanism. Glucose-dependent insulinotropic polypeptide (GIP) and glucagon like polypeptide-1 (GLP-1) are incretins also known as insulinotropic factors.

GIP, formerly known as gastric inhibitory polypeptide, is a single 42-amino acid peptide secreted from K-cells in the small intestine (duodenum and jejunum). Human GIP is derived from the processing of proGIP, a 153-amino acid precursor that is encoded by a gene localized to chromosome 17q. GIP secretion is induced by food ingestion GIP is an inhibiting hormone of the secretin family of hormones that, among other functions, stimulates insulin secretion. GIP has a number of physiological effects in tissues, including promotion of fat storage in adipocytes and promotion of pancreatic islet β-cell function and glucose-dependent insulin secretion.

GLP-1 is a 31-amino acid peptide derived from the proglucagon gene. It is secreted by intestinal L-cells and released in response to food ingestion to induce insulin secretion from pancreatic β-cells. In addition to the incretin effects, GLP-1 also decreases glucagon secretion, delays gastric emptying and reduces caloric intake. GLP-1 exerts its effects by activation of the GLP-1 receptor (GLP-1R). The function of GLP-1 is limited by rapid degradation by the DPP-IV enzyme, resulting in a half-life of approximately 2 minutes. Treatment of obesity or other diseases characterized by fatty tissue buildup would benefit from an extension of the GLP-1 half-life. Long-lasting GLP-1R agonists, such as exenatide, liraglutide, and dulaglutide, have been developed.

Although the signaling cascades involved in many metabolic disorders have been elucidated, effective treatments for these disorders remains elusive. There is an urgent need for new and innovative approaches to the treatment of metabolic disorders, especially given the health risk posed by not only the disorders themselves, but also by the common co-morbidities that increase the risk of overall negative outcomes. The present disclosure addresses these needs and others.

### BRIEF SUMMARY

In some embodiments, a method of treating a disease is provided, the method comprising administering an anti-glucose-dependent insulinotropic polypeptide (GIP) antibody, or an antigen-binding fragment thereof, and a biologically active peptide, wherein the biologically active peptide comprises a glucagon like peptide 1 receptor (GLP-1R) agonist.

In some embodiments, the method comprises co-administering the anti-GIP antibody, or antigen-binding fragment thereof, and the biologically active peptide.

In some embodiments, the anti-GIP antibody, or antigen-binding fragment thereof, and the biologically active peptide are conjugated. In some embodiments, the biologically active peptide is conjugated to the anti-GIP antibody, or antigen binding fragment thereof, through a substituted cysteine residue in the IgG Fc region. In some embodiments, the IgG Fc is an IgG1 Fc. In some embodiments, the IgG1 Fc comprises one or more substitutions selected from D70C, E89C, V109C, A111C, A118C, S124C, A140C, E152C, S239C, V265C, E272C, K290C, Y300C, E345C, T359C, N390C, or S442C. In some embodiments, the CL comprises one or more substitutions selected from K149C or V205C.

In some embodiments, the GLP-1R agonist is liraglutide, albiglutide, taspoglutide, semaglutide, LY2428757, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91, SEQ ID NO: 92, SEQ ID NO: 93, SEQ ID NO: 94, SEQ ID NO: 95, SEQ ID NO: 96, SEQ ID NO: 97, SEQ ID NO: 98, SEQ ID NO: 99, SEQ ID NO: 100, SEQ ID NO: 101, SEQ ID NO: 102, SEQ ID NO: 103, SEQ ID NO: 104, SEQ ID NO: 105, SEQ ID NO: 106, SEQ ID NO: 107, SEQ ID NO: 108, SEQ ID NO: 109, SEQ ID NO: 110, SEQ ID NO: 111, SEQ ID NO: 112, SEQ ID NO: 113, SEQ ID NO: 114, SEQ ID NO: 115, SEQ ID NO: 116, SEQ ID NO: 117, SEQ ID NO: 118, SEQ ID NO: 119, SEQ ID NO: 120, SEQ ID NO: 121, SEQ ID NO: 145, or SEQ ID NO: 146, or a peptide that is 95% identical thereto.

In some embodiments, the anti-GIP antibody, or an antigen-binding fragment thereof, comprises a variable heavy chain region (VH) and a variable light chain region (VL), wherein the VH comprises a heavy chain complementarity domain 1 (HCDR1), a HCDR2, and a HCDR3 comprising amino acid sequences selected from: (a) SEQ ID NO: 15, SEQ ID NO: 26, and SEQ ID NO: 17, (b) SEQ ID NO: 1, SEQ ID NO: 2, and SEQ ID NO: 3, or (c) SEQ ID NO: 15, SEQ ID NO: 16, and SEQ ID NO: 17; and wherein the VL comprises a light chain complementarity domain 1 (LCDR1), a LCDR2, and a LCDR3 comprising amino acid sequences selected from: (d) SEQ ID NO: 27, SEQ ID NO: 28, and SEQ ID NO: 19, (c) SEQ ID NO: 4, SEQ ID NO: 5, and SEQ ID NO: 6, (f) SEQ ID NO: 18, SEQ ID NO: 5, and SEQ ID NO: 19, (g) SEQ ID NO: 27, SEQ ID NO: 31, and SEQ ID NO: 19, or (h) SEQ ID NO: 27, SEQ ID NO: 34, and SEQ ID NO: 19.

In some embodiments, the VH comprises an amino acid sequence having at least 90% identity to an amino acid sequence selected from SEQ ID NO: 29, SEQ ID NO: 13, SEQ ID NO: 24, SEQ ID NO: 32, or SEQ ID NO: 35. In some embodiments, the VL comprises an amino acid sequence having at least 90% identity to an amino acid sequence selected from SEQ ID NO: 30, SEQ ID NO: 14, SEQ ID NO: 25, SEQ ID NO: 33, or SEQ ID NO: 36.

In some embodiments, a method of treating a disease is provided, the method comprising administering an anti-glucose-dependent insulinotropic polypeptide (GIP) antibody, or an antigen-binding fragment thereof, and semaglutide. In some embodiments, the anti-GIP antibody and semaglutide are co-administered. In some embodiments, the anti-GIP antibody and semaglutide are co-formulated. In some embodiments, the semaglutide is conjugated to the anti-GIP antibody.

In some embodiments, a pharmaceutical composition is provided, the pharmaceutical composition comprising an anti-glucose-dependent insulinotropic polypeptide (GIP) antibody, or an antigen-binding fragment thereof, as provided for herein, and a biologically active peptide as provided for herein, wherein the biologically active peptide comprises a glucagon like peptide 1 receptor (GLP-1R) agonist.

In some embodiments, an apparatus for the delivery of a medicament is provided, the apparatus comprising a first chamber and a second chamber, wherein the first chamber comprises a pharmaceutical composition comprising a biologically active peptide and wherein the second chamber comprises a pharmaceutical composition comprising an anti-GIP antibody, or antigen-binding fragment thereof, wherein the biologically active peptide comprises a glucagon like peptide 1 receptor (GLP-1R) agonist.

In some embodiments, an apparatus for the delivery of a medicament is provided, the apparatus comprising a first chamber, wherein the first chamber comprises a pharmaceutical composition comprising an anti-GIP antibody, or antigen-binding fragment thereof, conjugated to a biologically active peptide, wherein the biologically active peptide comprises a glucagon like peptide 1 receptor (GLP-1R) agonist.

In some embodiments, a conjugate is provided, the conjugate comprising an anti-glucose-dependent insulinotropic polypeptide (GIP) antibody, or an antigen-binding fragment thereof, conjugated to a biologically active peptide, wherein the biologically active peptide comprises a glucagon like peptide 1 receptor (GLP-1R) agonist.

In some embodiments, a method of treating a disease is provided, the method comprising administering a conjugate as provided for herein.

In some embodiments, a pharmaceutical composition is provided, the composition comprising a conjugate as provided for herein.

### BRIEF DESCRIPTION OF FIGURES

FIG. 1 illustrates the results of a binding affinity assay between an anti-GIP antibody as provided for herein and rat GIP.

FIG. 2 illustrates the results of a binding affinity assay between an anti-GIP antibody as provided for herein and human GIP.

FIG. 3 illustrates the results of a binding affinity assay between an anti-GIP antibody as provided for herein and mouse GIP.

FIG. 4 illustrates the results of a binding affinity assay between an anti-GIP antibody as provided for herein and monkey GIP.

FIG. 5 illustrates the results of a GIP neutralization assay with antibody conjugates as provided for herein. Conjugation of the biologically active peptide to E272C of the antibody does not affect the ability of the antibody to bind GIP.

FIGS. 6A and 6B illustrate the results of a GLP-1R activation assay with antibody conjugates as provided for herein. The biologically active peptide was conjugated at the recited HC or LC cysteine mutation sites. The order of the figure legend reflects the order of maximum luminescence at the highest conjugate concentration. FIG. 6A illustrates the results for LC-K149C-BrAc, HC-E272C-BrAc, HC-K290C-BrAc, LC-K149C-Mal, HC-S442C-BrAc, and HCR-188 (antibody alone control). FIG. 6B illustrates the results for LC-V205C-Mal, HC-S442C-Mal, HC-272C-Mal, Semaglutide, GLP-1, and HC-K290C-Mal.

FIG. 7 illustrates the results of a diet induced obesity (DIO) study in mice treated with various embodiments as provided for herein.

FIG. 8A-8D illustrate the results of a diet induced obesity (DIO) study in mice treated with various embodiments as provided for herein. FIG. 8A illustrates the results for 1 mg/kg treatment.

FIG. 8B illustrates the results for 2 mg/kg treatment. FIG. 8C illustrates the results for 5 mg/kg treatment. FIG. 8D illustrates the results for 10 mg/kg treatment.

FIGS. 9A and 9B illustrate the results of a DIO study in NHPs treated with various embodiments as provided for herein. FIG. 9A illustrates the mean % change in body weight (BW) for the various treatment groups. FIG. 9B illustrates the data of FIG. 9A adjusted for vehicle.

FIG. 10 illustrates the cumulative total energy intake (TEI) of each treatment group in a DIO study in NHPs treated with various embodiments as provided for herein.

FIG. 11A and FIG. 11B illustrate the results of a single-dose pharmacokinetics study (SDPK) in DIO NHPs. FIG. 11A illustrates the total change in body weight in the NHPs. FIG. 11B illustrates the TEI of each treatment group.

### DETAILED DESCRIPTION

Provided for herein are antibodies that bind glucose-dependent insulinotropic polypeptide (GIP) and methods of using the same. In some embodiments, the anti-GIP antibody is conjugated to a biologically active peptide. In some embodiments, the anti-GIP antibody is administered with a biologically active peptide. In some embodiments, the anti-GIP antibody is co-administered with a biologically active peptide.

Unless defined otherwise, all technical and scientific terms have the same meaning as is commonly understood by one of ordinary skill in the art to which the embodiments disclosed belongs.

As used herein, the terms “a” or “an” means that “at least one” or “one or more” unless the context clearly indicates otherwise.

As used herein, the term “about” means that the numerical value is approximate and small variations would not significantly affect the practice of the disclosed embodiments. Where a numerical limitation is used, unless indicated otherwise by the context, “about” means the numerical value can vary by ±10% and remain within the scope of the disclosed embodiments. Additionally, where a phrase recites “about x to y,” the term “about” modifies both x and y and can be used interchangeably with the phrase “about x to about y” unless context dictates differently.

As used herein, the term “individual” or “subject,” or “patient” used interchangeably, means any animal, including mammals, such as mice, rats, other rodents, rabbits, dogs, cats, swine, cattle, sheep, horses, or primates, such as humans.

As used herein, the terms “comprising” (and any form of comprising, such as “comprise”, “comprises”, and “comprised”), “having” (and any form of having, such as “have” and “has”), “including” (and any form of including, such as “includes” and “include”), or “containing” (and any form of containing, such as “contains” and “contain”), are inclusive or open-ended and do not exclude additional, unrecited elements or method steps. Any step or composition that uses the transitional phrase of “comprise” or “comprising” can also be said to describe the same with the transitional phase of “consisting of” or “consists.”

As used herein, the term “contacting” means bringing together of two elements in an in vitro system or an in vivo system. For example, “contacting” virus or vector described herein with an individual or patient or cell includes the administration of the virus to an individual or patient, such as a human, as well as, for example, introducing a compound into a sample containing a cellular or purified preparation containing the cell.

As used herein, the term “fused” or “linked” when used in reference to a protein having different domains or heterologous sequences or when used in reference to the joining of two polypeptides means that the protein domains are part of the same peptide chain that are connected to one another with either peptide bonds or other covalent bonding. The domains or section can be linked or fused directly to one another or another domain or peptide sequence can be between the two domains or sequences and such sequences would still be considered to be fused or linked to one another. In some embodiments, the various domains or proteins provided for herein are linked or fused directly to one another or a linker sequences, such as the glycine/serine sequences described herein link the two domains together.

A “disease” is a state of health of an animal wherein the animal cannot maintain homeostasis, and wherein if the disease is not ameliorated then the animal's health continues to deteriorate. In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

“Effective amount” or “therapeutically effective amount” are used interchangeably herein, and refer to an amount of a compound, formulation, material, or composition, as described herein effective to achieve a particular biological result or provides a therapeutic or prophylactic benefit. Such results may include, but are not limited to an amount that when administered to a mammal, has the ability to decrease blood glucose, the ability to increase glucose tolerance, the ability to increase insulin sensitivity the ability to decrease body weight or reduce body weight gain; the ability to decrease fat mass or decrease inflammation in fat tissue; the ability to decrease fasting insulin levels; the ability to decrease circulating cholesterol levels; the ability to decrease circulating triglyceride levels; the ability to decrease liver steatosis or reduce triglyceride level in liver; the ability to decrease AST, ALT, and/or ALP levels. The skilled artisan would understand that the amount of the composition administered herein varies and can be readily determined based on a number of factors such as the disease or condition being treated, the age and health and physical condition of the mammal being treated, the severity of the disease, the particular compound being administered, and the like.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

“Expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e.g., naked or contained in liposomes) and viruses (e.g., Sendai viruses, lentiviruses, retroviruses, adenoviruses, and adeno-associated viruses) that incorporate the recombinant polynucleotide.

As used herein, the phrase “ex vivo” in reference to a cell being transduced, transfected or transformed ex vivo, refers to a cell being transduced, transfected or transformed outside of the subject, that is with the cells being removed from the subject before such cells are transduced, transfected or transformed.

“Identity” as used herein refers to the subunit sequence identity between two polymeric molecules such as between two nucleic acid or amino acid molecules, such as, between two polynucleotide or polypeptide molecules. When two amino acid sequences have the same residues at the same positions; e.g., if a position in each of two polypeptide molecules is occupied by an Arginine, then they are identical at that position. The identity or extent to which two amino acid or two nucleic acid sequences have the same residues at the same positions in an alignment is often expressed as a percentage. The identity between two amino acid or two nucleic acid sequences is a direct function of the number of matching or identical positions; e.g., if half of the positions in two sequences are identical, the two sequences are 50% identical; if 90% of the positions (e.g., 9 of 10), are matched or identical, the two amino acids sequences are 90% identical.

By “substantially identical” is meant a polypeptide or nucleic acid molecule exhibiting at least 50% identity to a reference amino acid sequence (for example, any one of the amino acid sequences described herein) or nucleic acid sequence (for example, any one of the nucleic acid sequences described herein). In some embodiments, such a sequence is at least 60%, 80% or 85%, or 90%, 95% or even 99% identical at the amino acid level or nucleic acid to the sequence used for comparison. Other percentages of identity in reference to specific sequences are described herein.

Sequence identity can be measured/determined using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine. In an exemplary approach to determining the degree of identity, a BLAST program may be used, with a probability score between e3 and e100 indicating a closely related sequence. In some embodiments, sequence identity is determined by using BLAST with the default settings.

To the extent embodiments provided for herein, includes composition comprising various proteins, these proteins may, in some instances, comprise amino acid sequences that have sequence identity to the amino acid sequences disclosed herein. Therefore, in certain embodiments, depending on the particular sequence, the degree of sequence identity is preferably greater than 50% (e.g. 60%, 70%, 75%, 80%, 85%, 90%, 91%, 92%, 93%, 94%, 95%, 96%, 97%, 98%, 99% or more) to the SEQ ID NOs disclosed herein. In addition to these percentages, other percentages of identity are provided for herein. Identity between polypeptides can be determined by the Smith-Waterman homology search algorithm as implemented in the MPSRCH program (Oxford Molecular), using an affine gap search with parameters gap open penalty−12 and gap extension penalty=1.

These proteins may, compared to the disclosed proteins, include one or more (e.g. 1, 2, 3, 4, 5, 6, 7, 8, 9, 10, etc.) conservative amino acid replacements i.e. replacements of one amino acid with another which has a related side chain. Genetically-encoded amino acids are generally divided into four families: (1) acidic i.e. aspartate, glutamate; (2) basic i.e. lysine, arginine, histidine; (3) non polar i.e. alanine, valine, leucine, isoleucine, proline, phenylalanine, methionine, tryptophan; and (4) uncharged polar i.e. glycine, asparagine, glutamine, cysteine, serine, threonine, tyrosine. Phenylalanine, tryptophan, and tyrosine are sometimes classified jointly as aromatic amino acids. In general, Substitution of single amino acids within these families does not have a major effect on the biological activity. The proteins may have one or more (e.g. 1, 2, 3, 4, 5, 6, 7, 8, 9, 10, etc.) single amino acid deletions relative to the disclosed protein sequences. The proteins may also include one or more (e.g. 1, 2, 3, 4, 5, 6, 7, 8, 9, 10, etc.) insertions (e.g. each of 1, 2, 3, 4 or 5 amino acids) relative to the disclosed protein sequences.

Synthetic, rare, or modified amino acid residues having known similar physiochemical properties to those of an above-described grouping can be used as a “conservative” substitute for a particular amino acid residue in a sequence. For example, a D-Arg residue may serve as a substitute for a typical L-Arg residue. It also can be the case that a particular substitution can be described in terms of two or more of the above described classes (e.g., a substitution with a small and hydrophobic residue means substituting one amino acid with a residue(s) that is found in both of the above-described classes or other synthetic, rare, or modified residues that are known in the art to have similar physiochemical properties to such residues meeting both definitions).

As used herein, the phrase “in vivo” in reference to a cell being transduced, transfected or transformed in vivo, refers to a cell being transduced, transfected or transformed in the subject without the cells being removed from the subject before such cells are transduced, transfected or transformed.

“Isolated” means altered or removed from the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell.

By the term “modified” as used herein, is meant a changed state or structure of a molecule or cell as provided herein. Molecules may be modified in many ways, including chemically, structurally, and functionally, such as mutations, substitutions, insertions, or deletions (e.g. internal deletions truncations). Cells may be modified through the introduction of nucleic acids or the expression of heterologous proteins.

By the term “modulating,” as used herein, is meant mediating an increase or decrease in the level of a response in a subject compared with the level of a response in the subject in the absence of a treatment or compound, and/or compared with the level of a response in an otherwise identical but untreated subject. The term encompasses perturbing and/or affecting a native signal or response thereby mediating a beneficial therapeutic response in a subject, such as, a human.

Unless otherwise specified, a “nucleotide sequence encoding an amino acid sequence” includes all nucleotide sequences that are degenerate versions of each other and that encode the same amino acid sequence. The phrase nucleotide sequence that encodes a protein or an RNA may also include introns to the extent that the nucleotide sequence encoding the protein may in some version contain an intron(s).

The term “oligonucleotide” typically refers to short polynucleotides. It will be understood that when a nucleotide sequence is represented by a DNA sequence (i.e., A, T, C, G), this also provides the corresponding RNA sequence (i.e., A, U, C, G) in which “U” replaces “T.”

“Parenteral” administration of a composition includes, e.g., subcutaneous (s.c.), intravenous (i.v.), intramuscular (i.m.), or intrasternal injection, or infusion techniques.

The term “polynucleotide” as used herein is defined as a chain of nucleotides. Furthermore, nucleic acids are polymers of nucleotides. Thus, the terms “nucleic acids” and “polynucleotides” as used herein are interchangeable. As used herein polynucleotides include, but are not limited to, all nucleic acid sequences which are obtained by any methods available in the art, including, without limitation, recombinant methods, i.e., the cloning of nucleic acid sequences from a recombinant library or a cell genome, using cloning technology and PCR, and the like, and by synthetic means.

As used herein, the terms “peptide,” “polypeptide,” and “protein” are used interchangeably, and refer to a compound comprised of a plurality of amino acid residues covalently linked by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof.

As used herein, the term “antibody” refers to a broad sense and includes immunoglobulin or antibody molecules including polyclonal antibodies, monoclonal antibodies including murine, human, humanized and chimeric monoclonal antibodies and antibody fragments, such as, but not limited to, single chain variable fragments (scFv).

A “non-naturally occurring amino acid” is a compound that has the same basic chemical structure as a naturally occurring amino acid, but is not incorporated into a growing polypeptide chain by the translation complex. “Non-naturally occurring amino acid” also includes, but is not limited to, amino acids that occur by modification (e.g., posttranslational modifications) of a naturally encoded amino acid (including but not limited to, the 20 common amino acids) but are not themselves naturally incorporated into a growing polypeptide chain by the translation complex. A non-limiting lists of examples of non-naturally occurring amino acids that can be inserted into a polypeptide sequence or substituted for a wild-type residue in polypeptide sequence include β-amino acids, homoamino acids, cyclic amino acids and amino acids with derivatized side chains. Examples include (in the L-form or D-form; abbreviated as in parentheses): citrulline (Cit), homocitrulline (hCit), Nα-methylcitrulline (NMeCit), Nα-methylhomocitrulline (Nα-MeHoCit), ornithine (Orn), Nα-Methylornithine (Nα-MeOrn or NMeOrn), sarcosine (Sar), homolysine (hLys or hK), homoarginine (hArg or hR), homoglutamine (hQ), Nα-methylarginine (NMeR), Nα-methylleucine (Nα-MeL or NMeL), N-methylhomolysine (NMeHoK), Nα-methylglutamine (NMeQ), norleucine (Nle), norvaline (Nva), 1,2,3,4-tetrahydroisoquinoline (Tic), Octahydroindole-2-carboxylic acid (Oic), 3-(1-naphthyl)alanine (1-Nal), 3-(2-naphthyl)alanine (2-Nal), 1,2,3,4-tetrahydroisoquinoline (Tic), 2-indanylglycine (IgI), para-iodophenylalanine (pI-Phe), para-aminophenylalanine (4AmP or 4-Amino-Phe), 4-guanidino phenylalanine (Guf), glycyllysine (abbreviated “K(Nε-glycyl)” or “K(glycyl)” or “K(gly)”), nitrophenylalanine (nitrophe), aminophenylalanine (aminophe or Amino-Phe), benzylphenylalanine (benzylphe), γ-carboxyglutamic acid (γ-carboxyglu), hydroxyproline (hydroxypro), p-carboxyl-phenylalanine (Cpa), α-aminoadipic acid (Aad), Nα-methyl valine (NMeVal), N-α-methyl leucine (NMeLeu), Nα-methylnorleucine (NMeNle), cyclopentylglycine (Cpg), cyclohexylglycine (Chg), acetylarginine (acetylarg), α,β-diaminopropionoic acid (Dpr), α,γ-diaminobutyric acid (Dab), diaminopropionic acid (Dap), cyclohexylalanine (Cha), 4-methyl-phenylalanine (MePhe), β,β-diphenyl-alanine (BiPhA), aminobutyric acid (Abu), 4-phenyl-phenylalanine (or biphenylalanine; 4Bip), α-amino-isobutyric acid (Aib), beta-alanine, beta-aminopropionic acid, piperidinic acid, aminocaprioic acid, aminoheptanoic acid, aminopimelic acid, desmosine, diaminopimelic acid, N-ethylglycine, N-ethylaspargine, hydroxylysine, allo-hydroxylysine, isodesmosine, allo-isoleucine, N-methylglycine, N-methylisoleucine, N-methylvaline, 4-hydroxyproline (Hyp), γ-carboxyglutamate, ε-N,N,N-trimethyllysine, ε-N-acetyllysine, O-phosphoserine, N-acetylserine, N-formylmethionine, 3-methylhistidine, 5-hydroxylysine, ω-methylarginine, 4-Amino-O-Phthalic Acid (4APA), and other similar amino acids, and derivatized forms of any of those specifically listed.

In general, antibodies are proteins or polypeptides that exhibit binding specificity to a specific antigen. Intact antibodies are heterotetrameric glycoproteins, composed of two identical light chains and two identical heavy chains. Typically, each light chain is linked to a heavy chain by one covalent disulfide bond, while the number of disulfide linkages varies between the heavy chains of different immunoglobulin isotypes. Each heavy and light chain also has regularly spaced intrachain disulfide bridges. Each heavy chain has at one end a variable domain (VH) followed by a number of constant domains. Each light chain has a variable domain at one end (VL) and a constant domain at its other end; the constant domain of the light chain is aligned with the first constant domain of the heavy chain and the light chain variable domain is aligned with the variable domain of the heavy chain. Antibody light chains of any vertebrate species can be assigned to one of two clearly distinct types, namely kappa and lambda, based on the amino acid sequences of their constant domains. Immunoglobulins can be assigned to five major classes, namely IgA, IgD, IgE, IgG and IgM, depending on the heavy chain constant domain amino acid sequence. IgA and IgG are further sub-classified as the isotypes IgA1, IgA2, IgG1, IgG2, IgG3 and IgG4.

As used herein, the term “antibody fragment” refers to an intact antibody, generally the antigen-binding or variable region of the intact antibody. Examples of antibody fragments include Fab, Fab′, F(ab′)2 and Fv fragments, diabodies, single chain antibody molecules and multispecific antibodies formed from at least two intact antibodies or fragments thereof.

As used herein, the term “antigen” refers to any molecule that has the ability to generate antibodies either directly or indirectly.

By the term “specifically binds,” as used herein with respect to an antibody, is meant an antibody which recognizes a specific antigen, but does not substantially recognize or bind other molecules in a sample. For example, an antibody that specifically binds to an antigen from one species may also bind to that antigen from one or more species. But, such cross-species reactivity does not itself alter the classification of an antibody as specific. In another example, an antibody that specifically binds to an antigen may also bind to different allelic forms of the antigen. However, such cross reactivity does not itself alter the classification of an antibody as specific. In some instances, the terms “specific binding” or “specifically binding,” can be used in reference to the interaction of an antibody, a protein, or a peptide with a second chemical species, to mean that the interaction is dependent upon the presence of a particular structure (e.g., an antigenic determinant or epitope) on the chemical species; for example, an antibody recognizes and binds to a specific protein structure rather than to proteins generally. If an antibody is specific for epitope “A”, the presence of a molecule containing epitope A (or free, unlabeled A), in a reaction containing labeled “A” and the antibody, will reduce the amount of labeled A bound to the antibody.

The term “epitope” is the portion of a molecule that is bound by an antigen-binding protein (for example, an antibody). The term includes any determinant capable of specifically binding to an antigen-binding protein, such as an antibody. An epitope can be contiguous or non-contiguous (discontinuous) (e.g., in a polypeptide, amino acid residues that are not contiguous to one another in the polypeptide sequence but that within in context of the molecule are bound by the antigen-binding protein). A conformational epitope is an epitope that exists within the conformation of an active protein but is not present in a denatured protein. In certain embodiments, epitopes may be mimetic in that they comprise a three dimensional structure that is similar to an epitope used to generate the antigen-binding protein, yet comprise none or only some of the amino acid residues found in that epitope used to generate the antigen-binding protein. Most often, epitopes reside on proteins, but in some instances may reside on other kinds of molecules, such as nucleic acids. Epitope determinants may include chemically active surface groupings of molecules such as amino acids, sugar side chains, phosphoryl or sulfonyl groups, and may have specific three dimensional structural characteristics, and/or specific charge characteristics. Generally, antigen-binding proteins specific for a particular target antigen will preferentially recognize an epitope on the target antigen in a complex mixture of proteins and/or macromolecules.

The term “subject” includes living organisms, including those in which an immune response can be elicited (e.g., mammals). A “subject” or “patient,” as used therein, may be a human or non-human mammal. Non-human mammals include, for example, livestock and pets, such as ovine, bovine, porcine, canine, non-human primates, feline and murine mammals. In some embodiments, the subject is human.

The term “therapeutic” as used herein means a treatment and/or prophylaxis. A therapeutic effect is obtained by suppression, remission, or eradication of a disease state.

The term “transfected” or “transformed” or “transduced” as used herein refers to a process by which exogenous nucleic acid is transferred or introduced into a cell. A “transfected” or “transformed” or “transduced” cell is one which has been transfected, transformed or transduced with exogenous nucleic acid. The cell includes the primary subject cell and its progeny. In some embodiments, the transfection, transformation, or transduction is performed or occurs in vivo.

To “treat” a disease as the term is used herein, means to reduce the frequency or severity of at least one sign or symptom of a disease or disorder experienced by a subject.

A “vector” is a composition of matter which comprises an isolated nucleic acid encoding a protein or a peptide. Numerous vectors are known in the art including, but not limited to, linear polynucleotides, plasmids, DNA, and RNA. Examples of viral vectors include, but are not limited to, Sendai viral vectors, adenoviral vectors, adeno-associated virus vectors, retroviral vectors, lentiviral vectors, and the like.

A “carrier” or “delivery vehicle” includes viral particles, viruses, polylysine compounds, and liposomes, which facilitate transfer of nucleic acid into cells. A carrier or delivery vehicle can also be used to deliver a protein or peptide to a cell.

Ranges: throughout this disclosure, various aspects of the embodiments can be presented in a range format. It should be understood that the description in range format is merely for convenience and brevity and should not be construed as an inflexible limitation. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range. Unless otherwise explicitly stated to the contrary, a range that is disclosed also includes the endpoints of the range.

### Antibodies

Disclosed herein are antibodies, or antigen-binding fragments thereof, or equivalents thereof, and methods of use thereof. In some embodiments, the antibody, or antigen-binding fragment thereof is a monoclonal antibody, or antigen-binding fragment thereof. In some embodiments, the antibody, or antigen-binding fragment thereof specifically binds to glucose-dependent insulinotropic polypeptide (GIP). In some embodiments, the anti-GIP antibody, or antigen-binding fragment thereof, selectively binds GIP.

Human GIP (UniProt: P09681) has the amino acid sequence of SEQ ID NO: 122, shown below.

(SEQ ID NO: 122)
MVATKTFALLLLSLFLAVGLGEKKEGHFSALPSLPVGSHAKV
SSPQPRGPRYAEGTFISDYSIAMDKIHQQDFVNWLLAQKGKK
NDWKHNITQREARALELASQANRKEEEAVEPQSSPAKNPSDE
DLLRDLLIQELLACLLDQTNLCRLRSR

Full length GIP is processed prior to secretion resulting in a 42 amino acid circulating polypeptide. The processed form of human GIP has the amino acid sequence of SEQ ID NO: 123, shown below.

(SEQ ID NO: 123)
YAEGTFISDYSIAMDKIHQQDFVNWLLAQKGKKNDWKHNITQ

The gene sequence of GIP is highly conserved across species. The amino acid sequences of the processed forms of mouse (SEQ ID NO: 124), rat (SEQ ID NO: 125), and rhesus macaque (SEQ ID NO: 126) are provided below.

(SEQ ID NO: 124)
YAEGTFISDYSIAMDKIRQQDFVNWLLAQRGKKSDWKHNITQ
(SEQ ID NO: 125)
YAEGTFISDYSIAMDKIRQQDFVNWLLAQKGKKNDWKHNLTQ
(SEQ ID NO: 126)
YAEGTFISDYSIAMDKIHQQDFVNWLLAQKGKKNDWKHNITQ

In some embodiments, the anti-GIP antibody, or antigen-binding fragment thereof, binds to an amino acid sequence of SEQ ID NO: 122. In some embodiments, the anti-GIP antibody, or antigen-binding fragment thereof, binds to secreted GIP and binds to an amino acid sequence of SEQ ID NO: 123, SEQ ID NO: 124, SEQ ID NO: 125, or SEQ ID NO: 126. In some embodiments, the anti-GIP antibody, or antigen-binding fragment thereof, binds to an amino acid sequence of SEQ ID NO: 123. In some embodiments, the anti-GIP antibody, or antigen-binding fragment thereof, binds to an amino acid sequence of SEQ ID NO: 124. In some embodiments, the anti-GIP antibody, or antigen-binding fragment thereof, binds to an amino acid sequence of SEQ ID NO: 125. In some embodiments, the anti-GIP antibody, or antigen-binding fragment thereof, binds to an amino acid sequence of SEQ ID NO: 126.

In some embodiments, the anti-GIP antibody, or antigen-binding fragment thereof, does not substantially bind other native incretins or incretin analogues.

In some embodiments, the anti-GIP antibody, or antigen-binding fragment thereof, is monoclonal. In some embodiments, the monoclonal anti-GIP antibody, or antigen-binding fragment thereof, is a mouse, chimeric, humanized, or human monoclonal antibody or antigen-binding fragment thereof. In some embodiments, the monoclonal anti-GIP antibody, or antigen-binding fragment thereof, is a human monoclonal antibody or antigen-binding fragment thereof. In some embodiments, the monoclonal anti-GIP antibody, or antigen-binding fragment thereof, is a humanized monoclonal antibody or antigen-binding fragment thereof. In some embodiments, the monoclonal anti-GIP antibody, or antigen-binding fragment thereof, is a chimeric monoclonal antibody or antigen-binding fragment thereof. In some embodiments, the monoclonal anti-GIP antibody, or antigen-binding fragment thereof, is a mouse monoclonal antibody or antigen-binding fragment thereof.

In some embodiments, the anti-GIP antibody, or antigen-binding fragment thereof, comprises a CL, CH1, CH2, or CH3 region, or a combination thereof. In some embodiments, the anti-GIP antibody, or antigen-binding fragment thereof, comprises an Fc region. The Fc region can be linked to the heavy or light chain of the antibody. In some embodiments, the Fc region is an IgG Fc. In some embodiments, the IgG is selected from IgG1, IgG2, IgG3, or IgG4. In some embodiments, the IgG Fc is an IgG1 Fc. In some embodiments, the IgG1 Fc comprises the amino acid sequence of SEQ ID NO: 127 below.

(SEQ ID NO: 127)
ASTKGPSVFPLAPSSKSTSGGTAALGCLVKDYFPEPVTVSWNSGALTS
GVHTFPAVLQSSGLYSLSSVVTVPSSSLGTQTYICNVNHKPSNTKVDK
KVEPKSCDKTHTCPPCPAPELLGGPSVFLFPPKPKDTLMISRTPEVTC
VVVDVSHEDPEVKFNWYVDGVEVHNAKTKPREEQYNSTYRVVSVLTVL
HQDWLNGKEYKCKVSNKALPAPIEKTISKAKGQPREPQVYTLPPSRDE
LTKNQVSLTCLVKGFYPSDIAVEWESNGQPENNYKTTPPVLDSDGSFF
LYSKLTVDKSRWQQGNVFSCSVMHEALHNHYTQKSLSLSPGK

In some embodiments, the IgG Fc is IgG2 Fc. In some embodiments, the IgG2 Fc comprises the amino acid sequence of SEQ ID NO: 128 below.

(SEQ ID NO: 128)
STKGPSVFPLAPCSRSTSESTAALGCLVKDYFPEPVTVSWNSGALTSG
VHTFPAVLQSSGLYSLSSVVTVPSSSLGTQTYTCNVDHKPSNTKVDKT
VERKCCVECPPCPAPPVAGPSVFLFPPKPKDTLMISRTPEVTCVVVDV
SHEDPEVQFNWYVDGVEVHNAKTKPREEQFNSTFRVVSVLIVVHQDWL
NGKEYKCKVSNKGLPAPIEKTISKTKGQPREPQVYTLPPSREEMTKNQ
VSLTCLVKGFYPSDIAVEWESNGQPENNYKTTPPMLDSDGSFFLYSKL
IVDKSRWQQGNVFSCSVMHEALHNHYTQKSLSLSPGK

In some embodiments, the IgG Fc is IgG4 Fc. In some embodiments, the IgG4 Fc comprises the amino acid sequence of SEQ ID NO: 129 below.

(SEQ ID NO: 129)
STKGPSVFPLAPCSRSTSESTAALGCLVKDYFPEPVTVSWNSGALTSG
VHTFPAVLQSSGLYSLSSVVTVPSSSLGTKTYTCNVDHKPSNTKVDKR
VESKYGPPCPSCPAPEFLGGPSVFLFPPKPKDTLMISRTPEVTCVVVD
VSQEDPEVQFNWYVDGVEVHNAKTKPREEQFNSTYRVVSVLTVLHQDW
LNGKEYKCKVSNKGLPSSIEKTISKAKGQPREPQVYTLPPSQEEMTKN
QVSLTCLVKGFYPSDIAVEWESNGQPENNYKTTPPVLDSDGSFFLYSR
LTVDKSRWQEGNVFSCSVMHEALHNHYTQKSLSLSLGK

In some embodiments, the Fc domain comprises mutations that render the Fc region “effectorless” and unable to bind Fc receptors. The mutations that render Fc regions effectorless are known in the art and any mutation or combination of mutations can be used. In some embodiments, the Fc domain comprises mutations that impair or inhibits recycling of the Fc. In some embodiments, the Fc domain comprises mutations that impair FcRn-mediated recycling of the Fc. In some embodiments, the Fc domain comprises a mutation, or a set of mutations, that impair binding of the Fc to an FcRn. In some embodiments, the Fc domain does not bind to an FcRn. In some embodiments, the Fc domain has reduced affinity to an FcRn. In some embodiments, the Fc domain comprising a mutation, or a set of mutations, that impairs binding of the Fc to an FcRn, exhibit better (i.e., more or enhanced) degradation as compared to an Fc domain not comprising the mutation, or set of mutations, that impairs its binding to the FcRn. In some embodiments, the Fc domain comprising a mutation, or a set of mutations, that impairs binding of the Fc to an FcRn, exhibit better lysosomal degradation as compared to an Fc domain not comprising the mutation, or set of mutations, that impairs its binding to the FcRn. In some embodiments, the Fc domain comprising a mutation, or a set of mutations, that impairs binding of the Fc to an FcRn, exhibit improved lysosomal degradation as compared to an Fc domain not comprising the mutation, or set of mutations, that impairs its binding to the FcRn. Without being bound to any particular theory, a Fc molecule that has improved lysosomal degradation will lead to the antigen being released from the Fc molecule through the lysosomal degradation pathway, and, therefore, improved antigen presentation on the surface of a cell, such as those cells provided herein. Therefore, in some embodiments, methods of providing antigen presentation on the surface of a cell are provided, wherein the antigen is linked or fused to a Fc that comprises a mutation that impairs the Fc's binding to the FcRn. In some embodiments, the Fc domain comprises a mutation, or a set of mutations, selected from H310A, and/or G435A, wherein the mutation, or set of mutations impairs binding of the Fc to the FcRn. In some embodiments, the Fc region is an effectorless Fc region, and the Fc region does not bind, or has reduced affinity, to FcRn.

In some embodiments, the mutations in the Fc region, which is according to the known numbering system, are selected from the group consisting of: L234A, L235A, L234F, L235E, P329G, P329A, P331S, N297A, N297G, N297Q, G236A, A330S, S239D, 1332E, S267E, H268F, S324T, Y296W, T299A, V308P, H310A, R409K, Y435H, T307A, T309A, T309K, K322A, K326W, K334W, K326A, K334A, G237A, P238S, H268A, M252Y, S254T, T256E, M428L, N434S, or any combination thereof. In some embodiments, the Fc comprises a mutation a positions M242 and/or S254 and/or T256. In some embodiments, the Fc comprises M242Y and/or S254T and/or T256E mutations, which can be referred to as “YTE” mutations. In some embodiments, the Fc comprises a mutation at position M428 and/or N434. In some embodiments, the Fc comprises M428L and/or N434S mutations, which can be referred to as “LS” mutations. In some embodiments, the Fc comprises a mutation at L234 and/or L235 and/or G237. In some embodiments, the Fc comprises L234A and/or L235A mutations, which can be referred to as “LALA” mutations. In some embodiments, the Fc comprises L234A, L235A, and G237A mutations, which can be referred to as “LALAGA” or “AAA”. In some embodiments, the Fc comprises L234F, and L235E mutations, which can be referred to as “LAFE” mutations. In some embodiments, the Fc comprises L234A, L235A, and P329G mutations, which can be referred to as “LALAPG” mutations. In some embodiments, the Fc comprises L234A, L235A, P329G, and P331S mutations, which can be referred to as “LALAPGS” mutations. In some embodiments, the Fc comprises L234A, L235A, and P329S mutations, which can be referred to as “LALAPS” mutations. In some embodiments, the Fc comprises L234A, L235A, and P329A mutations, which can be referred to as “LALAPA” mutations. In some embodiments, the Fc comprises a N297A mutation. In some embodiments, the Fc mutations comprises a N297G mutation. In some embodiments, the Fc comprises a N297Q mutation. In some embodiments, the Fc comprises a P329G mutation. In some embodiments, the Fc comprises G236A, A330S, and P331S mutations, which can be referred to as “GASDALIE” mutations. In some embodiments, the Fc comprises S239D and 1332E mutations, which can be referred to as “SIE” mutations. In some embodiments, the Fc comprises S267E, H268F, S324T, and 1332E mutations, which can be referred to as “SEHF_STIE” mutations. In some embodiments, the Fc comprises Y296W, T299A, and V308P mutations, which can be referred to as “YTEV” mutations. In some embodiments, the Fc comprises H310A, R409K, and Y435H mutations, which can be referred to as “HRY” mutations. In some embodiments, the Fc comprises T307A and T309A mutations, which can be referred to as “TATA” mutations. In some embodiments, the Fc comprises T307A and T309K mutations, which can be referred to as “TAKA” mutations. In some embodiments, the Fc comprises a K322A mutation. In some embodiments, the Fc comprises K326W and K334W mutations, which can be referred to as “WKWK” mutations. In some embodiments, the Fc comprises K326A and K334A mutations, which can be referred to as “AA” mutations. In some embodiments, the Fc comprises L234A, L235A, G237A, P238S, H268A, A330S, and P331S mutations.

In some embodiments, the Fc comprises a H310A mutation. In some embodiments, the Fc comprises a H435A mutation. In some embodiments, the Fc comprises H310A and H435A mutations. In some embodiments, the Fc mutations comprise L234A, L235A, and H310A. In some embodiments, the Fc mutations comprise L234F, L235E, and H310A. In some embodiments, the Fc mutations comprise L234A, L235A, P329G, and H310A. In some embodiments, the Fc mutations comprise L234A, L235A, P329G, P331S, and H310A. In some embodiments, the Fc mutations comprise L234A, L235A, P329S, and H310A. In some embodiments, the Fc mutations comprise N297A, and H310A. In some embodiments, the Fc mutations comprise N297G, and H310A. In some embodiments, the Fc mutations comprise N297Q, and H310A. In some embodiments, the Fc mutations comprise P329G, and H310A. In some embodiments, the Fc mutations comprise G236A, A330S, P331S, and H310A. In some embodiments, the Fc mutations comprise S239D, 1332E, and H310A. In some embodiments, the Fc mutations comprise S267E, H268F, S324T, 1332E, and H310A. In some embodiments, the Fc mutations comprise Y296W, T299A, V308P, and H310A. In some embodiments, the Fc mutations comprise H310A, R409K, Y435H, and H310A. In some embodiments, the Fc mutations comprise T307A, T309A, and H310A. In some embodiments, the Fc mutations comprise T307A, T309K, and H310A. In some embodiments, the Fc mutations comprise K322A, and H310A. In some embodiments, the Fc mutations comprise K326W, K334W, and H310A. In some embodiments, the Fc mutations comprise K326A, K334A, and H310A. In some embodiments, the Fc mutations comprise L234A, L235A, and H435A. In some embodiments, the Fc mutations comprise L234F, L235E, and H435A. In some embodiments, the Fc mutations comprise L234A, L235A, P329G, and H435A. In some embodiments, the Fc mutations comprise L234A, L235A, P329G, P331S, and H435A. In some embodiments, the Fc mutations comprise L234A, L235A, P329S, and H435A. In some embodiments, the Fc mutations comprise N297A, and H435A. In some embodiments, the Fc mutations comprise N297G, and H435A. In some embodiments, the Fc mutations comprise N297Q, and H435A. In some embodiments, the Fc mutations comprise P329G, and H435A. In some embodiments, the Fc mutations comprise G236A, A330S, P331S, and H435A. In some embodiments, the Fc mutations comprise S239D, 1332E, and H435A. In some embodiments, the Fc mutations comprise S267E, H268F, S324T, 1332E, and H435A. In some embodiments, the Fc mutations comprise Y296W, T299A, V308P, and H435A. In some embodiments, the Fc mutations comprise H435A, R409K, Y435H, and H435A. In some embodiments, the Fc mutations comprise T307A, T309A, and H435A. In some embodiments, the Fc mutations comprise T307A, T309K, and H435A. In some embodiments, the Fc mutations comprise K322A, and H435A. In some embodiments, the Fc mutations comprise K326W, K334W, and H435A. In some embodiments, the Fc mutations comprise K326A, K334A, and H435A. In some embodiments, the Fc mutations comprise L234A, L235A, H310, and H435A. In some embodiments, the Fc mutations comprise L234F, L235E, H310, and H435A. In some embodiments, the Fc mutations comprise L234A, L235A, P329G, H310, and H435A. In some embodiments, the Fc mutations comprise L234A, L235A, P329G, P331S, H310, and H435A. In some embodiments, the Fc mutations comprise L234A, L235A, P329S, H310, and H435A. In some embodiments, the Fc mutations comprise N297A, H310, and H435A. In some embodiments, the Fc mutations comprise N297G, H310, and H435A. In some embodiments, the Fc mutations comprise N297Q, H310, and H435A. In some embodiments, the Fc mutations comprise P329G, H310, and H435A. In some embodiments, the Fc mutations comprise G236A, A330S, P331S, H310, and H435A. In some embodiments, the Fc mutations comprise S239D, 1332E, H310, and H435A. In some embodiments, the Fc mutations comprise S267E, H268F, S324T, 1332E, H310, and H435A. In some embodiments, the Fc mutations comprise Y296W, T299A, V308P, H310, and H435A. In some embodiments, the Fc mutations comprise H435A, R409K, Y435H, H310, and H435A. In some embodiments, the Fc mutations comprise T307A, T309A, H310, and H435A. In some embodiments, the Fc mutations comprise T307A, T309K, H310, and H435A. In some embodiments, the Fc mutations comprise K322A, H310, and H435A. In some embodiments, the Fc mutations comprise K326W, K334W, H310, and H435A. In some embodiments, the Fc mutations comprise K326A, K334A, H310, and H435A.

The mutations and positions of the Fc region, which can also be referred to as the Fc domain, are according to Kabat numbering.

In some embodiments, the anti-GIP antibody, or antigen-binding fragment thereof, comprises a variable heavy chain region (VH) and a variable light chain region (VL), wherein the VH comprises a heavy chain complementarity domain 1 (HCDR1), a HCDR2, and a HCDR3 and the VL comprises a light chain complementarity domain 1 (LCDR1) a LCDR2, and a LCDR3 as provided for in Table 1 below, which illustrates the CDR sequences based on Kabat numbering.

TABLE 1
CDRs of Exemplary anti-GIP antibodies-
Kabat numbering
Kabat CDRs

| Ab ID No | HCDR1 | HCDR2 | HCDR3 | LCDR1 | LCDR2 | LCDR3 |
|---|---|---|---|---|---|---|
| 14B9 | DYYFH | WIDPENG | YGNNYFD | SASSSVSS | RTSNLAS | LQWSGFP |
| | (SEQ ID | DTEYAPN | Y | SYLH | (SEQ ID | FT |
| | NO: 1) | FQV | (SEQ ID | (SEQ ID | NO: 5) | (SEQ ID |
| | | (SEQ ID | NO: 3) | NO: 4) | | NO: 6) |
| | | NO: 2) | | | | |
| 10g10 | DYYLH | WIDPENG | YGIYFMD | SASSSISS | SEQ ID | QQGSSFP |
| | (SEQ ID | DTEYAPK | Y | NSLH | NO: 5 | RMLT |
| | NO: 15) | FQD | (SEQ ID | (SEQ ID | | (SEQ ID |
| | | (SEQ ID | NO: 17) | NO: 18) | | NO: 19) |
| | | NO: 16) | | | | |
| GIP AB1 | SEQ ID | WIDPENG | SEQ ID | RASSSISS | RTSNLQS | SEQ ID |
| | NO: 15 | DTEYAPK | NO: 17 | NSLH | (SEQ ID | NO: 19 |
| | | FQG | | (SEQ ID | NO: 28) | |
| | | (SEQ ID | | NO: 27) | | |
| | | NO: 26) | | | | |
| GIP AB2 | SEQ ID | SEQ ID | SEQ ID | SEQ ID | RTSSLQS | SEQ ID |
| | NO: 15 | NO: 26 | NO: 17 | NO: 27 | (SEQ ID | NO: 19 |
| | | | | | NO: 31) | |
| GIP AB3 | SEQ ID | SEQ ID | SEQ ID | SEQ ID | RTSNRAT | SEQ ID |
| | NO: 15 | NO: 26 | NO: 17 | NO: 27 | (SEQ ID | NO: 19 |
| | | | | | NO: 34) | |

In some embodiments, the anti-GIP antibody, or antigen-binding fragment thereof, comprises a HCDR1, HCDR2, HCDR3, LCDR1, LCDR2, and LCDR3 as provided for in Table 2 below, which illustrates the CDR sequences based on Chothia numbering.

TABLE 2
CDRs of Exemplary anti-GIP antibodies-
Chothia numbering
Chothia CDRs

| Ab | | | | | | |
|---|---|---|---|---|---|---|
| ID No | HCDR1 | HCDR2 | HCDR3 | LCDR1 | LCDR2 | LCDR3 |
| 14B9 | GFNIKDY | DPENGD | SEQ ID | SEQ ID | SEQ ID | SEQ ID |
| | (SEQ ID | (SEQ ID | NO: 3 | NO: 4 | NO: 5 | NO: 6 |
| | NO: 7) | NO: 8) | | | | |
| 10g10 | GFNIRDY | SEQ ID | SEQ ID | SEQ ID | SEQ ID | SEQ ID |
| | (SEQ ID | NO: 8 | NO: 17 | NO: 18 | NO: 5 | NO: 19 |
| | NO: 20) | | | | | |
| GIP AB1 | SEQ ID | SEQ ID | SEQ ID | SEQ ID | SEQ ID | SEQ ID |
| | NO: 20 | NO: 8 | NO: 17 | NO: 27 | NO: 28 | NO: 19 |
| GIP AB2 | SEQ ID | SEQ ID | SEQ ID | SEQ ID | SEQ ID | SEQ ID |
| | NO: 20 | NO: 8 | NO: 17 | NO: 27 | NO: 31 | NO: 19 |
| GIP AB3 | SEQ ID | SEQ ID | SEQ ID | SEQ ID | SEQ ID | SEQ ID |
| | NO: 20 | NO: 8 | NO: 17 | NO: 27 | NO: 34 | NO: 19 |

In some embodiments, the anti-GIP antibody, or antigen-binding fragment thereof, comprises a HCDR1, HCDR2, HCDR3, LCDR1, LCDR2, and LCDR3 as provided for in Table 3 below, which illustrates the CDR sequences based on IMGT numbering.

TABLE 3
CDRs of Exemplary anti-GIP antibodies-IMGT numbering
IMGT CDRs

| Ab | | | | | | |
|---|---|---|---|---|---|---|
| ID No | HCDR1 | HCDR2 | HCDR3 | LCDR1 | LCDR2 | LCDR3 |
| 14B9 | GFNIKDY | IDPENGDT | NSYGNNY | SSVSSSY | RT | SEQ ID |
| | Y (SEQ ID | (SEQ ID | FDY (SEQ | (SEQ ID | (dipeptide) | NO: 6 |
| | NO: 9) | NO: 10) | ID NO: 11) | NO: 12) | | |
| 10g10 | GFNIRDY | SEQ ID | NVYGIYF | SSISSNS | RT | SEQ ID |
| | Y (SEQ ID | NO: 10 | MDY (SEQ | (SEQ ID | (dipeptide) | NO: 19 |
| | NO: 21) | | ID NO: 22) | NO: 23) | | |
| GIP AB1 | SEQ ID NO: | SEQ ID NO: | SEQ ID NO: | SEQ ID NO: | RT | SEQ ID NO: |
| | 21 | 10 | 22 | 23 | (dipeptide) | 19 |
| GIP AB2 | SEQ ID NO: | SEQ ID NO: | SEQ ID NO: | SEQ ID NO: | RT | SEQ ID NO: |
| | 21 | 10 | 22 | 23 | (dipeptide) | 19 |
| GIP AB3 | SEQ ID NO: | SEQ ID NO: | SEQ ID NO: | SEQ ID NO: | RT | SEQ ID NO: |
| | 21 | 10 | 22 | 23 | (dipeptide) | 19 |

In some embodiments, the anti-GIP antibody, or antigen-binding fragment thereof, comprises a heavy or light chain CDR sequence as provided in the tables above. In some embodiments, the anti-GIP antibody, or antigen-binding fragment thereof, comprises a heavy or light chain CDR sequence as provided in the tables above and binds to non-human primate GIP. In some embodiments, the anti-GIP antibody, or antigen-binding fragment thereof, comprises a heavy or light chain CDR sequence as provided in the tables above and binds to human GIP.

In some embodiments, the anti-GIP antibody, or antigen-binding fragment thereof, comprises a light chain CDR having a sequence selected from SEQ ID NOs: 4-6, 18, 19, 27, 28, 31, or 34. In some embodiments, the anti-GIP antibody, or antigen-binding fragment thereof, comprises a light chain CDR having the amino acid sequence of SEQ ID NO: 4. In some embodiments, the anti-GIP antibody, or antigen-binding fragment thereof, comprises a light chain CDR having the amino acid sequence of SEQ ID NO: 5. In some embodiments, the anti-GIP antibody, or antigen-binding fragment thereof, comprises a light chain CDR having the amino acid sequence of SEQ ID NO: 6. In some embodiments, the anti-GIP antibody, or antigen-binding fragment thereof, comprises a light chain CDR having the amino acid sequence of SEQ ID NO: 18. In some embodiments, the anti-GIP antibody, or antigen-binding fragment thereof, comprises a light chain CDR having the amino acid sequence of SEQ ID NO: 19. In some embodiments, the anti-GIP antibody, or antigen-binding fragment thereof, comprises a light chain CDR having the amino acid sequence of SEQ ID NO: 27. In some embodiments, the anti-GIP antibody, or antigen-binding fragment thereof, comprises a light chain CDR having the amino acid sequence of SEQ ID NO: 28. In some embodiments, the anti-GIP antibody, or antigen-binding fragment thereof, comprises a light chain CDR having the amino acid sequence of SEQ ID NO: 31. In some embodiments, the anti-GIP antibody, or antigen-binding fragment thereof, comprises a light chain CDR having the amino acid sequence of SEQ ID NO: 34. The CDRs referenced in the embodiments throughout the present disclosure are primarily of Kabat numbering system. However, any such Kabat CDR may be interchanged with the CDRs that are characterized by different formats, such as Chothia and IMGT.

In some embodiments, the anti-GIP antibody, or antigen-binding fragment thereof, comprises a light chain variable region having a LCDR1, a LCDR2, and a LCDR3, wherein the LCDR1 has a sequence of SEQ ID NO: 4, the LCDR2 has a sequence of SEQ ID NO: 5, and the LCDR3 has a sequence of SEQ ID NO: 6. In some embodiments, the anti-GIP antibody, or antigen-binding fragment thereof, comprises a light chain variable region having a LCDR1, a LCDR2, and a LCDR3, wherein the LCDR1 has a sequence of SEQ ID NO: 12, the LCDR2 has a sequence of RT, and the LCDR3 has a sequence of SEQ ID NO: 6.

In some embodiments, the anti-GIP antibody, or antigen-binding fragment thereof, comprises a light chain variable region having a LCDR1, a LCDR2, and a LCDR3, wherein the LCDR1 has a sequence of SEQ ID NO: 18, the LCDR2 has a sequence of SEQ ID NO: 5, and the LCDR3 has a sequence of SEQ ID NO: 19. In some embodiments, the anti-GIP antibody, or antigen-binding fragment thereof, comprises a light chain variable region having a LCDR1, a LCDR2, and a LCDR3, wherein the LCDR1 has a sequence of SEQ ID NO: 23, the LCDR2 has a sequence of RT, and the LCDR3 has a sequence of SEQ ID NO: 19.

In some embodiments, the anti-GIP antibody, or antigen-binding fragment thereof, comprises a light chain variable region having a LCDR1, a LCDR2, and a LCDR3, wherein the LCDR1 has a sequence of SEQ ID NO: 27, the LCDR2 has a sequence of SEQ ID NO: 28, and the LCDR3 has a sequence of SEQ ID NO: 19. In some embodiments, the anti-GIP antibody, or antigen-binding fragment thereof, comprises a light chain variable region having a LCDR1, a LCDR2, and a LCDR3, wherein the LCDR1 has a sequence of SEQ ID NO: 23, the LCDR2 has a sequence of RT, and the LCDR3 has a sequence of SEQ ID NO: 19.

In some embodiments, the anti-GIP antibody, or antigen-binding fragment thereof, comprises a light chain variable region having a LCDR1, a LCDR2, and a LCDR3, wherein the LCDR1 has a sequence of SEQ ID NO: 27, the LCDR2 has a sequence of SEQ ID NO: 31, and the LCDR3 has a sequence of SEQ ID NO: 19. In some embodiments, the anti-GIP antibody, or antigen-binding fragment thereof, comprises a light chain variable region having a LCDR1, a LCDR2, and a LCDR3, wherein the LCDR1 has a sequence of SEQ ID NO: 23, the LCDR2 has a sequence of RT, and the LCDR3 has a sequence of SEQ ID NO: 19.

In some embodiments, the anti-GIP antibody, or antigen-binding fragment thereof, comprises a light chain variable region having a LCDR1, a LCDR2, and a LCDR3, wherein the LCDR1 has a sequence of SEQ ID NO: 27, the LCDR2 has a sequence of SEQ ID NO: 34, and the LCDR3 has a sequence of SEQ ID NO: 19. In some embodiments, the anti-GIP antibody, or antigen-binding fragment thereof, comprises a light chain variable region having a LCDR1, a LCDR2, and a LCDR3, wherein the LCDR1 has a sequence of SEQ ID NO: 23, the LCDR2 has a sequence of RT, and the LCDR3 has a sequence of SEQ ID NO: 19.

In some embodiments, the anti-GIP antibody, or antigen-binding fragment thereof, comprises a heavy chain CDR having a sequence selected from SEQ ID NOs: 1-3, 15-17, or 26. In some embodiments, the anti-GIP antibody, or antigen-binding fragment thereof, comprises a heavy chain CDR having the amino acid sequence of SEQ ID NO: 1. In some embodiments, the anti-GIP antibody, or antigen-binding fragment thereof, comprises a heavy chain CDR having the amino acid sequence of SEQ ID NO: 2. In some embodiments, the anti-GIP antibody, or antigen-binding fragment thereof, comprises a heavy chain CDR having the amino acid sequence of SEQ ID NO: 3. In some embodiments, the anti-GIP antibody, or antigen-binding fragment thereof, comprises a heavy chain CDR having the amino acid sequence of SEQ ID NO: 15. In some embodiments, the anti-GIP antibody, or antigen-binding fragment thereof, comprises a heavy chain CDR having the amino acid sequence of SEQ ID NO: 16. In some embodiments, the anti-GIP antibody, or antigen-binding fragment thereof, comprises a heavy chain CDR having the amino acid sequence of SEQ ID NO: 17. In some embodiments, the anti-GIP antibody, or antigen-binding fragment thereof, comprises a heavy chain CDR having the amino acid sequence of SEQ ID NO: 26. The CDRs referenced in the embodiments throughout the present disclosure are primarily of Kabat numbering system. However, any such Kabat CDR may be interchanged with the CDRs that are characterized by different formats, such as Chothia and IMGT.

In some embodiments, the anti-GIP antibody, or antigen-binding fragment thereof, comprises a heavy chain variable region having a HCDR1, a HCDR2, and a HCDR3, wherein the HCDR1 has a sequence of SEQ ID NO: 1, the HCDR2 has a sequence of SEQ ID NO: 2, and the HCDR3 has a sequence of SEQ ID NO: 3. In some embodiments, the anti-GIP antibody, or antigen-binding fragment thereof, comprises a heavy chain variable region having a HCDR1, a HCDR2, and a HCDR3, wherein the HCDR1 has a sequence of SEQ ID NO: 7, the HCDR2 has a sequence of SEQ ID NO: 8, and the HCDR3 has a sequence of SEQ ID NO: 3. In some embodiments, the anti-GIP antibody, or antigen-binding fragment thereof, comprises a heavy chain variable region having a HCDR1, a HCDR2, and a HCDR3, wherein the HCDR1 has a sequence of SEQ ID NO: 9, the HCDR2 has a sequence of SEQ ID NO: 10, and the HCDR3 has a sequence of SEQ ID NO: 11.

In some embodiments, the anti-GIP antibody, or antigen-binding fragment thereof, comprises a heavy chain variable region having a HCDR1, a HCDR2, and a HCDR3, wherein the HCDR1 has a sequence of SEQ ID NO: 15, the HCDR2 has a sequence of SEQ ID NO: 16, and the HCDR3 has a sequence of SEQ ID NO: 17. In some embodiments, the anti-GIP antibody, or antigen-binding fragment thereof, comprises a heavy chain variable region having a HCDR1, a HCDR2, and a HCDR3, wherein the HCDR1 has a sequence of SEQ ID NO: 20, the HCDR2 has a sequence of SEQ ID NO: 8, and the HCDR3 has a sequence of SEQ ID NO: 17. In some embodiments, the anti-GIP antibody, or antigen-binding fragment thereof, comprises a heavy chain variable region having a HCDR1, a HCDR2, and a HCDR3, wherein the HCDR1 has a sequence of SEQ ID NO: 21, the HCDR2 has a sequence of SEQ ID NO: 10, and the HCDR3 has a sequence of SEQ ID NO: 22.

In some embodiments, the anti-GIP antibody, or antigen-binding fragment thereof, comprises a heavy chain variable region having a HCDR1, a HCDR2, and a HCDR3, wherein the HCDR1 has a sequence of SEQ ID NO: 15, the HCDR2 has a sequence of SEQ ID NO: 26, and the HCDR3 has a sequence of SEQ ID NO: 17. In some embodiments, the anti-GIP antibody, or antigen-binding fragment thereof, comprises a heavy chain variable region having a HCDR1, a HCDR2, and a HCDR3, wherein the HCDR1 has a sequence of SEQ ID NO: 20, the HCDR2 has a sequence of SEQ ID NO: 8, and the HCDR3 has a sequence of SEQ ID NO: 17. In some embodiments, the anti-GIP antibody, or antigen-binding fragment thereof, comprises a heavy chain variable region having a HCDR1, a HCDR2, and a HCDR3, wherein the HCDR1 has a sequence of SEQ ID NO: 21, the HCDR2 has a sequence of SEQ ID NO: 10, and the HCDR3 has a sequence of SEQ ID NO: 22.

In some embodiments, the anti-GIP antibody, or antigen-binding fragment thereof, comprises: (i) a light chain having any one of the foregoing recited combination of LCDR1, LCDR2, and LCDR3 sequences; and (ii) a heavy chain having any one of the foregoing recited combinations of HCDR1, HCDR2, and HCDR3 sequences.

The different CDR motifs can be combined in any combination, including those not depicted in the table above. For example, the following embodiments are provided as non-limiting examples of such combinations.

In some embodiments, the anti-GIP antibody, or antigen-binding fragment thereof, comprises: (i) a light chain variable region comprising light chain CDR1, CDR2, and CDR3 sequences, wherein the light chain CDR1 sequence has the amino acid sequence of SEQ ID NO: 4; the light chain CDR2 sequence has the amino acid sequence of sequence of SEQ ID NO: 5; and the light chain CDR3 sequence has the amino acid sequence of SEQ ID NO: 6; and (ii) a heavy chain variable region comprising heavy chain CDR1, CDR2, and CDR3 sequences, wherein the heavy chain CDR1 sequence has the amino acid sequence of SEQ ID NO: 1, the heavy chain CDR2 sequence has the amino acid sequence of SEQ ID NO: 2, and the heavy chain CDR3 sequence has the amino acid sequence of SEQ ID NO: 3; or variants of any of the foregoing.

In some embodiments, the anti-GIP antibody, or antigen-binding fragment thereof, comprises: (i) a light chain variable region comprising light chain CDR1, CDR2, and CDR3 sequences, wherein the light chain CDR1 sequence has the amino acid sequence of SEQ ID NO: 4; the light chain CDR2 sequence has the amino acid sequence of sequence of SEQ ID NO: 5; and the light chain CDR3 sequence has the amino acid sequence of SEQ ID NO: 6; and (ii) a heavy chain variable region comprising heavy chain CDR1, CDR2, and CDR3 sequences, wherein the heavy chain CDR1 sequence has the amino acid sequence of SEQ ID NO: 7, the heavy chain CDR2 sequence has the amino acid sequence of SEQ ID NO: 8, and the heavy chain CDR3 sequence has the amino acid sequence of SEQ ID NO: 3; or variants of any of the foregoing.

In some embodiments, the anti-GIP antibody, or antigen-binding fragment thereof, comprises: (i) a light chain variable region comprising light chain CDR1, CDR2, and CDR3 sequences, wherein the light chain CDR1 sequence has the amino acid sequence of SEQ ID NO: 12; the light chain CDR2 sequence has the amino acid sequence of sequence of RT; and the light chain CDR3 sequence has the amino acid sequence of SEQ ID NO: 6; and (ii) a heavy chain variable region comprising heavy chain CDR1, CDR2, and CDR3 sequences, wherein the heavy chain CDR1 sequence has the amino acid sequence of SEQ ID NO: 9, the heavy chain CDR2 sequence has the amino acid sequence of SEQ ID NO: 10, and the heavy chain CDR3 sequence has the amino acid sequence of SEQ ID NO: 11; or variants of any of the foregoing.

In some embodiments, the anti-GIP antibody, or antigen-binding fragment thereof, comprises: (i) a light chain variable region comprising light chain CDR1, CDR2, and CDR3 sequences, wherein the light chain CDR1 sequence has the amino acid sequence of SEQ ID NO: 18; the light chain CDR2 sequence has the amino acid sequence of sequence of SEQ ID NO: 5; and the light chain CDR3 sequence has the amino acid sequence of SEQ ID NO: 19; and (ii) a heavy chain variable region comprising heavy chain CDR1, CDR2, and CDR3 sequences, wherein the heavy chain CDR1 sequence has the amino acid sequence of SEQ ID NO: 15, the heavy chain CDR2 sequence has the amino acid sequence of SEQ ID NO: 16, and the heavy chain CDR3 sequence has the amino acid sequence of SEQ ID NO: 17; or variants of any of the foregoing.

In some embodiments, the anti-GIP antibody, or antigen-binding fragment thereof, comprises: (i) a light chain variable region comprising light chain CDR1, CDR2, and CDR3 sequences, wherein the light chain CDR1 sequence has the amino acid sequence of SEQ ID NO: 18; the light chain CDR2 sequence has the amino acid sequence of sequence of SEQ ID NO: 5; and the light chain CDR3 sequence has the amino acid sequence of SEQ ID NO: 19; and (ii) a heavy chain variable region comprising heavy chain CDR1, CDR2, and CDR3 sequences, wherein the heavy chain CDR1 sequence has the amino acid sequence of SEQ ID NO: 20, the heavy chain CDR2 sequence has the amino acid sequence of SEQ ID NO: 8, and the heavy chain CDR3 sequence has the amino acid sequence of SEQ ID NO: 17; or variants of any of the foregoing.

In some embodiments, the anti-GIP antibody, or antigen-binding fragment thereof, comprises: (i) a light chain variable region comprising light chain CDR1, CDR2, and CDR3 sequences, wherein the light chain CDR1 sequence has the amino acid sequence of SEQ ID NO: 23; the light chain CDR2 sequence has the amino acid sequence of sequence of RT; and the light chain CDR3 sequence has the amino acid sequence of SEQ ID NO: 19; and (ii) a heavy chain variable region comprising heavy chain CDR1, CDR2, and CDR3 sequences, wherein the heavy chain CDR1 sequence has the amino acid sequence of SEQ ID NO: 21, the heavy chain CDR2 sequence has the amino acid sequence of SEQ ID NO: 10, and the heavy chain CDR3 sequence has the amino acid sequence of SEQ ID NO: 22; or variants of any of the foregoing.

In some embodiments, the anti-GIP antibody, or antigen-binding fragment thereof, comprises: (i) a light chain variable region comprising light chain CDR1, CDR2, and CDR3 sequences, wherein the light chain CDR1 sequence has the amino acid sequence of SEQ ID NO: 27; the light chain CDR2 sequence has the amino acid sequence of sequence of SEQ ID NO: 28; and the light chain CDR3 sequence has the amino acid sequence of SEQ ID NO: 19; and (ii) a heavy chain variable region comprising heavy chain CDR1, CDR2, and CDR3 sequences, wherein the heavy chain CDR1 sequence has the amino acid sequence of SEQ ID NO: 15, the heavy chain CDR2 sequence has the amino acid sequence of SEQ ID NO: 26, and the heavy chain CDR3 sequence has the amino acid sequence of SEQ ID NO: 17; or variants of any of the foregoing.

In some embodiments, the anti-GIP antibody, or antigen-binding fragment thereof, comprises: (i) a light chain variable region comprising light chain CDR1, CDR2, and CDR3 sequences, wherein the light chain CDR1 sequence has the amino acid sequence of SEQ ID NO: 27; the light chain CDR2 sequence has the amino acid sequence of sequence of SEQ ID NO: 28; and the light chain CDR3 sequence has the amino acid sequence of SEQ ID NO: 19; and (ii) a heavy chain variable region comprising heavy chain CDR1, CDR2, and CDR3 sequences, wherein the heavy chain CDR1 sequence has the amino acid sequence of SEQ ID NO: 20, the heavy chain CDR2 sequence has the amino acid sequence of SEQ ID NO: 8, and the heavy chain CDR3 sequence has the amino acid sequence of SEQ ID NO: 17; or variants of any of the foregoing.

In some embodiments, the anti-GIP antibody, or antigen-binding fragment thereof, comprises: (i) a light chain variable region comprising light chain CDR1, CDR2, and CDR3 sequences, wherein the light chain CDR1 sequence has the amino acid sequence of SEQ ID NO: 27; the light chain CDR2 sequence has the amino acid sequence of sequence of SEQ ID NO: 31; and the light chain CDR3 sequence has the amino acid sequence of SEQ ID NO: 19; and (ii) a heavy chain variable region comprising heavy chain CDR1, CDR2, and CDR3 sequences, wherein the heavy chain CDR1 sequence has the amino acid sequence of SEQ ID NO: 15, the heavy chain CDR2 sequence has the amino acid sequence of SEQ ID NO: 26, and the heavy chain CDR3 sequence has the amino acid sequence of SEQ ID NO: 17; or variants of any of the foregoing.

In some embodiments, the anti-GIP antibody, or antigen-binding fragment thereof, comprises: (i) a light chain variable region comprising light chain CDR1, CDR2, and CDR3 sequences, wherein the light chain CDR1 sequence has the amino acid sequence of SEQ ID NO: 27; the light chain CDR2 sequence has the amino acid sequence of sequence of SEQ ID NO: 31; and the light chain CDR3 sequence has the amino acid sequence of SEQ ID NO: 19; and (ii) a heavy chain variable region comprising heavy chain CDR1, CDR2, and CDR3 sequences, wherein the heavy chain CDR1 sequence has the amino acid sequence of SEQ ID NO: 20, the heavy chain CDR2 sequence has the amino acid sequence of SEQ ID NO: 8, and the heavy chain CDR3 sequence has the amino acid sequence of SEQ ID NO: 17; or variants of any of the foregoing.

In some embodiments, the anti-GIP antibody, or antigen-binding fragment thereof, comprises: (i) a light chain variable region comprising light chain CDR1, CDR2, and CDR3 sequences, wherein the light chain CDR1 sequence has the amino acid sequence of SEQ ID NO: 27; the light chain CDR2 sequence has the amino acid sequence of sequence of SEQ ID NO: 34; and the light chain CDR3 sequence has the amino acid sequence of SEQ ID NO: 19; and (ii) a heavy chain variable region comprising heavy chain CDR1, CDR2, and CDR3 sequences, wherein the heavy chain CDR1 sequence has the amino acid sequence of SEQ ID NO: 15, the heavy chain CDR2 sequence has the amino acid sequence of SEQ ID NO: 26, and the heavy chain CDR3 sequence has the amino acid sequence of SEQ ID NO: 17; or variants of any of the foregoing.

In some embodiments, the anti-GIP antibody, or antigen-binding fragment thereof, comprises: (i) a light chain variable region comprising light chain CDR1, CDR2, and CDR3 sequences, wherein the light chain CDR1 sequence has the amino acid sequence of SEQ ID NO: 27; the light chain CDR2 sequence has the amino acid sequence of sequence of SEQ ID NO: 34; and the light chain CDR3 sequence has the amino acid sequence of SEQ ID NO: 19; and (ii) a heavy chain variable region comprising heavy chain CDR1, CDR2, and CDR3 sequences, wherein the heavy chain CDR1 sequence has the amino acid sequence of SEQ ID NO: 20, the heavy chain CDR2 sequence has the amino acid sequence of SEQ ID NO: 8, and the heavy chain CDR3 sequence has the amino acid sequence of SEQ ID NO: 17; or variants of any of the foregoing.

In some embodiments, the light chain variable region CDR1 is replaced with any of the other light chain CDR1 sequences. In some embodiments, the light chain variable region CDR2 is replaced with any of the other light chain CDR2 sequences. In some embodiments, the light chain variable region CDR3 is replaced with any of the other light chain CDR3 sequences. In some embodiments, the heavy chain variable region CDR1 is replaced with any of the other heavy chain CDR1 sequences. In some embodiments, the heavy chain variable region CDR2 is replaced with any of the other heavy chain CDR2 sequences. In some embodiments, the heavy chain variable region CDR3 is replaced with any of the other heavy chain CDR3 sequences.

In some embodiments, the anti-GIP antibody, or antigen-binding fragment thereof, comprises a heavy chain variable region (VH) having one of the following sequences, or a variant thereof:

TABLE 4
Sequences of the VH region
of exemplary anti-GIP antibodies

| SEQ | AB | |
|---|---|---|
| ID NO: | ID NO: | Sequence |
| 13 | 14B9 | QVQLQESGAALVRSGASVKLSCTASGFN |
| | | IKDYYFHWVKQRPDQGLEWIGWIDPENG |
| | | DTEYAPNFQVKATMTADTYSNTAYLHLS |
| | | SLTSEDTAVYYCNSYGNNYFDYWGQGTT |
| | | VTVSS |
| 24 | 10g10 | QVQLQESGAELVRSGASVKLSCTASGFN |
| | | IRDYYLHWIKQRPEQGLEWIGWIDPENG |
| | | DTEYAPKFQDKATVTADTSSNTAYLQLN |
| | | SLTSEDTAVYYCNVYGIYFMDYWGQGTT |
| | | VTVSS |
| 29 | GIP AB1 | QVQLVQSGAEVKKPGATVKISCKASGFN |
| | | IRDYYLHWVQQAPGKGLEWMGWIDPENG |
| | | DTEYAPKFQGRVTITADTSTNTAYMELS |
| | | SLRSEDTAVYYCNVYGIYFMDYWGQGTM |
| | | VTVSS |
| 32 | GIP AB2 | QVQLVQSGAEVKKPGATVKISCKVSGFN |
| | | IRDYYLHWVQQAPGKGLEWMGWIDPENG |
| | | DTEYAPKFQGRVTITADTSTDTAYMELS |
| | | SLRSEDTAVYYCNVYGIYFMDYWGQGTM |
| | | VTVSS |
| 35 | GIP AB3 | QVQLQESGAEVKKPGASVKVSCKASGFN |
| | | IRDYYLHWVRQAPGQGLEWMGWIDPENG |
| | | DTEYAPKFQGRVTTTADTSISTAYMELS |
| | | RLRSDDTAVYYCNVYGIYFMDYWGQGTL |
| | | VTVSS |

In some embodiments, the anti-GIP antibody, or antigen-binding fragment thereof, comprises a light chain variable region (VL) having one of the following sequences, or a variant thereof:

TABLE 5
Sequences of the VL region
of exemplary anti-GIP antibodies

| SEQ | AB | |
|---|---|---|
| ID NO: | ID NO: | Sequence |
| 14 | 14B9 | DIQLTQSPAIMAASLGQKVTMTCSAS |
| | | SSVSSSYLHWYQQKSGASPKSLIHRT |
| | | SNLASGVPARFSGSGSGTSYSLTISS |
| | | VEAEDDATYYCLQWSGFPFTFGSGTK |
| | | LEIKR |
| 25 | 10g10 | DIQLTQSPSTMAASPGEKITITCSAS |
| | | SSISSNSLHWYQQKPGFSPKLLIYRT |
| | | SNLASGVPGRFSGSGSGTSYSLTIGT |
| | | METEDVATYYCQQGSSFPRMLTFGTG |
| | | TKLEIKR |
| 30 | GIP AB1 | DIQLTQSPSSVSASVGDRVTITCRAS |
| | | SSISSNSLHWYQQKPGKAPKLLIYRT |
| | | SNLQSGVPSRFSGSGSGTDYTLTISS |
| | | LQPEDFATYYCQQGSSFPRMLTFGQG |
| | | TKLEIKR |
| 33 | GIP AB2 | DIQMTQSPSSVSASVGDRVTITCRAS |
| | | SSISSNSLHWYQQKPGKAPKLLIYRT |
| | | SSLQSGVPSRFSGSGSGTDYTLTISS |
| | | LQPEDFATYYCQQGSSFPRMLTFGQG |
| | | TKLEIKR |
| 36 | GIP AB3 | DIQMTQSPGTLSLSPGERATLSCRAS |
| | | SSISSNSLHWYQQKPGQAPRLLIYRT |
| | | SNRATGIPARFSGSGSGTDYTLTISS |
| | | LEPEDFAVYYCQQGSSFPRMLTFGGG |
| | | TKVEIKR |

In some embodiments, the anti-GIP antibody, or antigen-binding fragment thereof, comprises a VH comprising an amino acid sequence having at least 85%, 86%, 87%, 88%, 89%, 90%, 91%, 92%, 93%, 94%, 95%, 96%, 97%, 98%, or 99% identity to the amino acid sequence of SEQ ID NO: 29, SEQ ID NO: 13, SEQ ID NO: 24, SEQ ID NO: 32, or SEQ ID NO: 35. In some embodiments, the anti-GIP antibody, or antigen-binding fragment thereof, comprises a V_{H} comprising an amino acid sequence having at least 85% identity to the amino acid sequence of SEQ ID NO: 29, SEQ ID NO: 13, SEQ ID NO: 24, SEQ ID NO: 32, or SEQ ID NO: 35. In some embodiments, the anti-GIP antibody, or antigen-binding fragment thereof, comprises a V_{H} comprising an amino acid sequence having at least 90% identity to the amino acid sequence of SEQ ID NO: 29, SEQ ID NO: 13, SEQ ID NO: 24, SEQ ID NO: 32, or SEQ ID NO: 35. In some embodiments, the anti-GIP antibody, or antigen-binding fragment thereof, comprises a V_{H} comprising an amino acid sequence having at least 95% identity to the amino acid sequence of SEQ ID NO: 29, SEQ ID NO: 13, SEQ ID NO: 24, SEQ ID NO: 32, or SEQ ID NO: 35. In some embodiments, the anti-GIP antibody, or antigen-binding fragment thereof, comprises a V_{H} comprising an amino acid sequence having at least 98% identity to the amino acid sequence of SEQ ID NO: 29, SEQ ID NO: 13, SEQ ID NO: 24, SEQ ID NO: 32, or SEQ ID NO: 35. In some embodiments, the anti-GIP antibody, or antigen-binding fragment thereof, comprises a V_{H} comprising an amino acid sequence of SEQ ID NO: 29, SEQ ID NO: 13, SEQ ID NO: 24, SEQ ID NO: 32, or SEQ ID NO: 35. In some embodiments, the anti-GIP antibody, or antigen-binding fragment thereof, comprises a V_{H} comprising an amino acid sequence of SEQ ID NO: 29. In some embodiments, the anti-GIP antibody, or antigen-binding fragment thereof, comprises a V_{H} comprising an amino acid sequence of SEQ ID NO: 13. In some embodiments, the anti-GIP antibody, or antigen-binding fragment thereof, comprises a V_{H} comprising an amino acid sequence of SEQ ID NO: 24. In some embodiments, the anti-GIP antibody, or antigen-binding fragment thereof, comprises a V_{H} comprising an amino acid sequence of SEQ ID NO: 32. In some embodiments, the anti-GIP antibody, or antigen-binding fragment thereof, comprises a V_{H} comprising an amino acid sequence of SEQ ID NO: 35.

In some embodiments, the anti-GIP antibody, or antigen-binding fragment thereof, comprises a V_{L} comprising an amino acid sequence having at least 85%, 86%, 87%, 88%, 89%, 90%, 91%, 92%, 93%, 94%, 95%, 96%, 97%, 98%, or 99% identity to the amino acid sequence of SEQ ID NO: 30, SEQ ID NO: 14, SEQ ID NO: 25, SEQ ID NO: 33, or SEQ ID NO: 36. In some embodiments, the anti-GIP antibody, or antigen-binding fragment thereof, comprises a V_{L} comprising an amino acid sequence having at least 85% identity to the amino acid sequence of SEQ ID NO: 30, SEQ ID NO: 14, SEQ ID NO: 25, SEQ ID NO: 33, or SEQ ID NO: 36. In some embodiments, the anti-GIP antibody, or antigen-binding fragment thereof, comprises a V_{L} comprising an amino acid sequence having at least 90% identity to the amino acid sequence of SEQ ID NO: 30, SEQ ID NO: 14, SEQ ID NO: 25, SEQ ID NO: 33, or SEQ ID NO: 36. In some embodiments, the anti-GIP antibody, or antigen-binding fragment thereof, comprises a V_{L} comprising an amino acid sequence having at least 95% identity to the amino acid sequence of SEQ ID NO: 30, SEQ ID NO: 14, SEQ ID NO: 25, SEQ ID NO: 33, or SEQ ID NO: 36. In some embodiments, the anti-GIP antibody, or antigen-binding fragment thereof, comprises a V_{L} comprising an amino acid sequence having at least 98% identity to the amino acid sequence of SEQ ID NO: 30, SEQ ID NO: 14, SEQ ID NO: 25, SEQ ID NO: 33, or SEQ ID NO: 36. In some embodiments, the anti-GIP antibody, or antigen-binding fragment thereof, comprises a V_{L} comprising an amino acid sequence of SEQ ID NO: 30, SEQ ID NO: 14, SEQ ID NO: 25, SEQ ID NO: 33, or SEQ ID NO: 36. In some embodiments, the anti-GIP antibody, or antigen-binding fragment thereof, comprises a V_{L} comprising an amino acid sequence of SEQ ID NO: 30. In some embodiments, the anti-GIP antibody, or antigen-binding fragment thereof, comprises a V_{L} comprising an amino acid sequence of SEQ ID NO: 14. In some embodiments, the anti-GIP antibody, or antigen-binding fragment thereof, comprises a V_{L} comprising an amino acid sequence of SEQ ID NO: 25. In some embodiments, the anti-GIP antibody, or antigen-binding fragment thereof, comprises a V_{L} comprising an amino acid sequence of SEQ ID NO: 33. In some embodiments, the anti-GIP antibody, or antigen-binding fragment thereof, comprises a V_{L} comprising an amino acid sequence of SEQ ID NO: 36.

In some embodiments, the anti-GIP antibody, or antigen-binding fragment thereof, comprises a V_{H} and a V_{L}, wherein the V_{H} comprises an amino acid sequence having at least 85%, 86%, 87%, 88%, 89%, 90%, 91%, 92%, 93%, 94%, 95%, 96%, 97%, 98%, or 99% identity to the amino acid sequence of SEQ ID NO: 13; and the V_{L} comprises an amino acid sequence having at least 85%, 86%, 87%, 88%, 89%, 90%, 91%, 92%, 93%, 94%, 95%, 96%, 97%, 98%, or 99% identity to the amino acid sequence of SEQ ID NO: 14. In some embodiments, the anti-GIP antibody, or antigen-binding fragment thereof, comprises a V_{H} and a V_{L}, wherein the V_{H} comprises an amino acid sequence having at least 85%, 86%, 87%, 88%, 89%, 90%, 91%, 92%, 93%, 94%, 95%, 96%, 97%, 98%, or 99% identity to the amino acid sequence of SEQ ID NO: 13; and the V_{L} comprises an amino acid sequence having at least 85%, 86%, 87%, 88%, 89%, 90%, 91%, 92%, 93%, 94%, 95%, 96%, 97%, 98%, or 99% identity to the amino acid sequence of SEQ ID NO: 14; provided that the V_{H} comprises a HCDR1 having an amino acid sequence of SEQ ID NO: 1, a HCDR2 having an amino acid sequence of SEQ ID NO: 2, and a HCDR3 having an amino acid sequence of SEQ ID NO: 3. In some embodiments, the anti-GIP antibody, or antigen-binding fragment thereof, comprises a V_{H} and a V_{L}, wherein the V_{H} comprises an amino acid sequence having at least 85%, 86%, 87%, 88%, 89%, 90%, 91%, 92%, 93%, 94%, 95%, 96%, 97%, 98%, or 99% identity to the amino acid sequence of SEQ ID NO: 13; and the V_{L} comprises an amino acid sequence having at least 85%, 86%, 87%, 88%, 89%, 90%, 91%, 92%, 93%, 94%, 95%, 96%, 97%, 98%, or 99% identity to the amino acid sequence of SEQ ID NO: 14; provided that the V_{L} comprises a LCDR1 having an amino acid sequence of SEQ ID NO: 4, a LCDR2 having an amino acid sequence of SEQ ID NO: 5, and a LCDR3 having an amino acid sequence of SEQ ID NO: 6. In some embodiments, the anti-GIP antibody, or antigen-binding fragment thereof, comprises a V_{H} and a V_{L}, wherein the V_{H} comprises an amino acid sequence having at least 85%, 86%, 87%, 88%, 89%, 90%, 91%, 92%, 93%, 94%, 95%, 96%, 97%, 98%, or 99% identity to the amino acid sequence of SEQ ID NO: 13; and the V_{L} comprises an amino acid sequence having at least 85%, 86%, 87%, 88%, 89%, 90%, 91%, 92%, 93%, 94%, 95%, 96%, 97%, 98%, or 99% identity to the amino acid sequence of SEQ ID NO: 14; provided that the V_{H} comprises a HCDR1 having an amino acid sequence of SEQ ID NO: 1, a HCDR2 having an amino acid sequence of SEQ ID NO: 2, and a HCDR3 having an amino acid sequence of SEQ ID NO: 3; and provided that the V_{L} comprises a LCDR1 having an amino acid sequence of SEQ ID NO: 4, a LCDR2 having an amino acid sequence of SEQ ID NO: 5, and a LCDR3 having an amino acid sequence of SEQ ID NO: 6. In some embodiments, the CDRs in the V_{H} or V_{L} are as set forth in the combinations provided for herein.

In some embodiments, the anti-GIP antibody, or antigen-binding fragment thereof, comprises a V_{H} and a V_{L}, wherein the V_{H} comprises an amino acid sequence having at least 85%, 86%, 87%, 88%, 89%, 90%, 91%, 92%, 93%, 94%, 95%, 96%, 97%, 98%, or 99% identity to the amino acid sequence of SEQ ID NO: 13; and the V_{L} comprises an amino acid sequence having at least 85%, 86%, 87%, 88%, 89%, 90%, 91%, 92%, 93%, 94%, 95%, 96%, 97%, 98%, or 99% identity to the amino acid sequence of SEQ ID NO: 14; provided that the V_{H} comprises a HCDR1 having an amino acid sequence of SEQ ID NO: 1, wherein the HCDR1 comprises at most 1 conservative amino acid substitution, a HCDR2 having an amino acid sequence of SEQ ID NO: 2, wherein the HCDR2 comprises at most 1 conservative amino acid substitution, and a HCDR3 having an amino acid sequence of SEQ ID NO: 3 wherein the HCDR3 comprises at most 1 conservative amino acid substitution; and provided that the V_{L} comprises a LCDR1 having an amino acid sequence of SEQ ID NO: 4, wherein the LCDR1 comprises at most 1 conservative amino acid substitution, a LCDR2 having an amino acid sequence of SEQ ID NO: 5, wherein the LCDR2 comprises at most 1 conservative amino acid substitution, and a LCDR3 having an amino acid sequence of SEQ ID NO: 6, wherein the LCDR3 comprises at most 1 conservative amino acid substitution.

In some embodiments, the anti-GIP antibody, or antigen-binding fragment thereof, comprises a V_{H} and a V_{L}, wherein the V_{H} comprises an amino acid sequence having at least 90%, identity to the amino acid sequence of SEQ ID NO: 13; and the V_{L} comprises an amino acid sequence having at least 90% identity to the amino acid sequence of SEQ ID NO: 14; provided that the V_{H} comprises a HCDR1 having an amino acid sequence of SEQ ID NO: 1, a HCDR2 having an amino acid sequence of SEQ ID NO: 2, and a HCDR3 having an amino acid sequence of SEQ ID NO: 3; and provided that the V_{L} comprises a LCDR1 having an amino acid sequence of SEQ ID NO: 4, a LCDR2 having an amino acid sequence of SEQ ID NO: 5, and a LCDR3 having an amino acid sequence of SEQ ID NO: 6. In some embodiments, the anti-GIP antibody, or antigen-binding fragment thereof, comprises a V_{H} and a V_{L}, wherein the V_{H} comprises an amino acid sequence having at least 95%, identity to the amino acid sequence of SEQ ID NO: 13; and the V_{L} comprises an amino acid sequence having at least 95% identity to the amino acid sequence of SEQ ID NO: 14; provided that the V_{H} comprises a HCDR1 having an amino acid sequence of SEQ ID NO: 1, a HCDR2 having an amino acid sequence of SEQ ID NO: 2, and a HCDR3 having an amino acid sequence of SEQ ID NO: 3; and provided that the V_{L} comprises a LCDR1 having an amino acid sequence of SEQ ID NO: 4, a LCDR2 having an amino acid sequence of SEQ ID NO: 5, and a LCDR3 having an amino acid sequence of SEQ ID NO: 6. In some embodiments, the anti-GIP antibody, or antigen-binding fragment thereof, comprises a V_{H} and a V_{L}, wherein the V_{H} comprises an amino acid sequence having at least 98%, identity to the amino acid sequence of SEQ ID NO: 13; and the V_{L} comprises an amino acid sequence having at least 98% identity to the amino acid sequence of SEQ ID NO: 14; provided that the V_{H} comprises a HCDR1 having an amino acid sequence of SEQ ID NO: 1, a HCDR2 having an amino acid sequence of SEQ ID NO: 2, and a HCDR3 having an amino acid sequence of SEQ ID NO: 3; and provided that the V_{L} comprises a LCDR1 having an amino acid sequence of SEQ ID NO: 4, a LCDR2 having an amino acid sequence of SEQ ID NO: 5, and a LCDR3 having an amino acid sequence of SEQ ID NO: 6. In some embodiments, the anti-GIP antibody, or antigen-binding fragment thereof, comprises a V_{H} and a V_{L}, wherein the V_{H} comprises an amino acid sequence of SEQ ID NO: 13 and the V_{L} comprises an amino acid of SEQ ID NO: 14.

In some embodiments, the anti-GIP antibody, or antigen-binding fragment thereof, comprises a V_{H} and a V_{L}, wherein the V_{H} comprises an amino acid sequence having at least 85%, 86%, 87%, 88%, 89%, 90%, 91%, 92%, 93%, 94%, 95%, 96%, 97%, 98%, or 99% identity to the amino acid sequence of SEQ ID NO: 24; and the V_{L} comprises an amino acid sequence having at least 85%, 86%, 87%, 88%, 89%, 90%, 91%, 92%, 93%, 94%, 95%, 96%, 97%, 98%, or 99% identity to the amino acid sequence of SEQ ID NO: 25. In some embodiments, the anti-GIP antibody, or antigen-binding fragment thereof, comprises a V_{H} and a V_{L}, wherein the V_{H} comprises an amino acid sequence having at least 85%, 86%, 87%, 88%, 89%, 90%, 91%, 92%, 93%, 94%, 95%, 96%, 97%, 98%, or 99% identity to the amino acid sequence of SEQ ID NO: 24; and the V_{L} comprises an amino acid sequence having at least 85%, 86%, 87%, 88%, 89%, 90%, 91%, 92%, 93%, 94%, 95%, 96%, 97%, 98%, or 99% identity to the amino acid sequence of SEQ ID NO: 25; provided that the V_{H} comprises a HCDR1 having an amino acid sequence of SEQ ID NO: 15, a HCDR2 having an amino acid sequence of SEQ ID NO: 16, and a HCDR3 having an amino acid sequence of SEQ ID NO: 17. In some embodiments, the anti-GIP antibody, or antigen-binding fragment thereof, comprises a V_{H} and a V_{L}, wherein the V_{H} comprises an amino acid sequence having at least 85%, 86%, 87%, 88%, 89%, 90%, 91%, 92%, 93%, 94%, 95%, 96%, 97%, 98%, or 99% identity to the amino acid sequence of SEQ ID NO: 24; and the V_{L} comprises an amino acid sequence having at least 85%, 86%, 87%, 88%, 89%, 90%, 91%, 92%, 93%, 94%, 95%, 96%, 97%, 98%, or 99% identity to the amino acid sequence of SEQ ID NO: 25; provided that the V_{L} comprises a LCDR1 having an amino acid sequence of SEQ ID NO: 18, a LCDR2 having an amino acid sequence of SEQ ID NO: 5, and a LCDR3 having an amino acid sequence of SEQ ID NO: 19. In some embodiments, the anti-GIP antibody, or antigen-binding fragment thereof, comprises a V_{H} and a V_{L}, wherein the V_{H} comprises an amino acid sequence having at least 85%, 86%, 87%, 88%, 89%, 90%, 91%, 92%, 93%, 94%, 95%, 96%, 97%, 98%, or 99% identity to the amino acid sequence of SEQ ID NO: 24; and the V_{L} comprises an amino acid sequence having at least 85%, 86%, 87%, 88%, 89%, 90%, 91%, 92%, 93%, 94%, 95%, 96%, 97%, 98%, or 99% identity to the amino acid sequence of SEQ ID NO: 25; provided that the V_{H} comprises a HCDR1 having an amino acid sequence of SEQ ID NO: 15, a HCDR2 having an amino acid sequence of SEQ ID NO: 16, and a HCDR3 having an amino acid sequence of SEQ ID NO: 17; and provided that the V_{L} comprises a LCDR1 having an amino acid sequence of SEQ ID NO: 18, a LCDR2 having an amino acid sequence of SEQ ID NO: 5, and a LCDR3 having an amino acid sequence of SEQ ID NO: 19. In some embodiments, the CDRs in the V_{H} or V_{L} are as set forth in the combinations provided for herein.

In some embodiments, the anti-GIP antibody, or antigen-binding fragment thereof, comprises a V_{H} and a V_{L}, wherein the V_{H} comprises an amino acid sequence having at least 85%, 86%, 87%, 88%, 89%, 90%, 91%, 92%, 93%, 94%, 95%, 96%, 97%, 98%, or 99% identity to the amino acid sequence of SEQ ID NO: 24; and the V_{L} comprises an amino acid sequence having at least 85%, 86%, 87%, 88%, 89%, 90%, 91%, 92%, 93%, 94%, 95%, 96%, 97%, 98%, or 99% identity to the amino acid sequence of SEQ ID NO: 25; provided that the V_{H} comprises a HCDR1 having an amino acid sequence of SEQ ID NO: 15, wherein the HCDR1 comprises at most 1 conservative amino acid substitution, a HCDR2 having an amino acid sequence of SEQ ID NO: 16, wherein the HCDR2 comprises at most 1 conservative amino acid substitution, and a HCDR3 having an amino acid sequence of SEQ ID NO: 17, wherein the HCDR3 comprises at most 1 conservative amino acid substitution; and provided that the V_{L} comprises a LCDR1 having an amino acid sequence of SEQ ID NO: 18, wherein the LCDR1 comprises at most 1 conservative amino acid substitution, a LCDR2 having an amino acid sequence of SEQ ID NO: 5, wherein the LCDR2 comprises at most 1 conservative amino acid substitution, and a LCDR3 having an amino acid sequence of SEQ ID NO: 19, wherein the LCDR3 comprises at most 1 conservative amino acid substitution.

In some embodiments, the anti-GIP antibody, or antigen-binding fragment thereof, comprises a V_{H} and a V_{L}, wherein the V_{H} comprises an amino acid sequence having at least 90%, identity to the amino acid sequence of SEQ ID NO: 24; and the V_{L} comprises an amino acid sequence having at least 90% identity to the amino acid sequence of SEQ ID NO: 25; provided that the V_{H} comprises a HCDR1 having an amino acid sequence of SEQ ID NO: 15, a HCDR2 having an amino acid sequence of SEQ ID NO: 16, and a HCDR3 having an amino acid sequence of SEQ ID NO: 17; and provided that the V_{L} comprises a LCDR1 having an amino acid sequence of SEQ ID NO: 18, a LCDR2 having an amino acid sequence of SEQ ID NO: 5, and a LCDR3 having an amino acid sequence of SEQ ID NO: 19. In some embodiments, the anti-GIP antibody, or antigen-binding fragment thereof, comprises a V_{H} and a V_{L}, wherein the V_{H} comprises an amino acid sequence having at least 95%, identity to the amino acid sequence of SEQ ID NO: 24; and the V_{L} comprises an amino acid sequence having at least 95% identity to the amino acid sequence of SEQ ID NO: 25; provided that the V_{H} comprises a HCDR1 having an amino acid sequence of SEQ ID NO: 15, a HCDR2 having an amino acid sequence of SEQ ID NO: 16, and a HCDR3 having an amino acid sequence of SEQ ID NO: 17; and provided that the V_{L} comprises a LCDR1 having an amino acid sequence of SEQ ID NO: 18, a LCDR2 having an amino acid sequence of SEQ ID NO: 5, and a LCDR3 having an amino acid sequence of SEQ ID NO: 19. In some embodiments, the anti-GIP antibody, or antigen-binding fragment thereof, comprises a V_{H} and a V_{L}, wherein the V_{H} comprises an amino acid sequence having at least 98%, identity to the amino acid sequence of SEQ ID NO: 24; and the V_{L} comprises an amino acid sequence having at least 98% identity to the amino acid sequence of SEQ ID NO: 25; provided that the V_{H} comprises a HCDR1 having an amino acid sequence of SEQ ID NO: 15, a HCDR2 having an amino acid sequence of SEQ ID NO: 16, and a HCDR3 having an amino acid sequence of SEQ ID NO: 17; and provided that the V_{L} comprises a LCDR1 having an amino acid sequence of SEQ ID NO: 18, a LCDR2 having an amino acid sequence of SEQ ID NO: 5, and a LCDR3 having an amino acid sequence of SEQ ID NO: 19. In some embodiments, the anti-GIP antibody, or antigen-binding fragment thereof, comprises a V_{H} and a V_{L}, wherein the V_{H} comprises an amino acid sequence of SEQ ID NO: 24 and the V_{L} comprises an amino acid sequence of SEQ ID NO: 25.

In some embodiments, the anti-GIP antibody, or antigen-binding fragment thereof, comprises a V_{H} and a V_{L}, wherein the V_{H} comprises an amino acid sequence having at least 85%, 86%, 87%, 88%, 89%, 90%, 91%, 92%, 93%, 94%, 95%, 96%, 97%, 98%, or 99% identity to the amino acid sequence of SEQ ID NO: 29; and the V_{L} comprises an amino acid sequence having at least 85%, 86%, 87%, 88%, 89%, 90%, 91%, 92%, 93%, 94%, 95%, 96%, 97%, 98%, or 99% identity to the amino acid sequence of SEQ ID NO: 30. In some embodiments, the anti-GIP antibody, or antigen-binding fragment thereof, comprises a V_{H} and a V_{L}, wherein the V_{H} comprises an amino acid sequence having at least 85%, 86%, 87%, 88%, 89%, 90%, 91%, 92%, 93%, 94%, 95%, 96%, 97%, 98%, or 99% identity to the amino acid sequence of SEQ ID NO: 29; and the V_{L} comprises an amino acid sequence having at least 85%, 86%, 87%, 88%, 89%, 90%, 91%, 92%, 93%, 94%, 95%, 96%, 97%, 98%, or 99% identity to the amino acid sequence of SEQ ID NO: 30; provided that the V_{H} comprises a HCDR1 having an amino acid sequence of SEQ ID NO: 15, a HCDR2 having an amino acid sequence of SEQ ID NO: 26, and a HCDR3 having an amino acid sequence of SEQ ID NO: 17. In some embodiments, the anti-GIP antibody, or antigen-binding fragment thereof, comprises a V_{H} and a V_{L}, wherein the V_{H} comprises an amino acid sequence having at least 85%, 86%, 87%, 88%, 89%, 90%, 91%, 92%, 93%, 94%, 95%, 96%, 97%, 98%, or 99% identity to the amino acid sequence of SEQ ID NO: 29; and the V_{L} comprises an amino acid sequence having at least 85%, 86%, 87%, 88%, 89%, 90%, 91%, 92%, 93%, 94%, 95%, 96%, 97%, 98%, or 99% identity to the amino acid sequence of SEQ ID NO: 30; provided that the V_{L} comprises a LCDR1 having an amino acid sequence of SEQ ID NO: 27, a LCDR2 having an amino acid sequence of SEQ ID NO: 28, and a LCDR3 having an amino acid sequence of SEQ ID NO: 19. In some embodiments, the anti-GIP antibody, or antigen-binding fragment thereof, comprises a V_{H} and a V_{L}, wherein the V_{H} comprises an amino acid sequence having at least 85%, 86%, 87%, 88%, 89%, 90%, 91%, 92%, 93%, 94%, 95%, 96%, 97%, 98%, or 99% identity to the amino acid sequence of SEQ ID NO: 29; and the V_{L} comprises an amino acid sequence having at least 85%, 86%, 87%, 88%, 89%, 90%, 91%, 92%, 93%, 94%, 95%, 96%, 97%, 98%, or 99% identity to the amino acid sequence of SEQ ID NO: 30; provided that the V_{H} comprises a HCDR1 having an amino acid sequence of SEQ ID NO: 15, a HCDR2 having an amino acid sequence of SEQ ID NO: 26, and a HCDR3 having an amino acid sequence of SEQ ID NO: 17; and provided that the V_{L} comprises a LCDR1 having an amino acid sequence of SEQ ID NO: 27, a LCDR2 having an amino acid sequence of SEQ ID NO: 28, and a LCDR3 having an amino acid sequence of SEQ ID NO: 19. In some embodiments, the CDRs in the V_{H} or V_{L} are as set forth in the combinations provided for herein.

In some embodiments, the anti-GIP antibody, or antigen-binding fragment thereof, comprises a V_{H} and a V_{L}, wherein the V_{H} comprises an amino acid sequence having at least 85%, 86%, 87%, 88%, 89%, 90%, 91%, 92%, 93%, 94%, 95%, 96%, 97%, 98%, or 99% identity to the amino acid sequence of SEQ ID NO: 29; and the V_{L} comprises an amino acid sequence having at least 85%, 86%, 87%, 88%, 89%, 90%, 91%, 92%, 93%, 94%, 95%, 96%, 97%, 98%, or 99% identity to the amino acid sequence of SEQ ID NO: 30; provided that the V_{H} comprises a HCDR1 having an amino acid sequence of SEQ ID NO: 15, wherein the HCDR1 comprises at most 1 conservative amino acid substitution, a HCDR2 having an amino acid sequence of SEQ ID NO: 26, wherein the HCDR2 comprises at most 1 conservative amino acid substitution, and a HCDR3 having an amino acid sequence of SEQ ID NO: 17, wherein the HCDR3 comprises at most 1 conservative amino acid substitution; and provided that the V_{L} comprises a LCDR1 having an amino acid sequence of SEQ ID NO: 27, wherein the LCDR1 comprises at most 1 conservative amino acid substitution, a LCDR2 having an amino acid sequence of SEQ ID NO: 28, wherein the LCDR2 comprises at most 1 conservative amino acid substitution, and a LCDR3 having an amino acid sequence of SEQ ID NO: 19, wherein the LCDR3 comprises at most 1 conservative amino acid substitution.

In some embodiments, the anti-GIP antibody, or antigen-binding fragment thereof, comprises a V_{H} and a V_{L}, wherein the V_{H} comprises an amino acid sequence having at least 90% identity to the amino acid sequence of SEQ ID NO: 29; and the V_{L} comprises an amino acid sequence having at least 90% identity to the amino acid sequence of SEQ ID NO: 30; provided that the V_{H} comprises a HCDR1 having an amino acid sequence of SEQ ID NO: 15, a HCDR2 having an amino acid sequence of SEQ ID NO: 26, and a HCDR3 having an amino acid sequence of SEQ ID NO: 17; and provided that the V_{L} comprises a LCDR1 having an amino acid sequence of SEQ ID NO: 27, a LCDR2 having an amino acid sequence of SEQ ID NO: 28, and a LCDR3 having an amino acid sequence of SEQ ID NO: 19. In some embodiments, the anti-GIP antibody, or antigen-binding fragment thereof, comprises a V_{H} and a V_{L}, wherein the V_{H} comprises an amino acid sequence having at least 95% identity to the amino acid sequence of SEQ ID NO: 29; and the V_{L} comprises an amino acid sequence having at least 95% identity to the amino acid sequence of SEQ ID NO: 30; provided that the V_{H} comprises a HCDR1 having an amino acid sequence of SEQ ID NO: 15, a HCDR2 having an amino acid sequence of SEQ ID NO: 26, and a HCDR3 having an amino acid sequence of SEQ ID NO: 17; and provided that the V_{L} comprises a LCDR1 having an amino acid sequence of SEQ ID NO: 27, a LCDR2 having an amino acid sequence of SEQ ID NO: 28, and a LCDR3 having an amino acid sequence of SEQ ID NO: 19. In some embodiments, the anti-GIP antibody, or antigen-binding fragment thereof, comprises a V_{H} and a V_{L}, wherein the V_{H} comprises an amino acid sequence having at least 98% identity to the amino acid sequence of SEQ ID NO: 29; and the V_{L} comprises an amino acid sequence having at least 98% identity to the amino acid sequence of SEQ ID NO: 30; provided that the V_{H} comprises a HCDR1 having an amino acid sequence of SEQ ID NO: 15, a HCDR2 having an amino acid sequence of SEQ ID NO: 26, and a HCDR3 having an amino acid sequence of SEQ ID NO: 17; and provided that the V_{L} comprises a LCDR1 having an amino acid sequence of SEQ ID NO: 27, a LCDR2 having an amino acid sequence of SEQ ID NO: 28, and a LCDR3 having an amino acid sequence of SEQ ID NO: 19. In some embodiments, the anti-GIP antibody, or antigen-binding fragment thereof, comprises a V_{H} and a V_{L}, wherein the V_{H} comprises an amino acid sequence of SEQ ID NO: 29; and the V_{L} comprises an amino acid sequence of SEQ ID NO: 30.

In some embodiments, the anti-GIP antibody, or antigen-binding fragment thereof, comprises a V_{H} and a V_{L}, wherein the V_{H} comprises an amino acid sequence having at least 85%, 86%, 87%, 88%, 89%, 90%, 91%, 92%, 93%, 94%, 95%, 96%, 97%, 98%, or 99% identity to the amino acid sequence of SEQ ID NO: 32; and the V_{L} comprises an amino acid sequence having at least 85%, 86%, 87%, 88%, 89%, 90%, 91%, 92%, 93%, 94%, 95%, 96%, 97%, 98%, or 99% identity to the amino acid sequence of SEQ ID NO: 33. In some embodiments, the anti-GIP antibody, or antigen-binding fragment thereof, comprises a V_{H} and a V_{L}, wherein the V_{H} comprises an amino acid sequence having at least 85%, 86%, 87%, 88%, 89%, 90%, 91%, 92%, 93%, 94%, 95%, 96%, 97%, 98%, or 99% identity to the amino acid sequence of SEQ ID NO: 32; and the V_{L} comprises an amino acid sequence having at least 85%, 86%, 87%, 88%, 89%, 90%, 91%, 92%, 93%, 94%, 95%, 96%, 97%, 98%, or 99% identity to the amino acid sequence of SEQ ID NO: 33; provided that the V_{H} comprises a HCDR1 having an amino acid sequence of SEQ ID NO: 15, a HCDR2 having an amino acid sequence of SEQ ID NO: 26, and a HCDR3 having an amino acid sequence of SEQ ID NO: 17. In some embodiments, the anti-GIP antibody, or antigen-binding fragment thereof, comprises a V_{H} and a V_{L}, wherein the V_{H} comprises an amino acid sequence having at least 85%, 86%, 87%, 88%, 89%, 90%, 91%, 92%, 93%, 94%, 95%, 96%, 97%, 98%, or 99% identity to the amino acid sequence of SEQ ID NO: 32; and the V_{L} comprises an amino acid sequence having at least 85%, 86%, 87%, 88%, 89%, 90%, 91%, 92%, 93%, 94%, 95%, 96%, 97%, 98%, or 99% identity to the amino acid sequence of SEQ ID NO: 33; provided that the V_{L} comprises a LCDR1 having an amino acid sequence of SEQ ID NO: 27, a LCDR2 having an amino acid sequence of SEQ ID NO: 31, and a LCDR3 having an amino acid sequence of SEQ ID NO: 19. In some embodiments, the anti-GIP antibody, or antigen-binding fragment thereof, comprises a V_{H} and a V_{L}, wherein the V_{H} comprises an amino acid sequence having at least 85%, 86%, 87%, 88%, 89%, 90%, 91%, 92%, 93%, 94%, 95%, 96%, 97%, 98%, or 99% identity to the amino acid sequence of SEQ ID NO: 32; and the V_{L} comprises an amino acid sequence having at least 85%, 86%, 87%, 88%, 89%, 90%, 91%, 92%, 93%, 94%, 95%, 96%, 97%, 98%, or 99% identity to the amino acid sequence of SEQ ID NO: 33; provided that the V_{H} comprises a HCDR1 having an amino acid sequence of SEQ ID NO: 15, a HCDR2 having an amino acid sequence of SEQ ID NO: 26, and a HCDR3 having an amino acid sequence of SEQ ID NO: 17; and provided that the V_{L} comprises a LCDR1 having an amino acid sequence of SEQ ID NO: 27, a LCDR2 having an amino acid sequence of SEQ ID NO: 31, and a LCDR3 having an amino acid sequence of SEQ ID NO: 19. In some embodiments, the CDRs in the V_{H} or V_{L} are as set forth in the combinations provided for herein.

In some embodiments, the anti-GIP antibody, or antigen-binding fragment thereof, comprises a V_{H} and a V_{L}, wherein the V_{H} comprises an amino acid sequence having at least 85%, 86%, 87%, 88%, 89%, 90%, 91%, 92%, 93%, 94%, 95%, 96%, 97%, 98%, or 99% identity to the amino acid sequence of SEQ ID NO: 32; and the V_{L} comprises an amino acid sequence having at least 85%, 86%, 87%, 88%, 89%, 90%, 91%, 92%, 93%, 94%, 95%, 96%, 97%, 98%, or 99% identity to the amino acid sequence of SEQ ID NO: 33; provided that the V_{H} comprises a HCDR1 having an amino acid sequence of SEQ ID NO: 15, wherein the HCDR1 comprises at most 1 conservative amino acid substitution, a HCDR2 having an amino acid sequence of SEQ ID NO: 26, wherein the HCDR2 comprises at most 1 conservative amino acid substitution, and a HCDR3 having an amino acid sequence of SEQ ID NO: 17, wherein the HCDR3 comprises at most 1 conservative amino acid substitution; and provided that the V_{L} comprises a LCDR1 having an amino acid sequence of SEQ ID NO: 27, wherein the LCDR1 comprises at most 1 conservative amino acid substitution, a LCDR2 having an amino acid sequence of SEQ ID NO: 31, wherein the LCDR2 comprises at most 1 conservative amino acid substitution, and a LCDR3 having an amino acid sequence of SEQ ID NO: 19, wherein the LCDR3 comprises at most 1 conservative amino acid substitution.

In some embodiments, the anti-GIP antibody, or antigen-binding fragment thereof, comprises a V_{H} and a V_{L}, wherein the V_{H} comprises an amino acid sequence having at least 90% identity to the amino acid sequence of SEQ ID NO: 32; and the V_{L} comprises an amino acid sequence having at least 90% identity to the amino acid sequence of SEQ ID NO: 33; provided that the V_{H} comprises a HCDR1 having an amino acid sequence of SEQ ID NO: 15, a HCDR2 having an amino acid sequence of SEQ ID NO: 26, and a HCDR3 having an amino acid sequence of SEQ ID NO: 17; and provided that the V_{L} comprises a LCDR1 having an amino acid sequence of SEQ ID NO: 27, a LCDR2 having an amino acid sequence of SEQ ID NO: 31, and a LCDR3 having an amino acid sequence of SEQ ID NO: 19. In some embodiments, the anti-GIP antibody, or antigen-binding fragment thereof, comprises a V_{H} and a V_{L}, wherein the V_{H} comprises an amino acid sequence having at least 95% identity to the amino acid sequence of SEQ ID NO: 32; and the V_{L} comprises an amino acid sequence having at least 95% identity to the amino acid sequence of SEQ ID NO: 33; provided that the V_{H} comprises a HCDR1 having an amino acid sequence of SEQ ID NO: 15, a HCDR2 having an amino acid sequence of SEQ ID NO: 26, and a HCDR3 having an amino acid sequence of SEQ ID NO: 17; and provided that the V_{L} comprises a LCDR1 having an amino acid sequence of SEQ ID NO: 27, a LCDR2 having an amino acid sequence of SEQ ID NO: 31, and a LCDR3 having an amino acid sequence of SEQ ID NO: 19. In some embodiments, the anti-GIP antibody, or antigen-binding fragment thereof, comprises a V_{H} and a V_{L}, wherein the V_{H} comprises an amino acid sequence having at least 98% identity to the amino acid sequence of SEQ ID NO: 32; and the V_{L} comprises an amino acid sequence having at least 98% identity to the amino acid sequence of SEQ ID NO: 33; provided that the V_{H} comprises a HCDR1 having an amino acid sequence of SEQ ID NO: 15, a HCDR2 having an amino acid sequence of SEQ ID NO: 26, and a HCDR3 having an amino acid sequence of SEQ ID NO: 17; and provided that the V_{L} comprises a LCDR1 having an amino acid sequence of SEQ ID NO: 27, a LCDR2 having an amino acid sequence of SEQ ID NO: 31, and a LCDR3 having an amino acid sequence of SEQ ID NO: 19. In some embodiments, the anti-GIP antibody, or antigen-binding fragment thereof, comprises a V_{H} and a V_{L}, wherein the V_{H} comprises an amino acid sequence of SEQ ID NO: 32 and the V_{L} comprises an amino acid sequence of SEQ ID NO: 33.

In some embodiments, the anti-GIP antibody, or antigen-binding fragment thereof, comprises a V_{H} and a V_{L}, wherein the V_{H} comprises an amino acid sequence having at least 85%, 86%, 87%, 88%, 89%, 90%, 91%, 92%, 93%, 94%, 95%, 96%, 97%, 98%, or 99% identity to the amino acid sequence of SEQ ID NO: 35; and the V_{L} comprises an amino acid sequence having at least 85%, 86%, 87%, 88%, 89%, 90%, 91%, 92%, 93%, 94%, 95%, 96%, 97%, 98%, or 99% identity to the amino acid sequence of SEQ ID NO: 36. In some embodiments, the anti-GIP antibody, or antigen-binding fragment thereof, comprises a V_{H} and a V_{L}, wherein the V_{H} comprises an amino acid sequence having at least 85%, 86%, 87%, 88%, 89%, 90%, 91%, 92%, 93%, 94%, 95%, 96%, 97%, 98%, or 99% identity to the amino acid sequence of SEQ ID NO: 35; and the V_{L} comprises an amino acid sequence having at least 85%, 86%, 87%, 88%, 89%, 90%, 91%, 92%, 93%, 94%, 95%, 96%, 97%, 98%, or 99% identity to the amino acid sequence of SEQ ID NO: 36; provided that the V_{H} comprises a HCDR1 having an amino acid sequence of SEQ ID NO: 15, a HCDR2 having an amino acid sequence of SEQ ID NO: 26, and a HCDR3 having an amino acid sequence of SEQ ID NO: 17. In some embodiments, the anti-GIP antibody, or antigen-binding fragment thereof, comprises a V_{H} and a V_{L}, wherein the V_{H} comprises an amino acid sequence having at least 85%, 86%, 87%, 88%, 89%, 90%, 91%, 92%, 93%, 94%, 95%, 96%, 97%, 98%, or 99% identity to the amino acid sequence of SEQ ID NO: 35; and the V_{L} comprises an amino acid sequence having at least 85%, 86%, 87%, 88%, 89%, 90%, 91%, 92%, 93%, 94%, 95%, 96%, 97%, 98%, or 99% identity to the amino acid sequence of SEQ ID NO: 36; provided that the V_{L} comprises a LCDR1 having an amino acid sequence of SEQ ID NO: 27, a LCDR2 having an amino acid sequence of SEQ ID NO: 34, and a LCDR3 having an amino acid sequence of SEQ ID NO: 19. In some embodiments, the anti-GIP antibody, or antigen-binding fragment thereof, comprises a V_{H} and a V_{L}, wherein the V_{H} comprises an amino acid sequence having at least 85%, 86%, 87%, 88%, 89%, 90%, 91%, 92%, 93%, 94%, 95%, 96%, 97%, 98%, or 99% identity to the amino acid sequence of SEQ ID NO: 35; and the V_{L} comprises an amino acid sequence having at least 85%, 86%, 87%, 88%, 89%, 90%, 91%, 92%, 93%, 94%, 95%, 96%, 97%, 98%, or 99% identity to the amino acid sequence of SEQ ID NO: 36; provided that the V_{H} comprises a HCDR1 having an amino acid sequence of SEQ ID NO: 15, a HCDR2 having an amino acid sequence of SEQ ID NO: 26, and a HCDR3 having an amino acid sequence of SEQ ID NO: 17; and provided that the V_{L} comprises a LCDR1 having an amino acid sequence of SEQ ID NO: 27, a LCDR2 having an amino acid sequence of SEQ ID NO: 34, and a LCDR3 having an amino acid sequence of SEQ ID NO: 19. In some embodiments, the CDRs in the V_{H} or V_{L} are as set forth in the combinations provided for herein.

In some embodiments, the anti-GIP antibody, or antigen-binding fragment thereof, comprises a V_{H} and a V_{L}, wherein the V_{H} comprises an amino acid sequence having at least 85%, 86%, 87%, 88%, 89%, 90%, 91%, 92%, 93%, 94%, 95%, 96%, 97%, 98%, or 99% identity to the amino acid sequence of SEQ ID NO: 35; and the V_{L} comprises an amino acid sequence having at least 85%, 86%, 87%, 88%, 89%, 90%, 91%, 92%, 93%, 94%, 95%, 96%, 97%, 98%, or 99% identity to the amino acid sequence of SEQ ID NO: 36; provided that the V_{H} comprises a HCDR1 having an amino acid sequence of SEQ ID NO: 15, wherein the HCDR1 comprises at most 1 conservative amino acid substitution, a HCDR2 having an amino acid sequence of SEQ ID NO: 26, wherein the HCDR2 comprises at most 1 conservative amino acid substitution, and a HCDR3 having an amino acid sequence of SEQ ID NO: 17, wherein the HCDR3 comprises at most 1 conservative amino acid substitution; and provided that the V_{L} comprises a LCDR1 having an amino acid sequence of SEQ ID NO: 27, wherein the LCDR1 comprises at most 1 conservative amino acid substitution, a LCDR2 having an amino acid sequence of SEQ ID NO: 34, wherein the LCDR2 comprises at most 1 conservative amino acid substitution, and a LCDR3 having an amino acid sequence of SEQ ID NO: 19, wherein the LCDR3 comprises at most 1 conservative amino acid substitution.

In some embodiments, the anti-GIP antibody, or antigen-binding fragment thereof, comprises a V_{H} and a V_{L}, wherein the V_{H} comprises an amino acid sequence having at least 90% identity to the amino acid sequence of SEQ ID NO: 35; and the V_{L} comprises an amino acid sequence having at least 90% identity to the amino acid sequence of SEQ ID NO: 36; provided that the V_{H} comprises a HCDR1 having an amino acid sequence of SEQ ID NO: 15, a HCDR2 having an amino acid sequence of SEQ ID NO: 26, and a HCDR3 having an amino acid sequence of SEQ ID NO: 17; and provided that the V_{L} comprises a LCDR1 having an amino acid sequence of SEQ ID NO: 27, a LCDR2 having an amino acid sequence of SEQ ID NO: 34, and a LCDR3 having an amino acid sequence of SEQ ID NO: 19. In some embodiments, the anti-GIP antibody, or antigen-binding fragment thereof, comprises a V_{H} and a V_{L}, wherein the V_{H} comprises an amino acid sequence having at least 95% identity to the amino acid sequence of SEQ ID NO: 35; and the V_{L} comprises an amino acid sequence having at least 95% identity to the amino acid sequence of SEQ ID NO: 36; provided that the V_{H} comprises a HCDR1 having an amino acid sequence of SEQ ID NO: 15, a HCDR2 having an amino acid sequence of SEQ ID NO: 26, and a HCDR3 having an amino acid sequence of SEQ ID NO: 17; and provided that the V_{L} comprises a LCDR1 having an amino acid sequence of SEQ ID NO: 27, a LCDR2 having an amino acid sequence of SEQ ID NO: 34, and a LCDR3 having an amino acid sequence of SEQ ID NO: 19. In some embodiments, the anti-GIP antibody, or antigen-binding fragment thereof, comprises a V_{H} and a V_{L}, wherein the V_{H} comprises an amino acid sequence having at least 98% identity to the amino acid sequence of SEQ ID NO: 35; and the V_{L} comprises an amino acid sequence having at least 98% identity to the amino acid sequence of SEQ ID NO: 36; provided that the V_{H} comprises a HCDR1 having an amino acid sequence of SEQ ID NO: 15, a HCDR2 having an amino acid sequence of SEQ ID NO: 26, and a HCDR3 having an amino acid sequence of SEQ ID NO: 17; and provided that the V_{L} comprises a LCDR1 having an amino acid sequence of SEQ ID NO: 27, a LCDR2 having an amino acid sequence of SEQ ID NO: 34, and a LCDR3 having an amino acid sequence of SEQ ID NO: 19. In some embodiments, the anti-GIP antibody, or antigen-binding fragment thereof, comprises a V_{H} and a V_{L}, wherein the V_{H} comprises an amino acid sequence of SEQ ID NO: 35 and the V_{L} comprises an amino acid sequence of SEQ ID NO: 36.

In some embodiments, the anti-GIP antibody, or antigen binding fragment thereof, comprises a heavy chain (HC) and a light chain (LC), wherein the HC and the LC comprise one of the following sequences, or a variant thereof:

| SEQ ID | AB | |
|---|---|---|
| NO | ID NO | Sequence |
| SEQ ID | GIP AB1- | QVQLVQSGAEVKKPGATVKISCKASG |
| NO: 37 | HC | FNIRDYYLHWVRQAPGKGLEWMGWID |
| | | PENGDTEYAPKFQGRVTITADTSTNT |
| | | AYMELSSLRSEDTAVYYCNVYGIYFM |
| | | DYWGQGTMVTVSSASTKGPSVFPLAP |
| | | SSKSTSGGTAALGCLVKDYFPEPVTV |
| | | SWNSGALTSGVHTFPAVLQSSGLYSL |
| | | SSVVTVPSSSLGTQTYICNVNHKPSN |
| | | TKVDKKVEPKSCDKTHTCPPCPAPEL |
| | | LGGPSVFLFPPKPKDTLMISRTPEVT |
| | | CVVVDVSHEDPEVKFNWYVDGVEVHN |
| | | AKTKPREEQYNSTYRVVSVLTVLHQD |
| | | WLNGKEYKCKVSNKALPAPIEKTISK |
| | | AKGQPREPQVYTLPPSRDELTKNQVS |
| | | LTCLVKGFYPSDIAVEWESNGQPENN |
| | | YKTTPPVLDSDGSFFLYSKLTVDKSR |
| | | WQQGNVFSCSVMHEALHNHYTQKSLS |
| | | LSPGK |
| SEQ ID | GIP AB1- | DIQLTQSPSSVSASVGDRVTITCRAS |
| NO: 144 | LC | SSISSNSLHWYQQKPGKAPKLLIYRT |
| | | SNLQSGVPSRFSGSGSGTDYTLTISS |
| | | LQPEDFATYYCQQGSSFPRMLTFGQG |
| | | TKLEIKRTVAAPSVFIFPPSDEQLKS |
| | | GTASVVCLLNNFYPREAKVQWKVDNA |
| | | LQSGNSQESVTEQDSKDSTYSLSSTL |
| | | TLSKADYEKHKVYACEVTHQGLSSPV |
| | | TKSFNRGEC |

In some embodiments, the anti-GIP antibody, or antigen binding fragment thereof, comprises a HC comprising an amino acid sequence having at least 90%, 91%, 92%, 93%, 94%, 95%, 96%, 97%, 98%, or 99% identity to the amino acid sequence of SEQ ID NO: 37 and a LC comprising an amino acid sequence having at least 90%, 91%, 92%, 93%, 94%, 95%, 96%, 97%, 98%, or 99% identity to the amino acid sequence of SEQ ID NO: 144. In some embodiments, the anti-GIP antibody, or antigen binding fragment thereof, comprises a HC comprising an amino acid sequence having at least 90% identity to the amino acid sequence of SEQ ID NO: 37 and a LC comprising an amino acid sequence having at least 90% identity to the amino acid sequence of SEQ ID NO: 144. In some embodiments, the anti-GIP antibody, or antigen binding fragment thereof, comprises a HC comprising an amino acid sequence having at least 95% identity to the amino acid sequence of SEQ ID NO: 37 and a LC comprising an amino acid sequence having at least 95% identity to the amino acid sequence of SEQ ID NO: 144. In some embodiments, the anti-GIP antibody, or antigen binding fragment thereof, comprises a HC comprising an amino acid sequence having at least 98% identity to the amino acid sequence of SEQ ID NO: 37 and a LC comprising an amino acid sequence having at least 98% identity to the amino acid sequence of SEQ ID NO: 144. In some embodiments, the anti-GIP antibody, or antigen binding fragment thereof, comprises a HC comprising the amino acid sequence of SEQ ID NO: 37 and a LC comprising the amino acid sequence of SEQ ID NO: 144.

In some embodiments, the anti-GIP antibody, or antigen binding fragment thereof, comprises a HC comprising an amino acid sequence having at least 90%, 91%, 92%, 93%, 94%, 95%, 96%, 97%, 98%, or 99% identity to the amino acid sequence of SEQ ID NO: 37 and a LC comprising an amino acid sequence having at least 90%, 91%, 92%, 93%, 94%, 95%, 96%, 97%, 98%, or 99% identity to the amino acid sequence of SEQ ID NO: 144, provided that the HC comprises an HCDR1 comprising the amino acid sequence of SEQ ID NO: 15, an HCDR2 comprising the amino acid sequence of SEQ ID NO: 26, and an HCDR3 comprising the amino acid sequence of SEQ ID NO: 17, and provided that the LC comprising a LCDR1 comprising the amino acid sequence of SEQ ID NO: 27, an LCDR2 comprising the amino acid sequence of SEQ ID NO: 28, and a LCDR3 comprising the amino acid sequence of SEQ ID NO: 19. In some embodiments, the anti-GIP antibody, or antigen binding fragment thereof, comprises a HC comprising an amino acid sequence having at least 90% identity to the amino acid sequence of SEQ ID NO: 37 and a LC comprising an amino acid sequence having at least 90% identity to the amino acid sequence of SEQ ID NO: 144, provided that the HC comprises an HCDR1 comprising the amino acid sequence of SEQ ID NO: 15, an HCDR2 comprising the amino acid sequence of SEQ ID NO: 26, and an HCDR3 comprising the amino acid sequence of SEQ ID NO: 17, and provided that the LC comprising a LCDR1 comprising the amino acid sequence of SEQ ID NO: 27, an LCDR2 comprising the amino acid sequence of SEQ ID NO: 28, and a LCDR3 comprising the amino acid sequence of SEQ ID NO: 19. In some embodiments, the anti-GIP antibody, or antigen binding fragment thereof, comprises a HC comprising an amino acid sequence having at least 95% identity to the amino acid sequence of SEQ ID NO: 37 and a LC comprising an amino acid sequence having at least 95% identity to the amino acid sequence of SEQ ID NO: 144, provided that the HC comprises an HCDR1 comprising the amino acid sequence of SEQ ID NO: 15, an HCDR2 comprising the amino acid sequence of SEQ ID NO: 26, and an HCDR3 comprising the amino acid sequence of SEQ ID NO: 17, and provided that the LC comprising a LCDR1 comprising the amino acid sequence of SEQ ID NO: 27, an LCDR2 comprising the amino acid sequence of SEQ ID NO: 28, and a LCDR3 comprising the amino acid sequence of SEQ ID NO: 19. In some embodiments, the anti-GIP antibody, or antigen binding fragment thereof, comprises a HC comprising an amino acid sequence having at least 98% identity to the amino acid sequence of SEQ ID NO: 37 and a LC comprising an amino acid sequence having at least 98% identity to the amino acid sequence of SEQ ID NO: 144, provided that the HC comprises an HCDR1 comprising the amino acid sequence of SEQ ID NO: 15, an HCDR2 comprising the amino acid sequence of SEQ ID NO: 26, and an HCDR3 comprising the amino acid sequence of SEQ ID NO: 17, and provided that the LC comprising a LCDR1 comprising the amino acid sequence of SEQ ID NO: 27, an LCDR2 comprising the amino acid sequence of SEQ ID NO: 28, and a LCDR3 comprising the amino acid sequence of SEQ ID NO: 19. In some embodiments, the anti-GIP antibody, or antigen binding fragment thereof, comprises a HC comprising the amino acid sequence of SEQ ID NO: 37 and a LC comprising the amino acid sequence of SEQ ID NO: 144.

### Biologically Active Peptides

In some embodiments, an anti-GIP antibody, or antigen-binding fragment thereof, as provided for herein, is conjugated to a biologically active peptide. In some embodiments, an anti-GIP antibody, or antigen-binding fragment thereof, as provided for herein, is not conjugated to a biologically active peptide. In some embodiments, an anti-GIP antibody, or antigen-binding fragment thereof, as provided for herein, is administered with a biologically active peptide. In some embodiments, an anti-GIP antibody, or antigen-binding fragment thereof, as provided for herein, is co-administered with a biologically active peptide. In some embodiments, an anti-GIP antibody, or antigen-binding fragment thereof, as provided for herein, is co-formulated with a biologically active peptide.

In some embodiments, the biologically active peptide comprises a glucagon like peptide 1 receptor (GLP-1R) agonist, a glucagon like peptide 2 receptor (GLP-2R) agonist, a glucagon receptor agonist. GLP-1R agonists are known in the art, and any such GLP-1R agonist is within the scope of the present disclosure. Non-limiting examples of GLP-1R agonists include liraglutide, albiglutide, taspoglutide, semaglutide, LY2428757, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91, SEQ ID NO: 92, SEQ ID NO: 93, SEQ ID NO: 94, SEQ ID NO: 95, SEQ ID NO: 96, SEQ ID NO: 97, SEQ ID NO: 98, SEQ ID NO: 99, SEQ ID NO: 100, SEQ ID NO: 101, SEQ ID NO: 102, SEQ ID NO: 103, SEQ ID NO: 104, SEQ ID NO: 105, SEQ ID NO: 106, SEQ ID NO: 107, SEQ ID NO: 108, SEQ ID NO: 109, SEQ ID NO: 110, SEQ ID NO: 111, SEQ ID NO: 112, SEQ ID NO: 113, SEQ ID NO: 114, SEQ ID NO: 115, SEQ ID NO: 116, SEQ ID NO: 117, SEQ ID NO: 118, SEQ ID NO: 119, SEQ ID NO: 120, and SEQ ID NO: 121. The sequences of SEQ ID NOs: 38-121 are as provided in Table 6 below:

TABLE 6
Sequences of exemplary GLP-1R agonists

| SEQ | | | |
|---|---|---|---|
| ID | | | Variable identity |
| NO | Sequence | Alternate Nomenclature | (if applicable) |
| 38 | HGEGTFTSDLSKQMEEEAVRLFIE | Exendin-4 | |
| | WLKNGGPSSGAPPPS | | |
| 39 | HSDGTFTSDLSKQMEEEAVRLFIE | Exendin-3 | |
| | WLKNGGPSSGAPPPS | | |
| 40 | HGEGTFTSDLSKQLEEEAVRLFIE | Leu¹⁴-exendin-4 | |
| | WLKNGGPSSGAPPPS | | |
| 41 | HGEGTFTSDLSKQLEEEAVRLFIE | Leu¹⁴, Phe²⁵-exendin-4 | |
| | FLKNGGPSSGAPPPS | | |
| 42 | HGEGTFTSDLSKQLEEEAARLFIE | Leu¹⁴, Ala¹⁹, Phe²⁵- | |
| | FLKNGGPSSGAPPPS | exendin-4 | |
| 43 | HGEGTFTSDLSKQMEEEAVRLFIE | exendin-4(1-30) | |
| | WLKNGG | | |
| 44 | HGEGTFTSDLSKQLEEEAVRLFIE | Leu¹⁴-exendin-4(1-30) | |
| | WLKNGG | | |
| 45 | HGEGTFTSDLSKQLEEEAVRLFIE | Leu¹⁴, Phe²⁵-exendin- | |
| | FLKNGG | 4(1-30) | |
| 46 | HGEGTFTSDLSKQLEEEAARLFIE | Leu¹⁴, Ala¹⁹, Phe²⁵_ | |
| | FLKNGG | exendin-4(1-30) | |
| 47 | HGEGTFTSDLSKQMEEEAVRLFIE | exendin-4(1-28) | |
| | WLKN | | |
| 48 | HGEGTFTSDLSKQLEEEAVRLFIE | Leu¹⁴-exendin-4(1-28) | |
| | WLKN | | |
| 49 | HGEGTFTSDLSKQLEEEAVRLFIE | Leu¹⁴, Phe²⁵-exendin- | |
| | FLKN | 4(1-28) | |
| 50 | HGEGTFTSDLSKQLEEEAARLFIE | Leu¹⁴, Ala¹⁹, Phe²⁵- | |
| | FLKN | exendin-4(1-28) | |
| 51 | HGEGTFTSDLSKQLEEKAAKEFIE | Leu¹⁴, Lys^{17,20}, Ala¹⁹, Glu²¹, | |
| | FLKQGGPSSGAPPPS | Phe⁵, Gln²⁸-exendin-4 | |
| 52 | HGEGTFTSDLSKQLEEKAAKEFIE | Leu¹⁴, Lys^{17,20}, Ala¹⁹, Glu²¹, | |
| | WLKQGGPSSGAPPPS | Gln²⁸-exendin-4 | |
| 53 | HGEGTFTSDLSKQGEEEAVRLFIE | octylGly¹⁴, Gln²⁸- | |
| | WLKQGGPSSGAPPPS | exendin-4 | |
| 54 | HGEGTFTSDLSKQLEEEAVRLFIE | Leu¹⁴, Gln²⁸, octylGly³⁴- | |
| | WLKQGGPSSGAPPPS | exendin-4 | |
| 55 | HGEFTFTSDLSKQLEEEAVRLFIE | Phe⁴, Leu¹⁴, Gln²⁸, Lys³³, | |
| | WLKQGGPSKEIIS | Glu³⁴, Ile^{35,36}, Ser³⁷- | |
| | | exendin-4(1-37) | |
| 56 | HGEFTFTSDLSKQLEEKAAKEFIE | Phe⁴, Leu¹⁴, Lys^{17,20}, | |
| | WLKQGGPSSGAPPPS | Ala19, Glu21, Gln28- | |
| | | exendin-4 | |
| 57 | HGEGTFTSDLVKILEAEAVRKFIE | Val¹¹, Ile¹³, Leu¹⁴, Ala¹⁶, | |
| | FLKNGGPSSGAPPPS | Lys²¹, Phe²⁵-exendin-4 | |
| 58 | HGEGTFTSDLSKQMEEEAVRLFIE | exendin-4-Lys⁴⁰ | |
| | WLKNGGPSSGAPPPSK | | |
| 59 | HAEGTFTSDVSSYLEGQAAKEFI | GLP-1(7-37) | |
| | AWLVKGRG | | |
| 60 | HAEGTFTSDVSSYLEGQAAKEFI | GLP-1(7-36)-NH₂ | |
| | AWLVKGR | | |
| 61 | HXEGTFTSDVSSYLEGQAAREFI | Aib^{8,35}, Arg^{26,34}, Phe³¹- | 2-Aminoisobutyric |
| | AFLVRXR | GLP-1(7-36) | acid |
| 62 | HX₂EGTFTSDVSSYLEXXAAKEFI | HXaa₈EGTFTSDVSSY | Xaa8 = A, V, or G; |
| | XWLXXGXX | LEXaa₂₂Xaa₂₃AAKEF | Xaa22 = G, K, or E; |
| | | IXaa₃₀WLXaa₃₃Xaa₃₄ | Xaa23 = Q or K; |
| | | GXaa₃₆Xaa₃₇ | Xaa30 = A or E; |
| | | | Xaa33 = V or K |
| | | | Xaa34 = K, N, or R |
| | | | Xaa36 = R or G |
| | | | Xaa37 = G, H, P, or |
| | | | absent |
| 63 | HAEGTFTSDVSSYLEGQAAKEFI | Arg³⁴-GLP-1(7-37) | |
| | AWLVRGRG | | |
| 64 | HAEGTFTSDVSSYLEGQAAKEFIE | Glu³⁰-GLP-1(7-37) | |
| | WLVKGRG | | |
| 65 | HAEGTFTSDVSSYLEKQAAKEFI | Lys²²-GLP-1(7-37) | |
| | AWLVKGRG | | |
| 66 | HGEGTFTSDVSSYLEEQAAKEFIA | Gly^{8,36}, Glu²²-GLP- | |
| | WLVKGGG | 1(7-37) | |
| 67 | HVEGTFTSDVSSYLEEQAAKEFIA | Val⁸, Glu²², Gly³⁶-GLP- | |
| | WLVKGGG | 1(7-37) | |
| 68 | HGEGTFTSDVSSYLEEQAAKEFIA | Gly^{8,36}, Glu²², Lys³³, Asn³⁴- | |
| | WLKNGGG | GLP-(7-37) | |
| 69 | HVEGTFTSDVSSYLEEQAAKEFIA | Val⁸, Glu²², Lys³³, Asn³⁴, | |
| | WLKNGGG | Gly³⁶-GLP-1(7-37) | |
| 70 | HGEGTFTSDVSSYLEEQAAKEFIA | Gly^{8,36}, Glu²², Pro³⁷-GLP- | |
| | WLVKGGP | 1(7-37) | |
| 71 | HVEGTFTSDVSSYLEEQAAKEFIA | Val⁸, Glu²², Gly³⁶, Pro³⁷- | |
| | WLVKGGP | GLP-1(7-37) | |
| 72 | HGEGTFTSDVSSYLEEQAAKEFIA | Gly^{8,36}, Glu²², Lys³³, Asn³⁴, | |
| | WLKNGGP | Pro³⁷-GLP-1(7-37) | |
| 73 | HVEGTFTSDVSSYLEEQAAKEFIA | Val⁸, Glu²², Lys³³, Asn³⁴, | |
| | WLKNGGP | Gly³⁶, Pro³⁷-GLP-1(7-37) | |
| 74 | HGEGTFTSDVSSYLEEQAAKEFIA | Gly^{8,36}, Glu²²-GLP- | |
| | WLVKGG | 1(7-36) | |
| 75 | HVEGTFTSDVSSYLEEQAAKEFIA | Val⁸, Glu²², Gly³⁶-GLP- | |
| | WLVKGG | 1(7-36) | |
| 76 | HVEGTFTSDVSSYLEEQAAKEFIA | Val⁸, Glu²², Asn³⁴, Gly³⁶- | |
| | WLVNGG | GLP-1(7-36) | |
| 77 | HGEGTFTSDVSSYLEEQAAKEFIA | Gly^{8,36}, Glu²², Asn³⁴-GLP- | |
| | WLVNGG | 1(7-36) | |
| 78 | HXEGTFTSDVSSYLEXQAAKEFI | GLP-1 Analog | Xaa2 = D-Ala, Gly, |
| | AWLXKGGPSSGAPPPCC | | Val, Leu, Ile, Ser or |
| | | | Thr; |
| | | | Xaa16 = Gly, Glu, Asp |
| | | | or Lys; |
| | | | Xaa27 = Val or Ile |
| 79 | HVEGTFTSDVSSYLEEQAAKEFIA | GLP-1 Analog | |
| | WLIKGGPSSGAPPPCC | | |
| 80 | HGEGTFTSDLSKQMEEEAVKLFIE | [N^{e}-(17- | |
| | WLKNGGPSSGAPPPS | carboxyheptadecanoic | |
| | | acid)Lys²⁰]exendin-4- | |
| | | NH₂ | |
| 81 | HGEGTFTSDLSKQMEEEAVRLFIE | [N^{e}-(17- | |
| | WLKNGGPKSGAPPPS | carboxyheptadecanoyl) | |
| | | Lys³²]exendin-4-NH₂ | |
| 82 | GEGTFTSDLSKQMEEEAVKLFIE | [desamino-His¹, N^{e}-(17- | |
| | WLKNGGPSSGAPPPS | carboxyheptadecanoyl) | |
| | | Lys²⁰]exendin-4-NH₂ | |
| 83 | HGEGTFTSDLSRQXEEEAVRLFIE | [Arg^{12,27}, NLe¹⁴, N^{e}-(17- | Xaa14 = Norleucine |
| | WLRNGGPKSGAPPPS | carboxyheptadecanoyl) | |
| | | Lys³²]exendin-4-NH₂ | |
| 84 | HGEGTFTSDLSKQMEEEAVKLFIE | [N^{e}-(19-carboxy- | |
| | WLKNGGPSSGAPPPS | nonadecanoylamino) | |
| | | Lys²⁰]-exendin-4-NH₂ | |
| 85 | HGEGTFTSDLSKQMEEEAVKLFIE | [N^{e}-(15- | |
| | WLKNGGPSSGAPPPS | carboxypentadecanoylamino) | |
| | | Lys²⁰]-exendin-4- | |
| | | NH₂ | |
| 86 | HGEGTFTSDLSKQMEEEAVKLFIE | [N^{e}-(13- | |
| | WLKNGGPSSGAPPPS | carboxytridecanoylamino) | |
| | | Lys²⁰]-exendin-4-NH₂ | |
| 87 | HGEGTFTSDLSKQMEEEAVKLFIE | [N^{e}-(11-carboxy- | |
| | WLKNGGPSSGAPPPS | undecanoyl- | |
| | | amino)Lys²⁰]-exendin-4- | |
| | | NH₂ | |
| 88 | HGEGTFTSDLSKQMEEEAVRLFIE | exendin-4-Lys⁴⁰(e- | |
| | WLKNGGPSSGAPPPSK | MPA)-NH₂ | |
| 89 | HGEGTFTSDLSKQMEEEAVRLFIE | exendin-4-Lys⁴⁰ (e- | |
| | WLKNGGPSSGAPPPSK | AEEA-AEEA-MPA)- | |
| | | NH₂ | |
| 90 | HGEGTFTSDLSKQMEEEAVRLFIE | exendin-4-Lys⁴⁰ (e- | |
| | WLKNGGPSSGAPPPSK | AEEA-MPA)-NH₂ | |
| 91 | HGEGTFTSDLSKQMEEEAVRLFIE | exendin-4-Lys⁴⁰ (e- | |
| | WLKNGGPSSGAPPPSK | MPA)-albumin | |
| 92 | HGEGTFTSDLSKQMEEEAVRLFIE | exendin-4-Lys⁴⁰ (e- | |
| | WLKNGGPSSGAPPPSK | AEEA-AEEA-MPA)- | |
| | | albumin | |
| 93 | HGEGTFTSDLSKQMEEEAVRLFIE | exendin-4-Lys⁴⁰ (e- | |
| | WLKNGGPSSGAPPPSK | AEEA-MPA)-albumin | |
| 94 | AEGTFTSDVSSYLEGQAAREFIA | desamino-His⁷ | |
| | WLVKGRG | Arg²⁶, Lys³⁴(N^{e}-(y- | |
| | | Glu(N-α-hexadecanoyl)))- | |
| | | GLP-1(7-37) | |
| 95 | AEGTFTSDVSSYLEGQAAREFIA | desamino-His⁷ | |
| | WLVKGRG | Arg²⁶, Lys³⁴(N^{e}-octanoyl))- | |
| | | GLP-1(7-37) | |
| 96 | HAEGTFTSDVSSYLEGQAAREFI | Arg²⁶, Lys³⁴(N^{e}-(ω- | |
| | AWLVRGRGK | carboxypentadecanoyl))- | |
| | | GLP-1(7-38) | |
| 97 | HAEGTFTSDVSSYLEGQAAREFI | Arg^{26,34}, Lys³⁶(N^{e}-(y- | |
| | AWLVRGRGK | Glu(N-α- | |
| | | hexadecanoyl)))-GLP- | |
| | | 1(7-36) | |
| 98 | HXEGTFTSDVSSYLEGQAAKEFI | [Aib⁸, Lys³⁷]GLP- | 2-Aminoisobutyric |
| | AWLVKGRK | 1_(7-37) | acid |
| 99 | HXEGTFTSDVSSYLEGQAAKEFI | [Aib⁸, Lys²⁶]GLP- | 2-Aminoisobutyric |
| | AWLVKGKG | 1_(7-37) | acid |
| 100 | HXEGTFTSDVSSYLEXQAAKEFI | [Aib^{8,22}, Lys³⁶]GLP- | 2-Aminoisobutyric |
| | AWLVKGK | 1_(7-36)Amide | acid |
| 101 | HXEGTFTSDVSSYLEXQAAKEFI | [Aib^{8,22}, BLeu³², Lys³⁶] | Xaa2 and 16 = 2- |
| | AWXVKGK | GLP-1_(7-36)Amide | Aminoisobutyric acid; |
| | | | Xaa26 = beta leucine |
| 102 | HXEGTFTSDVSSYLEXQAAKEFI | [Aib^{8,22}, Lys³⁷]GLP- | |
| | AWLVKGRK | 1_(7-37)Amide | |
| 103 | HXEGTFTSDVSSYLEXQAAKEFI | [Aib^{8,22}, BLeu³², Lys³⁷] | Xaa2 and 16 = 2- |
| | AWXVKGRK | GLP-1_(7-37)Amide | Aminoisobutyric acid; |
| | | | Xaa26 = beta leucine |
| 104 | HXEGTFTSDVSSYLEXQAAKEFI | [Aib^{8,22}, aMeLeu³², Lys³⁷] | Xaa2 and 16 = 2- |
| | AWXVKGRK | GLP-1_(7-37)Amide | Aminoisobutyric acid; |
| | | | Xaa26 = alpha methyl |
| | | | leucine |
| 105 | HXEGTXTSDVSSYLEXQAAKEFI | [Aib^{8,22}, AMEF¹², Lys³⁷] | Xaa2 and 16 = 2- |
| | AWLVKGRK | GLP-1_(7-37)-Amide | Aminoisobutyric acid; |
| | | | Xaa6 = AMEF |
| 106 | HXEGTFTSDXSSYLEXQAAKEFI | [Aib^{8,22}, BLeu¹⁶, Lys³⁷] | Xaa2 and 16 = 2- |
| | AWLVKGRK | GLP-1_(7-37)Amide | Aminoisobutyric acid; |
| | | | Xaa10 = beta leucine |
| 107 | HXEGTFTSDVSSYLEXQAAKEFI | [Aib^{8,22}, Gly³⁶, Lys³⁷] | Xaa2 and 16 = 2- |
| | AWLVKGGK | GLP-1_(7-37)Amide | Aminoisobutyric acid |
| 108 | HXEGTFTSDVSSYLEXQAAKEFI | [Aib^{8,22}, Lys^{32,37}, | Xaa2 and 16 = 2- |
| | AWLKNGGK | Asn³⁴, Gly³⁶]GLP- | Aminoisobutyric acid |
| | | 1_(7-37)Amide | |
| 109 | HXEGTFTSDVSSYLEXQAAKEFI | [Aib^{8,22}, Lys³³, | Xaa2 and 16 = 2- |
| | AWLKNGGX | Asn³⁴, Gly³⁶, | Aminoisobutyric acid; |
| | | Aeea³⁷]GLP-1_(7-37)- | Xaa31 = Aeea |
| | | Acea-Lys-Amide | (Aminoethylethanolamine) |
| 110 | HXEGTFTSDVSSYLEXQAAKEFI | [Aib^{8,22}, Gly³⁶]GLP- | Xaa2 and 16 = 2- |
| | AWLVKGGG | 1_(7-37)-Amide | Aminoisobutyric acid |
| 111 | HXEGTFTSDVSSYLEGQAAKEFI | cyclo[E²³- | Xaa2 = 2- |
| | AWLVKGGG | K²⁷][Aib⁸; Gly¹⁶]GLP- | Aminoisobutyric acid |
| | | 1(7-37) | |
| 112 | HXEGTFTSDVSSYLEGQAAKEFI | cyclo[E-K²⁶][Aib⁸; Gly³⁶; | Xaa2 = 2- |
| | AWLVKGGK | Lys³⁷]GLP-1(7-37) | Aminoisobutyric acid |
| 113 | HXEGTFTSDVSSYLEXEAVRLFIE | [Aib^{8,22}]GLP-1(7-22)- | Xaa2 and 16 = 2- |
| | WLKNGGPSSGAPPPS | Ex4(17-39) | Aminoisobutyric acid |
| 114 | HGEGTFTSDVSSYLEEQAAKEFIA | [Gly^{8,36}; Glu²²]GLP-1 | |
| | WLVKGGG | (7-37) | |
| 115 | HXEGTFTSDVSSYLEEQAAKEFIA | [Aib⁸; Glu²²; Gly³⁶]GLP- | Xaa2 = 2- |
| | WLVKGGG | 1(7-37)Amide | Aminoisobutyric acid |
| 116 | HXEGTFTSDYSSYLEEQAAKEFIA | [Aib⁸; Tyr¹⁶; Glu²²; Gly³⁶] | Xaa2 = 2- |
| | WLVKGGG | GLP-1_(7-37) | Aminoisobutyric acid |
| 117 | HXEGTFTSDVSKYLEEEAVRLFIE | [Aib⁸, Lys^{18,33}, Glu^{22,23,30}, | Xaa2 = 2- |
| | WLKNGGG | Val²⁵, Arg²⁶, Leu²⁷, Asn³⁴, | Aminoisobutyric acid |
| | | Gly³⁶]GLP-1_(7-37) | |
| 118 | HXEGTFTSDVSKYLEEEAAKLFIE | [Aib⁸, Lys^{18,33}, | Xaa2 = 2- |
| | WLKNGGG | Glu^{22,23,30}, Leu²⁷, Asn³⁴, | Aminoisobutyric acid |
| | | Gly³⁶]GLP-1_(7-37) | |
| 119 | HXEGTFTSDVSKYLEEEAAKLFIE | [Aib⁸, Lys¹⁸, | Xaa2 = 2- |
| | WLVKGGG | Glu^{22,23,30}, Leu²⁷, | Aminoisobutyric acid |
| | | Gly³⁶]GLP-1_(7-37) | |
| 120 | HXIGTFTSDVSSYLEXQAAKEFIA | [Aib²², Ile⁹, Gly³⁶]GLP- | Xaa2 and 16 = 2- |
| | WLVKGG | 1_(7-36) | Aminoisobutyric acid |
| 121 | HXEGTFTSEVSSYLEXQAAKEFIA | [Aib^{8,22}, Glu¹⁵, | Xaa2 and 16 = 2- |
| | WLVKGG | Gly³⁶]GLP-1_(7-36) | Aminoisobutyric acid |

In some embodiments, the GLP-1R agonist is liraglutide. In some embodiments, the GLP-1R agonist is albiglutide. In some embodiments, the GLP-1R agonist is taspoglutide. In some embodiments, the GLP-1R agonist is semaglutide. In some embodiments, the GLP-1R agonist is LY2428757. In some embodiments, the GLP-1R agonist is SEQ ID NO: 38. In some embodiments, the GLP-1R agonist is SEQ ID NO: 39. In some embodiments, the GLP-1R agonist is SEQ ID NO: 40. In some embodiments, the GLP-1R agonist is SEQ ID NO: 41. In some embodiments, the GLP-1R agonist is SEQ ID NO: 42. In some embodiments, the GLP-1R agonist is SEQ ID NO: 43. In some embodiments, the GLP-1R agonist is SEQ ID NO: 44. In some embodiments, the GLP-1R agonist is SEQ ID NO: 45. In some embodiments, the GLP-1R agonist is SEQ ID NO: 46. In some embodiments, the GLP-1R agonist is SEQ ID NO: 47. In some embodiments, the GLP-1R agonist is SEQ ID NO: 48. In some embodiments, the GLP-1R agonist is SEQ ID NO: 49. In some embodiments, the GLP-1R agonist is SEQ ID NO: 50. In some embodiments, the GLP-1R agonist is SEQ ID NO: 51. In some embodiments, the GLP-1R agonist is SEQ ID NO: 52. In some embodiments, the GLP-1R agonist is SEQ ID NO: 53. In some embodiments, the GLP-1R agonist is SEQ ID NO: 54. In some embodiments, the GLP-1R agonist is SEQ ID NO: 55. In some embodiments, the GLP-1R agonist is SEQ ID NO: 56. In some embodiments, the GLP-1R agonist is SEQ ID NO: 57. In some embodiments, the GLP-1R agonist is SEQ ID NO: 58. In some embodiments, the GLP-1R agonist is SEQ ID NO: 59. In some embodiments, the GLP-1R agonist is SEQ ID NO: 60. In some embodiments, the GLP-1R agonist is SEQ ID NO: 61. In some embodiments, the GLP-1R agonist is SEQ ID NO: 62. In some embodiments, the GLP-1R agonist is SEQ ID NO: 63. In some embodiments, the GLP-1R agonist is SEQ ID NO: 64. In some embodiments, the GLP-1R agonist is SEQ ID NO: 65. In some embodiments, the GLP-1R agonist is SEQ ID NO: 66. In some embodiments, the GLP-1R agonist is SEQ ID NO: 67. In some embodiments, the GLP-1R agonist is SEQ ID NO: 68. In some embodiments, the GLP-1R agonist is SEQ ID NO: 69. In some embodiments, the GLP-1R agonist is SEQ ID NO: 70. In some embodiments, the GLP-1R agonist is SEQ ID NO: 71. In some embodiments, the GLP-1R agonist is SEQ ID NO: 72. In some embodiments, the GLP-1R agonist is SEQ ID NO: 73. In some embodiments, the GLP-1R agonist is SEQ ID NO: 74. In some embodiments, the GLP-1R agonist is SEQ ID NO: 75. In some embodiments, the GLP-1R agonist is SEQ ID NO: 76. In some embodiments, the GLP-1R agonist is SEQ ID NO: 77. In some embodiments, the GLP-1R agonist is SEQ ID NO: 78. In some embodiments, the GLP-1R agonist is SEQ ID NO: 79. In some embodiments, the GLP-1R agonist is SEQ ID NO: 80. In some embodiments, the GLP-1R agonist is SEQ ID NO: 81. In some embodiments, the GLP-1R agonist is SEQ ID NO: 82. In some embodiments, the GLP-1R agonist is SEQ ID NO: 83. In some embodiments, the GLP-1R agonist is SEQ ID NO: 84. In some embodiments, the GLP-1R agonist is SEQ ID NO: 85. In some embodiments, the GLP-1R agonist is SEQ ID NO: 86. In some embodiments, the GLP-1R agonist is SEQ ID NO: 87. In some embodiments, the GLP-1R agonist is SEQ ID NO: 88. In some embodiments, the GLP-1R agonist is SEQ ID NO: 89. In some embodiments, the GLP-1R agonist is SEQ ID NO: 90. In some embodiments, the GLP-1R agonist is SEQ ID NO: 91. In some embodiments, the GLP-1R agonist is SEQ ID NO: 92. In some embodiments, the GLP-1R agonist is SEQ ID NO: 93. In some embodiments, the GLP-1R agonist is SEQ ID NO: 94. In some embodiments, the GLP-1R agonist is SEQ ID NO: 95. In some embodiments, the GLP-1R agonist is SEQ ID NO: 96. In some embodiments, the GLP-1R agonist is SEQ ID NO: 97. In some embodiments, the GLP-1R agonist is SEQ ID NO: 98. In some embodiments, the GLP-1R agonist is SEQ ID NO: 99. In some embodiments, the GLP-1R agonist is SEQ ID NO: 100. In some embodiments, the GLP-1R agonist is SEQ ID NO: 101. In some embodiments, the GLP-1R agonist is SEQ ID NO: 102. In some embodiments, the GLP-1R agonist is SEQ ID NO: 103. In some embodiments, the GLP-1R agonist is SEQ ID NO: 104. In some embodiments, the GLP-1R agonist is SEQ ID NO: 105. In some embodiments, the GLP-1R agonist is SEQ ID NO: 106. In some embodiments, the GLP-1R agonist is SEQ ID NO: 107. In some embodiments, the GLP-1R agonist is SEQ ID NO: 108. In some embodiments, the GLP-1R agonist is SEQ ID NO: 109. In some embodiments, the GLP-1R agonist is SEQ ID NO: 110. In some embodiments, the GLP-1R agonist is SEQ ID NO: 111. In some embodiments, the GLP-1R agonist is SEQ ID NO: 112. In some embodiments, the GLP-1R agonist is SEQ ID NO: 113. In some embodiments, the GLP-1R agonist is SEQ ID NO: 114. In some embodiments, the GLP-1R agonist is SEQ ID NO: 115. In some embodiments, the GLP-1R agonist is SEQ ID NO: 116. In some embodiments, the GLP-1R agonist is SEQ ID NO: 117. In some embodiments, the GLP-1R agonist is SEQ ID NO: 118. In some embodiments, the GLP-1R agonist is SEQ ID NO: 119. In some embodiments, the GLP-1R agonist is SEQ ID NO: 120. In some embodiments, the GLP-1R agonist is SEQ ID NO: 121. In some embodiments, the GLP-1R agonist is at least 90 or 95% identical to any one of the foregoing peptides. In some embodiments, the GLP-1R agonist comprises any one of the foregoing peptides provided the peptide has 1, 2, or 3 substitutions or deletions as compared to the reference sequence. The reference sequence can be any of the foregoing peptides.

In some embodiments, the GLP-1R agonist comprises a peptide having the following formula:

X₁X₂X₃X₄X₅X₆X₇X₈X₉X₁₀X₁₁X₁₂X₁₃X₁₄X₁₅X₁₆X₁₇X₁₈X₁₉X₂₀X₂₁X₂₂X₂₃X₂₄X₂₅X₂₆X₂₇X₂₈X₂₉X₃₀X₃₁X₃₂X₃₃X₃₄X₃₅X₃₆X₃₇X₃₈X₃₉X₄₀, wherein:

X₁ is absent or H; X₂ is any amino acid (natural or unnatural), G, S, A, or V; X₃ is E, D, or I; X₄ is F or G; X₅ is T; X₆ is F or any hydrophobic amino acid or a modified amino acid, such as those provided for herein at a similar position; X₇ is T; X₈ is S; X₉ is E or D; X₁₀ is L, V, Y, or any hydrophobic amino acid or a modified amino acid, such as those provided for herein at a similar position; X₁₁ is S or V; X₁₂ is K, R, or S; X₁₃ is Q, I or Y; X₁₄ is any modified amino acid, such as those provided for herein at a similar position, or L, M, or G; X₁₅ is E; X₁₆ is E, A, G, K, or any modified amino acid, such as those provided for herein at a similar position; X₁₇ is K, E, Q, or any modified amino acid, such as those provided for herein at a similar position; X₁₈ is A; X₁₉ is A or V; X₂₀ is K or R; X₂₁ is E, K, or L; X₂₂ is F; X₂₃ is I; X₂₄ is E or A, or any modified amino acid, such as those provided for herein at a similar position; X₂₅ is F or W; X₂₆ is L or any modified amino acid, such as those provided for herein at a similar position; X₂₇ is K, R, I, or V, or any modified amino acid, such as those provided for herein at a similar position; X₂₈ is Q, N, R, K; X₂₉ is G, R, or absent; X₃₀ is G, R, or absent; X₃₁ is P, G, K, or absent; X₃₂ is S, K, or absent; X₃₃ is S, K, or absent; X₃₄ is G, E, or absent; X₃s is A, I, or absent; X₃₆ is P, I, or absent; X₃₇ is P, S, or absent; X₃₈ is P or absent; X₃₉ is S, C, or absent; X₄₀ is K, C, or absent. In some embodiments, X₃₂-X₄₀ are absent. In some embodiments, X₃₁-X₃₉ comprise the amino acid sequence of PSSGAPPPS and X₄₀ is absent. In some embodiments, X₃₁-X₄₀ comprise the amino acid sequence of PSSGAPPPSK or PSSGAPPPCC.

In some embodiments, the GLP-1R agonist comprises a peptide means for agonizing GLP-1R.

While certain embodiments herein refer to the biologically active peptide as a GLP-1R agonist, other biologically active peptides are also considered and are within the scope of the present disclosure. Non-limiting examples of additional biologically active peptides that are within the scope of the present disclosure include, but are not limited to, GLP-1 analogues, GIP, GIP analogues, and GLP-1/glucagon peptides.

The foregoing biologically active peptides are exemplary only and are not meant to be limiting in any way. Additional exemplary biologically active peptides may be found at least in WO2017/112824, WO2018/136440, US2021/0154318, US2022/0025059, US2025/0270196, U.S. Pat. Nos. 10,294,303, 10,905,772, and 11,046,774, each of which are incorporated herein by reference in their entirety.

### Anti-GIP Antibody: Biologically Active Peptide Conjugate

In some embodiments, the biologically active peptide is conjugated to the anti-GIP antibody, or antigen-binding fragment thereof. In some embodiments, the biologically active peptide is as provided for herein. In some embodiments, the anti-GIP antibody, or antigen-binding fragment thereof, is as provided for herein.

In some embodiments, the biologically active peptide is conjugated to the anti-GIP antibody, or antigen-binding fragment thereof, through a cysteine or non-canonical amino acid substitution at one or more conjugation sites. In some embodiments, the biologically active peptide is conjugated to the anti-GIP antibody, or antigen-binding fragment thereof, through a cysteine substitution at one or more conjugation sites. In some embodiments, the biologically active peptide is conjugated to the anti-GIP antibody, or antigen-binding fragment thereof, through a non-canonical amino acid substitution at one or more conjugation sites.

In some embodiments, the biologically active peptide is conjugated to the anti-GIP antibody, or antigen-binding fragment thereof, within the CL, CH1, CH2, or CH3 region of the anti-GIP antibody, or antigen-binding fragment thereof, or any combination of the CL, CH1, CH2, or CH3 region of the anti-GIP antibody, or antigen-binding fragment thereof. In some embodiments, the biologically active peptide is conjugated to the anti-GIP antibody, or antigen-binding fragment thereof, within the CL region of the anti-GIP antibody, or antigen-binding fragment thereof. In some embodiments, the biologically active peptide is conjugated to the anti-GIP antibody, or antigen-binding fragment thereof, within the CH1 region of the anti-GIP antibody, or antigen-binding fragment thereof. In some embodiments, the biologically active peptide is conjugated to the anti-GIP antibody, or antigen-binding fragment thereof, within the CH2 region of the anti-GIP antibody, or antigen-binding fragment thereof. In some embodiments, the biologically active peptide is conjugated to the anti-GIP antibody, or antigen-binding fragment thereof, within the CH3 region of the anti-GIP antibody, or antigen-binding fragment thereof. In some embodiments, the biologically active peptide is conjugated to the anti-GIP antibody, or antigen-binding fragment thereof, within any combination of the CL, CH1, CH2, or CH3 region of the anti-GIP antibody, or antigen-binding fragment thereof.

In some embodiments, the biologically active peptide is conjugated to the anti-GIP antibody, or antigen-binding fragment thereof, within the Fc region of the anti-GIP antibody, or antigen-binding fragment thereof. In some embodiments, the Fc region is an IgG Fc, as provided for herein. In some embodiments, the biologically active peptide is conjugated to the anti-GIP antibody, or antigen-binding fragment thereof, within the IgG region of the anti-GIP antibody, or antigen-binding fragment thereof. In some embodiments, the IgG region comprises one or more substitutions of a canonical amino acid to a cysteine. In some embodiments, the biologically active peptide is conjugated to the anti-GIP antibody, or antigen-binding fragment thereof, through a substituted cysteine residue on the anti-GIP antibody, or antigen-binding fragment thereof.

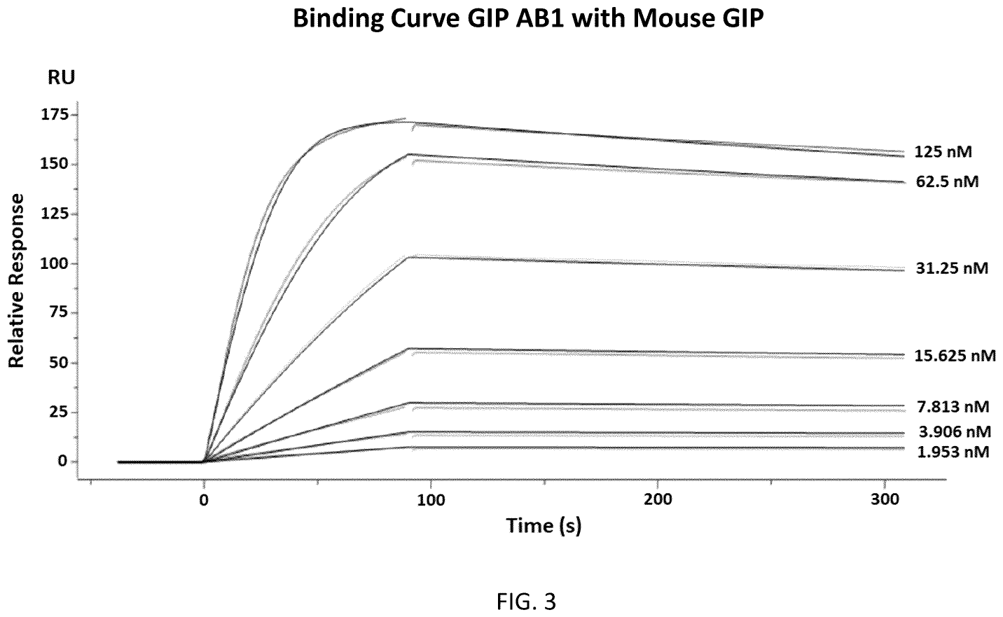

In some embodiments, the IgG Fc is an IgG1 Fc. In some embodiments, the IgG1 Fc comprises one or more substitutions of a canonical amino acid to a cysteine at position 70, 89, 109, 111, 118, 124, 140, 152, 239, 265, 272, 290, 300, 345, 359, 390, 442, or any combination thereof, wherein the amino acid positions refer to the EU numbering of the Fc region. The EU numbering of residues in the Fc domain is according to the EU index, which is provided for herein and can be found in Kabat et al. (1991) Sequences of Proteins of Immunological Interest, National Institutes of Health, Bethesda, MD, and according to FIGS. 3c-3f of U.S. Pat. App. Pub. No. 2008/0248028. The “EU index” may also be referred to as “EU numbering”. Unless specified otherwise, the amino acid positions of canonical amino acid to cysteine mutations provided for herein are provided under the EU numbering system. In some embodiments, the IgG1 Fc comprises a substitution of a canonical amino acid to a cysteine at position 70, 89, 109, 111, 118, 124, 140, 152, 239, 265, 272, 290, 300, 345, 359, 390, or 442. In some embodiments, the IgG1 Fc comprises a substitution of a canonical amino acid to a cysteine at position 70. In some embodiments, the IgG1 Fc comprises a substitution of a canonical amino acid to a cysteine at position 89. In some embodiments, the IgG1 Fc comprises a substitution of a canonical amino acid to a cysteine at position 109. In some embodiments, the IgG1 Fc comprises a substitution of a canonical amino acid to a cysteine at position 111. In some embodiments, the IgG1 Fc comprises a substitution of a canonical amino acid to a cysteine at position 118. In some embodiments, the IgG1 Fc comprises a substitution of a canonical amino acid to a cysteine at position 124. In some embodiments, the IgG1 Fc comprises a substitution of a canonical amino acid to a cysteine at position 140. In some embodiments, the IgG1 Fc comprises a substitution of a canonical amino acid to a cysteine at position 152. In some embodiments, the IgG1 Fc comprises a substitution of a canonical amino acid to a cysteine at position 239. In some embodiments, the IgG1 Fc comprises a substitution of a canonical amino acid to a cysteine at position 265. In some embodiments, the IgG1 Fc comprises a substitution of a canonical amino acid to a cysteine at position 272. In some embodiments, the IgG1 Fc comprises a substitution of a canonical amino acid to a cysteine at position 290. In some embodiments, the IgG1 Fc comprises a substitution of a canonical amino acid to a cysteine at position 300. In some embodiments, the IgG1 Fc comprises a substitution of a canonical amino acid to a cysteine at position 345. In some embodiments, the IgG1 Fc comprises a substitution of a canonical amino acid to a cysteine at position or 359. In some embodiments, the IgG1 Fc comprises a substitution of a canonical amino acid to a cysteine at position 390. In some embodiments, the IgG1 Fc comprises a substitution of a canonical amino acid to a cysteine at position 442. In some embodiments, the IgG1 Fc comprises more than one substitutions of a canonical amino acid to a cysteine at any combination of position 88, 99, 150, 152, 154, 156, 189, 377, 384, 421, 473, 487 and 526.

In some embodiments, the substitution of a canonical amino acid to a cysteine at position 70 comprises a D70C substitution. In some embodiments, the anti-GIP antibody, or antigen-binding fragment thereof, comprises an IgG1 Fc domain, wherein the IgG1 Fc comprises a D70C substitution. In some embodiments, the biologically active peptide is conjugated to the anti-GIP antibody, or antigen-binding fragment thereof, through the D70C substitution.

In some embodiments, the substitution of a canonical amino acid to a cysteine at position 89 comprises a E89C substitution. In some embodiments, the anti-GIP antibody, or antigen-binding fragment thereof, comprises an IgG1 Fc domain, wherein the IgG1 Fc comprises a E89C substitution. In some embodiments, the biologically active peptide is conjugated to the anti-GIP antibody, or antigen-binding fragment thereof, through the E89C substitution.

In some embodiments, the substitution of a canonical amino acid to a cysteine at position 109 comprises a V109C substitution. In some embodiments, the anti-GIP antibody, or antigen-binding fragment thereof, comprises an IgG1 Fc domain, wherein the IgG1 Fc comprises a V109C substitution. In some embodiments, the biologically active peptide is conjugated to the anti-GIP antibody, or antigen-binding fragment thereof, through the V109C substitution.3

In some embodiments, the substitution of a canonical amino acid to a cysteine at position 111 comprises a A111C substitution. In some embodiments, the anti-GIP antibody, or antigen-binding fragment thereof, comprises an IgG1 Fc domain, wherein the IgG1 Fc comprises a A111C substitution. In some embodiments, the biologically active peptide is conjugated to the anti-GIP antibody, or antigen-binding fragment thereof, through the A111C substitution.

In some embodiments, the substitution of a canonical amino acid to a cysteine at position 118 comprises a A118C substitution. In some embodiments, the anti-GIP antibody, or antigen-binding fragment thereof, comprises an IgG1 Fc domain, wherein the IgG1 Fc comprises a A118C substitution. In some embodiments, the biologically active peptide is conjugated to the anti-GIP antibody, or antigen-binding fragment thereof, through the A118C substitution.

In some embodiments, the substitution of a canonical amino acid to a cysteine at position 124 comprises a S124C substitution. In some embodiments, the anti-GIP antibody, or antigen-binding fragment thereof, comprises an IgG1 Fc domain, wherein the IgG1 Fc comprises a S124C substitution. In some embodiments, the biologically active peptide is conjugated to the anti-GIP antibody, or antigen-binding fragment thereof, through the S124C substitution.

In some embodiments, the substitution of a canonical amino acid to a cysteine at position 140 comprises a A140C substitution. In some embodiments, the anti-GIP antibody, or antigen-binding fragment thereof, comprises an IgG1 Fc domain, wherein the IgG1 Fc comprises a A140C substitution. In some embodiments, the biologically active peptide is conjugated to the anti-GIP antibody, or antigen-binding fragment thereof, through the A140C substitution.

In some embodiments, the substitution of a canonical amino acid to a cysteine at position 152 comprises a E152C substitution. In some embodiments, the anti-GIP antibody, or antigen-binding fragment thereof, comprises an IgG1 Fc domain, wherein the IgG1 Fc comprises a E152C substitution. In some embodiments, the biologically active peptide is conjugated to the anti-GIP antibody, or antigen-binding fragment thereof, through the E152C substitution.

In some embodiments, the substitution of a canonical amino acid to a cysteine at position 239 comprises a S239C substitution. In some embodiments, the anti-GIP antibody, or antigen-binding fragment thereof, comprises an IgG1 Fc domain, wherein the IgG1 Fc comprises a S239C substitution. In some embodiments, the biologically active peptide is conjugated to the anti-GIP antibody, or antigen-binding fragment thereof, through the S239C substitution.

In some embodiments, the substitution of a canonical amino acid to a cysteine at position 265 comprises a V265C substitution. In some embodiments, the anti-GIP antibody, or antigen-binding fragment thereof, comprises an IgG1 Fc domain, wherein the IgG1 Fc comprises a V265C substitution. In some embodiments, the biologically active peptide is conjugated to the anti-GIP antibody, or antigen-binding fragment thereof, through the V265C substitution.

In some embodiments, the substitution of a canonical amino acid to a cysteine at position 272 comprises a E272C substitution. In some embodiments, the anti-GIP antibody, or antigen-binding fragment thereof, comprises an IgG1 Fc domain, wherein the IgG1 Fc comprises a E272C substitution. In some embodiments, the biologically active peptide is conjugated to the anti-GIP antibody, or antigen-binding fragment thereof, through the E272C substitution.

In some embodiments, the substitution of a canonical amino acid to a cysteine at position 290 comprises a K290C substitution. In some embodiments, the anti-GIP antibody, or antigen-binding fragment thereof, comprises an IgG1 Fc domain, wherein the IgG1 Fc comprises a K290C substitution. In some embodiments, the biologically active peptide is conjugated to the anti-GIP antibody, or antigen-binding fragment thereof, through the K290C substitution.

In some embodiments, the substitution of a canonical amino acid to a cysteine at position 300 comprises a Y300C substitution. In some embodiments, the anti-GIP antibody, or antigen-binding fragment thereof, comprises an IgG1 Fc domain, wherein the IgG1 Fc comprises a Y300C substitution. In some embodiments, the biologically active peptide is conjugated to the anti-GIP antibody, or antigen-binding fragment thereof, through the Y300C substitution.

In some embodiments, the substitution of a canonical amino acid to a cysteine at position 345 comprises a E345C substitution. In some embodiments, the anti-GIP antibody, or antigen-binding fragment thereof, comprises an IgG1 Fc domain, wherein the IgG1 Fc comprises a E345C substitution. In some embodiments, the biologically active peptide is conjugated to the anti-GIP antibody, or antigen-binding fragment thereof, through the E345C substitution.

In some embodiments, the substitution of a canonical amino acid to a cysteine at position 359 comprises a T359C substitution. In some embodiments, the anti-GIP antibody, or antigen-binding fragment thereof, comprises an IgG1 Fc domain, wherein the IgG1 Fc comprises a T359C substitution. In some embodiments, the biologically active peptide is conjugated to the anti-GIP antibody, or antigen-binding fragment thereof, through the T359C substitution.

In some embodiments, the substitution of a canonical amino acid to a cysteine at position 390 comprises a N390C substitution. In some embodiments, the anti-GIP antibody, or antigen-binding fragment thereof, comprises an IgG1 Fc domain, wherein the IgG1 Fc comprises a N390C substitution. In some embodiments, the biologically active peptide is conjugated to the anti-GIP antibody, or antigen-binding fragment thereof, through the N390C substitution.

In some embodiments, the substitution of a canonical amino acid to a cysteine at position 442 comprises a S442C substitution. In some embodiments, the anti-GIP antibody, or antigen-binding fragment thereof, comprises an IgG1 Fc domain, wherein the IgG1 Fc comprises a S442C substitution. In some embodiments, the biologically active peptide is conjugated to the anti-GIP antibody, or antigen-binding fragment thereof, through the S442C substitution.

In some embodiments, the biologically active peptide is conjugated to the anti-GIP antibody, or antigen-binding fragment thereof, within the CL region of the anti-GIP antibody, or antigen-binding fragment thereof. In some embodiments, the CL region of the anti-GIP antibody, or antigen-binding fragment thereof comprises one or more substitutions of a canonical amino acid to a cysteine at position 149, 205, or any combination thereof, wherein the amino acid positions refer to the EU numbering. Unless specified otherwise, the amino acid positions of canonical amino acid to cysteine mutations provided for herein are provided under the EU numbering system. In some embodiments, the CL region of the anti-GIP antibody, or antigen-binding fragment thereof comprises a substitution of a canonical amino acid to a cysteine at position 149. In some embodiments, the CL region of the anti-GIP antibody, or antigen-binding fragment thereof comprises a substitution of a canonical amino acid to a cysteine at position 205.

In some embodiments, the substitution of a canonical amino acid to a cysteine at position 149 of the CL region comprises a K149C substitution. In some embodiments, the anti-GIP antibody, or antigen binding fragment thereof, comprises a CL wherein the CL comprises a K149C mutation. In some embodiments, the biologically active peptide is conjugated to the anti-GIP antibody, or antigen binding fragment thereof, through the K149C mutation.

In some embodiments, the substitution of a canonical amino acid to a cysteine at position 205 of the CL region comprises a V205C substitution. In some embodiments, the anti-GIP antibody, or antigen binding fragment thereof, comprises a CL wherein the CL comprises a V205C mutation. In some embodiments, the biologically active peptide is conjugated to the anti-GIP antibody, or antigen binding fragment thereof, through the V205C mutation.

In some embodiments, the anti-GIP antibody, or antigen binding fragment thereof, comprises one or more substitutions of a canonical amino acid to a cysteine in an IgG1 Fc domain at a position as provided for herein and comprises one or more substitutions of a canonical amino acid to a cysteine in an CL at a position as provided for herein.

The conjugation site must be amenable to conjugation of an additional functional moiety (e.g. a biologically active peptide as provided for herein) by a defined conjugation chemistry through the side chain of an amino acid residue at the conjugation site. Achieving highly selective, site specific conjugation to the anti-GIP antibody, or antigen-binding fragment thereof, in accordance with the present disclosure, requires consideration of a diverse variety of design criteria. First, a conjugation or coupling chemistry must be defined or predetermined. Functional moieties, such as biologically active peptides, can be conjugated or coupled to the selected conjugation site of the anti-GIP antibody, or antigen-binding fragment thereof, through an assortment of different conjugation chemistries known in the art. For example, a maleimide activated conjugation partner targeting an accessible cysteine thiol on the anti-GIP antibody, or antigen-binding fragment thereof, is one embodiment, but numerous conjugation or coupling chemistries targeting the side chains of either canonical or non-canonical, e.g., unnatural amino acids in the anti-GIP antibody sequence, or the sequence of the anti-GIP antigen-binding fragment, are within the scope of the present disclosure.

Chemistries for the chemoselective conjugation include, but are not limited to, copper(I)-catalyzed azidealkyne [3+2] dipolar cycloadditions, Staudinger ligation, other acyl transfers processes (S→N; X→N), oximations, hydrazone bonding formation and other suitable organic chemistry reactions such as cross couplings using water-soluble palladium catalysts (E.g., Bong et al., Chemoselective Pd(0)-catalyzed peptide coupling in water, Organic Letters 3(16): 2509-11 (2001); Dibowski et al., Bioconjugation of peptides by palladium-catalyzed C—C cross-coupling in water, Angew. Chem. Int. Ed. 37(4): 476-78 (1998); DeVasher et al., Aqueous-phase, palladium-catalyzed cross-coupling of aryl bromides under mild conditions, using water-soluble, sterically demanding alkylphosphines, J. Org. Chem. 69:7919-27 (2004); Shaugnessy et al., J. Org. Chem, 2003, 68, 6767-6774; Prescher, J A and Bertozzi C R, Chemistry in living system, Nature Chemical Biology 1(1); 13-21 (2005)).

As mentioned above, in some embodiments, the conjugation (or covalent binding) to the anti-GIP antibody, or antigen-binding fragment thereof, is through the side chain of an amino acid residue at the conjugation site, for example, but not limited to, a cysteinyl residue. The amino acid residue, for example, a cysteinyl residue, at the internal conjugation site that is selected can be one that occupies the same amino acid residue position in a native Fc domain sequence, or the amino acid residue can be engineered into the Fc domain sequence by substitution or insertion.

Other examples of unnatural amino acid residues that can be particularly useful as the conjugation site include, but are not limited to, azido-containing amino acid residues, e.g., azidohomoalanine, p-azido-phenylalanine; keto-containing amino acid residues, e.g., p-acetyl-phenylalanine; alkyne-containing amino acid residues, e.g., p-ethynylphenylalanine, homopropargylglycine, p-(prop-2-ynyl)-tyrosine; alkene-containing amino acid residues e.g., homoallylglycine; aryl halide-containing amino acid residues e.g. p-iodophenylalanine, p-bromophenylalanine; and 1,2-aminothiol containing amino acid residues.

The non-canonical amino acid residues can be incorporated by amino acid substitution or insertion. Non-canonical amino acid residues can be incorporated into the peptide by chemical peptide synthesis rather than by synthesis in biological systems, such as recombinantly expressing cells, or alternatively the skilled artisan can employ known techniques of protein engineering that use recombinantly expressing cells. (See, e.g., Link et al., Non-canonical amino acids in protein engineering, Current Opinion in Biotechnology, 14(6): 603-609 (2003); Schultz et al., In vivo incorporation of unnatural amino acids, U.S. Pat. No. 7,045,337).

The selection of the placement of the conjugation site in the overall anti-GIP antibody, or antigen-binding fragment thereof, is another important facet of selecting an internal conjugation site in accordance with the present disclosure. Any of the exposed amino acid residues on the anti-GIP antibody, or antigen-binding fragment thereof, can be potentially useful conjugation sites and can be mutated to cysteine or some other reactive amino acid for site-selective coupling, if not already present at the selected conjugation site of the anti-GIP antibody, or antigen-binding fragment thereof, protein sequence. While any of the exposed amino acid residues can be potentially useful, other factors must also be taken into consideration, such as potential steric constraints that may perturb the activity of the conjugated biologically active peptide or limit the reactivity of the engineered mutation.

Accordingly, in some embodiments, the biologically active peptide is conjugated directly to the anti-GIP antibody, or antigen-binding fragment thereof. In some embodiments, the conjugation site is at a substitution of a canonical amino acid for a cysteine or non-canonical amino acid, as provided for herein. In some embodiments, the conjugation site comprises a substitution at one or more of positions 70, 89, 109, 111, 118, 124, 140, 152, 239, 265, 272, 290, 300, 345, 359, 390, or 442 of an IgG1 Fc domain as provided for herein or at one or more of positions 149 or 205 of a CL domain as provided for herein, or any combination thereof. In some embodiments, the conjugation is through a chemistry as provided for herein or an equivalent thereof.

In some embodiments, the biologically active peptide is conjugated indirectly to the anti-GIP antibody, or antigen-binding fragment thereof, such as through a linker moiety. In some embodiments, the linker is a polypeptide linker. Polypeptide linkers are known in the art, and any such linker is within the scope of the present disclosure. Nonlimiting examples of polypeptide linkers are provided in Table 7 below:

TABLE 7
Sequences of exemplary polypeptide linkers

| SEQ | | Variable Values, |
|---|---|---|
| ID NO: | Sequence | if applicable |
| 130 | (GGGGA)ₙ | n = 1, 2, |
| | | 3, 4, or 5 |
| 131 | (GGGGS)ₙ | n = 1, 2, |
| | | 3, 4, or 5 |
| 132 | (Gly)₈ | |
| 133 | (Gly)₆ | |
| 134 | (EAAAK)ₙ | n = 1, 2, |
| | | 3, 4, or 5 |
| 135 | A(EAAAK)ₙA | n = 1, 2, |
| | | 3, 4, or 5 |
| 136 | AEAAAKEAAAKA | |
| 137 | A(EAAAK)₄ALEA(EAAAK)₄ | |
| 138 | PAPAP | |
| 139 | (XP)ₙ | n = 1, 2, |
| | | 3, 4, or 5 |
| | | X = A, K, |
| | | or E |
| 140 | GSAGSAAGSGEF | |
| 141 | KESGSVSSEQLAQFRSLD | |
| 142 | EGKSSGSGSESKST | |
| 143 | AEAAAKEAAAKA | |

The foregoing linker peptides are exemplary only and are not meant to be limiting in any way. Additional exemplary linker peptides may be found at least in WO2025/184315 and US2025/0276080, each of which are incorporated herein by reference in their entirety.

In some embodiments, the polypeptide linker is located at the N-terminus of the biologically active peptide. In some embodiments, the polypeptide linker is located at the C-terminus of the biologically active peptide. In some embodiments, the polypeptide linker contains one or more additional amino acid residues to facilitate the conjugation of the polypeptide linker to the anti-GIP antibody, or antigen-binding fragment thereof. In some embodiments, the polypeptide linker is conjugated to the anti-GIP antibody, or antigen-binding fragment thereof, through a chemistry as provided for herein or an equivalent thereof.

In some embodiments, a composition is provided, the composition comprising an anti-GIP antibody, or antigen binding fragment thereof, as provided for herein, conjugated or linked to a biologically active peptide, as provided for herein, wherein the biologically active peptide is conjugated or linked to the anti-GIP antibody, or antigen binding fragment thereof, at a residue on the antibody which reduces the potency of the biologically active peptide. In some embodiments, the reduced potency of the biologically active peptide is as compared to the same biologically active peptide which is not conjugated or linked to the anti-GIP antibody, or antigen binding fragment thereof. In some embodiments, the reduced potency of the biologically active peptide is as compared to the same biologically active peptide which is conjugated or linked to the anti-GIP antibody, or antigen binding fragment thereof, at another residue of the antibody or antigen binding fragment thereof. In some embodiments, the biologically active peptide is conjugated or linked to the anti-GIP antibody, or antigen binding fragment thereof, at a residue as provided for herein. In some embodiments, the biologically active peptide is conjugated or linked to the anti-GIP antibody, or antigen binding fragment thereof, at a substitution of a canonical amino acid to a cysteine, wherein the canonical amino acid substituted to a cysteine is as provided for herein. In some embodiments, the biologically active peptide is conjugated or linked to the anti-GIP antibody, or antigen binding fragment thereof in the Fc region of the antibody. In some embodiments, the Fc region is an IgG1 Fc. In some embodiments, the IgG1 Fc comprises one or more substitutions of a canonical amino acid to a cysteine at position 70, 89, 109, 111, 118, 124, 140, 152, 239, 265, 272, 290, 300, 345, 359, 390, 442, or any combination thereof, wherein the amino acid positions refer to the EU numbering of the Fc region. The EU numbering of residues in the Fc domain is according to the EU index, which is provided for herein. In some embodiments, the one substitutions of a canonical amino acid to a cysteine at position 70, 89, 109, 111, 118, 124, 140, 152, 239, 265, 272, 290, 300, 345, 359, 390, 442, or any combination thereof, comprises a D70C, E89C, V109C, A111C, A118C, S124C, A140C, E152C, S239C, V265C, E272C, K290C, Y300C, E345C, T359C, N390C, or S442C. In some embodiments, the biologically active peptide is conjugated or linked to the anti-GIP antibody, or antigen binding fragment thereof in the CL domain. In some embodiments, the CL region of the anti-GIP antibody, or antigen-binding fragment thereof comprises one or more substitutions of a canonical amino acid to a cysteine at position 149, 205, or any combination thereof, wherein the amino acid positions refer to the EU numbering. In some embodiments, the one substitutions of a canonical amino acid to a cysteine at position 149, 205, or any combination thereof comprise a K149C or V205C.

In some embodiments, the biologically active peptide is a GLP-1R agonist, such as a GLP-1R agonist as provided for herein, linked to conjugated to the anti-GIP antibody via a peptide linker, such as a peptide linker as provided for herein, at a location in the antibody wherein the canonical amino acid has been substituted to a cysteine, such as at a position as provided for herein. In some non-limiting embodiments, the GLP-1R agonist comprises an amino acid sequence of SEQ ID NO: 116 and is conjugated to the anti-GIP antibody through a linker comprising the amino acid sequence of SEQ ID NO: 131 (n=3), wherein the linker sequence is at the C-terminus of the GLP-1R agonist sequence. In some embodiments, the linker sequence further comprises a C-terminal lysine. Accordingly, in some non-limiting embodiments, the GLP-1R agonist comprising a linker sequence comprises the amino acid sequence of HXEGTFTSDYSSYLEEQAAKEFIAWLVKGGG(GGGGS)₃K (SEQ ID NO: 145, wherein X=2-Aminoisobutyric acid). In some embodiments, the linker sequence may further be modified to comprise a C-terminal bromoacetyl group (BrAc). In some embodiments, the GLP-1R agonist comprising a linker sequence comprises the amino acid sequence of HXEGTFTSDYSSYLEEQAAKEFIAWLVKGGG(GGGGS)₃K—[BrAc] (SEQ ID NO: 146, wherein X=2-Aminoisobutyric acid).

Without being bound to any particular theory, the potency of a biologically active peptide may be modified by attaching it to a specific residue of the anti-GIP antibody, or antigen binding fragment thereof. The modified potency of the biologically active peptide may be different depending on which residue of the anti-GIP antibody, or antigen binding fragment thereof, the biologically active peptide is attached to. The modified potency may be beneficial, for example, in scenarios where a reduced potency helps to abrogate unwanted side effects of the biologically active peptide, wherein a reduced potency increases subject tolerability of the biologically active peptide and/or conjugate thereof, and/or wherein a reduced potency enhances the efficacy of the biologically active peptide conjugated to the anti-GIP antibody, or antigen binding fragment thereof.

In some embodiments, the biologically active peptide is appended to the anti-GIP antibody, or antigen binding fragment thereof, as a genetic fusion. In the context of the present disclosure, the term “genetic fusion” is defined as meaning that the anti-GIP antibody and biologically active peptide conjugate is produced recombinantly through the translation contiguous nucleic acid sequence. In some embodiments, the nucleic acid sequence encoding the biologically active peptide is located at the 5′ end of the nucleic acid sequence encoding the anti-GIP antibody, or antigen binding fragment thereof. In some embodiments, the nucleic acid sequence encoding the biologically active peptide is located at the 3′ end of the nucleic acid sequence encoding the anti-GIP antibody, or antigen binding fragment thereof. In some embodiments, the nucleic acid sequence encoding the biologically active peptide is located within the nucleic acid sequence encoding the anti-GIP antibody, or antigen binding fragment thereof in a manner such that the anti-GIP antibody, or antigen binding fragment thereof, still forms a functional molecule. Methods for the generation and expression of genetic fusions are known in the art, and any such method is within the scope of the present disclosure and may be used to produce the biologically active peptide: anti-GIP antibody, or antigen binding fragment thereof, fusion.

### Pharmaceutical Compositions

In some embodiments, a pharmaceutical composition is provided comprising an anti-GIP antibody, or an antigen-binding fragment thereof, and biologically active peptide. In some embodiments, the anti-GIP antibody is an antibody means for binding to the GIP ligand. In some embodiments, the anti-GIP antibody is an antibody means for binding to the GIP ligand and inhibiting its binding to its cognate receptor GIP1R.

In some embodiments, the pharmaceutical composition comprises an anti-GIP antibody, or an antigen-binding fragment thereof, a biologically active peptide, and a pharmaceutically acceptable excipient, wherein the anti-GIP antibody, or antigen-binding fragment thereof, comprises a variable heavy chain region (VH) and a variable light chain region (VL), wherein the VH comprises a HCDR1 having the amino acid sequence of SEQ ID NO: 15, a HCDR2 having the amino acid sequence of SEQ ID NO: 26, and an HCDR3 having the amino acid sequence of SEQ ID NO: 17, and wherein the VL comprises a LCDR1 having the amino acid sequence of SEQ ID NO: 27, a LCDR2 having the amino acid sequence of SEQ ID NO: 28, and a LCDR3 having the amino acid sequence of SEQ ID NO: 19, and wherein the biologically active peptide is a GLP-1R agonist, liraglutide, albiglutide, taspoglutide, semaglutide, LY2428757, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91, SEQ ID NO: 92, SEQ ID NO: 93, SEQ ID NO: 94, SEQ ID NO: 95, SEQ ID NO: 96, SEQ ID NO: 97, SEQ ID NO: 98, SEQ ID NO: 99, SEQ ID NO: 100, SEQ ID NO: 101, SEQ ID NO: 102, SEQ ID NO: 103, SEQ ID NO: 104, SEQ ID NO: 105, SEQ ID NO: 106, SEQ ID NO: 107, SEQ ID NO: 108, SEQ ID NO: 109, SEQ ID NO: 110, SEQ ID NO: 111, SEQ ID NO: 112, SEQ ID NO: 113, SEQ ID NO: 114, SEQ ID NO: 115, SEQ ID NO: 116, SEQ ID NO: 117, SEQ ID NO: 118, SEQ ID NO: 119, SEQ ID NO: 120, or SEQ ID NO: 121, or a combination of any of the foregoing. In some embodiments, the pharmaceutical composition comprises an anti-GIP antibody, or an antigen-binding fragment thereof, a biologically active peptide, and a pharmaceutically acceptable excipient, wherein the anti-GIP antibody, or antigen-binding fragment thereof, comprises a variable heavy chain region (VH) and a variable light chain region (VL), wherein the VH comprises a HCDR1 having the amino acid sequence of SEQ ID NO: 15, a HCDR2 having the amino acid sequence of SEQ ID NO: 26, and an HCDR3 having the amino acid sequence of SEQ ID NO: 17, and wherein the VL comprises a LCDR1 having the amino acid sequence of SEQ ID NO: 27, a LCDR2 having the amino acid sequence of SEQ ID NO: 28, and a LCDR3 having the amino acid sequence of SEQ ID NO: 19, and wherein the biologically active peptide is semaglutide.

In some embodiments, the pharmaceutical composition comprises an anti-GIP antibody, or an antigen-binding fragment thereof, a biologically active peptide, and a pharmaceutically acceptable excipient, wherein the anti-GIP antibody, or antigen-binding fragment thereof, comprises a variable heavy chain region (VH) and a variable light chain region (VL), wherein the VH comprises a HCDR1 having the amino acid sequence of SEQ ID NO: 15, a HCDR2 having the amino acid sequence of SEQ ID NO: 26, and an HCDR3 having the amino acid sequence of SEQ ID NO: 17, and wherein the VL comprises a LCDR1 having the amino acid sequence of SEQ ID NO: 27, a LCDR2 having the amino acid sequence of SEQ ID NO: 31, and a LCDR3 having the amino acid sequence of SEQ ID NO: 19, and wherein the biologically active peptide is a GLP-1R agonist, liraglutide, albiglutide, taspoglutide, semaglutide, LY2428757, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91, SEQ ID NO: 92, SEQ ID NO: 93, SEQ ID NO: 94, SEQ ID NO: 95, SEQ ID NO: 96, SEQ ID NO: 97, SEQ ID NO: 98, SEQ ID NO: 99, SEQ ID NO: 100, SEQ ID NO: 101, SEQ ID NO: 102, SEQ ID NO: 103, SEQ ID NO: 104, SEQ ID NO: 105, SEQ ID NO: 106, SEQ ID NO: 107, SEQ ID NO: 108, SEQ ID NO: 109, SEQ ID NO: 110, SEQ ID NO: 111, SEQ ID NO: 112, SEQ ID NO: 113, SEQ ID NO: 114, SEQ ID NO: 115, SEQ ID NO: 116, SEQ ID NO: 117, SEQ ID NO: 118, SEQ ID NO: 119, SEQ ID NO: 120, or SEQ ID NO: 121, or a combination of any of the foregoing. In some embodiments, the pharmaceutical composition comprises an anti-GIP antibody, or an antigen-binding fragment thereof, a biologically active peptide, and a pharmaceutically acceptable excipient, wherein the anti-GIP antibody, or antigen-binding fragment thereof, comprises a variable heavy chain region (VH) and a variable light chain region (VL), wherein the VH comprises a HCDR1 having the amino acid sequence of SEQ ID NO: 15, a HCDR2 having the amino acid sequence of SEQ ID NO: 26, and an HCDR3 having the amino acid sequence of SEQ ID NO: 17, and wherein the VL comprises a LCDR1 having the amino acid sequence of SEQ ID NO: 27, a LCDR2 having the amino acid sequence of SEQ ID NO: 31, and a LCDR3 having the amino acid sequence of SEQ ID NO: 19, and wherein the biologically active peptide is semaglutide.

In some embodiments, the pharmaceutical composition comprises an anti-GIP antibody, or an antigen-binding fragment thereof, a biologically active peptide, and a pharmaceutically acceptable excipient, wherein the anti-GIP antibody, or antigen-binding fragment thereof, comprises a variable heavy chain region (VH) and a variable light chain region (VL), wherein the VH comprises a HCDR1 having the amino acid sequence of SEQ ID NO: 15, a HCDR2 having the amino acid sequence of SEQ ID NO: 26, and an HCDR3 having the amino acid sequence of SEQ ID NO: 17, and wherein the VL comprises a LCDR1 having the amino acid sequence of SEQ ID NO: 27, a LCDR2 having the amino acid sequence of SEQ ID NO: 34, and a LCDR3 having the amino acid sequence of SEQ ID NO: 19, and wherein the biologically active peptide is a GLP-1R agonist, liraglutide, albiglutide, taspoglutide, semaglutide, LY2428757, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91, SEQ ID NO: 92, SEQ ID NO: 93, SEQ ID NO: 94, SEQ ID NO: 95, SEQ ID NO: 96, SEQ ID NO: 97, SEQ ID NO: 98, SEQ ID NO: 99, SEQ ID NO: 100, SEQ ID NO: 101, SEQ ID NO: 102, SEQ ID NO: 103, SEQ ID NO: 104, SEQ ID NO: 105, SEQ ID NO: 106, SEQ ID NO: 107, SEQ ID NO: 108, SEQ ID NO: 109, SEQ ID NO: 110, SEQ ID NO: 111, SEQ ID NO: 112, SEQ ID NO: 113, SEQ ID NO: 114, SEQ ID NO: 115, SEQ ID NO: 116, SEQ ID NO: 117, SEQ ID NO: 118, SEQ ID NO: 119, SEQ ID NO: 120, or SEQ ID NO: 121, or a combination of any of the foregoing. In some embodiments, the pharmaceutical composition comprises an anti-GIP antibody, or an antigen-binding fragment thereof, a biologically active peptide, and a pharmaceutically acceptable excipient, wherein the anti-GIP antibody, or antigen-binding fragment thereof, comprises a variable heavy chain region (VH) and a variable light chain region (VL), wherein the VH comprises a HCDR1 having the amino acid sequence of SEQ ID NO: 15, a HCDR2 having the amino acid sequence of SEQ ID NO: 26, and an HCDR3 having the amino acid sequence of SEQ ID NO: 17, and wherein the VL comprises a LCDR1 having the amino acid sequence of SEQ ID NO: 27, a LCDR2 having the amino acid sequence of SEQ ID NO: 34, and a LCDR3 having the amino acid sequence of SEQ ID NO: 19, and wherein the biologically active peptide is semaglutide.

In some embodiments, the pharmaceutical composition comprises an anti-GIP antibody, or an antigen-binding fragment thereof, a biologically active peptide, and a pharmaceutically acceptable excipient, wherein the anti-GIP antibody, or antigen-binding fragment thereof, comprises a variable heavy chain region (VH) and a variable light chain region (VL), wherein the VH comprises a HCDR1 having the amino acid sequence of SEQ ID NO: 15, a HCDR2 having the amino acid sequence of SEQ ID NO: 16, and an HCDR3 having the amino acid sequence of SEQ ID NO: 17, and wherein the VL comprises a LCDR I having the amino acid sequence of SEQ ID NO: 18, a LCDR2 having the amino acid sequence of SEQ ID NO: 5, and a LCDR3 having the amino acid sequence of SEQ ID NO: 19, and wherein the biologically active peptide is a GLP-1R agonist, liraglutide, albiglutide, taspoglutide, semaglutide, LY2428757, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91, SEQ ID NO: 92, SEQ ID NO: 93, SEQ ID NO: 94, SEQ ID NO: 95, SEQ ID NO: 96, SEQ ID NO: 97, SEQ ID NO: 98, SEQ ID NO: 99, SEQ ID NO: 100, SEQ ID NO: 101, SEQ ID NO: 102, SEQ ID NO: 103, SEQ ID NO: 104, SEQ ID NO: 105, SEQ ID NO: 106, SEQ ID NO: 107, SEQ ID NO: 108, SEQ ID NO: 109, SEQ ID NO: 110, SEQ ID NO: 111, SEQ ID NO: 112, SEQ ID NO: 113, SEQ ID NO: 114, SEQ ID NO: 115, SEQ ID NO: 116, SEQ ID NO: 117, SEQ ID NO: 118, SEQ ID NO: 119, SEQ ID NO: 120, or SEQ ID NO: 121, or a combination of any of the foregoing. In some embodiments, the pharmaceutical composition comprises an anti-GIP antibody, or an antigen-binding fragment thereof, a biologically active peptide, and a pharmaceutically acceptable excipient, wherein the anti-GIP antibody, or antigen-binding fragment thereof, comprises a variable heavy chain region (VH) and a variable light chain region (VL), wherein the VH comprises a HCDR1 having the amino acid sequence of SEQ ID NO: 15, a HCDR2 having the amino acid sequence of SEQ ID NO: 16, and an HCDR3 having the amino acid sequence of SEQ ID NO: 17, and wherein the VL comprises a LCDR1 having the amino acid sequence of SEQ ID NO: 18, a LCDR2 having the amino acid sequence of SEQ ID NO: 5, and a LCDR3 having the amino acid sequence of SEQ ID NO: 19, and wherein the biologically active peptide is semaglutide.

In some embodiments, the pharmaceutical composition comprises an anti-GIP antibody, or an antigen-binding fragment thereof, a biologically active peptide, and a pharmaceutically acceptable excipient, wherein the anti-GIP antibody, or antigen-binding fragment thereof, comprises a variable heavy chain region (VH) and a variable light chain region (VL), wherein the VH comprises a HCDR1 having the amino acid sequence of SEQ ID NO: 1, a HCDR2 having the amino acid sequence of SEQ ID NO: 2, and an HCDR3 having the amino acid sequence of SEQ ID NO: 3, and wherein the VL comprises a LCDR1 having the amino acid sequence of SEQ ID NO: 4, a LCDR2 having the amino acid sequence of SEQ ID NO: 5, and a LCDR3 having the amino acid sequence of SEQ ID NO: 6, and wherein the biologically active peptide a GLP-1R agonist, liraglutide, albiglutide, taspoglutide, semaglutide, LY2428757, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91, SEQ ID NO: 92, SEQ ID NO: 93, SEQ ID NO: 94, SEQ ID NO: 95, SEQ ID NO: 96, SEQ ID NO: 97, SEQ ID NO: 98, SEQ ID NO: 99, SEQ ID NO: 100, SEQ ID NO: 101, SEQ ID NO: 102, SEQ ID NO: 103, SEQ ID NO: 104, SEQ ID NO: 105, SEQ ID NO: 106, SEQ ID NO: 107, SEQ ID NO: 108, SEQ ID NO: 109, SEQ ID NO: 110, SEQ ID NO: 111, SEQ ID NO: 112, SEQ ID NO: 113, SEQ ID NO: 114, SEQ ID NO: 115, SEQ ID NO: 116, SEQ ID NO: 117, SEQ ID NO: 118, SEQ ID NO: 119, SEQ ID NO: 120, or SEQ ID NO: 121, or a combination of any of the foregoing. In some embodiments, the pharmaceutical composition comprises an anti-GIP antibody, or an antigen-binding fragment thereof, a biologically active peptide, and a pharmaceutically acceptable excipient, wherein the anti-GIP antibody, or antigen-binding fragment thereof, comprises a variable heavy chain region (VH) and a variable light chain region (VL), wherein the VH comprises a HCDR1 having the amino acid sequence of SEQ ID NO: 1, a HCDR2 having the amino acid sequence of SEQ ID NO: 2, and an HCDR3 having the amino acid sequence of SEQ ID NO: 3, and wherein the VL comprises a LCDR1 having the amino acid sequence of SEQ ID NO: 4, a LCDR2 having the amino acid sequence of SEQ ID NO: 5, and a LCDR3 having the amino acid sequence of SEQ ID NO: 6, and wherein the biologically active peptide is semaglutide.

In some embodiments, the pharmaceutical composition comprises an anti-GIP antibody, or an antigen-binding fragment thereof, a biologically active peptide, and a pharmaceutically acceptable excipient, wherein the anti-GIP antibody, or antigen-binding fragment thereof, comprises a variable heavy chain region (VH) and a variable light chain region (VL), wherein the VH comprises an amino acid sequence of SEQ ID NO: 29, and wherein the VL comprises an amino acid sequence of SEQ ID NO: 30, and wherein the biologically active peptide is a GLP-1R agonist, liraglutide, albiglutide, taspoglutide, semaglutide, LY2428757, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91, SEQ ID NO: 92, SEQ ID NO: 93, SEQ ID NO: 94, SEQ ID NO: 95, SEQ ID NO: 96, SEQ ID NO: 97, SEQ ID NO: 98, SEQ ID NO: 99, SEQ ID NO: 100, SEQ ID NO: 101, SEQ ID NO: 102, SEQ ID NO: 103, SEQ ID NO: 104, SEQ ID NO: 105, SEQ ID NO: 106, SEQ ID NO: 107, SEQ ID NO: 108, SEQ ID NO: 109, SEQ ID NO: 110, SEQ ID NO: 111, SEQ ID NO: 112, SEQ ID NO: 113, SEQ ID NO: 114, SEQ ID NO: 115, SEQ ID NO: 116, SEQ ID NO: 117, SEQ ID NO: 118, SEQ ID NO: 119, SEQ ID NO: 120, or SEQ ID NO: 121, or a combination of any of the foregoing. In some embodiments, the pharmaceutical composition comprises an anti-GIP antibody, or an antigen-binding fragment thereof, a biologically active peptide, and a pharmaceutically acceptable excipient, wherein the anti-GIP antibody, or antigen-binding fragment thereof, comprises a variable heavy chain region (VH) and a variable light chain region (VL), wherein the VH comprises an amino acid sequence of SEQ ID NO: 29, and wherein the VL comprises an amino acid sequence of SEQ ID NO: 30, and wherein the biologically active peptide is semaglutide.

In some embodiments, the pharmaceutical composition comprises an anti-GIP antibody, or an antigen-binding fragment thereof, a biologically active peptide, and a pharmaceutically acceptable excipient, wherein the anti-GIP antibody, or antigen-binding fragment thereof, comprises a variable heavy chain region (VH) and a variable light chain region (VL), wherein the VH comprises an amino acid sequence of SEQ ID NO: 32, and wherein the VL comprises an amino acid sequence of SEQ ID NO: 33, and wherein the biologically active peptide is a GLP-1R agonist, liraglutide, albiglutide, taspoglutide, semaglutide, LY2428757, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91, SEQ ID NO: 92, SEQ ID NO: 93, SEQ ID NO: 94, SEQ ID NO: 95, SEQ ID NO: 96, SEQ ID NO: 97, SEQ ID NO: 98, SEQ ID NO: 99, SEQ ID NO: 100, SEQ ID NO: 101, SEQ ID NO: 102, SEQ ID NO: 103, SEQ ID NO: 104, SEQ ID NO: 105, SEQ ID NO: 106, SEQ ID NO: 107, SEQ ID NO: 108, SEQ ID NO: 109, SEQ ID NO: 110, SEQ ID NO: 111, SEQ ID NO: 112, SEQ ID NO: 113, SEQ ID NO: 114, SEQ ID NO: 115, SEQ ID NO: 116, SEQ ID NO: 117, SEQ ID NO: 118, SEQ ID NO: 119, SEQ ID NO: 120, or SEQ ID NO: 121, or a combination of any of the foregoing. In some embodiments, the pharmaceutical composition comprises an anti-GIP antibody, or an antigen-binding fragment thereof, a biologically active peptide, and a pharmaceutically acceptable excipient, wherein the anti-GIP antibody, or antigen-binding fragment thereof, comprises a variable heavy chain region (VH) and a variable light chain region (VL), wherein the VH comprises an amino acid sequence of SEQ ID NO: 32, and wherein the VL comprises an amino acid sequence of SEQ ID NO: 33, and wherein the biologically active peptide is semaglutide.

In some embodiments, the pharmaceutical composition comprises an anti-GIP antibody, or an antigen-binding fragment thereof, a biologically active peptide, and a pharmaceutically acceptable excipient, wherein the anti-GIP antibody, or antigen-binding fragment thereof, comprises a variable heavy chain region (VH) and a variable light chain region (VL), wherein the VH comprises an amino acid sequence of SEQ ID NO: 35, and wherein the VL comprises an amino acid sequence of SEQ ID NO: 36, and wherein the biologically active peptide a GLP-1R agonist, liraglutide, albiglutide, taspoglutide, semaglutide, LY2428757, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91, SEQ ID NO: 92, SEQ ID NO: 93, SEQ ID NO: 94, SEQ ID NO: 95, SEQ ID NO: 96, SEQ ID NO: 97, SEQ ID NO: 98, SEQ ID NO: 99, SEQ ID NO: 100, SEQ ID NO: 101, SEQ ID NO: 102, SEQ ID NO: 103, SEQ ID NO: 104, SEQ ID NO: 105, SEQ ID NO: 106, SEQ ID NO: 107, SEQ ID NO: 108, SEQ ID NO: 109, SEQ ID NO: 110, SEQ ID NO: 111, SEQ ID NO: 112, SEQ ID NO: 113, SEQ ID NO: 114, SEQ ID NO: 115, SEQ ID NO: 116, SEQ ID NO: 117, SEQ ID NO: 118, SEQ ID NO: 119, SEQ ID NO: 120, or SEQ ID NO: 121, or a combination of any of the foregoing. In some embodiments, the pharmaceutical composition comprises an anti-GIP antibody, or an antigen-binding fragment thereof, a biologically active peptide, and a pharmaceutically acceptable excipient, wherein the anti-GIP antibody, or antigen-binding fragment thereof, comprises a variable heavy chain region (VH) and a variable light chain region (VL), wherein the VH comprises an amino acid sequence of SEQ ID NO: 35, and wherein the VL comprises an amino acid sequence of SEQ ID NO: 36, and wherein the biologically active peptide is semaglutide.

In some embodiments, the pharmaceutical composition comprises an anti-GIP antibody, or an antigen-binding fragment thereof, a biologically active peptide, and a pharmaceutically acceptable excipient, wherein the anti-GIP antibody, or antigen-binding fragment thereof, comprises a variable heavy chain region (VH) and a variable light chain region (VL), wherein the VH comprises an amino acid sequence of SEQ ID NO: 24, and wherein the VL comprises an amino acid sequence of SEQ ID NO: 25, and wherein the biologically active peptide a GLP-1R agonist, liraglutide, albiglutide, taspoglutide, semaglutide, LY2428757, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91, SEQ ID NO: 92, SEQ ID NO: 93, SEQ ID NO: 94, SEQ ID NO: 95, SEQ ID NO: 96, SEQ ID NO: 97, SEQ ID NO: 98, SEQ ID NO: 99, SEQ ID NO: 100, SEQ ID NO: 101, SEQ ID NO: 102, SEQ ID NO: 103, SEQ ID NO: 104, SEQ ID NO: 105, SEQ ID NO: 106, SEQ ID NO: 107, SEQ ID NO: 108, SEQ ID NO: 109, SEQ ID NO: 110, SEQ ID NO: 111, SEQ ID NO: 112, SEQ ID NO: 113, SEQ ID NO: 114, SEQ ID NO: 115, SEQ ID NO: 116, SEQ ID NO: 117, SEQ ID NO: 118, SEQ ID NO: 119, SEQ ID NO: 120, or SEQ ID NO: 121, or a combination of any of the foregoing. In some embodiments, the pharmaceutical composition comprises an anti-GIP antibody, or an antigen-binding fragment thereof, a biologically active peptide, and a pharmaceutically acceptable excipient, wherein the anti-GIP antibody, or antigen-binding fragment thereof, comprises a variable heavy chain region (VH) and a variable light chain region (VL), wherein the VH comprises an amino acid sequence of SEQ ID NO: 24, and wherein the VL comprises an amino acid sequence of SEQ ID NO: 25, and wherein the biologically active peptide is semaglutide.

In some embodiments, the pharmaceutical composition comprises an anti-GIP antibody, or an antigen-binding fragment thereof, a biologically active peptide, and a pharmaceutically acceptable excipient, wherein the anti-GIP antibody, or antigen-binding fragment thereof, comprises a variable heavy chain region (VH) and a variable light chain region (VL), wherein the VH comprises an amino acid sequence of SEQ ID NO: 13, and wherein the VL comprises an amino acid sequence of SEQ ID NO: 14, and wherein the biologically active peptide is a GLP-1R agonist, liraglutide, albiglutide, taspoglutide, semaglutide, LY2428757, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91, SEQ ID NO: 92, SEQ ID NO: 93, SEQ ID NO: 94, SEQ ID NO: 95, SEQ ID NO: 96, SEQ ID NO: 97, SEQ ID NO: 98, SEQ ID NO: 99, SEQ ID NO: 100, SEQ ID NO: 101, SEQ ID NO: 102, SEQ ID NO: 103, SEQ ID NO: 104, SEQ ID NO: 105, SEQ ID NO: 106, SEQ ID NO: 107, SEQ ID NO: 108, SEQ ID NO: 109, SEQ ID NO: 110, SEQ ID NO: 111, SEQ ID NO: 112, SEQ ID NO: 113, SEQ ID NO: 114, SEQ ID NO: 115, SEQ ID NO: 116, SEQ ID NO: 117, SEQ ID NO: 118, SEQ ID NO: 119, SEQ ID NO: 120, or SEQ ID NO: 121, or a combination of any of the foregoing. In some embodiments, the pharmaceutical composition comprises an anti-GIP antibody, or an antigen-binding fragment thereof, a biologically active peptide, and a pharmaceutically acceptable excipient, wherein the anti-GIP antibody, or antigen-binding fragment thereof, comprises a variable heavy chain region (VH) and a variable light chain region (VL), wherein the VH comprises an amino acid sequence of SEQ ID NO: 13, and wherein the VL comprises an amino acid sequence of SEQ ID NO: 14, and wherein the biologically active peptide is semaglutide.

In some embodiments, the pharmaceutical composition comprises an anti-GIP antibody, or an antigen-binding fragment thereof, a biologically active peptide, and a pharmaceutically acceptable excipient, wherein the anti-GIP antibody, or antigen-binding fragment thereof, comprises a heavy chain (HC) and a light chain (LC), wherein the HC comprises an amino acid sequence of SEQ ID NO: 37, and wherein the LC comprises an amino acid sequence of SEQ ID NO: 144, and wherein the biologically active peptide is a GLP-1R agonist, liraglutide, albiglutide, taspoglutide, semaglutide, LY2428757, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91, SEQ ID NO: 92, SEQ ID NO: 93, SEQ ID NO: 94, SEQ ID NO: 95, SEQ ID NO: 96, SEQ ID NO: 97, SEQ ID NO: 98, SEQ ID NO: 99, SEQ ID NO: 100, SEQ ID NO: 101, SEQ ID NO: 102, SEQ ID NO: 103, SEQ ID NO: 104, SEQ ID NO: 105, SEQ ID NO: 106, SEQ ID NO: 107, SEQ ID NO: 108, SEQ ID NO: 109, SEQ ID NO: 110, SEQ ID NO: 111, SEQ ID NO: 112, SEQ ID NO: 113, SEQ ID NO: 114, SEQ ID NO: 115, SEQ ID NO: 116, SEQ ID NO: 117, SEQ ID NO: 118, SEQ ID NO: 119, SEQ ID NO: 120, or SEQ ID NO: 121, or a combination of any of the foregoing. In some embodiments, the pharmaceutical composition comprises an anti-GIP antibody, or an antigen-binding fragment thereof, a biologically active peptide, and a pharmaceutically acceptable excipient, wherein the anti-GIP antibody, or antigen-binding fragment thereof, comprises a HC and a LC, wherein the HC comprises an amino acid sequence of SEQ ID NO: 37, and wherein the LC comprises an amino acid sequence of SEQ ID NO: 144, and wherein the biologically active peptide is semaglutide.

In some embodiments, the pharmaceutical composition comprises an anti-GIP antibody, or an antigen-binding fragment thereof, conjugated to a biologically active peptide, and a pharmaceutically acceptable excipient, wherein the anti-GIP antibody, or antigen-binding fragment thereof, comprises a variable heavy chain region (VH) and a variable light chain region (VL), wherein the VH comprises a HCDR1 having the amino acid sequence of SEQ ID NO: 15, a HCDR2 having the amino acid sequence of SEQ ID NO: 26, and an HCDR3 having the amino acid sequence of SEQ ID NO: 17, and wherein the VL comprises a LCDR1 having the amino acid sequence of SEQ ID NO: 27, a LCDR2 having the amino acid sequence of SEQ ID NO: 28, and a LCDR3 having the amino acid sequence of SEQ ID NO: 19, and wherein the biologically active peptide is a GLP-1R agonist, liraglutide, albiglutide, taspoglutide, semaglutide, LY2428757, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91, SEQ ID NO: 92, SEQ ID NO: 93, SEQ ID NO: 94, SEQ ID NO: 95, SEQ ID NO: 96, SEQ ID NO: 97, SEQ ID NO: 98, SEQ ID NO: 99, SEQ ID NO: 100, SEQ ID NO: 101, SEQ ID NO: 102, SEQ ID NO: 103, SEQ ID NO: 104, SEQ ID NO: 105, SEQ ID NO: 106, SEQ ID NO: 107, SEQ ID NO: 108, SEQ ID NO: 109, SEQ ID NO: 110, SEQ ID NO: 111, SEQ ID NO: 112, SEQ ID NO: 113, SEQ ID NO: 114, SEQ ID NO: 115, SEQ ID NO: 116, SEQ ID NO: 117, SEQ ID NO: 118, SEQ ID NO: 119, SEQ ID NO: 120, SEQ ID NO: 121, SEQ ID NO: 145, or SEQ ID NO: 146, or a peptide that is 85%, 90%, 91%, 92%, 93%, 94%, 95%, 96%, 97%, 98%, or 99% identical thereto, or a combination of any of the foregoing. In some embodiments, the pharmaceutical composition comprises an anti-GIP antibody, or an antigen-binding fragment thereof, conjugated to a biologically active peptide, and a pharmaceutically acceptable excipient, wherein the anti-GIP antibody, or antigen-binding fragment thereof, comprises a variable heavy chain region (VH) and a variable light chain region (VL), wherein the VH comprises a HCDR1 having the amino acid sequence of SEQ ID NO: 15, a HCDR2 having the amino acid sequence of SEQ ID NO: 26, and an HCDR3 having the amino acid sequence of SEQ ID NO: 17, and wherein the VL comprises a LCDR1 having the amino acid sequence of SEQ ID NO: 27, a LCDR2 having the amino acid sequence of SEQ ID NO: 28, and a LCDR3 having the amino acid sequence of SEQ ID NO: 19, and wherein the biologically active peptide is semaglutide.

In some embodiments, the pharmaceutical composition comprises an anti-GIP antibody, or an antigen-binding fragment thereof, conjugated to a biologically active peptide, and a pharmaceutically acceptable excipient, wherein the anti-GIP antibody, or antigen-binding fragment thereof, comprises a variable heavy chain region (VH) and a variable light chain region (VL), wherein the VH comprises a HCDR1 having the amino acid sequence of SEQ ID NO: 15, a HCDR2 having the amino acid sequence of SEQ ID NO: 26, and an HCDR3 having the amino acid sequence of SEQ ID NO: 17, and wherein the VL comprises a LCDR1 having the amino acid sequence of SEQ ID NO: 27, a LCDR2 having the amino acid sequence of SEQ ID NO: 31, and a LCDR3 having the amino acid sequence of SEQ ID NO: 19, and wherein the biologically active peptide is a GLP-1R agonist, liraglutide, albiglutide, taspoglutide, semaglutide, LY2428757, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91, SEQ ID NO: 92, SEQ ID NO: 93, SEQ ID NO: 94, SEQ ID NO: 95, SEQ ID NO: 96, SEQ ID NO: 97, SEQ ID NO: 98, SEQ ID NO: 99, SEQ ID NO: 100, SEQ ID NO: 101, SEQ ID NO: 102, SEQ ID NO: 103, SEQ ID NO: 104, SEQ ID NO: 105, SEQ ID NO: 106, SEQ ID NO: 107, SEQ ID NO: 108, SEQ ID NO: 109, SEQ ID NO: 110, SEQ ID NO: 111, SEQ ID NO: 112, SEQ ID NO: 113, SEQ ID NO: 114, SEQ ID NO: 115, SEQ ID NO: 116, SEQ ID NO: 117, SEQ ID NO: 118, SEQ ID NO: 119, SEQ ID NO: 120, SEQ ID NO: 121, SEQ ID NO: 145, or SEQ ID NO: 146, or a peptide that is 85%, 90%, 91%, 92%, 93%, 94%, 95%, 96%, 97%, 98%, or 99% identical thereto, or a combination of any of the foregoing. In some embodiments, the pharmaceutical composition comprises an anti-GIP antibody, or an antigen-binding fragment thereof, conjugated to a biologically active peptide, and a pharmaceutically acceptable excipient, wherein the anti-GIP antibody, or antigen-binding fragment thereof, comprises a variable heavy chain region (VH) and a variable light chain region (VL), wherein the VH comprises a HCDR1 having the amino acid sequence of SEQ ID NO: 15, a HCDR2 having the amino acid sequence of SEQ ID NO: 26, and an HCDR3 having the amino acid sequence of SEQ ID NO: 17, and wherein the VL comprises a LCDR1 having the amino acid sequence of SEQ ID NO: 27, a LCDR2 having the amino acid sequence of SEQ ID NO: 31, and a LCDR3 having the amino acid sequence of SEQ ID NO: 19, and wherein the biologically active peptide is semaglutide.

In some embodiments, the pharmaceutical composition comprises an anti-GIP antibody, or an antigen-binding fragment thereof, conjugated to a biologically active peptide, and a pharmaceutically acceptable excipient, wherein the anti-GIP antibody, or antigen-binding fragment thereof, comprises a variable heavy chain region (VH) and a variable light chain region (VL), wherein the VH comprises a HCDR1 having the amino acid sequence of SEQ ID NO: 15, a HCDR2 having the amino acid sequence of SEQ ID NO: 26, and an HCDR3 having the amino acid sequence of SEQ ID NO: 17, and wherein the VL comprises a LCDR1 having the amino acid sequence of SEQ ID NO: 27, a LCDR2 having the amino acid sequence of SEQ ID NO: 34, and a LCDR3 having the amino acid sequence of SEQ ID NO: 19, and wherein the biologically active peptide is a GLP-1R agonist, liraglutide, albiglutide, taspoglutide, semaglutide, LY2428757, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91, SEQ ID NO: 92, SEQ ID NO: 93, SEQ ID NO: 94, SEQ ID NO: 95, SEQ ID NO: 96, SEQ ID NO: 97, SEQ ID NO: 98, SEQ ID NO: 99, SEQ ID NO: 100, SEQ ID NO: 101, SEQ ID NO: 102, SEQ ID NO: 103, SEQ ID NO: 104, SEQ ID NO: 105, SEQ ID NO: 106, SEQ ID NO: 107, SEQ ID NO: 108, SEQ ID NO: 109, SEQ ID NO: 110, SEQ ID NO: 111, SEQ ID NO: 112, SEQ ID NO: 113, SEQ ID NO: 114, SEQ ID NO: 115, SEQ ID NO: 116, SEQ ID NO: 117, SEQ ID NO: 118, SEQ ID NO: 119, SEQ ID NO: 120, SEQ ID NO: 121, SEQ ID NO: 145, or SEQ ID NO: 146, or a peptide that is 85%, 90%, 91%, 92%, 93%, 94%, 95%, 96%, 97%, 98%, or 99% identical thereto, or a combination of any of the foregoing. In some embodiments, the pharmaceutical composition comprises an anti-GIP antibody, or an antigen-binding fragment thereof, conjugated to a biologically active peptide, and a pharmaceutically acceptable excipient, wherein the anti-GIP antibody, or antigen-binding fragment thereof, comprises a variable heavy chain region (VH) and a variable light chain region (VL), wherein the VH comprises a HCDR1 having the amino acid sequence of SEQ ID NO: 15, a HCDR2 having the amino acid sequence of SEQ ID NO: 26, and an HCDR3 having the amino acid sequence of SEQ ID NO: 17, and wherein the VL comprises a LCDR1 having the amino acid sequence of SEQ ID NO: 27, a LCDR2 having the amino acid sequence of SEQ ID NO: 34, and a LCDR3 having the amino acid sequence of SEQ ID NO: 19, and wherein the biologically active peptide is semaglutide.

In some embodiments, the pharmaceutical composition comprises an anti-GIP antibody, or an antigen-binding fragment thereof, conjugated to a biologically active peptide, and a pharmaceutically acceptable excipient, wherein the anti-GIP antibody, or antigen-binding fragment thereof, comprises a variable heavy chain region (VH) and a variable light chain region (VL), wherein the VH comprises a HCDR1 having the amino acid sequence of SEQ ID NO: 15, a HCDR2 having the amino acid sequence of SEQ ID NO: 16, and an HCDR3 having the amino acid sequence of SEQ ID NO: 17, and wherein the VL comprises a LCDR1 having the amino acid sequence of SEQ ID NO: 18, a LCDR2 having the amino acid sequence of SEQ ID NO: 5, and a LCDR3 having the amino acid sequence of SEQ ID NO: 19, and wherein the biologically active peptide is a GLP-1R agonist, liraglutide, albiglutide, taspoglutide, semaglutide, LY2428757, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91, SEQ ID NO: 92, SEQ ID NO: 93, SEQ ID NO: 94, SEQ ID NO: 95, SEQ ID NO: 96, SEQ ID NO: 97, SEQ ID NO: 98, SEQ ID NO: 99, SEQ ID NO: 100, SEQ ID NO: 101, SEQ ID NO: 102, SEQ ID NO: 103, SEQ ID NO: 104, SEQ ID NO: 105, SEQ ID NO: 106, SEQ ID NO: 107, SEQ ID NO: 108, SEQ ID NO: 109, SEQ ID NO: 110, SEQ ID NO: 111, SEQ ID NO: 112, SEQ ID NO: 113, SEQ ID NO: 114, SEQ ID NO: 115, SEQ ID NO: 116, SEQ ID NO: 117, SEQ ID NO: 118, SEQ ID NO: 119, SEQ ID NO: 120, SEQ ID NO: 121, SEQ ID NO: 145, or SEQ ID NO: 146, or a peptide that is 85%, 90%, 91%, 92%, 93%, 94%, 95%, 96%, 97%, 98%, or 99% identical thereto, or a combination of any of the foregoing. In some embodiments, the pharmaceutical composition comprises an anti-GIP antibody, or an antigen-binding fragment thereof, conjugated to a biologically active peptide, and a pharmaceutically acceptable excipient, wherein the anti-GIP antibody, or antigen-binding fragment thereof, comprises a variable heavy chain region (VH) and a variable light chain region (VL), wherein the VH comprises a HCDR1 having the amino acid sequence of SEQ ID NO: 15, a HCDR2 having the amino acid sequence of SEQ ID NO: 16, and an HCDR3 having the amino acid sequence of SEQ ID NO: 17, and wherein the VL comprises a LCDR1 having the amino acid sequence of SEQ ID NO: 18, a LCDR2 having the amino acid sequence of SEQ ID NO: 5, and a LCDR3 having the amino acid sequence of SEQ ID NO: 19, and wherein the biologically active peptide is semaglutide.

In some embodiments, the pharmaceutical composition comprises an anti-GIP antibody, or an antigen-binding fragment thereof, conjugated to a biologically active peptide, and a pharmaceutically acceptable excipient, wherein the anti-GIP antibody, or antigen-binding fragment thereof, comprises a variable heavy chain region (VH) and a variable light chain region (VL), wherein the VH comprises a HCDR1 having the amino acid sequence of SEQ ID NO: 1, a HCDR2 having the amino acid sequence of SEQ ID NO: 2, and an HCDR3 having the amino acid sequence of SEQ ID NO: 3, and wherein the VL comprises a LCDR1 having the amino acid sequence of SEQ ID NO: 4, a LCDR2 having the amino acid sequence of SEQ ID NO: 5, and a LCDR3 having the amino acid sequence of SEQ ID NO: 6, and wherein the biologically active peptide a GLP-1R agonist, liraglutide, albiglutide, taspoglutide, semaglutide, LY2428757, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91, SEQ ID NO: 92, SEQ ID NO: 93, SEQ ID NO: 94, SEQ ID NO: 95, SEQ ID NO: 96, SEQ ID NO: 97, SEQ ID NO: 98, SEQ ID NO: 99, SEQ ID NO: 100, SEQ ID NO: 101, SEQ ID NO: 102, SEQ ID NO: 103, SEQ ID NO: 104, SEQ ID NO: 105, SEQ ID NO: 106, SEQ ID NO: 107, SEQ ID NO: 108, SEQ ID NO: 109, SEQ ID NO: 110, SEQ ID NO: 111, SEQ ID NO: 112, SEQ ID NO: 113, SEQ ID NO: 114, SEQ ID NO: 115, SEQ ID NO: 116, SEQ ID NO: 117, SEQ ID NO: 118, SEQ ID NO: 119, SEQ ID NO: 120, SEQ ID NO: 121, SEQ ID NO: 145, or SEQ ID NO: 146, or a peptide that is 85%, 90%, 91%, 92%, 93%, 94%, 95%, 96%, 97%, 98%, or 99% identical thereto, or a combination of any of the foregoing. In some embodiments, the pharmaceutical composition comprises an anti-GIP antibody, or an antigen-binding fragment thereof, conjugated to a biologically active peptide, and a pharmaceutically acceptable excipient, wherein the anti-GIP antibody, or antigen-binding fragment thereof, comprises a variable heavy chain region (VH) and a variable light chain region (VL), wherein the VH comprises a HCDR1 having the amino acid sequence of SEQ ID NO: 1, a HCDR2 having the amino acid sequence of SEQ ID NO: 2, and an HCDR3 having the amino acid sequence of SEQ ID NO: 3, and wherein the VL comprises a LCDR1 having the amino acid sequence of SEQ ID NO: 4, a LCDR2 having the amino acid sequence of SEQ ID NO: 5, and a LCDR3 having the amino acid sequence of SEQ ID NO: 6, and wherein the biologically active peptide is semaglutide.

In some embodiments, the pharmaceutical composition comprises an anti-GIP antibody, or an antigen-binding fragment thereof, conjugated to a biologically active peptide, and a pharmaceutically acceptable excipient, wherein the anti-GIP antibody, or antigen-binding fragment thereof, comprises a variable heavy chain region (VH) and a variable light chain region (VL), wherein the VH comprises an amino acid sequence of SEQ ID NO: 29, and wherein the VL comprises an amino acid sequence of SEQ ID NO: 30, and wherein the biologically active peptide is a GLP-1R agonist, liraglutide, albiglutide, taspoglutide, semaglutide, LY2428757, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91, SEQ ID NO: 92, SEQ ID NO: 93, SEQ ID NO: 94, SEQ ID NO: 95, SEQ ID NO: 96, SEQ ID NO: 97, SEQ ID NO: 98, SEQ ID NO: 99, SEQ ID NO: 100, SEQ ID NO: 101, SEQ ID NO: 102, SEQ ID NO: 103, SEQ ID NO: 104, SEQ ID NO: 105, SEQ ID NO: 106, SEQ ID NO: 107, SEQ ID NO: 108, SEQ ID NO: 109, SEQ ID NO: 110, SEQ ID NO: 111, SEQ ID NO: 112, SEQ ID NO: 113, SEQ ID NO: 114, SEQ ID NO: 115, SEQ ID NO: 116, SEQ ID NO: 117, SEQ ID NO: 118, SEQ ID NO: 119, SEQ ID NO: 120, SEQ ID NO: 121, SEQ ID NO: 145, or SEQ ID NO: 146, or a peptide that is 85%, 90%, 91%, 92%, 93%, 94%, 95%, 96%, 97%, 98%, or 99% identical thereto, or a combination of any of the foregoing. In some embodiments, the pharmaceutical composition comprises an anti-GIP antibody, or an antigen-binding fragment thereof, conjugated to a biologically active peptide, and a pharmaceutically acceptable excipient, wherein the anti-GIP antibody, or antigen-binding fragment thereof, comprises a variable heavy chain region (VH) and a variable light chain region (VL), wherein the VH comprises an amino acid sequence of SEQ ID NO: 29, and wherein the VL comprises an amino acid sequence of SEQ ID NO: 30, and wherein the biologically active peptide is semaglutide.

In some embodiments, the pharmaceutical composition comprises an anti-GIP antibody, or an antigen-binding fragment thereof, conjugated to a biologically active peptide, and a pharmaceutically acceptable excipient, wherein the anti-GIP antibody, or antigen-binding fragment thereof, comprises a variable heavy chain region (VH) and a variable light chain region (VL), wherein the VH comprises an amino acid sequence of SEQ ID NO: 32, and wherein the VL comprises an amino acid sequence of SEQ ID NO: 33, and wherein the biologically active peptide is a GLP-1R agonist, liraglutide, albiglutide, taspoglutide, semaglutide, LY2428757, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91, SEQ ID NO: 92, SEQ ID NO: 93, SEQ ID NO: 94, SEQ ID NO: 95, SEQ ID NO: 96, SEQ ID NO: 97, SEQ ID NO: 98, SEQ ID NO: 99, SEQ ID NO: 100, SEQ ID NO: 101, SEQ ID NO: 102, SEQ ID NO: 103, SEQ ID NO: 104, SEQ ID NO: 105, SEQ ID NO: 106, SEQ ID NO: 107, SEQ ID NO: 108, SEQ ID NO: 109, SEQ ID NO: 110, SEQ ID NO: 111, SEQ ID NO: 112, SEQ ID NO: 113, SEQ ID NO: 114, SEQ ID NO: 115, SEQ ID NO: 116, SEQ ID NO: 117, SEQ ID NO: 118, SEQ ID NO: 119, SEQ ID NO: 120, SEQ ID NO: 121, SEQ ID NO: 145, or SEQ ID NO: 146, or a peptide that is 85%, 90%, 91%, 92%, 93%, 94%, 95%, 96%, 97%, 98%, or 99% identical thereto, or a combination of any of the foregoing. In some embodiments, the pharmaceutical composition comprises an anti-GIP antibody, or an antigen-binding fragment thereof, conjugated to a biologically active peptide, and a pharmaceutically acceptable excipient, wherein the anti-GIP antibody, or antigen-binding fragment thereof, comprises a variable heavy chain region (VH) and a variable light chain region (VL), wherein the VH comprises an amino acid sequence of SEQ ID NO: 32, and wherein the VL comprises an amino acid sequence of SEQ ID NO: 33, and wherein the biologically active peptide is semaglutide.

In some embodiments, the pharmaceutical composition comprises an anti-GIP antibody, or an antigen-binding fragment thereof, conjugated to a biologically active peptide, and a pharmaceutically acceptable excipient, wherein the anti-GIP antibody, or antigen-binding fragment thereof, comprises a variable heavy chain region (VH) and a variable light chain region (VL), wherein the VH comprises an amino acid sequence of SEQ ID NO: 35, and wherein the VL comprises an amino acid sequence of SEQ ID NO: 36, and wherein the biologically active peptide a GLP-1R agonist, liraglutide, albiglutide, taspoglutide, semaglutide, LY2428757, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91, SEQ ID NO: 92, SEQ ID NO: 93, SEQ ID NO: 94, SEQ ID NO: 95, SEQ ID NO: 96, SEQ ID NO: 97, SEQ ID NO: 98, SEQ ID NO: 99, SEQ ID NO: 100, SEQ ID NO: 101, SEQ ID NO: 102, SEQ ID NO: 103, SEQ ID NO: 104, SEQ ID NO: 105, SEQ ID NO: 106, SEQ ID NO: 107, SEQ ID NO: 108, SEQ ID NO: 109, SEQ ID NO: 110, SEQ ID NO: 111, SEQ ID NO: 112, SEQ ID NO: 113, SEQ ID NO: 114, SEQ ID NO: 115, SEQ ID NO: 116, SEQ ID NO: 117, SEQ ID NO: 118, SEQ ID NO: 119, SEQ ID NO: 120, SEQ ID NO: 121, SEQ ID NO: 145, or SEQ ID NO: 146, or a peptide that is 85%, 90%, 91%, 92%, 93%, 94%, 95%, 96%, 97%, 98%, or 99% identical thereto, or a combination of any of the foregoing. In some embodiments, the pharmaceutical composition comprises an anti-GIP antibody, or an antigen-binding fragment thereof, conjugated to a biologically active peptide, and a pharmaceutically acceptable excipient, wherein the anti-GIP antibody, or antigen-binding fragment thereof, comprises a variable heavy chain region (VH) and a variable light chain region (VL), wherein the VH comprises an amino acid sequence of SEQ ID NO: 35, and wherein the VL comprises an amino acid sequence of SEQ ID NO: 36, and wherein the biologically active peptide is semaglutide.

In some embodiments, the pharmaceutical composition comprises an anti-GIP antibody, or an antigen-binding fragment thereof, conjugated to a biologically active peptide, and a pharmaceutically acceptable excipient, wherein the anti-GIP antibody, or antigen-binding fragment thereof, comprises a variable heavy chain region (VH) and a variable light chain region (VL), wherein the VH comprises an amino acid sequence of SEQ ID NO: 24, and wherein the VL comprises an amino acid sequence of SEQ ID NO: 25, and wherein the biologically active peptide a GLP-1R agonist, liraglutide, albiglutide, taspoglutide, semaglutide, LY2428757, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91, SEQ ID NO: 92, SEQ ID NO: 93, SEQ ID NO: 94, SEQ ID NO: 95, SEQ ID NO: 96, SEQ ID NO: 97, SEQ ID NO: 98, SEQ ID NO: 99, SEQ ID NO: 100, SEQ ID NO: 101, SEQ ID NO: 102, SEQ ID NO: 103, SEQ ID NO: 104, SEQ ID NO: 105, SEQ ID NO: 106, SEQ ID NO: 107, SEQ ID NO: 108, SEQ ID NO: 109, SEQ ID NO: 110, SEQ ID NO: 111, SEQ ID NO: 112, SEQ ID NO: 113, SEQ ID NO: 114, SEQ ID NO: 115, SEQ ID NO: 116, SEQ ID NO: 117, SEQ ID NO: 118, SEQ ID NO: 119, SEQ ID NO: 120, SEQ ID NO: 121, SEQ ID NO: 145, or SEQ ID NO: 146, or a peptide that is 85%, 90%, 91%, 92%, 93%, 94%, 95%, 96%, 97%, 98%, or 99% identical thereto, or a combination of any of the foregoing. In some embodiments, the pharmaceutical composition comprises an anti-GIP antibody, or an antigen-binding fragment thereof, conjugated to a biologically active peptide, and a pharmaceutically acceptable excipient, wherein the anti-GIP antibody, or antigen-binding fragment thereof, comprises a variable heavy chain region (VH) and a variable light chain region (VL), wherein the VH comprises an amino acid sequence of SEQ ID NO: 24, and wherein the VL comprises an amino acid sequence of SEQ ID NO: 25, and wherein the biologically active peptide is semaglutide.

In some embodiments, the pharmaceutical composition comprises an anti-GIP antibody, or an antigen-binding fragment thereof, conjugated to a biologically active peptide, and a pharmaceutically acceptable excipient, wherein the anti-GIP antibody, or antigen-binding fragment thereof, comprises a variable heavy chain region (VH) and a variable light chain region (VL), wherein the VH comprises an amino acid sequence of SEQ ID NO: 13, and wherein the VL comprises an amino acid sequence of SEQ ID NO: 14, and wherein the biologically active peptide is a GLP-1R agonist, liraglutide, albiglutide, taspoglutide, semaglutide, LY2428757, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91, SEQ ID NO: 92, SEQ ID NO: 93, SEQ ID NO: 94, SEQ ID NO: 95, SEQ ID NO: 96, SEQ ID NO: 97, SEQ ID NO: 98, SEQ ID NO: 99, SEQ ID NO: 100, SEQ ID NO: 101, SEQ ID NO: 102, SEQ ID NO: 103, SEQ ID NO: 104, SEQ ID NO: 105, SEQ ID NO: 106, SEQ ID NO: 107, SEQ ID NO: 108, SEQ ID NO: 109, SEQ ID NO: 110, SEQ ID NO: 111, SEQ ID NO: 112, SEQ ID NO: 113, SEQ ID NO: 114, SEQ ID NO: 115, SEQ ID NO: 116, SEQ ID NO: 117, SEQ ID NO: 118, SEQ ID NO: 119, SEQ ID NO: 120, SEQ ID NO: 121, SEQ ID NO: 145, or SEQ ID NO: 146, or a peptide that is 85%, 90%, 91%, 92%, 93%, 94%, 95%, 96%, 97%, 98%, or 99% identical thereto, or a combination of any of the foregoing. In some embodiments, the pharmaceutical composition comprises an anti-GIP antibody, or an antigen-binding fragment thereof, conjugated to a biologically active peptide, and a pharmaceutically acceptable excipient, wherein the anti-GIP antibody, or antigen-binding fragment thereof, comprises a variable heavy chain region (VH) and a variable light chain region (VL), wherein the VH comprises an amino acid sequence of SEQ ID NO: 13, and wherein the VL comprises an amino acid sequence of SEQ ID NO: 14, and wherein the biologically active peptide is semaglutide.

In some embodiments, the pharmaceutical composition comprises an anti-GIP antibody, or an antigen-binding fragment thereof, conjugated to a biologically active peptide, and a pharmaceutically acceptable excipient, wherein the anti-GIP antibody, or antigen-binding fragment thereof, comprises a heavy chain (HC) and a light chain (LC), wherein the HC comprises an amino acid sequence of SEQ ID NO: 37, and wherein the LC comprises an amino acid sequence of SEQ ID NO: 144, and wherein the biologically active peptide is a GLP-1R agonist, liraglutide, albiglutide, taspoglutide, semaglutide, LY2428757, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91, SEQ ID NO: 92, SEQ ID NO: 93, SEQ ID NO: 94, SEQ ID NO: 95, SEQ ID NO: 96, SEQ ID NO: 97, SEQ ID NO: 98, SEQ ID NO: 99, SEQ ID NO: 100, SEQ ID NO: 101, SEQ ID NO: 102, SEQ ID NO: 103, SEQ ID NO: 104, SEQ ID NO: 105, SEQ ID NO: 106, SEQ ID NO: 107, SEQ ID NO: 108, SEQ ID NO: 109, SEQ ID NO: 110, SEQ ID NO: 111, SEQ ID NO: 112, SEQ ID NO: 113, SEQ ID NO: 114, SEQ ID NO: 115, SEQ ID NO: 116, SEQ ID NO: 117, SEQ ID NO: 118, SEQ ID NO: 119, SEQ ID NO: 120, SEQ ID NO: 121, SEQ ID NO: 145, or SEQ ID NO: 146, or a peptide that is 85%, 90%, 91%, 92%, 93%, 94%, 95%, 96%, 97%, 98%, or 99% identical thereto, or a combination of any of the foregoing. In some embodiments, the pharmaceutical composition comprises an anti-GIP antibody, or an antigen-binding fragment thereof, conjugated to a biologically active peptide, and a pharmaceutically acceptable excipient, wherein the anti-GIP antibody, or antigen-binding fragment thereof, comprises a HC and a LC, wherein the HC comprises an amino acid sequence of SEQ ID NO: 37, and wherein the LC comprises an amino acid sequence of SEQ ID NO: 144, and wherein the biologically active peptide is semaglutide.

In certain embodiments, the pharmaceutical composition comprises an anti-GIP antibody, or an antigen-binding fragment thereof, a biologically active peptide, and a pharmaceutically acceptable excipient, wherein the anti-GIP antibody, or antigen-binding fragment thereof, and the biologically active peptide are conjugated. In some embodiments, the conjugation is as described herein. In certain embodiments, the pharmaceutical composition comprises an anti-GIP antibody, or antigen-binding fragment thereof, and the pharmaceutical composition is co-administered with a pharmaceutical composition comprising a biologically active peptide.

The compositions disclosed herein can comprise a pharmaceutical composition, and for example include a pharmaceutically acceptable carrier, and/or a pharmaceutical formulation.

The term “pharmaceutical formulation” refers to a preparation which is in such form as to permit the biological activity of an active ingredient contained therein to be effective, and which contains no additional components which are unacceptably toxic to a subject to which the formulation would be administered. A “pharmaceutically acceptable excipient” refers to an ingredient in a pharmaceutical formulation, other than an active ingredient, which is nontoxic to a subject. A pharmaceutically acceptable carrier includes, but is not limited to, a buffer, excipient, stabilizer, or preservative. In some aspects, the choice of carrier is determined in part by the particular cell and/or by the method of administration. Accordingly, there are a variety of suitable formulations. For example, the pharmaceutical composition can contain preservatives. Suitable preservatives may include, for example, methylparaben, propylparaben, sodium benzoate, and benzalkonium chloride. In some aspects, a mixture of two or more preservatives is used. The preservative or mixtures thereof are typically present in an amount of about 0.0001% to about 2% by weight of the total composition. Carriers are described, e.g., by Remington's Pharmaceutical Sciences 16th edition, Osol, A. Ed. (1980). Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG).

Buffering agents in some aspects are included in the compositions. Suitable buffering agents include, for example, citric acid, sodium citrate, phosphoric acid, potassium phosphate, and various other acids and salts. In some aspects, a mixture of two or more buffering agents is used. The buffering agent or mixtures thereof are typically present in an amount of about 0.001% to about 4% by weight of the total composition. Methods for preparing administrable pharmaceutical compositions are known. Exemplary methods are described in more detail in, for example, Remington: The Science and Practice of Pharmacy, Lippincott Williams & Wilkins; 21st ed. (May 1, 2005).

The formulations can include aqueous solutions. The formulation or composition may also contain more than one active ingredient useful for the particular indication, disease, or condition being treated with the composition, preferably those with activities complementary to the composition, where the respective activities do not adversely affect one another. Such active ingredients are suitably present in combination in amounts that are effective for the purpose intended. Thus, in some embodiments, the pharmaceutical composition further includes other pharmaceutically active agents or drugs. The pharmaceutical composition in some embodiments contains the composition in amounts effective to treat or prevent the disease or condition, such as a therapeutically effective or prophylactically effective amount. Therapeutic or prophylactic efficacy in some embodiments is monitored by periodic assessment of treated subjects. The desired dosage can be delivered by a single bolus administration of the composition, by multiple bolus administrations of the composition, or by continuous infusion administration of the composition.

Formulations include those for oral, intravenous, intraperitoneal, subcutaneous, pulmonary, transdermal, intramuscular, intranasal, buccal, sublingual, or suppository administration. In some embodiments, the composition is administered parenterally. The term “parenteral,” as used herein, includes intravenous, intramuscular, subcutaneous, rectal, vaginal, and intraperitoneal administration. In some embodiments, the composition is administered to the subject using peripheral systemic delivery by intravenous, intraperitoneal, or subcutaneous injection. Compositions in some embodiments are provided as sterile liquid preparations, e.g., isotonic aqueous solutions, suspensions, emulsions, dispersions, or viscous compositions, which may in some aspects be buffered to a selected pH. Liquid preparations are normally easier to prepare than gels, other viscous compositions, and solid compositions. Additionally, liquid compositions are somewhat more convenient to administer, especially by injection. Viscous compositions, on the other hand, can be formulated within the appropriate viscosity range to provide longer contact periods with specific tissues. Liquid or viscous compositions can comprise carriers, which can be a solvent or dispersing medium containing, for example, water, saline, phosphate buffered saline, polyol (for example, glycerol, propylene glycol, liquid polyethylene glycol) and suitable mixtures thereof.

Sterile injectable solutions can be prepared by incorporating the composition in a solvent, such as in admixture with a suitable carrier, diluent, or excipient such as sterile water, physiological saline, glucose, dextrose, or the like. The compositions can contain auxiliary substances such as wetting, dispersing, or emulsifying agents (e.g., methylcellulose), pH buffering agents, gelling or viscosity enhancing additives, preservatives, flavoring agents, and/or colors, depending upon the route of administration and the preparation desired. Standard texts may in some aspects be consulted to prepare suitable preparations.

Various additives which enhance the stability and sterility of the compositions, including antimicrobial preservatives, antioxidants, chelating agents, and buffers, can be added. Prevention of the action of microorganisms can be ensured by various antibacterial and antifungal agents, for example, parabens, chlorobutanol, phenol, and sorbic acid. Prolonged absorption of the injectable pharmaceutical form can be brought about by the use of agents delaying absorption, for example, aluminum monostearate and gelatin.

The formulations to be used for in vivo administration are generally sterile. Sterility may be readily accomplished, e.g., by filtration through sterile filtration membranes.

### Methods

In some embodiments, a method of treating disease is provided, the method comprising administering to a subject in need thereof a pharmaceutical composition comprising an anti-GIP antibody, or an antigen-binding fragment thereof, and a biologically active peptide. In some embodiments, the method of treating a disease comprises administering to a subject in need thereof a pharmaceutical composition comprising an anti-GIP antibody, or an antigen-binding fragment thereof, a biologically active peptide, and a pharmaceutically acceptable excipient. In certain embodiments, the disease is a metabolic disorder. In certain embodiments, the disease is selected from the group consisting of, but not limited to, obesity, type 2 diabetes, coronary heart disease, lipodystrophy, hyperlipidemia, hypercholesterolemia, hypertriglyceridemia, Cushing's syndrome, fatty liver disease, hyperphagia, Prader-Willi syndrome, metabolic syndrome, Kleine-Levin syndrome, Bardet-Biedl syndrome, Graves' disease, cancer, Kluver-Bucy syndrome, Pick's disease, Huntington's chorea, Parkinson's disease, hyperthyroidism, ischemic heart disease, stable angina pectoris, unstable angina, acute coronary syndrome, ST elevation myocardial infarction, and non-ST elevation myocardial infarction.

In some embodiments, the method comprises administering an anti-GIP antibody, or an antigen-binding fragment thereof, and a biologically active peptide, thereby treating the disease. In some embodiments, the anti-GIP antibody, or antigen-binding fragment thereof, is as provided for herein. In some embodiments, the biologically active peptide is as provided for herein. In some embodiments, the anti-GIP antibody, or an antigen-binding fragment thereof, is administered at a concentration sufficient to illicit the desired therapeutic effect. In some embodiments, the biologically active peptide is administered at a concentration sufficient to illicit the desired therapeutic effect.

In some embodiments, the method of treating a disease in a subject in need thereof comprises administering a pharmaceutical composition comprising an anti-GIP antibody, or an antigen-binding fragment thereof, a biologically active peptide, and a pharmaceutically acceptable excipient, wherein the anti-GIP antibody, or antigen-binding fragment thereof, comprises a variable heavy chain region (VH) and a variable light chain region (VL), wherein the VH comprises a HCDR1 having the amino acid sequence of SEQ ID NO: 15, a HCDR2 having the amino acid sequence of SEQ ID NO: 26, and an HCDR3 having the amino acid sequence of SEQ ID NO: 17, and wherein the VL comprises a LCDR1 having the amino acid sequence of SEQ ID NO: 27, a LCDR2 having the amino acid sequence of SEQ ID NO: 28, and a LCDR3 having the amino acid sequence of SEQ ID NO: 19, and wherein the biologically active peptide is a GLP-1R agonist, liraglutide, albiglutide, taspoglutide, semaglutide, LY2428757, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91, SEQ ID NO: 92, SEQ ID NO: 93, SEQ ID NO: 94, SEQ ID NO: 95, SEQ ID NO: 96, SEQ ID NO: 97, SEQ ID NO: 98, SEQ ID NO: 99, SEQ ID NO: 100, SEQ ID NO: 101, SEQ ID NO: 102, SEQ ID NO: 103, SEQ ID NO: 104, SEQ ID NO: 105, SEQ ID NO: 106, SEQ ID NO: 107, SEQ ID NO: 108, SEQ ID NO: 109, SEQ ID NO: 110, SEQ ID NO: 111, SEQ ID NO: 112, SEQ ID NO: 113, SEQ ID NO: 114, SEQ ID NO: 115, SEQ ID NO: 116, SEQ ID NO: 117, SEQ ID NO: 118, SEQ ID NO: 119, SEQ ID NO: 120, or SEQ ID NO: 121, or a combination of any of the foregoing. In some embodiments, the method of treating a disease in a subject in need thereof comprises administering a pharmaceutical composition comprising an anti-GIP antibody, or an antigen-binding fragment thereof, a biologically active peptide, and a pharmaceutically acceptable excipient, wherein the anti-GIP antibody, or antigen-binding fragment thereof, comprises a variable heavy chain region (VH) and a variable light chain region (VL), wherein the VH comprises a HCDR1 having the amino acid sequence of SEQ ID NO: 15, a HCDR2 having the amino acid sequence of SEQ ID NO: 26, and an HCDR3 having the amino acid sequence of SEQ ID NO: 17, and wherein the VL comprises a LCDR1 having the amino acid sequence of SEQ ID NO: 27, a LCDR2 having the amino acid sequence of SEQ ID NO: 28, and a LCDR3 having the amino acid sequence of SEQ ID NO: 19, and wherein the biologically active peptide is semaglutide.

In some embodiments, the method of treating a disease in a subject in need thereof comprises administering a pharmaceutical composition comprising an anti-GIP antibody, or an antigen-binding fragment thereof, a biologically active peptide, and a pharmaceutically acceptable excipient, wherein the anti-GIP antibody, or antigen-binding fragment thereof, comprises a variable heavy chain region (VH) and a variable light chain region (VL), wherein the VH comprises a HCDR1 having the amino acid sequence of SEQ ID NO: 15, a HCDR2 having the amino acid sequence of SEQ ID NO: 26, and an HCDR3 having the amino acid sequence of SEQ ID NO: 17, and wherein the VL comprises a LCDR1 having the amino acid sequence of SEQ ID NO: 27, a LCDR2 having the amino acid sequence of SEQ ID NO: 31, and a LCDR3 having the amino acid sequence of SEQ ID NO: 19, and wherein the biologically active peptide a GLP-1R agonist, liraglutide, albiglutide, taspoglutide, semaglutide, LY2428757, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91, SEQ ID NO: 92, SEQ ID NO: 93, SEQ ID NO: 94, SEQ ID NO: 95, SEQ ID NO: 96, SEQ ID NO: 97, SEQ ID NO: 98, SEQ ID NO: 99, SEQ ID NO: 100, SEQ ID NO: 101, SEQ ID NO: 102, SEQ ID NO: 103, SEQ ID NO: 104, SEQ ID NO: 105, SEQ ID NO: 106, SEQ ID NO: 107, SEQ ID NO: 108, SEQ ID NO: 109, SEQ ID NO: 110, SEQ ID NO: 111, SEQ ID NO: 112, SEQ ID NO: 113, SEQ ID NO: 114, SEQ ID NO: 115, SEQ ID NO: 116, SEQ ID NO: 117, SEQ ID NO: 118, SEQ ID NO: 119, SEQ ID NO: 120, or SEQ ID NO: 121, or a combination of any of the foregoing. In some embodiments, the method of treating a disease in a subject in need thereof comprises administering a pharmaceutical composition comprising an anti-GIP antibody, or an antigen-binding fragment thereof, a biologically active peptide, and a pharmaceutically acceptable excipient, wherein the anti-GIP antibody, or antigen-binding fragment thereof, comprises a variable heavy chain region (VH) and a variable light chain region (VL), wherein the VH comprises a HCDR1 having the amino acid sequence of SEQ ID NO: 15, a HCDR2 having the amino acid sequence of SEQ ID NO: 26, and an HCDR3 having the amino acid sequence of SEQ ID NO: 17, and wherein the VL comprises a LCDR1 having the amino acid sequence of SEQ ID NO: 27, a LCDR2 having the amino acid sequence of SEQ ID NO: 31, and a LCDR3 having the amino acid sequence of SEQ ID NO: 19, and wherein the biologically active peptide is semaglutide.

In some embodiments, the method of treating a disease in a subject in need thereof comprises administering a pharmaceutical composition comprising an anti-GIP antibody, or an antigen-binding fragment thereof, a biologically active peptide, and a pharmaceutically acceptable excipient, wherein the anti-GIP antibody, or antigen-binding fragment thereof, comprises a variable heavy chain region (VH) and a variable light chain region (VL), wherein the VH comprises a HCDR1 having the amino acid sequence of SEQ ID NO: 15, a HCDR2 having the amino acid sequence of SEQ ID NO: 26, and an HCDR3 having the amino acid sequence of SEQ ID NO: 17, and wherein the VL comprises a LCDR1 having the amino acid sequence of SEQ ID NO: 27, a LCDR2 having the amino acid sequence of SEQ ID NO: 34, and a LCDR3 having the amino acid sequence of SEQ ID NO: 19, and wherein the biologically active peptide is a GLP-1R agonist, liraglutide, albiglutide, taspoglutide, semaglutide, LY2428757, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91, SEQ ID NO: 92, SEQ ID NO: 93, SEQ ID NO: 94, SEQ ID NO: 95, SEQ ID NO: 96, SEQ ID NO: 97, SEQ ID NO: 98, SEQ ID NO: 99, SEQ ID NO: 100, SEQ ID NO: 101, SEQ ID NO: 102, SEQ ID NO: 103, SEQ ID NO: 104, SEQ ID NO: 105, SEQ ID NO: 106, SEQ ID NO: 107, SEQ ID NO: 108, SEQ ID NO: 109, SEQ ID NO: 110, SEQ ID NO: 111, SEQ ID NO: 112, SEQ ID NO: 113, SEQ ID NO: 114, SEQ ID NO: 115, SEQ ID NO: 116, SEQ ID NO: 117, SEQ ID NO: 118, SEQ ID NO: 119, SEQ ID NO: 120, or SEQ ID NO: 121, or a combination of any of the foregoing. In some embodiments, the method of treating a disease in a subject in need thereof comprises administering a pharmaceutical composition comprising an anti-GIP antibody, or an antigen-binding fragment thereof, a biologically active peptide, and a pharmaceutically acceptable excipient, wherein the anti-GIP antibody, or antigen-binding fragment thereof, comprises a variable heavy chain region (VH) and a variable light chain region (VL), wherein the VH comprises a HCDR1 having the amino acid sequence of SEQ ID NO: 15, a HCDR2 having the amino acid sequence of SEQ ID NO: 26, and an HCDR3 having the amino acid sequence of SEQ ID NO: 17, and wherein the VL comprises a LCDR1 having the amino acid sequence of SEQ ID NO: 27, a LCDR2 having the amino acid sequence of SEQ ID NO: 34, and a LCDR3 having the amino acid sequence of SEQ ID NO: 19, and wherein the biologically active peptide is semaglutide.

In some embodiments, the method of treating a disease in a subject in need thereof comprises administering a pharmaceutical composition comprising an anti-GIP antibody, or an antigen-binding fragment thereof, a biologically active peptide, and a pharmaceutically acceptable excipient, wherein the anti-GIP antibody, or antigen-binding fragment thereof, comprises a variable heavy chain region (VH) and a variable light chain region (VL), wherein the VH comprises a HCDR1 having the amino acid sequence of SEQ ID NO: 15, a HCDR2 having the amino acid sequence of SEQ ID NO: 16, and an HCDR3 having the amino acid sequence of SEQ ID NO: 17, and wherein the VL comprises a LCDR1 having the amino acid sequence of SEQ ID NO: 18, a LCDR2 having the amino acid sequence of SEQ ID NO: 5, and a LCDR3 having the amino acid sequence of SEQ ID NO: 19, and wherein the biologically active peptide is a GLP-1R agonist, liraglutide, albiglutide, taspoglutide, semaglutide, LY2428757, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91, SEQ ID NO: 92, SEQ ID NO: 93, SEQ ID NO: 94, SEQ ID NO: 95, SEQ ID NO: 96, SEQ ID NO: 97, SEQ ID NO: 98, SEQ ID NO: 99, SEQ ID NO: 100, SEQ ID NO: 101, SEQ ID NO: 102, SEQ ID NO: 103, SEQ ID NO: 104, SEQ ID NO: 105, SEQ ID NO: 106, SEQ ID NO: 107, SEQ ID NO: 108, SEQ ID NO: 109, SEQ ID NO: 110, SEQ ID NO: 111, SEQ ID NO: 112, SEQ ID NO: 113, SEQ ID NO: 114, SEQ ID NO: 115, SEQ ID NO: 116, SEQ ID NO: 117, SEQ ID NO: 118, SEQ ID NO: 119, SEQ ID NO: 120, or SEQ ID NO: 121, or a combination of any of the foregoing. In some embodiments, the method of treating a disease in a subject in need thereof comprises administering a pharmaceutical composition comprising an anti-GIP antibody, or an antigen-binding fragment thereof, a biologically active peptide, and a pharmaceutically acceptable excipient, wherein the anti-GIP antibody, or antigen-binding fragment thereof, comprises a variable heavy chain region (VH) and a variable light chain region (VL), wherein the VH comprises a HCDR1 having the amino acid sequence of SEQ ID NO: 15, a HCDR2 having the amino acid sequence of SEQ ID NO: 16, and an HCDR3 having the amino acid sequence of SEQ ID NO: 17, and wherein the VL comprises a LCDR1 having the amino acid sequence of SEQ ID NO: 18, a LCDR2 having the amino acid sequence of SEQ ID NO: 5, and a LCDR3 having the amino acid sequence of SEQ ID NO: 19, and wherein the biologically active peptide is semaglutide.

In some embodiments, the method of treating a disease in a subject in need thereof comprises administering a pharmaceutical composition comprising an anti-GIP antibody, or an antigen-binding fragment thereof, a biologically active peptide, and a pharmaceutically acceptable excipient, wherein the anti-GIP antibody, or antigen-binding fragment thereof, comprises a variable heavy chain region (VH) and a variable light chain region (VL), wherein the VH comprises a HCDR1 having the amino acid sequence of SEQ ID NO: 1, a HCDR2 having the amino acid sequence of SEQ ID NO: 2, and an HCDR3 having the amino acid sequence of SEQ ID NO: 3, and wherein the VL comprises a LCDR1 having the amino acid sequence of SEQ ID NO: 4, a LCDR2 having the amino acid sequence of SEQ ID NO: 5, and a LCDR3 having the amino acid sequence of SEQ ID NO: 6, and wherein the biologically active peptide is a GLP-1R agonist, liraglutide, albiglutide, taspoglutide, semaglutide, LY2428757, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91, SEQ ID NO: 92, SEQ ID NO: 93, SEQ ID NO: 94, SEQ ID NO: 95, SEQ ID NO: 96, SEQ ID NO: 97, SEQ ID NO: 98, SEQ ID NO: 99, SEQ ID NO: 100, SEQ ID NO: 101, SEQ ID NO: 102, SEQ ID NO: 103, SEQ ID NO: 104, SEQ ID NO: 105, SEQ ID NO: 106, SEQ ID NO: 107, SEQ ID NO: 108, SEQ ID NO: 109, SEQ ID NO: 110, SEQ ID NO: 111, SEQ ID NO: 112, SEQ ID NO: 113, SEQ ID NO: 114, SEQ ID NO: 115, SEQ ID NO: 116, SEQ ID NO: 117, SEQ ID NO: 118, SEQ ID NO: 119, SEQ ID NO: 120, or SEQ ID NO: 121, or a combination of any of the foregoing. In some embodiments, the method of treating a disease in a subject in need thereof comprises administering a pharmaceutical composition comprising an anti-GIP antibody, or an antigen-binding fragment thereof, a biologically active peptide, and a pharmaceutically acceptable excipient, wherein the anti-GIP antibody, or antigen-binding fragment thereof, comprises a variable heavy chain region (VH) and a variable light chain region (VL), wherein the VH comprises a HCDR1 having the amino acid sequence of SEQ ID NO: 1, a HCDR2 having the amino acid sequence of SEQ ID NO: 2, and an HCDR3 having the amino acid sequence of SEQ ID NO: 3, and wherein the VL comprises a LCDR1 having the amino acid sequence of SEQ ID NO: 4, a LCDR2 having the amino acid sequence of SEQ ID NO: 5, and a LCDR3 having the amino acid sequence of SEQ ID NO: 6, and wherein the biologically active peptide is semaglutide.

In some embodiments, the method of treating a disease in a subject in need thereof comprises administering a pharmaceutical composition comprising an anti-GIP antibody, or an antigen-binding fragment thereof, a biologically active peptide, and a pharmaceutically acceptable excipient, wherein the anti-GIP antibody, or antigen-binding fragment thereof, comprises a variable heavy chain region (VH) and a variable light chain region (VL), wherein the VH comprises an amino acid sequence of SEQ ID NO: 29, and wherein the VL comprises an amino acid sequence of SEQ ID NO: 30, and wherein the biologically active peptide is a GLP-1R agonist, liraglutide, albiglutide, taspoglutide, semaglutide, LY2428757, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91, SEQ ID NO: 92, SEQ ID NO: 93, SEQ ID NO: 94, SEQ ID NO: 95, SEQ ID NO: 96, SEQ ID NO: 97, SEQ ID NO: 98, SEQ ID NO: 99, SEQ ID NO: 100, SEQ ID NO: 101, SEQ ID NO: 102, SEQ ID NO: 103, SEQ ID NO: 104, SEQ ID NO: 105, SEQ ID NO: 106, SEQ ID NO: 107, SEQ ID NO: 108, SEQ ID NO: 109, SEQ ID NO: 110, SEQ ID NO: 111, SEQ ID NO: 112, SEQ ID NO: 113, SEQ ID NO: 114, SEQ ID NO: 115, SEQ ID NO: 116, SEQ ID NO: 117, SEQ ID NO: 118, SEQ ID NO: 119, SEQ ID NO: 120, or SEQ ID NO: 121, or a combination of any of the foregoing. In some embodiments, the method of treating a disease in a subject in need thereof comprises administering a pharmaceutical composition comprising an anti-GIP antibody, or an antigen-binding fragment thereof, a biologically active peptide, and a pharmaceutically acceptable excipient, wherein the anti-GIP antibody, or antigen-binding fragment thereof, comprises a variable heavy chain region (VH) and a variable light chain region (VL), wherein the VH comprises an amino acid sequence of SEQ ID NO: 29, and wherein the VL comprises an amino acid sequence of SEQ ID NO: 30, and wherein the biologically active peptide is semaglutide.

In some embodiments, the method of treating a disease in a subject in need thereof comprises administering a pharmaceutical composition comprising an anti-GIP antibody, or an antigen-binding fragment thereof, a biologically active peptide, and a pharmaceutically acceptable excipient, wherein the anti-GIP antibody, or antigen-binding fragment thereof, comprises a variable heavy chain region (VH) and a variable light chain region (VL), wherein the VH comprises an amino acid sequence of SEQ ID NO: 32, and wherein the VL comprises an amino acid sequence of SEQ ID NO: 33, and wherein the biologically active peptide is a GLP-1R agonist, liraglutide, albiglutide, taspoglutide, semaglutide, LY2428757, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91, SEQ ID NO: 92, SEQ ID NO: 93, SEQ ID NO: 94, SEQ ID NO: 95, SEQ ID NO: 96, SEQ ID NO: 97, SEQ ID NO: 98, SEQ ID NO: 99, SEQ ID NO: 100, SEQ ID NO: 101, SEQ ID NO: 102, SEQ ID NO: 103, SEQ ID NO: 104, SEQ ID NO: 105, SEQ ID NO: 106, SEQ ID NO: 107, SEQ ID NO: 108, SEQ ID NO: 109, SEQ ID NO: 110, SEQ ID NO: 111, SEQ ID NO: 112, SEQ ID NO: 113, SEQ ID NO: 114, SEQ ID NO: 115, SEQ ID NO: 116, SEQ ID NO: 117, SEQ ID NO: 118, SEQ ID NO: 119, SEQ ID NO: 120, or SEQ ID NO: 121, or a combination of any of the foregoing. In some embodiments, the method of treating a disease in a subject in need thereof comprises administering a pharmaceutical composition comprising an anti-GIP antibody, or an antigen-binding fragment thereof, a biologically active peptide, and a pharmaceutically acceptable excipient, wherein the anti-GIP antibody, or antigen-binding fragment thereof, comprises a variable heavy chain region (VH) and a variable light chain region (VL), wherein the VH comprises an amino acid sequence of SEQ ID NO: 32, and wherein the VL comprises an amino acid sequence of SEQ ID NO: 33, and wherein the biologically active peptide is semaglutide.

In some embodiments, the method of treating a disease in a subject in need thereof comprises administering a pharmaceutical composition comprising an anti-GIP antibody, or an antigen-binding fragment thereof, a biologically active peptide, and a pharmaceutically acceptable excipient, wherein the anti-GIP antibody, or antigen-binding fragment thereof, comprises a variable heavy chain region (VH) and a variable light chain region (VL), wherein the VH comprises an amino acid sequence of SEQ ID NO: 35, and wherein the VL comprises an amino acid sequence of SEQ ID NO: 36, and wherein the biologically active peptide a GLP-1R agonist, liraglutide, albiglutide, taspoglutide, semaglutide, LY2428757, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91, SEQ ID NO: 92, SEQ ID NO: 93, SEQ ID NO: 94, SEQ ID NO: 95, SEQ ID NO: 96, SEQ ID NO: 97, SEQ ID NO: 98, SEQ ID NO: 99, SEQ ID NO: 100, SEQ ID NO: 101, SEQ ID NO: 102, SEQ ID NO: 103, SEQ ID NO: 104, SEQ ID NO: 105, SEQ ID NO: 106, SEQ ID NO: 107, SEQ ID NO: 108, SEQ ID NO: 109, SEQ ID NO: 110, SEQ ID NO: 111, SEQ ID NO: 112, SEQ ID NO: 113, SEQ ID NO: 114, SEQ ID NO: 115, SEQ ID NO: 116, SEQ ID NO: 117, SEQ ID NO: 118, SEQ ID NO: 119, SEQ ID NO: 120, or SEQ ID NO: 121, or a combination of any of the foregoing. In some embodiments, the method of treating a disease in a subject in need thereof comprises administering a pharmaceutical composition comprising an anti-GIP antibody, or an antigen-binding fragment thereof, a biologically active peptide, and a pharmaceutically acceptable excipient, wherein the anti-GIP antibody, or antigen-binding fragment thereof, comprises a variable heavy chain region (VH) and a variable light chain region (VL), wherein the VH comprises an amino acid sequence of SEQ ID NO: 35, and wherein the VL comprises an amino acid sequence of SEQ ID NO: 36, and wherein the biologically active peptide is semaglutide.

In some embodiments, the method of treating a disease in a subject in need thereof comprises administering a pharmaceutical composition comprising an anti-GIP antibody, or an antigen-binding fragment thereof, a biologically active peptide, and a pharmaceutically acceptable excipient, wherein the anti-GIP antibody, or antigen-binding fragment thereof, comprises a variable heavy chain region (VH) and a variable light chain region (VL), wherein the VH comprises an amino acid sequence of SEQ ID NO: 24, and wherein the VL comprises an amino acid sequence of SEQ ID NO: 25, and wherein the biologically active peptide is a GLP-1R agonist, liraglutide, albiglutide, taspoglutide, semaglutide, LY2428757, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91, SEQ ID NO: 92, SEQ ID NO: 93, SEQ ID NO: 94, SEQ ID NO: 95, SEQ ID NO: 96, SEQ ID NO: 97, SEQ ID NO: 98, SEQ ID NO: 99, SEQ ID NO: 100, SEQ ID NO: 101, SEQ ID NO: 102, SEQ ID NO: 103, SEQ ID NO: 104, SEQ ID NO: 105, SEQ ID NO: 106, SEQ ID NO: 107, SEQ ID NO: 108, SEQ ID NO: 109, SEQ ID NO: 110, SEQ ID NO: 111, SEQ ID NO: 112, SEQ ID NO: 113, SEQ ID NO: 114, SEQ ID NO: 115, SEQ ID NO: 116, SEQ ID NO: 117, SEQ ID NO: 118, SEQ ID NO: 119, SEQ ID NO: 120, or SEQ ID NO: 121, or a combination of any of the foregoing. In some embodiments, the method of treating a disease in a subject in need thereof comprises administering a pharmaceutical composition comprising an anti-GIP antibody, or an antigen-binding fragment thereof, a biologically active peptide, and a pharmaceutically acceptable excipient, wherein the anti-GIP antibody, or antigen-binding fragment thereof, comprises a variable heavy chain region (VH) and a variable light chain region (VL), wherein the VH comprises an amino acid sequence of SEQ ID NO: 24, and wherein the VL comprises an amino acid sequence of SEQ ID NO: 25, and wherein the biologically active peptide is semaglutide.

In some embodiments, the method of treating a disease in a subject in need thereof comprises administering a pharmaceutical composition comprising an anti-GIP antibody, or an antigen-binding fragment thereof, a biologically active peptide, and a pharmaceutically acceptable excipient, wherein the anti-GIP antibody, or antigen-binding fragment thereof, comprises a variable heavy chain region (VH) and a variable light chain region (VL), wherein the VH comprises an amino acid sequence of SEQ ID NO: 13, and wherein the VL comprises an amino acid sequence of SEQ ID NO: 14, and wherein the biologically active peptide is a GLP-1R agonist, liraglutide, albiglutide, taspoglutide, semaglutide, LY2428757, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91, SEQ ID NO: 92, SEQ ID NO: 93, SEQ ID NO: 94, SEQ ID NO: 95, SEQ ID NO: 96, SEQ ID NO: 97, SEQ ID NO: 98, SEQ ID NO: 99, SEQ ID NO: 100, SEQ ID NO: 101, SEQ ID NO: 102, SEQ ID NO: 103, SEQ ID NO: 104, SEQ ID NO: 105, SEQ ID NO: 106, SEQ ID NO: 107, SEQ ID NO: 108, SEQ ID NO: 109, SEQ ID NO: 110, SEQ ID NO: 111, SEQ ID NO: 112, SEQ ID NO: 113, SEQ ID NO: 114, SEQ ID NO: 115, SEQ ID NO: 116, SEQ ID NO: 117, SEQ ID NO: 118, SEQ ID NO: 119, SEQ ID NO: 120, or SEQ ID NO: 121, or a combination of any of the foregoing. In some embodiments, the method of treating a disease in a subject in need thereof comprises administering a pharmaceutical composition comprising an anti-GIP antibody, or an antigen-binding fragment thereof, a biologically active peptide, and a pharmaceutically acceptable excipient, wherein the anti-GIP antibody, or antigen-binding fragment thereof, comprises a variable heavy chain region (VH) and a variable light chain region (VL), wherein the VH comprises an amino acid sequence of SEQ ID NO: 13, and wherein the VL comprises an amino acid sequence of SEQ ID NO: 14, and wherein the biologically active peptide is semaglutide.

In some embodiments, the method of treating a disease in a subject in need thereof comprises administering a pharmaceutical composition comprising an anti-GIP antibody, or an antigen-binding fragment thereof, a biologically active peptide, and a pharmaceutically acceptable excipient, wherein the anti-GIP antibody, or antigen-binding fragment thereof, comprises a heavy chain (HC) and a light chain (LC), wherein the HC comprises an amino acid sequence of SEQ ID NO: 37, and wherein the LC comprises an amino acid sequence of SEQ ID NO: 144, and wherein the biologically active peptide is a GLP-1R agonist, liraglutide, albiglutide, taspoglutide, semaglutide, LY2428757, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91, SEQ ID NO: 92, SEQ ID NO: 93, SEQ ID NO: 94, SEQ ID NO: 95, SEQ ID NO: 96, SEQ ID NO: 97, SEQ ID NO: 98, SEQ ID NO: 99, SEQ ID NO: 100, SEQ ID NO: 101, SEQ ID NO: 102, SEQ ID NO: 103, SEQ ID NO: 104, SEQ ID NO: 105, SEQ ID NO: 106, SEQ ID NO: 107, SEQ ID NO: 108, SEQ ID NO: 109, SEQ ID NO: 110, SEQ ID NO: 111, SEQ ID NO: 112, SEQ ID NO: 113, SEQ ID NO: 114, SEQ ID NO: 115, SEQ ID NO: 116, SEQ ID NO: 117, SEQ ID NO: 118, SEQ ID NO: 119, SEQ ID NO: 120, or SEQ ID NO: 121, or a combination of any of the foregoing. In some embodiments, the method of treating a disease in a subject in need thereof comprises administering a pharmaceutical composition comprising an anti-GIP antibody, or an antigen-binding fragment thereof, a biologically active peptide, and a pharmaceutically acceptable excipient, wherein the anti-GIP antibody, or antigen-binding fragment thereof, comprises a HC and a LC, wherein the HC comprises an amino acid sequence of SEQ ID NO: 37, and wherein the LC comprises an amino acid sequence of SEQ ID NO: 144, and wherein the biologically active peptide is semaglutide.

In some embodiments, the method of treating a disease in a subject in need thereof comprises the co-administration of the anti-GIP antibody, or an antigen-binding fragment thereof, and the biologically active peptide. In the context of the present disclosure, “co-administration” is understood to encompass any embodiments wherein the anti-GIP antibody, or an antigen-binding fragment thereof, and the biologically active peptide are administered during a time period wherein both the anti-GIP antibody, or an antigen-binding fragment thereof, and the biologically active peptide will be circulating within the subject simultaneously. For example, in some embodiments, the method of treating a disease in a subject in need thereof comprises the administration of a first pharmaceutical composition comprising the anti-GIP antibody, or an antigen-binding fragment thereof, and a pharmaceutically acceptable excipient and the administration of a second pharmaceutical composition comprising the biologically active peptide and a pharmaceutically acceptable excipient, wherein the first pharmaceutical composition and the second pharmaceutical composition are administered simultaneously. In some embodiments, the method of treating a disease in a subject in need thereof comprises the administration of a first pharmaceutical composition comprising the anti-GIP antibody, or an antigen-binding fragment thereof, and a pharmaceutically acceptable excipient and the administration of a second pharmaceutical composition comprising the biologically active peptide and a pharmaceutically acceptable excipient, wherein the first pharmaceutical composition is administered either immediately prior to or immediately after the second pharmaceutical composition. In some embodiments, the method of treating a disease in a subject in need thereof comprises the administration of a first pharmaceutical composition comprising the anti-GIP antibody, or an antigen-binding fragment thereof, and a pharmaceutically acceptable excipient and the administration of a second pharmaceutical composition comprising the biologically active peptide and a pharmaceutically acceptable excipient, wherein the first pharmaceutical composition is administered first and the second pharmaceutical composition is administered at a time period after the first pharmaceutical composition, such that the anti-GIP antibody, or an antigen-binding fragment thereof of the first pharmaceutical composition is still circulating within the subject at the time of administering the second pharmaceutical composition. In some embodiments, the method of treating a disease in a subject in need thereof comprises the administration of a first pharmaceutical composition comprising the anti-GIP antibody, or an antigen-binding fragment thereof, and a pharmaceutically acceptable excipient and the administration of a second pharmaceutical composition comprising the biologically active peptide and a pharmaceutically acceptable excipient, wherein the second pharmaceutical composition is administered first and the first pharmaceutical composition is administered at a time period after the second pharmaceutical composition, such that the biologically active peptide of the second pharmaceutical composition is still circulating within the subject at the time of administering the first pharmaceutical composition.

In some embodiments, the anti-GIP antibody, or an antigen-binding fragment thereof, and the biologically active peptide are co-formulated. In the context of the present disclosure, “co-formulated” is understood to encompass any embodiments wherein the anti-GIP antibody, or an antigen-binding fragment thereof, and the biologically active peptide are simultaneously present in the same composition.

In some embodiments, the method comprises administering an anti-GIP antibody, or antigen binding fragment thereof, conjugated to a biologically active peptide. In some embodiments, the anti-GIP antibody, or antigen binding fragment thereof, is conjugated to the biologically active peptide as provided for herein.

In some embodiments, the method of treating a disease in a subject in need thereof comprises administering a pharmaceutical composition comprising an anti-GIP antibody, or an antigen-binding fragment thereof, conjugated to a biologically active peptide, and a pharmaceutically acceptable excipient, wherein the anti-GIP antibody, or antigen-binding fragment thereof, comprises a variable heavy chain region (VH) and a variable light chain region (VL), wherein the VH comprises a HCDR1 having the amino acid sequence of SEQ ID NO: 15, a HCDR2 having the amino acid sequence of SEQ ID NO: 26, and an HCDR3 having the amino acid sequence of SEQ ID NO: 17, and wherein the VL comprises a LCDR1 having the amino acid sequence of SEQ ID NO: 27, a LCDR2 having the amino acid sequence of SEQ ID NO: 28, and a LCDR3 having the amino acid sequence of SEQ ID NO: 19, and wherein the biologically active peptide is a GLP-1R agonist, liraglutide, albiglutide, taspoglutide, semaglutide, LY2428757, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91, SEQ ID NO: 92, SEQ ID NO: 93, SEQ ID NO: 94, SEQ ID NO: 95, SEQ ID NO: 96, SEQ ID NO: 97, SEQ ID NO: 98, SEQ ID NO: 99, SEQ ID NO: 100, SEQ ID NO: 101, SEQ ID NO: 102, SEQ ID NO: 103, SEQ ID NO: 104, SEQ ID NO: 105, SEQ ID NO: 106, SEQ ID NO: 107, SEQ ID NO: 108, SEQ ID NO: 109, SEQ ID NO: 110, SEQ ID NO: 111, SEQ ID NO: 112, SEQ ID NO: 113, SEQ ID NO: 114, SEQ ID NO: 115, SEQ ID NO: 116, SEQ ID NO: 117, SEQ ID NO: 118, SEQ ID NO: 119, SEQ ID NO: 120, SEQ ID NO: 121, SEQ ID NO: 145, or SEQ ID NO: 146, or a peptide that is 85%, 90%, 91%, 92%, 93%, 94%, 95%, 96%, 97%, 98%, or 99% identical thereto, or a combination of any of the foregoing. In some embodiments, the method of treating a disease in a subject in need thereof comprises administering a pharmaceutical composition comprising an anti-GIP antibody, or an antigen-binding fragment thereof, conjugated to a biologically active peptide, and a pharmaceutically acceptable excipient, wherein the anti-GIP antibody, or antigen-binding fragment thereof, comprises a variable heavy chain region (VH) and a variable light chain region (VL), wherein the VH comprises a HCDR1 having the amino acid sequence of SEQ ID NO: 15, a HCDR2 having the amino acid sequence of SEQ ID NO: 26, and an HCDR3 having the amino acid sequence of SEQ ID NO: 17, and wherein the VL comprises a LCDR1 having the amino acid sequence of SEQ ID NO: 27, a LCDR2 having the amino acid sequence of SEQ ID NO: 28, and a LCDR3 having the amino acid sequence of SEQ ID NO: 19, and wherein the biologically active peptide is semaglutide.

In some embodiments, the method of treating a disease in a subject in need thereof comprises administering a pharmaceutical composition comprising an anti-GIP antibody, or an antigen-binding fragment thereof, conjugated to a biologically active peptide, and a pharmaceutically acceptable excipient, wherein the anti-GIP antibody, or antigen-binding fragment thereof, comprises a variable heavy chain region (VH) and a variable light chain region (VL), wherein the VH comprises a HCDR1 having the amino acid sequence of SEQ ID NO: 15, a HCDR2 having the amino acid sequence of SEQ ID NO: 26, and an HCDR3 having the amino acid sequence of SEQ ID NO: 17, and wherein the VL comprises a LCDR1 having the amino acid sequence of SEQ ID NO: 27, a LCDR2 having the amino acid sequence of SEQ ID NO: 31, and a LCDR3 having the amino acid sequence of SEQ ID NO: 19, and wherein the biologically active peptide a GLP-1R agonist, liraglutide, albiglutide, taspoglutide, semaglutide, LY2428757, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91, SEQ ID NO: 92, SEQ ID NO: 93, SEQ ID NO: 94, SEQ ID NO: 95, SEQ ID NO: 96, SEQ ID NO: 97, SEQ ID NO: 98, SEQ ID NO: 99, SEQ ID NO: 100, SEQ ID NO: 101, SEQ ID NO: 102, SEQ ID NO: 103, SEQ ID NO: 104, SEQ ID NO: 105, SEQ ID NO: 106, SEQ ID NO: 107, SEQ ID NO: 108, SEQ ID NO: 109, SEQ ID NO: 110, SEQ ID NO: 111, SEQ ID NO: 112, SEQ ID NO: 113, SEQ ID NO: 114, SEQ ID NO: 115, SEQ ID NO: 116, SEQ ID NO: 117, SEQ ID NO: 118, SEQ ID NO: 119, SEQ ID NO: 120, SEQ ID NO: 121, SEQ ID NO: 145, or SEQ ID NO: 146, or a peptide that is 85%, 90%, 91%, 92%, 93%, 94%, 95%, 96%, 97%, 98%, or 99% identical thereto, or a combination of any of the foregoing. In some embodiments, the method of treating a disease in a subject in need thereof comprises administering a pharmaceutical composition comprising an anti-GIP antibody, or an antigen-binding fragment thereof, conjugated to a biologically active peptide, and a pharmaceutically acceptable excipient, wherein the anti-GIP antibody, or antigen-binding fragment thereof, comprises a variable heavy chain region (VH) and a variable light chain region (VL), wherein the VH comprises a HCDR1 having the amino acid sequence of SEQ ID NO: 15, a HCDR2 having the amino acid sequence of SEQ ID NO: 26, and an HCDR3 having the amino acid sequence of SEQ ID NO: 17, and wherein the VL comprises a LCDR1 having the amino acid sequence of SEQ ID NO: 27, a LCDR2 having the amino acid sequence of SEQ ID NO: 31, and a LCDR3 having the amino acid sequence of SEQ ID NO: 19, and wherein the biologically active peptide is semaglutide.

In some embodiments, the method of treating a disease in a subject in need thereof comprises administering a pharmaceutical composition comprising an anti-GIP antibody, or an antigen-binding fragment thereof, conjugated to a biologically active peptide, and a pharmaceutically acceptable excipient, wherein the anti-GIP antibody, or antigen-binding fragment thereof, comprises a variable heavy chain region (VH) and a variable light chain region (VL), wherein the VH comprises a HCDR1 having the amino acid sequence of SEQ ID NO: 15, a HCDR2 having the amino acid sequence of SEQ ID NO: 26, and an HCDR3 having the amino acid sequence of SEQ ID NO: 17, and wherein the VL comprises a LCDR1 having the amino acid sequence of SEQ ID NO: 27, a LCDR2 having the amino acid sequence of SEQ ID NO: 34, and a LCDR3 having the amino acid sequence of SEQ ID NO: 19, and wherein the biologically active peptide is a GLP-1R agonist, liraglutide, albiglutide, taspoglutide, semaglutide, LY2428757, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91, SEQ ID NO: 92, SEQ ID NO: 93, SEQ ID NO: 94, SEQ ID NO: 95, SEQ ID NO: 96, SEQ ID NO: 97, SEQ ID NO: 98, SEQ ID NO: 99, SEQ ID NO: 100, SEQ ID NO: 101, SEQ ID NO: 102, SEQ ID NO: 103, SEQ ID NO: 104, SEQ ID NO: 105, SEQ ID NO: 106, SEQ ID NO: 107, SEQ ID NO: 108, SEQ ID NO: 109, SEQ ID NO: 110, SEQ ID NO: 111, SEQ ID NO: 112, SEQ ID NO: 113, SEQ ID NO: 114, SEQ ID NO: 115, SEQ ID NO: 116, SEQ ID NO: 117, SEQ ID NO: 118, SEQ ID NO: 119, SEQ ID NO: 120, SEQ ID NO: 121, SEQ ID NO: 145, or SEQ ID NO: 146, or a peptide that is 85%, 90%, 91%, 92%, 93%, 94%, 95%, 96%, 97%, 98%, or 99% identical thereto, or a combination of any of the foregoing. In some embodiments, the method of treating a disease in a subject in need thereof comprises administering a pharmaceutical composition comprising an anti-GIP antibody, or an antigen-binding fragment thereof, conjugated to a biologically active peptide, and a pharmaceutically acceptable excipient, wherein the anti-GIP antibody, or antigen-binding fragment thereof, comprises a variable heavy chain region (VH) and a variable light chain region (VL), wherein the VH comprises a HCDR1 having the amino acid sequence of SEQ ID NO: 15, a HCDR2 having the amino acid sequence of SEQ ID NO: 26, and an HCDR3 having the amino acid sequence of SEQ ID NO: 17, and wherein the VL comprises a LCDR1 having the amino acid sequence of SEQ ID NO: 27, a LCDR2 having the amino acid sequence of SEQ ID NO: 34, and a LCDR3 having the amino acid sequence of SEQ ID NO: 19, and wherein the biologically active peptide is semaglutide.

In some embodiments, the method of treating a disease in a subject in need thereof comprises administering a pharmaceutical composition comprising an anti-GIP antibody, or an antigen-binding fragment thereof, conjugated to a biologically active peptide, and a pharmaceutically acceptable excipient, wherein the anti-GIP antibody, or antigen-binding fragment thereof, comprises a variable heavy chain region (VH) and a variable light chain region (VL), wherein the VH comprises a HCDR1 having the amino acid sequence of SEQ ID NO: 15, a HCDR2 having the amino acid sequence of SEQ ID NO: 16, and an HCDR3 having the amino acid sequence of SEQ ID NO: 17, and wherein the VL comprises a LCDR1 having the amino acid sequence of SEQ ID NO: 18, a LCDR2 having the amino acid sequence of SEQ ID NO: 5, and a LCDR3 having the amino acid sequence of SEQ ID NO: 19, and wherein the biologically active peptide is a GLP-1R agonist, liraglutide, albiglutide, taspoglutide, semaglutide, LY2428757, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91, SEQ ID NO: 92, SEQ ID NO: 93, SEQ ID NO: 94, SEQ ID NO: 95, SEQ ID NO: 96, SEQ ID NO: 97, SEQ ID NO: 98, SEQ ID NO: 99, SEQ ID NO: 100, SEQ ID NO: 101, SEQ ID NO: 102, SEQ ID NO: 103, SEQ ID NO: 104, SEQ ID NO: 105, SEQ ID NO: 106, SEQ ID NO: 107, SEQ ID NO: 108, SEQ ID NO: 109, SEQ ID NO: 110, SEQ ID NO: 111, SEQ ID NO: 112, SEQ ID NO: 113, SEQ ID NO: 114, SEQ ID NO: 115, SEQ ID NO: 116, SEQ ID NO: 117, SEQ ID NO: 118, SEQ ID NO: 119, SEQ ID NO: 120, SEQ ID NO: 121, SEQ ID NO: 145, or SEQ ID NO: 146, or a peptide that is 85%, 90%, 91%, 92%, 93%, 94%, 95%, 96%, 97%, 98%, or 99% identical thereto, or a combination of any of the foregoing. In some embodiments, the method of treating a disease in a subject in need thereof comprises administering a pharmaceutical composition comprising an anti-GIP antibody, or an antigen-binding fragment thereof, conjugated to a biologically active peptide, and a pharmaceutically acceptable excipient, wherein the anti-GIP antibody, or antigen-binding fragment thereof, comprises a variable heavy chain region (VH) and a variable light chain region (VL), wherein the VH comprises a HCDR1 having the amino acid sequence of SEQ ID NO: 15, a HCDR2 having the amino acid sequence of SEQ ID NO: 16, and an HCDR3 having the amino acid sequence of SEQ ID NO: 17, and wherein the VL comprises a LCDR1 having the amino acid sequence of SEQ ID NO: 18, a LCDR2 having the amino acid sequence of SEQ ID NO: 5, and a LCDR3 having the amino acid sequence of SEQ ID NO: 19, and wherein the biologically active peptide is semaglutide.

In some embodiments, the method of treating a disease in a subject in need thereof comprises administering a pharmaceutical composition comprising an anti-GIP antibody, or an antigen-binding fragment thereof, conjugated to a biologically active peptide, and a pharmaceutically acceptable excipient, wherein the anti-GIP antibody, or antigen-binding fragment thereof, comprises a variable heavy chain region (VH) and a variable light chain region (VL), wherein the VH comprises a HCDR1 having the amino acid sequence of SEQ ID NO: 1, a HCDR2 having the amino acid sequence of SEQ ID NO: 2, and an HCDR3 having the amino acid sequence of SEQ ID NO: 3, and wherein the VL comprises a LCDR1 having the amino acid sequence of SEQ ID NO: 4, a LCDR2 having the amino acid sequence of SEQ ID NO: 5, and a LCDR3 having the amino acid sequence of SEQ ID NO: 6, and wherein the biologically active peptide is a GLP-1R agonist, liraglutide, albiglutide, taspoglutide, semaglutide, LY2428757, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91, SEQ ID NO: 92, SEQ ID NO: 93, SEQ ID NO: 94, SEQ ID NO: 95, SEQ ID NO: 96, SEQ ID NO: 97, SEQ ID NO: 98, SEQ ID NO: 99, SEQ ID NO: 100, SEQ ID NO: 101, SEQ ID NO: 102, SEQ ID NO: 103, SEQ ID NO: 104, SEQ ID NO: 105, SEQ ID NO: 106, SEQ ID NO: 107, SEQ ID NO: 108, SEQ ID NO: 109, SEQ ID NO: 110, SEQ ID NO: 111, SEQ ID NO: 112, SEQ ID NO: 113, SEQ ID NO: 114, SEQ ID NO: 115, SEQ ID NO: 116, SEQ ID NO: 117, SEQ ID NO: 118, SEQ ID NO: 119, SEQ ID NO: 120, SEQ ID NO: 121, SEQ ID NO: 145, or SEQ ID NO: 146, or a peptide that is 85%, 90%, 91%, 92%, 93%, 94%, 95%, 96%, 97%, 98%, or 99% identical thereto, or a combination of any of the foregoing. In some embodiments, the method of treating a disease in a subject in need thereof comprises administering a pharmaceutical composition comprising an anti-GIP antibody, or an antigen-binding fragment thereof, conjugated to a biologically active peptide, and a pharmaceutically acceptable excipient, wherein the anti-GIP antibody, or antigen-binding fragment thereof, comprises a variable heavy chain region (VH) and a variable light chain region (VL), wherein the VH comprises a HCDR1 having the amino acid sequence of SEQ ID NO: 1, a HCDR2 having the amino acid sequence of SEQ ID NO: 2, and an HCDR3 having the amino acid sequence of SEQ ID NO: 3, and wherein the VL comprises a LCDR1 having the amino acid sequence of SEQ ID NO: 4, a LCDR2 having the amino acid sequence of SEQ ID NO: 5, and a LCDR3 having the amino acid sequence of SEQ ID NO: 6, and wherein the biologically active peptide is semaglutide.

In some embodiments, the method of treating a disease in a subject in need thereof comprises administering a pharmaceutical composition comprising an anti-GIP antibody, or an antigen-binding fragment thereof, conjugated to a biologically active peptide, and a pharmaceutically acceptable excipient, wherein the anti-GIP antibody, or antigen-binding fragment thereof, comprises a variable heavy chain region (VH) and a variable light chain region (VL), wherein the VH comprises an amino acid sequence of SEQ ID NO: 29, and wherein the VL comprises an amino acid sequence of SEQ ID NO: 30, and wherein the biologically active peptide is a GLP-1R agonist, liraglutide, albiglutide, taspoglutide, semaglutide, LY2428757, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91, SEQ ID NO: 92, SEQ ID NO: 93, SEQ ID NO: 94, SEQ ID NO: 95, SEQ ID NO: 96, SEQ ID NO: 97, SEQ ID NO: 98, SEQ ID NO: 99, SEQ ID NO: 100, SEQ ID NO: 101, SEQ ID NO: 102, SEQ ID NO: 103, SEQ ID NO: 104, SEQ ID NO: 105, SEQ ID NO: 106, SEQ ID NO: 107, SEQ ID NO: 108, SEQ ID NO: 109, SEQ ID NO: 110, SEQ ID NO: 111, SEQ ID NO: 112, SEQ ID NO: 113, SEQ ID NO: 114, SEQ ID NO: 115, SEQ ID NO: 116, SEQ ID NO: 117, SEQ ID NO: 118, SEQ ID NO: 119, SEQ ID NO: 120, SEQ ID NO: 121, SEQ ID NO: 145, or SEQ ID NO: 146, or a peptide that is 95% identical thereto, or a combination of any of the foregoing. In some embodiments, the method of treating a disease in a subject in need thereof comprises administering a pharmaceutical composition comprising an anti-GIP antibody, or an antigen-binding fragment thereof, conjugated to a biologically active peptide, and a pharmaceutically acceptable excipient, wherein the anti-GIP antibody, or antigen-binding fragment thereof, comprises a variable heavy chain region (VH) and a variable light chain region (VL), wherein the VH comprises an amino acid sequence of SEQ ID NO: 29, and wherein the VL comprises an amino acid sequence of SEQ ID NO: 30, and wherein the biologically active peptide is semaglutide.

In some embodiments, the method of treating a disease in a subject in need thereof comprises administering a pharmaceutical composition comprising an anti-GIP antibody, or an antigen-binding fragment thereof, conjugated to a biologically active peptide, and a pharmaceutically acceptable excipient, wherein the anti-GIP antibody, or antigen-binding fragment thereof, comprises a variable heavy chain region (VH) and a variable light chain region (VL), wherein the VH comprises an amino acid sequence of SEQ ID NO: 32, and wherein the VL comprises an amino acid sequence of SEQ ID NO: 33, and wherein the biologically active peptide is a GLP-1R agonist, liraglutide, albiglutide, taspoglutide, semaglutide, LY2428757, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91, SEQ ID NO: 92, SEQ ID NO: 93, SEQ ID NO: 94, SEQ ID NO: 95, SEQ ID NO: 96, SEQ ID NO: 97, SEQ ID NO: 98, SEQ ID NO: 99, SEQ ID NO: 100, SEQ ID NO: 101, SEQ ID NO: 102, SEQ ID NO: 103, SEQ ID NO: 104, SEQ ID NO: 105, SEQ ID NO: 106, SEQ ID NO: 107, SEQ ID NO: 108, SEQ ID NO: 109, SEQ ID NO: 110, SEQ ID NO: 111, SEQ ID NO: 112, SEQ ID NO: 113, SEQ ID NO: 114, SEQ ID NO: 115, SEQ ID NO: 116, SEQ ID NO: 117, SEQ ID NO: 118, SEQ ID NO: 119, SEQ ID NO: 120, SEQ ID NO: 121, SEQ ID NO: 145, or SEQ ID NO: 146, or a peptide that is 95% identical thereto, or a combination of any of the foregoing. In some embodiments, the method of treating a disease in a subject in need thereof comprises administering a pharmaceutical composition comprising an anti-GIP antibody, or an antigen-binding fragment thereof, conjugated to a biologically active peptide, and a pharmaceutically acceptable excipient, wherein the anti-GIP antibody, or antigen-binding fragment thereof, comprises a variable heavy chain region (VH) and a variable light chain region (VL), wherein the VH comprises an amino acid sequence of SEQ ID NO: 32, and wherein the VL comprises an amino acid sequence of SEQ ID NO: 33, and wherein the biologically active peptide is semaglutide.

In some embodiments, the method of treating a disease in a subject in need thereof comprises administering a pharmaceutical composition comprising an anti-GIP antibody, or an antigen-binding fragment thereof, conjugated to a biologically active peptide, and a pharmaceutically acceptable excipient, wherein the anti-GIP antibody, or antigen-binding fragment thereof, comprises a variable heavy chain region (VH) and a variable light chain region (VL), wherein the VH comprises an amino acid sequence of SEQ ID NO: 35, and wherein the VL comprises an amino acid sequence of SEQ ID NO: 36, and wherein the biologically active peptide a GLP-1R agonist, liraglutide, albiglutide, taspoglutide, semaglutide, LY2428757, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91, SEQ ID NO: 92, SEQ ID NO: 93, SEQ ID NO: 94, SEQ ID NO: 95, SEQ ID NO: 96, SEQ ID NO: 97, SEQ ID NO: 98, SEQ ID NO: 99, SEQ ID NO: 100, SEQ ID NO: 101, SEQ ID NO: 102, SEQ ID NO: 103, SEQ ID NO: 104, SEQ ID NO: 105, SEQ ID NO: 106, SEQ ID NO: 107, SEQ ID NO: 108, SEQ ID NO: 109, SEQ ID NO: 110, SEQ ID NO: 111, SEQ ID NO: 112, SEQ ID NO: 113, SEQ ID NO: 114, SEQ ID NO: 115, SEQ ID NO: 116, SEQ ID NO: 117, SEQ ID NO: 118, SEQ ID NO: 119, SEQ ID NO: 120, SEQ ID NO: 121, SEQ ID NO: 145, or SEQ ID NO: 146, or a peptide that is 95% identical thereto, or a combination of any of the foregoing. In some embodiments, the method of treating a disease in a subject in need thereof comprises administering a pharmaceutical composition comprising an anti-GIP antibody, or an antigen-binding fragment thereof, conjugated to a biologically active peptide, and a pharmaceutically acceptable excipient, wherein the anti-GIP antibody, or antigen-binding fragment thereof, comprises a variable heavy chain region (VH) and a variable light chain region (VL), wherein the VH comprises an amino acid sequence of SEQ ID NO: 35, and wherein the VL comprises an amino acid sequence of SEQ ID NO: 36, and wherein the biologically active peptide is semaglutide.

In some embodiments, the method of treating a disease in a subject in need thereof comprises administering a pharmaceutical composition comprising an anti-GIP antibody, or an antigen-binding fragment thereof, conjugated to a biologically active peptide, and a pharmaceutically acceptable excipient, wherein the anti-GIP antibody, or antigen-binding fragment thereof, comprises a variable heavy chain region (VH) and a variable light chain region (VL), wherein the VH comprises an amino acid sequence of SEQ ID NO: 24, and wherein the VL comprises an amino acid sequence of SEQ ID NO: 25, and wherein the biologically active peptide is a GLP-1R agonist, liraglutide, albiglutide, taspoglutide, semaglutide, LY2428757, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91, SEQ ID NO: 92, SEQ ID NO: 93, SEQ ID NO: 94, SEQ ID NO: 95, SEQ ID NO: 96, SEQ ID NO: 97, SEQ ID NO: 98, SEQ ID NO: 99, SEQ ID NO: 100, SEQ ID NO: 101, SEQ ID NO: 102, SEQ ID NO: 103, SEQ ID NO: 104, SEQ ID NO: 105, SEQ ID NO: 106, SEQ ID NO: 107, SEQ ID NO: 108, SEQ ID NO: 109, SEQ ID NO: 110, SEQ ID NO: 111, SEQ ID NO: 112, SEQ ID NO: 113, SEQ ID NO: 114, SEQ ID NO: 115, SEQ ID NO: 116, SEQ ID NO: 117, SEQ ID NO: 118, SEQ ID NO: 119, SEQ ID NO: 120, SEQ ID NO: 121, SEQ ID NO: 145, or SEQ ID NO: 146, or a peptide that is 95% identical thereto, or a combination of any of the foregoing. In some embodiments, the method of treating a disease in a subject in need thereof comprises administering a pharmaceutical composition comprising an anti-GIP antibody, or an antigen-binding fragment thereof, conjugated to a biologically active peptide, and a pharmaceutically acceptable excipient, wherein the anti-GIP antibody, or antigen-binding fragment thereof, comprises a variable heavy chain region (VH) and a variable light chain region (VL), wherein the VH comprises an amino acid sequence of SEQ ID NO: 24, and wherein the VL comprises an amino acid sequence of SEQ ID NO: 25, and wherein the biologically active peptide is semaglutide.

In some embodiments, the method of treating a disease in a subject in need thereof comprises administering a pharmaceutical composition comprising an anti-GIP antibody, or an antigen-binding fragment thereof, conjugated to a biologically active peptide, and a pharmaceutically acceptable excipient, wherein the anti-GIP antibody, or antigen-binding fragment thereof, comprises a variable heavy chain region (VH) and a variable light chain region (VL), wherein the VH comprises an amino acid sequence of SEQ ID NO: 13, and wherein the VL comprises an amino acid sequence of SEQ ID NO: 14, and wherein the biologically active peptide is a GLP-1R agonist, liraglutide, albiglutide, taspoglutide, semaglutide, LY2428757, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91, SEQ ID NO: 92, SEQ ID NO: 93, SEQ ID NO: 94, SEQ ID NO: 95, SEQ ID NO: 96, SEQ ID NO: 97, SEQ ID NO: 98, SEQ ID NO: 99, SEQ ID NO: 100, SEQ ID NO: 101, SEQ ID NO: 102, SEQ ID NO: 103, SEQ ID NO: 104, SEQ ID NO: 105, SEQ ID NO: 106, SEQ ID NO: 107, SEQ ID NO: 108, SEQ ID NO: 109, SEQ ID NO: 110, SEQ ID NO: 111, SEQ ID NO: 112, SEQ ID NO: 113, SEQ ID NO: 114, SEQ ID NO: 115, SEQ ID NO: 116, SEQ ID NO: 117, SEQ ID NO: 118, SEQ ID NO: 119, SEQ ID NO: 120, SEQ ID NO: 121, SEQ ID NO: 145, or SEQ ID NO: 146, or a peptide that is 95% identical thereto, or a combination of any of the foregoing. In some embodiments, the method of treating a disease in a subject in need thereof comprises administering a pharmaceutical composition comprising an anti-GIP antibody, or an antigen-binding fragment thereof, conjugated to a biologically active peptide, and a pharmaceutically acceptable excipient, wherein the anti-GIP antibody, or antigen-binding fragment thereof, comprises a variable heavy chain region (VH) and a variable light chain region (VL), wherein the VH comprises an amino acid sequence of SEQ ID NO: 13, and wherein the VL comprises an amino acid sequence of SEQ ID NO: 14, and wherein the biologically active peptide is semaglutide.

In some embodiments, the method of treating a disease in a subject in need thereof comprises administering a pharmaceutical composition comprising an anti-GIP antibody, or an antigen-binding fragment thereof, conjugated to a biologically active peptide, and a pharmaceutically acceptable excipient, wherein the anti-GIP antibody, or antigen-binding fragment thereof, comprises a heavy chain (HC) and a light chain (LC), wherein the HC comprises an amino acid sequence of SEQ ID NO: 37, and wherein the LC comprises an amino acid sequence of SEQ ID NO: 144, and wherein the biologically active peptide is a GLP-1R agonist, liraglutide, albiglutide, taspoglutide, semaglutide, LY2428757, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91, SEQ ID NO: 92, SEQ ID NO: 93, SEQ ID NO: 94, SEQ ID NO: 95, SEQ ID NO: 96, SEQ ID NO: 97, SEQ ID NO: 98, SEQ ID NO: 99, SEQ ID NO: 100, SEQ ID NO: 101, SEQ ID NO: 102, SEQ ID NO: 103, SEQ ID NO: 104, SEQ ID NO: 105, SEQ ID NO: 106, SEQ ID NO: 107, SEQ ID NO: 108, SEQ ID NO: 109, SEQ ID NO: 110, SEQ ID NO: 111, SEQ ID NO: 112, SEQ ID NO: 113, SEQ ID NO: 114, SEQ ID NO: 115, SEQ ID NO: 116, SEQ ID NO: 117, SEQ ID NO: 118, SEQ ID NO: 119, SEQ ID NO: 120, SEQ ID NO: 121, SEQ ID NO: 145, or SEQ ID NO: 146, or a peptide that is 95% identical thereto, or a combination of any of the foregoing. In some embodiments, the method of treating a disease in a subject in need thereof comprises administering a pharmaceutical composition comprising an anti-GIP antibody, or an antigen-binding fragment thereof, conjugated to a biologically active peptide, and a pharmaceutically acceptable excipient, wherein the anti-GIP antibody, or antigen-binding fragment thereof, comprises a HC and a LC, wherein the HC comprises an amino acid sequence of SEQ ID NO: 37, and wherein the LC comprises an amino acid sequence of SEQ ID NO: 144, and wherein the biologically active peptide is semaglutide.

In some embodiments, a method of treating a disease is provided. In some embodiments, the method comprises administering one or more pharmaceutical compositions as provided for herein. In some embodiments, the method comprises administering a pharmaceutical composition comprising an anti-GIP antibody, or antigen-binding fragment thereof, as provided for herein and a pharmaceutical composition comprising the biologically active peptide as provided for herein. In some embodiments, the method comprises administering a pharmaceutical composition comprising an anti-GIP antibody, or antigen-binding fragment thereof, as provided for herein and a biologically active peptide as provided for herein. In some embodiments, the method comprises administering a pharmaceutical composition comprising an anti-GIP antibody, or antigen-binding fragment thereof, as provided for herein conjugated to a biologically active peptide as provided for herein.

In some embodiments, the compositions provided herein can be used in methods of treating various diseases or disorders in a patient in need thereof. In some embodiments, the methods comprise administering one or more pharmaceutical compositions as provided for herein to the patient. In some embodiments, the patient is diabetic. In some embodiments, the diabetic is a Type II diabetic. In some embodiments, the diabetic patient is non-obese. In some embodiments, the patient has a body mass index (BMI) that is less than 30. In some embodiments, the patient has a BMI that is less than 25.

In some embodiments, the diabetic patient also has one or more of coronary heart disease, lipodystrophy, hyperlipidemia, hypercholesterolemia, hypertriglyceridemia, fatty liver disease, hyperphagia, metabolic syndrome, hyperthyroidism, ischemic heart disease, hypertension, stable angina pectoris, unstable angina, acute coronary syndrome, ST elevation myocardial infarction, non-ST elevation myocardial infarction.

In some embodiments, the patient is diabetic and obese. In some embodiments, the patient that is obese has a BMI that is 30 or higher. In some embodiments, the patient that is diabetic and b the disease or disorder is coronary heart disease. In some embodiments, the patient is diabetic and the disease or disorder is lipodystrophy. In some embodiments, the patient is diabetic and the disease or disorder is hyperlipidemia. In some embodiments, the patient is diabetic and the disease or disorder is hypercholesterolemia. In some embodiments, the patient is diabetic and the disease or disorder is hypertriglyceridemia. In some embodiments, the patient is diabetic and the disease or disorder is fatty liver disease. In some embodiments, the patient is diabetic and the disease or disorder is hyperphagia. In some embodiments, the patient is diabetic and the disease or disorder is metabolic syndrome. In some embodiments, the patient is diabetic and the disease or disorder is hyperthyroidism. In some embodiments, the patient is diabetic and the disease or disorder is ischemic heart disease. In some embodiments, the patient is diabetic and the disease or disorder is hypertension. In some embodiments, the patient is diabetic and the disease or disorder is stable angina pectoris. In some embodiments, the patient is diabetic and the disease or disorder is unstable angina. In some embodiments, the patient is diabetic and the disease or disorder is acute coronary syndrome. In some embodiments, the patient is diabetic and the disease or disorder is ST elevation myocardial infarction. In some embodiments, the patient is diabetic and the disease or disorder is non-ST elevation myocardial infarction. In some embodiments, the disease or disorder is treated and the patient experiences significant weight loss. In some embodiments, the disease or disorder is treated and the patient does not experience significant weight loss. In some embodiments, the method comprises administering a pharmaceutical composition comprising an anti-GIP antibody, or antigen-binding fragment thereof, as provided for herein and a pharmaceutical composition comprising the biologically active peptide as provided for herein. In some embodiments, the method comprises administering a pharmaceutical composition comprising an anti-GIP antibody, or antigen-binding fragment thereof, as provided for herein and a biologically active peptide as provided for herein. In some embodiments, the method comprises administering a pharmaceutical composition comprising an anti-GIP antibody, or antigen-binding fragment thereof, as provided for herein conjugated to a biologically active peptide as provided for herein.

In some embodiments, a method of blunting or reducing cravings is provided. In some embodiments, the cravings are food cravings. In some embodiments, the carvings are related to an addictive behavior, such as addiction to alcohol or opioids. In some embodiments, the method comprises administering one or more pharmaceutical compositions as provided for herein. In some embodiments, the method comprises administering a pharmaceutical composition comprising an anti-GIP antibody, or antigen-binding fragment thereof, as provided for herein and a pharmaceutical composition comprising the biologically active peptide as provided for herein. In some embodiments, the method comprises administering a pharmaceutical composition comprising an anti-GIP antibody, or antigen-binding fragment thereof, as provided for herein and a biologically active peptide as provided for herein. In some embodiments, the method comprises administering a pharmaceutical composition comprising an anti-GIP antibody, or antigen-binding fragment thereof, as provided for herein conjugated to a biologically active peptide as provided for herein.

### Exemplary Methods

The following methods are exemplary only and are not meant to limit the scope of the present disclosure in any way.

In some embodiments, the method of treating a disease in a subject in need thereof comprises administering a pharmaceutical composition comprising an anti-GIP antibody, or an antigen-binding fragment thereof, and semaglutide, thereby treating the disease, wherein the anti-GIP antibody, or antigen-binding fragment thereof, comprises a VH and a VL, wherein the VH comprises a HCDR1 comprising an amino acid sequence of SEQ ID NO: 15, a HCDR2 comprising an amino acid sequence of SEQ ID NO: 26, and a HCDR3 comprising an amino acid sequence of SEQ ID NO: 17, and the VL comprises a LCDR1 comprising an amino acid sequence of SEQ ID NO: 27, a LCDR2 comprising an amino acid sequence of SEQ ID NO: 28, and a LCDR3 comprising an amino acid sequence of SEQ ID NO: 19. In some embodiments, the method of treating a disease in a subject in need thereof comprises administering a pharmaceutical composition comprising an anti-GIP antibody, or an antigen-binding fragment thereof, and semaglutide, thereby treating the disease, wherein the anti-GIP antibody, or antigen-binding fragment thereof, comprises a VH having at least 90% identity to the amino acid sequence of SEQ ID NO: 29 and a VL having at least 90% identity to the amino acid sequence of SEQ ID NO: 30. In some embodiments, the method of treating a disease in a subject in need thereof comprises administering a pharmaceutical composition comprising an anti-GIP antibody, or an antigen-binding fragment thereof, and semaglutide, thereby treating the disease, wherein the anti-GIP antibody, or antigen-binding fragment thereof, comprises a VH having at least 95% identity to the amino acid sequence of SEQ ID NO: 29 and a VL having at least 95% identity to the amino acid sequence of SEQ ID NO: 30. In some embodiments, the method of treating a disease in a subject in need thereof comprises administering a pharmaceutical composition comprising an anti-GIP antibody, or an antigen-binding fragment thereof, and semaglutide, thereby treating the disease, wherein the anti-GIP antibody, or antigen-binding fragment thereof, comprises a VH having at least 98% identity to the amino acid sequence of SEQ ID NO: 29 and a VL having at least 98% identity to the amino acid sequence of SEQ ID NO: 30. In some embodiments, the method of treating a disease in a subject in need thereof comprises administering a pharmaceutical composition comprising an anti-GIP antibody, or an antigen-binding fragment thereof, and semaglutide, thereby treating the disease, wherein the anti-GIP antibody, or antigen-binding fragment thereof, comprises a VH comprising the amino acid sequence of SEQ ID NO: 29 and a VL comprising the amino acid sequence of SEQ ID NO: 30.

In some embodiments, the method of treating a disease in a subject in need thereof comprises co-administering a pharmaceutical composition comprising an anti-GIP antibody, or an antigen-binding fragment thereof, and a pharmaceutical composition comprising semaglutide, wherein the anti-GIP antibody, or antigen-binding fragment thereof, comprises a VH and a VL, wherein the VH comprises a HCDR1 comprising an amino acid sequence of SEQ ID NO: 15, a HCDR2 comprising an amino acid sequence of SEQ ID NO: 26, and a HCDR3 comprising an amino acid sequence of SEQ ID NO: 17, and the VL comprises a LCDR1 comprising an amino acid sequence of SEQ ID NO: 27, a LCDR2 comprising an amino acid sequence of SEQ ID NO: 28, and a LCDR3 comprising an amino acid sequence of SEQ ID NO: 19. In some embodiments, the method of treating a disease in a subject in need thereof comprises co-administering a pharmaceutical composition comprising an anti-GIP antibody, or an antigen-binding fragment thereof, and a pharmaceutical composition comprising semaglutide, wherein the anti-GIP antibody, or antigen-binding fragment thereof, comprises a VH having at least 90% identity to the amino acid sequence of SEQ ID NO: 29 and a VL having at least 90% identity to the amino acid sequence of SEQ ID NO: 30. In some embodiments, the method of treating a disease in a subject in need thereof comprises co-administering a pharmaceutical composition comprising an anti-GIP antibody, or an antigen-binding fragment thereof, and a pharmaceutical composition comprising semaglutide, wherein the anti-GIP antibody, or antigen-binding fragment thereof, comprises a VH having at least 95% identity to the amino acid sequence of SEQ ID NO: 29 and a VL having at least 95% identity to the amino acid sequence of SEQ ID NO: 30. In some embodiments, the method of treating a disease in a subject in need thereof comprises co-administering a pharmaceutical composition comprising an anti-GIP antibody, or an antigen-binding fragment thereof, and a pharmaceutical composition comprising semaglutide, wherein the anti-GIP antibody, or antigen-binding fragment thereof, comprises a VH having at least 98% identity to the amino acid sequence of SEQ ID NO: 29 and a VL having at least 98% identity to the amino acid sequence of SEQ ID NO: 30. In some embodiments, the method of treating a disease in a subject in need thereof comprises co-administering a pharmaceutical composition comprising an anti-GIP antibody, or an antigen-binding fragment thereof, and a pharmaceutical composition comprising semaglutide, wherein the anti-GIP antibody, or antigen-binding fragment thereof, comprises a VH comprising the amino acid sequence of SEQ ID NO: 29 and a VL comprising the amino acid sequence of SEQ ID NO: 30.

In some embodiments, the method of treating a disease in a subject in need thereof comprises administering a co-formulation comprising an anti-GIP antibody, or an antigen-binding fragment thereof, and semaglutide, wherein the anti-GIP antibody, or antigen-binding fragment thereof, comprises a VH and a VL, wherein the VH comprises a HCDR1 comprising an amino acid sequence of SEQ ID NO: 15, a HCDR2 comprising an amino acid sequence of SEQ ID NO: 26, and a HCDR3 comprising an amino acid sequence of SEQ ID NO: 17, and the VL comprises a LCDR1 comprising an amino acid sequence of SEQ ID NO: 27, a LCDR2 comprising an amino acid sequence of SEQ ID NO: 28, and a LCDR3 comprising an amino acid sequence of SEQ ID NO: 19. In some embodiments, the method of treating a disease in a subject in need thereof comprises administering a co-formulation comprising an anti-GIP antibody, or an antigen-binding fragment thereof, and semaglutide, wherein the anti-GIP antibody, or antigen-binding fragment thereof, comprises a VH having at least 90% identity to the amino acid sequence of SEQ ID NO: 29 and a VL having at least 90% identity to the amino acid sequence of SEQ ID NO: 30. In some embodiments, the method of treating a disease in a subject in need thereof comprises administering a co-formulation comprising an anti-GIP antibody, or an antigen-binding fragment thereof, and semaglutide, wherein the anti-GIP antibody, or antigen-binding fragment thereof, comprises a VH having at least 95% identity to the amino acid sequence of SEQ ID NO: 29 and a VL having at least 95% identity to the amino acid sequence of SEQ ID NO: 30. In some embodiments, the method of treating a disease in a subject in need thereof comprises administering a co-formulation comprising an anti-GIP antibody, or an antigen-binding fragment thereof, and semaglutide, wherein the anti-GIP antibody, or antigen-binding fragment thereof, comprises a VH having at least 98% identity to the amino acid sequence of SEQ ID NO: 29 and a VL having at least 98% identity to the amino acid sequence of SEQ ID NO: 30. In some embodiments, the method of treating a disease in a subject in need thereof comprises administering a co-formulation comprising an anti-GIP antibody, or an antigen-binding fragment thereof, and semaglutide, wherein the anti-GIP antibody, or antigen-binding fragment thereof, comprises a VH comprising the amino acid sequence of SEQ ID NO: 29 and a VL comprising the amino acid sequence of SEQ ID NO: 30.

In some embodiments, the method of treating a disease in a subject in need thereof comprises administering a pharmaceutical composition comprising an anti-GIP antibody, or an antigen-binding fragment thereof, conjugated to semaglutide, wherein the anti-GIP antibody, or antigen-binding fragment thereof, comprises a VH and a VL, wherein the VH comprises a HCDR1 comprising an amino acid sequence of SEQ ID NO: 15, a HCDR2 comprising an amino acid sequence of SEQ ID NO: 26, and a HCDR3 comprising an amino acid sequence of SEQ ID NO: 17, and the VL comprises a LCDR1 comprising an amino acid sequence of SEQ ID NO: 27, a LCDR2 comprising an amino acid sequence of SEQ ID NO: 28, and a LCDR3 comprising an amino acid sequence of SEQ ID NO: 19. In some embodiments, the method of treating a disease in a subject in need thereof comprises administering a pharmaceutical composition comprising an anti-GIP antibody, or an antigen-binding fragment thereof, conjugated to semaglutide, wherein the anti-GIP antibody, or antigen-binding fragment thereof, comprises a VH having at least 90% identity to the amino acid sequence of SEQ ID NO: 29 and a VL having at least 90% identity to the amino acid sequence of SEQ ID NO: 30. In some embodiments, the method of treating a disease in a subject in need thereof comprises administering a pharmaceutical composition comprising an anti-GIP antibody, or an antigen-binding fragment thereof, conjugated to semaglutide, wherein the anti-GIP antibody, or antigen-binding fragment thereof, comprises a VH having at least 95% identity to the amino acid sequence of SEQ ID NO: 29 and a VL having at least 95% identity to the amino acid sequence of SEQ ID NO: 30. In some embodiments, the method of treating a disease in a subject in need thereof comprises administering a pharmaceutical composition comprising an anti-GIP antibody, or an antigen-binding fragment thereof, conjugated to semaglutide, wherein the anti-GIP antibody, or antigen-binding fragment thereof, comprises a VH having at least 98% identity to the amino acid sequence of SEQ ID NO: 29 and a VL having at least 98% identity to the amino acid sequence of SEQ ID NO: 30. In some embodiments, the method of treating a disease in a subject in need thereof comprises administering a pharmaceutical composition comprising an anti-GIP antibody, or an antigen-binding fragment thereof, conjugated to semaglutide, wherein the anti-GIP antibody, or antigen-binding fragment thereof, comprises a VH comprising the amino acid sequence of SEQ ID NO: 29 and a VL comprising the amino acid sequence of SEQ ID NO: 30.

### Apparatus for Delivery of a Medicament

In some embodiments, an apparatus for the delivery of a medicament is provided.

In some embodiments, the apparatus comprises a single chamber for the delivery of a pharmaceutical composition comprising an anti-GIP antibody, or antigen-binding fragment thereof. In some embodiments, the anti-GIP antibody, or antigen-binding fragment thereof, is as provided for herein.

In some embodiments, the apparatus comprises a single chamber for the delivery of a pharmaceutical composition comprising a biologically active peptide. In some embodiments, the biologically active peptide is as provided for herein.

In some embodiments, the apparatus comprises a single chamber for the delivery of a pharmaceutical composition comprising an anti-GIP antibody, or an antigen-binding fragment thereof, and a biologically active peptide, wherein the anti-GIP antibody, or an antigen-binding fragment thereof, and the biologically active peptide are co-formulated in the pharmaceutical composition. In some embodiments, the anti-GIP antibody, or antigen-binding fragment thereof, is as provided for herein. In some embodiments, the biologically active peptide is as provided for herein.

In some embodiments, the apparatus comprises a single chamber for the delivery of a pharmaceutical composition comprising an anti-GIP antibody, or an antigen-binding fragment thereof, conjugated to a biologically active peptide. In some embodiments, the anti-GIP antibody, or antigen-binding fragment thereof, is as provided for herein. In some embodiments, the biologically active peptide is as provided for herein.

In some embodiments, the apparatus comprises a first chamber and a second chamber, wherein the first chamber comprises a pharmaceutical composition comprising a biologically active peptide, and wherein the second chamber comprises a pharmaceutical composition comprising an anti-GIP antibody, or antigen-binding fragment thereof. In some embodiments, the biologically active peptide is as provided for herein. In some embodiments, the anti-GIP antibody, or antigen-binding fragment thereof is as provided for herein. In some embodiments, the pharmaceutical composition of the first chamber and the pharmaceutical composition of the second chamber are administered sequentially. In some embodiments, the pharmaceutical composition of the first chamber and the pharmaceutical composition of the second chamber are administered simultaneously.

In some embodiments, a method of treating a disease is provided, the method comprising using one or more apparatus as provided for herein to administer an anti-GIP antibody, or antigen-binding fragment thereof, and a biologically active peptide, thereby treating the disease or disorder. In some embodiments, the anti-GIP antibody, or antigen-binding fragment thereof, is as provided for herein. In some embodiments, the biologically active peptide is as provided for herein.

### Enumerated Embodiments

In some embodiments, the following embodiments are provided:

1. A method of treating a disease, comprising administering an anti-glucose-dependent insulinotropic polypeptide (GIP) antibody, or an antigen-binding fragment thereof, and a biologically active peptide, wherein the biologically active peptide comprises a glucagon like peptide 1 receptor (GLP-1R) agonist.
2. The method of embodiment 1, wherein the anti-GIP antibody, or antigen-binding fragment thereof, and the biologically active peptide are co-administered.
3. The method of embodiment 2, wherein the anti-GIP antibody, or antigen-binding fragment thereof, and the biologically active peptide are co-formulated.
4. The method of embodiment 2, wherein the anti-GIP antibody, or antigen-binding fragment thereof, and the biologically active peptide are not co-formulated.
5. The method of any one of embodiments 1-4, wherein the biologically active peptide is conjugated to the anti-GIP antibody, or antigen-binding fragment thereof.
6. The method of embodiment 5, wherein the biologically active peptide is conjugated to the anti-GIP antibody, or antigen-binding fragment thereof, through a cysteine or non-canonical amino acid substitution at one or more conjugation sites.
7. The method of any one of embodiments 1-6, wherein the anti-GIP antibody, or antigen-binding fragment thereof, comprises a CL, CH1, CH2, or CH3 region, or a combination thereof.
8. The method of embodiment 7, wherein the biologically active peptide is conjugated to the anti-GIP antibody, or antigen-binding fragment thereof, within the CL, CH1, CH2, or CH3 region of the anti-GIP antibody, or antigen-binding fragment thereof.
9. The method of any one of embodiments 1-8, wherein the anti-GIP antibody, or antigen-binding fragment thereof, comprises an Fc region.
10. The method of embodiment 9, wherein the Fc region is an IgG Fc.
11. The method of embodiment 10, wherein the IgG Fc is an IgG1, IgG2, IgG3, or IgG4 Fc.
12. The method of embodiment 10 or embodiment 11, wherein the IgG Fc comprises one or more substitutions of a canonical amino acid to a cysteine.
13. The method of embodiment 12, wherein the biologically active peptide is conjugated to the anti-GIP antibody, or antigen-binding fragment thereof, through the substituted cysteine residue on the anti-GIP antibody.
14. The method of any one of embodiments 9-13, wherein the IgG Fc is an IgG1 Fc.
15. The method of embodiment 14, wherein the IgG1 Fc comprises one or more substitutions of a canonical amino acid to a cysteine at position 70, 89, 109, 111, 118, 124, 140, 152, 239, 265, 272, 290, 300, 345, 359, 390, or 442.
16. The method of embodiment 15, wherein the substitution of a canonical amino acid to a cysteine comprises a D70C, E89C, V109C, A111C, A118C, S124C, A140C, E152C, S239C, V265C, E272C, K290C, Y300C, E345C, T359C, N390C, or S442C mutation.
17. The method of embodiment 16, wherein the substitution is a D70C substitution.
18. The method of embodiment 16, wherein the substitution is a E89C substitution.
19. The method of embodiment 16, wherein the substitution is a V109C substitution.
20. The method of embodiment 16, wherein the substitution is a A111C substitution.
21. The method of embodiment 16, wherein the substitution is a A118C substitution.
22. The method of embodiment 16, wherein the substitution is a S124C substitution.
23. The method of embodiment 16, wherein the substitution is a A140C substitution.
24. The method of embodiment 16, wherein the substitution is a E152C substitution.
25. The method of embodiment 16, wherein the substitution is a S239C substitution.
26. The method of embodiment 16, wherein the substitution is a V265C substitution.
27. The method of embodiment 16, wherein the substitution is a E272C substitution.
28. The method of embodiment 16, wherein the substitution is a K290C substitution.
29. The method of embodiment 16, wherein the substitution is a Y300C substitution.
30. The method of embodiment 16, wherein the substitution is a E345C substitution.
31. The method of embodiment 16, wherein the substitution is a T359C substitution.
32. The method of embodiment 16, wherein the substitution is a N390C substitution.
33. The method of embodiment 16, wherein the substitution is a S442C substitution.
34. The method of any one of embodiments 8-33, wherein the CL domain comprises one or more substitutions of a canonical amino acid to a cysteine at position 149, 205, or any combination thereof.
35. The method of embodiment 34, wherein the substitution of a canonical amino acid to a cysteine comprises a K149C or V205C substitution.
36. The method of embodiment 35, wherein the substitution is a K149C substitution.
37. The method of embodiment 35, wherein the substitution is a V205C substitution.
38. The method of any one of embodiments 12-37, wherein the biologically active peptide is conjugated directly to the anti-GIP antibody, or antigen-binding fragment thereof.
39. The method of any one of embodiments 8-38, wherein the biologically active peptide is conjugated indirectly to the anti-GIP antibody, or antigen-binding fragment thereof, such as through a linker molecule.
40. The method of embodiment 39, wherein the wherein the biologically active peptide is conjugated to the anti-GIP antibody, or antigen-binding fragment thereof, through a peptide linker.
41. The method of any one of embodiments 1-40, wherein the GLP-1R agonist is liraglutide, albiglutide, taspoglutide, semaglutide, LY2428757, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91, SEQ ID NO: 92, SEQ ID NO: 93, SEQ ID NO: 94, SEQ ID NO: 95, SEQ ID NO: 96, SEQ ID NO: 97, SEQ ID NO: 98, SEQ ID NO: 99, SEQ ID NO: 100, SEQ ID NO: 101, SEQ ID NO: 102, SEQ ID NO: 103, SEQ ID NO: 104, SEQ ID NO: 105, SEQ ID NO: 106, SEQ ID NO: 107, SEQ ID NO: 108, SEQ ID NO: 109, SEQ ID NO: 110, SEQ ID NO: 111, SEQ ID NO: 112, SEQ ID NO: 113, SEQ ID NO: 114, SEQ ID NO: 115, SEQ ID NO: 116, SEQ ID NO: 117, SEQ ID NO: 118, SEQ ID NO: 119, SEQ ID NO: 120, or SEQ ID NO: 121.
42. The method of embodiment 41, wherein the GLP-1R agonist is SEQ ID NO: 145 or SEQ ID NO: 146.
43. The method of any one of embodiments 1-42, wherein the anti-GIP antibody, or an antigen-binding fragment thereof, comprises a heavy chain (HC) comprising a variable heavy chain region (VH) and a light chain (LC) variable light chain region (VL), wherein the VH comprises a heavy chain complementarity domain 1 (HCDR1), a HCDR2, and a HCDR3 comprising amino acid sequences selected from:
a. SEQ ID NO: 15, SEQ ID NO: 26, and SEQ ID NO: 17,
b. SEQ ID NO: 1, SEQ ID NO: 2, and SEQ ID NO: 3, or
c. SEQ ID NO: 15, SEQ ID NO: 16, and SEQ ID NO: 17,
   and wherein the VL comprises a light chain complementarity domain 1 (LCDR1), a LCDR2, and a LCDR3 comprising amino acid sequences selected from:
d. SEQ ID NO: 27, SEQ ID NO: 28, and SEQ ID NO: 19,
e. SEQ ID NO: 4, SEQ ID NO: 5, and SEQ ID NO: 6,
f. SEQ ID NO: 18, SEQ ID NO: 5, and SEQ ID NO: 19,
g. SEQ ID NO: 27, SEQ ID NO: 31, and SEQ ID NO: 19, or
h. SEQ ID NO: 27, SEQ ID NO: 34, and SEQ ID NO: 19.
   44. The method of embodiment 43, wherein the VH comprises an amino acid sequence having at least 90% identity to an amino acid sequence selected from SEQ ID NO: 29, SEQ ID NO: 13, SEQ ID NO: 24, SEQ ID NO: 32, or SEQ ID NO: 35.
   45. The method of embodiment 43 or 44, wherein the VL comprises an amino acid sequence having at least 90% identity to an amino acid sequence selected from SEQ ID NO: 30, SEQ ID NO: 14, SEQ ID NO: 25, SEQ ID NO: 33, or SEQ ID NO: 36.
   46. The method of any one of embodiments 43-45, wherein the VH comprises an amino acid sequence having at least 90% identity to the amino acid sequence of SEQ ID NO: 29, wherein the VH comprises a HCDR1 comprising an amino acid sequence of SEQ ID NO: 15, a HCDR2 comprising an amino acid sequence of SEQ ID NO: 26, and a HCDR3 comprising an amino acid sequence of SEQ ID NO: 17.
   47. The method of embodiment 46, wherein the VH comprises an amino acid sequence having at least 95% identity to the amino acid sequence of SEQ ID NO: 29.
   48. The method of embodiment 47, wherein the VH comprises an amino acid sequence having at least 98% identity to the amino acid sequence of SEQ ID NO: 29.
   49. The method of embodiment 48, wherein the VH comprises amino acid sequence of SEQ ID NO: 29.
   50. The method of any one of embodiments 43-49, wherein the VL comprises an amino acid sequence having at least 90% identity to the amino acid sequence of SEQ ID NO: 30, wherein the VL comprises a LCDR1 comprising an amino acid sequence of SEQ ID NO: 27, a LCDR2 comprising an amino acid sequence of SEQ ID NO: 28, and a LCDR3 comprising an amino acid sequence of SEQ ID NO: 19.
   51. The method of embodiment 50, wherein the VL comprises an amino acid sequence having at least 95% identity to the amino acid sequence of SEQ ID NO: 30.
   52. The method of embodiment 51, wherein the VL comprises an amino acid sequence having at least 98% identity to the amino acid sequence of SEQ ID NO: 30.
   53. The method of embodiment 52, wherein the VL comprises the amino acid sequence of SEQ ID NO: 30.
   54. The method of any one of embodiments 43-53, wherein the HC comprises an amino acid sequence having at least 90%, 91%, 92%, 93%, 94%, 95%, 96%, 97%, 98%, 99%, or 100% identity to the amino acid sequence of SEQ ID NO: 37.
   55. The method of any one of embodiments 43-54, wherein the LC comprises an amino acid sequence having at least 90%, 91%, 92%, 93%, 94%, 95%, 96%, 97%, 98%, 99%, or 100% identity to the amino acid sequence of SEQ ID NO: 144.
   56. The method of any one of embodiments 43-45, wherein the VH comprises an amino acid sequence having at least 90% identity to the amino acid sequence of SEQ ID NO: 32, wherein the VH comprises a HCDR1 comprising an amino acid sequence of SEQ ID NO: 15, a HCDR2 comprising an amino acid sequence of SEQ ID NO: 26, and a HCDR3 comprising an amino acid sequence of SEQ ID NO: 17.
   57. The method of embodiment 56, wherein the VH comprises an amino acid sequence having at least 95% identity to the amino acid sequence of SEQ ID NO: 32.
   58. The method of embodiment 57, wherein the VH comprises an amino acid sequence having at least 98% identity to the amino acid sequence of SEQ ID NO: 32.
   59. The method of embodiment 58, wherein the VH comprises the amino acid sequence of SEQ ID NO: 32.
   60. The method of any one of embodiments 43-45 or 56-59, wherein the VL comprises an amino acid sequence having at least 90% identity to the amino acid sequence of SEQ ID NO: 33, wherein the VL comprises a LCDR1 comprising an amino acid sequence of SEQ ID NO: 27, a LCDR2 comprising an amino acid sequence of SEQ ID NO: 31, and a LCDR3 comprising an amino acid sequence of SEQ ID NO: 19.
   61. The method of embodiment 60, wherein the VL comprises an amino acid sequence having at least 95% identity to the amino acid sequence of SEQ ID NO: 33.
   62. The method of embodiment 61, wherein the VL comprises an amino acid sequence having at least 98% identity to the amino acid sequence of SEQ ID NO: 33.
   63. The method of embodiment 62, wherein the VL comprises the amino acid sequence of SEQ ID NO: 33.
   64. The method of any one of embodiments 43-45, wherein the VH comprises an amino acid sequence having at least 90% identity to the amino acid sequence of SEQ ID NO: 35, wherein the VH comprises a HCDR1 comprising an amino acid sequence of SEQ ID NO: 15, a HCDR2 comprising an amino acid sequence of SEQ ID NO: 26, and a HCDR3 comprising an amino acid sequence of SEQ ID NO: 17.
   65. The method of embodiment 64, wherein the VH comprises an amino acid sequence having at least 95% identity to the amino acid sequence of SEQ ID NO: 35.
   66. The method of embodiment 65, wherein the VH comprises an amino acid sequence having at least 98% identity to the amino acid sequence of SEQ ID NO: 35.
   67. The method of embodiment 66, wherein the VH comprises the amino acid sequence of SEQ ID NO: 35.
   68. The method of any one of embodiments 43-45 or 64-67, wherein the VL comprises an amino acid sequence having at least 90% identity to the amino acid sequence of SEQ ID NO: 36, wherein the VL comprises a LCDR1 comprising an amino acid sequence of SEQ ID NO: 27, a LCDR2 comprising an amino acid sequence of SEQ ID NO: 34, and a LCDR3 comprising an amino acid sequence of SEQ ID NO: 19.
   69. The method of embodiment 68, wherein the VL comprises an amino acid sequence having at least 95% identity to the amino acid sequence of SEQ ID NO: 36.
   70. The method of embodiment 69, wherein the VL comprises an amino acid sequence having at least 98% identity to the amino acid sequence of SEQ ID NO: 36.
   71. The method of embodiment 70, wherein the VL comprises the amino acid sequence of SEQ ID NO: 36.
   72. The method of any one of embodiments 43-45, wherein the VH comprises an amino acid sequence having at least 90% identity to the amino acid sequence of SEQ ID NO: 24, wherein the VH comprises a HCDR1 comprising an amino acid sequence of SEQ ID NO: 15, a HCDR2 comprising an amino acid sequence of SEQ ID NO: 16, and a HCDR3 comprising an amino acid sequence of SEQ ID NO: 17.
   73. The method of embodiment 72, wherein the VH comprises an amino acid sequence having at least 95% identity to the amino acid sequence of SEQ ID NO: 24.
   74. The method of embodiment 73, wherein the VH comprises an amino acid sequence having at least 98% identity to the amino acid sequence of SEQ ID NO: 24.
   75. The method of embodiment 74, wherein the VH comprises the amino acid sequence of SEQ ID NO: 24.
   76. The method of any one of embodiments 43-45 or 72-75, wherein the VL comprises an amino acid sequence having at least 90% identity to the amino acid sequence of SEQ ID NO: 25, wherein the VL comprises a LCDR1 comprising an amino acid sequence of SEQ ID NO: 18, a LCDR2 comprising an amino acid sequence of SEQ ID NO: 5, and a LCDR3 comprising an amino acid sequence of SEQ ID NO: 19.
   77. The method of embodiment 76, wherein the VL comprises an amino acid sequence having at least 95% identity to the amino acid sequence of SEQ ID NO: 25.
   78. The method of embodiment 77, wherein the VL comprises an amino acid sequence having at least 98% identity to the amino acid sequence of SEQ ID NO: 25.
   79. The method of embodiment 78, wherein the VL comprises the amino acid sequence of SEQ ID NO: 25.
   80. The method of any one of embodiments 43-45, wherein the VH comprises an amino acid sequence having at least 90% identity to the amino acid sequence of SEQ ID NO: 13, wherein the VH comprises a HCDR1 comprising an amino acid sequence of SEQ ID NO: 1, a HCDR2 comprising an amino acid sequence of SEQ ID NO: 2, and a HCDR3 comprising an amino acid sequence of SEQ ID NO: 3.
   81. The method of embodiment 80, wherein the VH comprises an amino acid sequence having at least 95% identity to the amino acid sequence of SEQ ID NO: 13.
   82. The method of embodiment 81, wherein the VH comprises an amino acid sequence having at least 98% identity to the amino acid sequence of SEQ ID NO: 13.
   83. The method of embodiment 82, wherein the VH comprises the amino acid sequence of SEQ ID NO: 13.
   84. The method of any one of embodiments 43-45 or 80-83, wherein the VL comprises an amino acid sequence having at least 90% identity to the amino acid sequence of SEQ ID NO: 14, wherein the VL comprises a LCDR1 comprising an amino acid sequence of SEQ ID NO: 4, a LCDR2 comprising an amino acid sequence of SEQ ID NO: 5, and a LCDR3 comprising an amino acid sequence of SEQ ID NO: 6.
   85. The method of embodiment 84, wherein the VL comprises an amino acid sequence having at least 95% identity to the amino acid sequence of SEQ ID NO: 14.
   86. The method of embodiment 85, wherein the VL comprises an amino acid sequence having at least 98% identity to the amino acid sequence of SEQ ID NO: 14.
   87. The method of embodiment 86, wherein the VL comprises the amino acid sequence of SEQ ID NO: 14.
   88. The method of any one of embodiments 43-45, wherein the VH comprises an amino acid sequence having at least 90% identity to the amino acid sequence of SEQ ID NO: 29 and the VL comprises an amino acid sequence having at least 90% identity to the amino acid sequence of SEQ ID NO: 30, wherein the VH comprises a HCDR1 comprising an amino acid sequence of SEQ ID NO: 15, a HCDR2 comprising an amino acid sequence of SEQ ID NO: 26, and a HCDR3 comprising an amino acid sequence of SEQ ID NO: 17, and the VL comprises a LCDR1 comprising an amino acid sequence of SEQ ID NO: 27, a LCDR2 comprising an amino acid sequence of SEQ ID NO: 28, and a LCDR3 comprising an amino acid sequence of SEQ ID NO: 19.
   89. The method of embodiment 88, wherein the VH comprises an amino acid sequence having at least 95% identity to the amino acid sequence of SEQ ID NO: 29 and the VL comprises an amino acid sequence having at least 95% identity to the amino acid sequence of SEQ ID NO: 30.
   90. The method of embodiment 89, wherein the VH comprises an amino acid sequence having at least 98% identity to the amino acid sequence of SEQ ID NO: 29 and the VL comprises an amino acid sequence having at least 98% identity to the amino acid sequence of SEQ ID NO: 30.
   91. The method of embodiment 90, wherein the VH comprises the amino acid sequence of SEQ ID NO: 29 and the VL comprises the amino acid sequence of SEQ ID NO: 30.
   92. The method of any one of embodiments 43-45, wherein the VH comprises an amino acid sequence having at least 90% identity to the amino acid sequence of SEQ ID NO: 32 and the VL comprises an amino acid sequence having at least 90% identity to the amino acid sequence of SEQ ID NO: 33, wherein the VH comprises a HCDR1 comprising an amino acid sequence of SEQ ID NO: 15, a HCDR2 comprising an amino acid sequence of SEQ ID NO: 26, and a HCDR3 comprising an amino acid sequence of SEQ ID NO: 17, and the VL comprises a LCDR1 comprising an amino acid sequence of SEQ ID NO: 27, a LCDR2 comprising an amino acid sequence of SEQ ID NO: 31, and a LCDR3 comprising an amino acid sequence of SEQ ID NO: 19.
   93. The method of embodiment 92, wherein the VH comprises an amino acid sequence having at least 95% identity to the amino acid sequence of SEQ ID NO: 32 and the VL comprises an amino acid sequence having at least 95% identity to the amino acid sequence of SEQ ID NO: 33.
   94. The method of embodiment 93, wherein the VH comprises an amino acid sequence having at least 98% identity to the amino acid sequence of SEQ ID NO: 32 and the VL comprises an amino acid sequence having at least 98% identity to the amino acid sequence of SEQ ID NO: 33.
   95. The method of embodiment 94, wherein the VH comprises the amino acid sequence of SEQ ID NO: 32 and the VL the amino acid sequence of SEQ ID NO: 33.
   96. The method of any one of embodiments 43-45, wherein the VH comprises an amino acid sequence having at least 90% identity to the amino acid sequence of SEQ ID NO: 35 and the VL comprises an amino acid sequence having at least 90% identity to the amino acid sequence of SEQ ID NO: 36, wherein the VH comprises a HCDR1 comprising an amino acid sequence of SEQ ID NO: 15, a HCDR2 comprising an amino acid sequence of SEQ ID NO: 26, and a HCDR3 comprising an amino acid sequence of SEQ ID NO: 17, and the VL comprises a LCDR1 comprising an amino acid sequence of SEQ ID NO: 27, a LCDR2 comprising an amino acid sequence of SEQ ID NO: 34, and a LCDR3 comprising an amino acid sequence of SEQ ID NO: 19.
   97. The method of embodiment 96, wherein the VH comprises an amino acid sequence having at least 95% identity to the amino acid sequence of SEQ ID NO: 35 and the VL comprises an amino acid sequence having at least 95% identity to the amino acid sequence of SEQ ID NO: 36.
   98. The method of embodiment 97, wherein the VH comprises an amino acid sequence having at least 98% identity to the amino acid sequence of SEQ ID NO: 35 and the VL comprises an amino acid sequence having at least 98% identity to the amino acid sequence of SEQ ID NO: 36.
   99. The method of embodiment 98, wherein the VH comprises the amino acid sequence of SEQ ID NO: 35 and the VL comprises the amino acid sequence of SEQ ID NO: 36.
   100. The method of any one of embodiments 43-45, wherein the VH comprises an amino acid sequence having at least 90% identity to the amino acid sequence of SEQ ID NO: 24 and the VL comprises an amino acid sequence having at least 90% identity to the amino acid sequence of SEQ ID NO: 25, wherein the VH comprises a HCDR1 comprising an amino acid sequence of SEQ ID NO: 15, a HCDR2 comprising an amino acid sequence of SEQ ID NO: 16, and a HCDR3 comprising an amino acid sequence of SEQ ID NO: 17, and the VL comprises a LCDR1 comprising an amino acid sequence of SEQ ID NO: 18, a LCDR2 comprising an amino acid sequence of SEQ ID NO: 5, and a LCDR3 comprising an amino acid sequence of SEQ ID NO: 19.
   101. The method of embodiment 100, wherein the VH comprises an amino acid sequence having at least 95% identity to the amino acid sequence of SEQ ID NO: 24 and the VL comprises an amino acid sequence having at least 95% identity to the amino acid sequence of SEQ ID NO: 25.
   102. The method of embodiment 101, wherein the VH comprises an amino acid sequence having at least 98% identity to the amino acid sequence of SEQ ID NO: 24 and the VL comprises an amino acid sequence having at least 98% identity to the amino acid sequence of SEQ ID NO: 25.
   103. The method of embodiment 102, wherein the VH comprises the amino acid sequence of SEQ ID NO: 24 and the VL comprises the amino acid sequence of SEQ ID NO: 25.
   104. The method of any one of embodiments 43-45, wherein the VH comprises an amino acid sequence having at least 90% identity to the amino acid sequence of SEQ ID NO: 13 and the VL comprises an amino acid sequence having at least 90% identity to the amino acid sequence of SEQ ID NO: 14, wherein the VH comprises a HCDR1 comprising an amino acid sequence of SEQ ID NO: 1, a HCDR2 comprising an amino acid sequence of SEQ ID NO: 2, and a HCDR3 comprising an amino acid sequence of SEQ ID NO: 3, and the VL comprises a LCDR1 comprising an amino acid sequence of SEQ ID NO: 4, a LCDR2 comprising an amino acid sequence of SEQ ID NO: 5, and a LCDR3 comprising an amino acid sequence of SEQ ID NO: 6.
   105. The method of embodiment 104, wherein the VH comprises an amino acid sequence having at least 95% identity to the amino acid sequence of SEQ ID NO: 13 and the VL comprises an amino acid sequence having at least 95% identity to the amino acid sequence of SEQ ID NO: 14.
   106. The method of embodiment 105, wherein the VH comprises an amino acid sequence having at least 98% identity to the amino acid sequence of SEQ ID NO: 13 and the VL comprises an amino acid sequence having at least 98% identity to the amino acid sequence of SEQ ID NO: 14.
   107. The method of embodiment 106, wherein the VH comprises the amino acid sequence of SEQ ID NO: 13 and the VL comprises the amino acid sequence of SEQ ID NO: 14.
   108. The method of any one of embodiments 1-107, wherein the anti-GIP antibody, or antigen-binding fragment thereof, selectively binds GIP.
   109. The method of embodiment 108, wherein the anti-GIP antibody, or antigen-binding fragment thereof, does not substantially bind other native incretins or incretin analogues.
   110. A method of treating a disease, comprising administering an anti-glucose-dependent insulinotropic polypeptide (GIP) antibody, or an antigen-binding fragment thereof, and semaglutide.
   111. A method of treating a disease, comprising co-administering an anti-glucose-dependent insulinotropic polypeptide (GIP) antibody, or an antigen-binding fragment thereof, and semaglutide.
   112. A method of treating a disease, comprising administering an effective amount of an anti-glucose-dependent insulinotropic polypeptide (GIP) antibody, or an antigen-binding fragment thereof, conjugated to semaglutide.
   113. A pharmaceutical composition comprising an anti-glucose-dependent insulinotropic polypeptide (GIP) antibody, or an antigen-binding fragment thereof, and a biologically active peptide, wherein the biologically active peptide comprises a glucagon like peptide 1 receptor (GLP-1R) agonist.
   114. The pharmaceutical composition of embodiment 113, wherein the anti-GIP antibody, or antigen-binding fragment thereof, and the biologically active peptide are not linked to each other.
   115. The pharmaceutical composition of embodiment 113, wherein the biologically active peptide is conjugated to the anti-GIP antibody, or antigen-binding fragment thereof.
   116. The pharmaceutical composition of embodiment 114, wherein the biologically active peptide is conjugated to the anti-GIP antibody, or antigen-binding fragment thereof, through a cysteine or non-canonical amino acid substitution at one or more conjugation sites.
   117. The pharmaceutical composition of any one of embodiments 113-116, wherein the anti-GIP antibody, or antigen-binding fragment thereof, comprises a CL, CH1, CH2, or CH3 region, or a combination thereof.
   118. The pharmaceutical composition of embodiment 117, wherein the biologically active peptide is conjugated to the anti-GIP antibody, or antigen-binding fragment thereof, within the CL, CH1, CH2, or CH3 region of the anti-GIP antibody, or antigen-binding fragment thereof.
   119. The pharmaceutical composition of any one of embodiments 113-118, wherein the anti-GIP antibody, or antigen-binding fragment thereof, comprises an Fc region.
   120. The pharmaceutical composition of embodiment 119, wherein the Fc region is an IgG Fc.
   121. The pharmaceutical composition of embodiment 120, wherein the IgG Fc is an IgG1, IgG2, IgG3, or IgG4 Fc.
   122. The pharmaceutical composition of embodiment 120 or embodiment 121, wherein the IgG Fc comprises one or more substitutions of a canonical amino acid to a cysteine.
   123. The pharmaceutical composition of embodiment 122, wherein the biologically active peptide is conjugated to the anti-GIP antibody, or antigen-binding fragment thereof, through the substituted cysteine residue on the anti-GIP antibody.
   124. The pharmaceutical composition of any one of embodiments 120-123, wherein the IgG Fc is an IgG1 Fc.
   125. The pharmaceutical composition of embodiment 124, wherein the IgG1 Fc comprises one or more substitutions of a canonical amino acid to a cysteine at position 70, 89, 109, 111, 118, 124, 140, 152, 239, 265, 272, 290, 300, 345, 359, 390, or 442.
   126. The pharmaceutical composition of embodiment 125, wherein the substitution of a canonical amino acid to a cysteine comprises a D70C, E89C, V109C, A111C, A118C, S124C, A140C, E152C, S239C, V265C, E272C, K290C, Y300C, E345C, T359C, N390C, or S442C mutation.
   127. The pharmaceutical composition of embodiment 126, wherein the substitution is a D70C substitution.
   128. The pharmaceutical composition of embodiment 126, wherein the substitution is a E89C substitution.
   129. The pharmaceutical composition of embodiment 126, wherein the substitution is a V109C substitution.
   130. The pharmaceutical composition of embodiment 126, wherein the substitution is a A111C substitution.
   131. The pharmaceutical composition of embodiment 126, wherein the substitution is a A118C substitution.
   132. The pharmaceutical composition of embodiment 126, wherein the substitution is a S124C substitution.
   133. The pharmaceutical composition of embodiment 126, wherein the substitution is a A140C substitution.
   134. The pharmaceutical composition of embodiment 126, wherein the substitution is a E152C substitution.
   135. The pharmaceutical composition of embodiment 126, wherein the substitution is a S239C substitution.
   136. The pharmaceutical composition of embodiment 126, wherein the substitution is a V265C substitution.
   137. The pharmaceutical composition of embodiment 126, wherein the substitution is a E272C substitution.
   138. The pharmaceutical composition of embodiment 126, wherein the substitution is a K290C substitution.
   139. The pharmaceutical composition of embodiment 126, wherein the substitution is a Y300C substitution.
   140. The pharmaceutical composition of embodiment 126, wherein the substitution is a E345C substitution.
   141. The pharmaceutical composition of embodiment 126, wherein the substitution is a T359C substitution.
   142. The pharmaceutical composition of embodiment 126, wherein the substitution is a N390C substitution.
   143. The pharmaceutical composition of embodiment 126, wherein the substitution is a S442C substitution.
   144. The pharmaceutical composition of any one of embodiments 118-143, wherein the CL domain comprises one or more substitutions of a canonical amino acid to a cysteine at position 149, 205, or any combination thereof.
   145. The pharmaceutical composition of embodiment 144, wherein the substitution of a canonical amino acid to a cysteine comprises a K149C or V205C substitution.
   146. The pharmaceutical composition of embodiment 145, wherein the substitution is a K149C substitution.
   147. The pharmaceutical composition of embodiment 145, wherein the substitution is a V205C substitution.
   148. The pharmaceutical composition of any one of embodiments 118-147, wherein the biologically active peptide is conjugated directly to the anti-GIP antibody, or antigen-binding fragment thereof.
   149. The pharmaceutical composition of any one of embodiments 118-147, wherein the biologically active peptide is conjugated indirectly to the anti-GIP antibody, or antigen-binding fragment thereof, such as through a linker molecule.
   150. The pharmaceutical composition of embodiment 149, wherein the wherein the biologically active peptide is conjugated to the anti-GIP antibody, or antigen-binding fragment thereof, through a peptide linker.
   151. The pharmaceutical composition of any one of embodiments 113-150, wherein the GLP-1R agonist is liraglutide, albiglutide, taspoglutide, semaglutide, LY2428757, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91, SEQ ID NO: 92, SEQ ID NO: 93, SEQ ID NO: 94, SEQ ID NO: 95, SEQ ID NO: 96, SEQ ID NO: 97, SEQ ID NO: 98, SEQ ID NO: 99, SEQ ID NO: 100, SEQ ID NO: 101, SEQ ID NO: 102, SEQ ID NO: 103, SEQ ID NO: 104, SEQ ID NO: 105, SEQ ID NO: 106, SEQ ID NO: 107, SEQ ID NO: 108, SEQ ID NO: 109, SEQ ID NO: 110, SEQ ID NO: 111, SEQ ID NO: 112, SEQ ID NO: 113, SEQ ID NO: 114, SEQ ID NO: 115, SEQ ID NO: 116, SEQ ID NO: 117, SEQ ID NO: 118, SEQ ID NO: 119, SEQ ID NO: 120, or SEQ ID NO: 121.
   152. The pharmaceutical composition of embodiment 151, wherein the GLP-1R agonist is SEQ ID NO: 145 or SEQ ID NO: 146.
   153. The pharmaceutical composition of any one of embodiments 113-152, wherein the anti-GIP antibody, or an antigen-binding fragment thereof, comprises a heavy chain (HC) comprising a variable heavy chain region (VH) and a light chain (LC) comprising a variable light chain region (VL), wherein the VH comprises a heavy chain complementarity domain 1 (HCDR1), a HCDR2, and a HCDR3 comprising amino acid sequences selected from:
a. SEQ ID NO: 15, SEQ ID NO: 26, and SEQ ID NO: 17,
b. SEQ ID NO: 1, SEQ ID NO: 2, and SEQ ID NO: 3, or
c. SEQ ID NO: 15, SEQ ID NO: 16, and SEQ ID NO: 17,
   and wherein the VL comprises a light chain complementarity domain 1 (LCDR1), a LCDR2, and a LCDR3 comprising amino acid sequences selected from:
d. SEQ ID NO: 27, SEQ ID NO: 28, and SEQ ID NO: 19,
c. SEQ ID NO: 4, SEQ ID NO: 5, and SEQ ID NO: 6,
f. SEQ ID NO: 18, SEQ ID NO: 5, and SEQ ID NO: 19,
g. SEQ ID NO: 27, SEQ ID NO: 31, and SEQ ID NO: 19, or
h. SEQ ID NO: 27, SEQ ID NO: 34, and SEQ ID NO: 19.
   154. The pharmaceutical composition of embodiment 153, wherein the VH comprises an amino acid sequence having at least 90% identity to an amino acid sequence selected from SEQ ID NO: 29, SEQ ID NO: 13, SEQ ID NO: 24, SEQ ID NO: 32, or SEQ ID NO: 35.
   155. The pharmaceutical composition of embodiment 153 or 154, wherein the VL comprises an amino acid sequence having at least 90% identity to an amino acid sequence selected from SEQ ID NO: 30, SEQ ID NO: 14, SEQ ID NO: 25, SEQ ID NO: 33, or SEQ ID NO: 36.
   156. The pharmaceutical composition of any one of embodiments 153-155, wherein the VH comprises an amino acid sequence having at least 90% identity to the amino acid sequence of SEQ ID NO: 29, wherein the VH comprises a HCDR1 comprising an amino acid sequence of SEQ ID NO: 15, a HCDR2 comprising an amino acid sequence of SEQ ID NO: 26, and a HCDR3 comprising an amino acid sequence of SEQ ID NO: 17.
   157. The pharmaceutical composition of embodiment 156, wherein the VH comprises an amino acid sequence having at least 95% identity to the amino acid sequence of SEQ ID NO: 29.
   158. The pharmaceutical composition of embodiment 157, wherein the VH comprises an amino acid sequence having at least 98% identity to the amino acid sequence of SEQ ID NO: 29.
   159. The pharmaceutical composition of embodiment 158, wherein the VH comprises amino acid sequence of SEQ ID NO: 29.
   160. The pharmaceutical composition of any one of embodiments 153-159, wherein the VL comprises an amino acid sequence having at least 90% identity to the amino acid sequence of SEQ ID NO: 30, wherein the VL comprises a LCDR1 comprising an amino acid sequence of SEQ ID NO: 27, a LCDR2 comprising an amino acid sequence of SEQ ID NO: 28, and a LCDR3 comprising an amino acid sequence of SEQ ID NO: 19.
   161. The pharmaceutical composition of embodiment 160, wherein the VL comprises an amino acid sequence having at least 95% identity to the amino acid sequence of SEQ ID NO: 30.
   162. The pharmaceutical composition of embodiment 161, wherein the VL comprises an amino acid sequence having at least 98% identity to the amino acid sequence of SEQ ID NO: 30.
   163. The pharmaceutical composition of embodiment 162, wherein the VL comprises the amino acid sequence of SEQ ID NO: 30.
   164. The pharmaceutical composition of any one of embodiments 153-163, wherein the HC comprises an amino acid sequence having at least 90%, 91%, 92%, 93%, 94%, 95%, 96%, 97%, 98%, 99%, or 100% identity to the amino acid sequence of SEQ ID NO: 37.
   165. The pharmaceutical composition of any one of embodiments 153-164, wherein the LC comprises an amino acid sequence having at least 90%, 91%, 92%, 93%, 94%, 95%, 96%, 97%, 98%, 99%, or 100% identity to the amino acid sequence of SEQ ID NO: 144.
   166. The pharmaceutical composition of any one of embodiments 153-155, wherein the VH comprises an amino acid sequence having at least 90% identity to the amino acid sequence of SEQ ID NO: 32, wherein the VH comprises a HCDR1 comprising an amino acid sequence of SEQ ID NO: 15, a HCDR2 comprising an amino acid sequence of SEQ ID NO: 26, and a HCDR3 comprising an amino acid sequence of SEQ ID NO: 17.
   167. The pharmaceutical composition of embodiment 166, wherein the VH comprises an amino acid sequence having at least 95% identity to the amino acid sequence of SEQ ID NO: 32.
   168. The pharmaceutical composition of embodiment 167, wherein the VH comprises an amino acid sequence having at least 98% identity to the amino acid sequence of SEQ ID NO: 32.
   169. The pharmaceutical composition of embodiment 168, wherein the VH comprises the amino acid sequence of SEQ ID NO: 32.
   170. The pharmaceutical composition of any one of embodiments 153-155 or 166-165, wherein the VL comprises an amino acid sequence having at least 90% identity to the amino acid sequence of SEQ ID NO: 33, wherein the VL comprises a LCDR1 comprising an amino acid sequence of SEQ ID NO: 27, a LCDR2 comprising an amino acid sequence of SEQ ID NO: 31, and a LCDR3 comprising an amino acid sequence of SEQ ID NO: 19.
   171. The pharmaceutical composition of embodiment 170, wherein the VL comprises an amino acid sequence having at least 95% identity to the amino acid sequence of SEQ ID NO: 33.
   172. The pharmaceutical composition of embodiment 171, wherein the VL comprises an amino acid sequence having at least 98% identity to the amino acid sequence of SEQ ID NO: 33.
   173. The pharmaceutical composition of embodiment 172, wherein the VL comprises the amino acid sequence of SEQ ID NO: 33.
   174. The pharmaceutical composition of any one of embodiments 153-155, wherein the VH comprises an amino acid sequence having at least 90% identity to the amino acid sequence of SEQ ID NO: 35, wherein the VH comprises a HCDR1 comprising an amino acid sequence of SEQ ID NO: 15, a HCDR2 comprising an amino acid sequence of SEQ ID NO: 26, and a HCDR3 comprising an amino acid sequence of SEQ ID NO: 17.
   175. The pharmaceutical composition of embodiment 174, wherein the VH comprises an amino acid sequence having at least 95% identity to the amino acid sequence of SEQ ID NO: 35.
   176. The pharmaceutical composition of embodiment 175, wherein the VH comprises an amino acid sequence having at least 98% identity to the amino acid sequence of SEQ ID NO: 35.
   177. The pharmaceutical composition of embodiment 176, wherein the VH comprises the amino acid sequence of SEQ ID NO: 35.
   178. The pharmaceutical composition of any one of embodiments 153-155 or 174-177, wherein the VL comprises an amino acid sequence having at least 90% identity to the amino acid sequence of SEQ ID NO: 36, wherein the VL comprises a LCDR1 comprising an amino acid sequence of SEQ ID NO: 27, a LCDR2 comprising an amino acid sequence of SEQ ID NO: 34, and a LCDR3 comprising an amino acid sequence of SEQ ID NO: 19.
   179. The pharmaceutical composition of embodiment 178, wherein the VL comprises an amino acid sequence having at least 95% identity to the amino acid sequence of SEQ ID NO: 36.
   180. The pharmaceutical composition of embodiment 179, wherein the VL comprises an amino acid sequence having at least 98% identity to the amino acid sequence of SEQ ID NO: 36.
   181. The pharmaceutical composition of embodiment 180, wherein the VL comprises the amino acid sequence of SEQ ID NO: 36.
   182. The pharmaceutical composition of any one of embodiments 153-155, wherein the VH comprises an amino acid sequence having at least 90% identity to the amino acid sequence of SEQ ID NO: 24, wherein the VH comprises a HCDR1 comprising an amino acid sequence of SEQ ID NO: 15, a HCDR2 comprising an amino acid sequence of SEQ ID NO: 16, and a HCDR3 comprising an amino acid sequence of SEQ ID NO: 17.
   183. The pharmaceutical composition of embodiment 182, wherein the VH comprises an amino acid sequence having at least 95% identity to the amino acid sequence of SEQ ID NO: 24.
   184. The pharmaceutical composition of embodiment 183, wherein the VH comprises an amino acid sequence having at least 98% identity to the amino acid sequence of SEQ ID NO: 24.
   185. The pharmaceutical composition of embodiment 184, wherein the VH comprises the amino acid sequence of SEQ ID NO: 24.
   186. The pharmaceutical composition of any one of embodiments 153-155 or 182-185, wherein the VL comprises an amino acid sequence having at least 90% identity to the amino acid sequence of SEQ ID NO: 25, wherein the VL comprises a LCDR1 comprising an amino acid sequence of SEQ ID NO: 18, a LCDR2 comprising an amino acid sequence of SEQ ID NO: 5, and a LCDR3 comprising an amino acid sequence of SEQ ID NO: 19.
   187. The pharmaceutical composition of embodiment 186, wherein the VL comprises an amino acid sequence having at least 95% identity to the amino acid sequence of SEQ ID NO: 25.
   188. The pharmaceutical composition of embodiment 187, wherein the VL comprises an amino acid sequence having at least 98% identity to the amino acid sequence of SEQ ID NO: 25.
   189. The pharmaceutical composition of embodiment 188, wherein the VL comprises the amino acid sequence of SEQ ID NO: 25.
   190. The pharmaceutical composition of any one of embodiments 153-155, wherein the VH comprises an amino acid sequence having at least 90% identity to the amino acid sequence of SEQ ID NO: 13, wherein the VH comprises a HCDR1 comprising an amino acid sequence of SEQ ID NO: 1, a HCDR2 comprising an amino acid sequence of SEQ ID NO: 2, and a HCDR3 comprising an amino acid sequence of SEQ ID NO: 3.
   191. The pharmaceutical composition of embodiment 190, wherein the VH comprises an amino acid sequence having at least 95% identity to the amino acid sequence of SEQ ID NO: 13.
   192. The pharmaceutical composition of embodiment 191, wherein the VH comprises an amino acid sequence having at least 98% identity to the amino acid sequence of SEQ ID NO: 13.
   193. The pharmaceutical composition of embodiment 192, wherein the VH comprises the amino acid sequence of SEQ ID NO: 13.
   194. The pharmaceutical composition of any one of embodiments 153-155 or 190-193, wherein the VL comprises an amino acid sequence having at least 90% identity to the amino acid sequence of SEQ ID NO: 14, wherein the VL comprises a LCDR1 comprising an amino acid sequence of SEQ ID NO: 4, a LCDR2 comprising an amino acid sequence of SEQ ID NO: 5, and a LCDR3 comprising an amino acid sequence of SEQ ID NO: 6.
   195. The pharmaceutical composition of embodiment 194, wherein the VL comprises an amino acid sequence having at least 95% identity to the amino acid sequence of SEQ ID NO: 14.
   196. The pharmaceutical composition of embodiment 195, wherein the VL comprises an amino acid sequence having at least 98% identity to the amino acid sequence of SEQ ID NO: 14.
   197. The pharmaceutical composition of embodiment 196, wherein the VL comprises the amino acid sequence of SEQ ID NO: 14.
   198. The pharmaceutical composition of any one of embodiments 153-155, wherein the VH comprises an amino acid sequence having at least 90% identity to the amino acid sequence of SEQ ID NO: 29 and the VL comprises an amino acid sequence having at least 90% identity to the amino acid sequence of SEQ ID NO: 30, wherein the VH comprises a HCDR1 comprising an amino acid sequence of SEQ ID NO: 15, a HCDR2 comprising an amino acid sequence of SEQ ID NO: 26, and a HCDR3 comprising an amino acid sequence of SEQ ID NO: 17, and the VL comprises a LCDR1 comprising an amino acid sequence of SEQ ID NO: 27, a LCDR2 comprising an amino acid sequence of SEQ ID NO: 28, and a LCDR3 comprising an amino acid sequence of SEQ ID NO: 19.
   199. The pharmaceutical composition of embodiment 198, wherein the VH comprises an amino acid sequence having at least 95% identity to the amino acid sequence of SEQ ID NO: 29 and the VL comprises an amino acid sequence having at least 95% identity to the amino acid sequence of SEQ ID NO: 30.
   200. The pharmaceutical composition of embodiment 199, wherein the VH comprises an amino acid sequence having at least 98% identity to the amino acid sequence of SEQ ID NO: 29 and the VL comprises an amino acid sequence having at least 98% identity to the amino acid sequence of SEQ ID NO: 30.
   201. The pharmaceutical composition of embodiment 200, wherein the VH comprises the amino acid sequence of SEQ ID NO: 29 and the VL comprises the amino acid sequence of SEQ ID NO: 30.
   202. The pharmaceutical composition of any one of embodiments 153-155, wherein the VH comprises an amino acid sequence having at least 90% identity to the amino acid sequence of SEQ ID NO: 32 and the VL comprises an amino acid sequence having at least 90% identity to the amino acid sequence of SEQ ID NO: 33, wherein the VH comprises a HCDR1 comprising an amino acid sequence of SEQ ID NO: 15, a HCDR2 comprising an amino acid sequence of SEQ ID NO: 26, and a HCDR3 comprising an amino acid sequence of SEQ ID NO: 17, and the VL comprises a LCDR1 comprising an amino acid sequence of SEQ ID NO: 27, a LCDR2 comprising an amino acid sequence of SEQ ID NO: 31, and a LCDR3 comprising an amino acid sequence of SEQ ID NO: 19.
   203. The pharmaceutical composition of embodiment 202, wherein the VH comprises an amino acid sequence having at least 95% identity to the amino acid sequence of SEQ ID NO: 32 and the VL comprises an amino acid sequence having at least 95% identity to the amino acid sequence of SEQ ID NO: 33.
   204. The pharmaceutical composition of embodiment 203, wherein the VH comprises an amino acid sequence having at least 98% identity to the amino acid sequence of SEQ ID NO: 32 and the VL comprises an amino acid sequence having at least 98% identity to the amino acid sequence of SEQ ID NO: 33.
   205. The pharmaceutical composition of embodiment 204, wherein the VH comprises the amino acid sequence of SEQ ID NO: 32 and the VL the amino acid sequence of SEQ ID NO: 33.
   206. The pharmaceutical composition of any one of embodiments 153-155, wherein the VH comprises an amino acid sequence having at least 90% identity to the amino acid sequence of SEQ ID NO: 35 and the VL comprises an amino acid sequence having at least 90% identity to the amino acid sequence of SEQ ID NO: 36, wherein the VH comprises a HCDR1 comprising an amino acid sequence of SEQ ID NO: 15, a HCDR2 comprising an amino acid sequence of SEQ ID NO: 26, and a HCDR3 comprising an amino acid sequence of SEQ ID NO: 17, and the VL comprises a LCDR1 comprising an amino acid sequence of SEQ ID NO: 27, a LCDR2 comprising an amino acid sequence of SEQ ID NO: 34, and a LCDR3 comprising an amino acid sequence of SEQ ID NO: 19.
   207. The pharmaceutical composition of embodiment 206, wherein the VH comprises an amino acid sequence having at least 95% identity to the amino acid sequence of SEQ ID NO: 35 and the VL comprises an amino acid sequence having at least 95% identity to the amino acid sequence of SEQ ID NO: 36.
   208. The pharmaceutical composition of embodiment 207, wherein the VH comprises an amino acid sequence having at least 98% identity to the amino acid sequence of SEQ ID NO: 35 and the VL comprises an amino acid sequence having at least 98% identity to the amino acid sequence of SEQ ID NO: 36.
   209. The pharmaceutical composition of embodiment 208, wherein the VH comprises the amino acid sequence of SEQ ID NO: 35 and the VL comprises the amino acid sequence of SEQ ID NO: 36.
   210. The pharmaceutical composition of any one of embodiments 153-155, wherein the VH comprises an amino acid sequence having at least 90% identity to the amino acid sequence of SEQ ID NO: 24 and the VL comprises an amino acid sequence having at least 90% identity to the amino acid sequence of SEQ ID NO: 25, wherein the VH comprises a HCDR1 comprising an amino acid sequence of SEQ ID NO: 15, a HCDR2 comprising an amino acid sequence of SEQ ID NO: 16, and a HCDR3 comprising an amino acid sequence of SEQ ID NO: 17, and the VL comprises a LCDR1 comprising an amino acid sequence of SEQ ID NO: 18, a LCDR2 comprising an amino acid sequence of SEQ ID NO: 5, and a LCDR3 comprising an amino acid sequence of SEQ ID NO: 19.
   211. The pharmaceutical composition of embodiment 210, wherein the VH comprises an amino acid sequence having at least 95% identity to the amino acid sequence of SEQ ID NO: 24 and the VL comprises an amino acid sequence having at least 95% identity to the amino acid sequence of SEQ ID NO: 25.
   212. The pharmaceutical composition of embodiment 211, wherein the VH comprises an amino acid sequence having at least 98% identity to the amino acid sequence of SEQ ID NO: 24 and the VL comprises an amino acid sequence having at least 98% identity to the amino acid sequence of SEQ ID NO: 25.
   213. The pharmaceutical composition of embodiment 212, wherein the VH comprises the amino acid sequence of SEQ ID NO: 24 and the VL comprises the amino acid sequence of SEQ ID NO: 25.
   214. The pharmaceutical composition of any one of embodiments 153-155, wherein the VH comprises an amino acid sequence having at least 90% identity to the amino acid sequence of SEQ ID NO: 13 and the VL comprises an amino acid sequence having at least 90% identity to the amino acid sequence of SEQ ID NO: 14, wherein the VH comprises a HCDR1 comprising an amino acid sequence of SEQ ID NO: 1, a HCDR2 comprising an amino acid sequence of SEQ ID NO: 2, and a HCDR3 comprising an amino acid sequence of SEQ ID NO: 3, and the VL comprises a LCDR1 comprising an amino acid sequence of SEQ ID NO: 4, a LCDR2 comprising an amino acid sequence of SEQ ID NO: 5, and a LCDR3 comprising an amino acid sequence of SEQ ID NO: 6.
   215. The pharmaceutical composition of embodiment 214, wherein the VH comprises an amino acid sequence having at least 95% identity to the amino acid sequence of SEQ ID NO: 13 and the VL comprises an amino acid sequence having at least 95% identity to the amino acid sequence of SEQ ID NO: 14.
   216. The pharmaceutical composition of embodiment 215, wherein the VH comprises an amino acid sequence having at least 98% identity to the amino acid sequence of SEQ ID NO: 13 and the VL comprises an amino acid sequence having at least 98% identity to the amino acid sequence of SEQ ID NO: 14.
   217. The pharmaceutical composition of embodiment 216, wherein the VH comprises the amino acid sequence of SEQ ID NO: 13 and the VL comprises the amino acid sequence of SEQ ID NO: 14.
   218. The pharmaceutical composition of any one of embodiments 113-217, wherein the anti-GIP antibody, or antigen-binding fragment thereof, selectively binds GIP.
   219. The pharmaceutical composition of embodiment 218, wherein the anti-GIP antibody, or antigen-binding fragment thereof, does not substantially bind other native incretins or incretin analogues.
   220. An apparatus for the delivery of a medicament, comprising a first chamber and a second chamber, wherein the first chamber comprises a pharmaceutical composition comprising a biologically active peptide and wherein the second chamber comprises a pharmaceutical composition comprising an anti-GIP antibody, or antigen-binding fragment thereof, wherein the biologically active peptide comprises a glucagon like peptide 1 receptor (GLP-1R) agonist.
   221. The apparatus of embodiment 220, wherein the pharmaceutical composition of the first chamber and the pharmaceutical composition of the second chamber are administered sequentially.
   222. The apparatus of embodiment 220, wherein the pharmaceutical composition of the first chamber and the pharmaceutical composition of the second chamber are administered simultaneously.
   223. An apparatus for the delivery of a medicament, comprising a first chamber, wherein the first chamber comprises a pharmaceutical composition comprising an anti-GIP antibody, or antigen-binding fragment thereof conjugated to a biologically active peptide, wherein the biologically active peptide comprises a glucagon like peptide 1 receptor (GLP-1R) agonist.
   224. The apparatus of any one of embodiments 220-223, wherein the GLP-1R agonist is liraglutide, albiglutide, taspoglutide, semaglutide, LY2428757, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91, SEQ ID NO: 92, SEQ ID NO: 93, SEQ ID NO: 94, SEQ ID NO: 95, SEQ ID NO: 96, SEQ ID NO: 97, SEQ ID NO: 98, SEQ ID NO: 99, SEQ ID NO: 100, SEQ ID NO: 101, SEQ ID NO: 102, SEQ ID NO: 103, SEQ ID NO: 104, SEQ ID NO: 105, SEQ ID NO: 106, SEQ ID NO: 107, SEQ ID NO: 108, SEQ ID NO: 109, SEQ ID NO: 110, SEQ ID NO: 111, SEQ ID NO: 112, SEQ ID NO: 113, SEQ ID NO: 114, SEQ ID NO: 115, SEQ ID NO: 116, SEQ ID NO: 117, SEQ ID NO: 118, SEQ ID NO: 119, SEQ ID NO: 120, or SEQ ID NO: 121.
   225. The apparatus of embodiment 224, wherein the GLP-1R agonist is SEQ ID NO: 145 or SEQ ID NO: 146.
   226. The apparatus of any one of embodiments 216-221, wherein the anti-GIP antibody, or antigen-binding fragment thereof comprises a heavy chain (HC) comprising a variable heavy chain region (VH) and a light chain (LC) comprising a variable light chain region (VL), wherein the VH comprises a heavy chain complementarity domain 1 (HCDR1), a HCDR2, and a HCDR3 comprising amino acid sequences selected from:
a. SEQ ID NO: 15, SEQ ID NO: 26, and SEQ ID NO: 17,
b. SEQ ID NO: 1, SEQ ID NO: 2, and SEQ ID NO: 3, or
c. SEQ ID NO: 15, SEQ ID NO: 16, and SEQ ID NO: 17,
   and wherein the VL comprises a light chain complementarity domain 1 (LCDR1), a LCDR2, and a LCDR3 comprising amino acid sequences selected from:
d. SEQ ID NO: 27, SEQ ID NO: 28, and SEQ ID NO: 19,
c. SEQ ID NO: 4, SEQ ID NO: 5, and SEQ ID NO: 6,
f. SEQ ID NO: 18, SEQ ID NO: 5, and SEQ ID NO: 19,
g. SEQ ID NO: 27, SEQ ID NO: 31, and SEQ ID NO: 19, or
h. SEQ ID NO: 27, SEQ ID NO: 34, and SEQ ID NO: 19.
   227. The apparatus of embodiment 226, wherein the VH comprises an amino acid sequence having at least 90% identity to an amino acid sequence selected from SEQ ID NO: 29, SEQ ID NO: 13, SEQ ID NO: 24, SEQ ID NO: 32, or SEQ ID NO: 35.
   228. The apparatus of embodiment 226 or 227, wherein the VL comprises an amino acid sequence having at least 90% identity to an amino acid sequence selected from SEQ ID NO: 30, SEQ ID NO: 14, SEQ ID NO: 25, SEQ ID NO: 33, or SEQ ID NO: 36.
   229. The apparatus of any one of embodiments 220-228, wherein the anti-GIP antibody, or antigen-binding fragment thereof, comprises an Fc region.
   230. The apparatus of embodiment 229, wherein the Fc region is an IgG Fc.
   231. The apparatus of embodiment 230, wherein the IgG Fc is an IgG1, IgG2, IgG3, or IgG4 Fc.
   232. A method of treating a disease, comprising using the apparatus of any one of embodiments 220-231 to administer an anti-glucose-dependent insulinotropic polypeptide (GIP) antibody, or an antigen-binding fragment thereof, and a biologically active peptide.
   233. A conjugate, comprising an anti-glucose-dependent insulinotropic polypeptide (GIP) antibody, or an antigen-binding fragment thereof, conjugated to a biologically active peptide, wherein the biologically active peptide comprises a glucagon like peptide 1 receptor (GLP-1R) agonist.
   234. The conjugate of embodiment 233, wherein the anti-GIP antibody, or antigen-binding fragment thereof comprises a heavy chain (HC) comprising a variable heavy chain region (VH) and a light chain (LC) comprising a variable light chain region (VL), wherein the VH comprises a heavy chain complementarity domain 1 (HCDR1), a HCDR2, and a HCDR3 comprising amino acid sequences selected from:
a. SEQ ID NO: 15, SEQ ID NO: 26, and SEQ ID NO: 17,
b. SEQ ID NO: 1, SEQ ID NO: 2, and SEQ ID NO: 3,
c. SEQ ID NO: 15, SEQ ID NO: 16, and SEQ ID NO: 17, or
   and wherein the VL comprises a light chain complementarity domain 1 (LCDR1), a LCDR2, and a LCDR3 comprising amino acid sequences selected from:
d. SEQ ID NO: 27, SEQ ID NO: 28, and SEQ ID NO: 19,
c. SEQ ID NO: 4, SEQ ID NO: 5, and SEQ ID NO: 6,
f. SEQ ID NO: 18, SEQ ID NO: 5, and SEQ ID NO: 19,
g. SEQ ID NO: 27, SEQ ID NO: 31, and SEQ ID NO: 19, or
h. SEQ ID NO: 27, SEQ ID NO: 34, and SEQ ID NO: 19.
   235. The conjugate of embodiment 234, wherein the VH comprises an amino acid sequence having at least 90% identity to an amino acid sequence selected from SEQ ID NO: 29, SEQ ID NO: 13, SEQ ID NO: 24, SEQ ID NO: 32, or SEQ ID NO: 35.
   236. The conjugate of embodiment 234 or 235, wherein the VL comprises an amino acid sequence having at least 90% identity to an amino acid sequence selected from SEQ ID NO: 30, SEQ ID NO: 14, SEQ ID NO: 25, SEQ ID NO: 33, or SEQ ID NO: 36.
   237. The conjugate of any one of embodiments 234-236, wherein the VH comprises an amino acid sequence having at least 90% identity to the amino acid sequence of SEQ ID NO: 29 and the VL comprises an amino acid sequence having at least 90% identity to the amino acid sequence of SEQ ID NO: 30, wherein the VH comprises a HCDR1 comprising an amino acid sequence of SEQ ID NO: 15, a HCDR2 comprising an amino acid sequence of SEQ ID NO: 26, and a HCDR3 comprising an amino acid sequence of SEQ ID NO: 17, and the VL comprises a LCDR1 comprising an amino acid sequence of SEQ ID NO: 27, a LCDR2 comprising an amino acid sequence of SEQ ID NO: 28, and a LCDR3 comprising an amino acid sequence of SEQ ID NO: 19.
   238. The conjugate of any one of embodiments 234-237, wherein the HC comprises an amino acid sequence having at least 90% identity to the amino acid sequence of SEQ ID NO: 37 and the LC comprises an amino acid sequence having at least 90% identity to the amino acid sequence of SEQ ID NO: 144.
   239. The conjugate of any one of embodiments 234-238, wherein the biologically active peptide is conjugated to the anti-GIP antibody, or antigen-binding fragment thereof, through the substituted cysteine residue on the anti-GIP antibody.
   240. The conjugate of any one of embodiments 233-239, wherein the GLP-1R agonist is liraglutide, albiglutide, taspoglutide, semaglutide, LY2428757, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91, SEQ ID NO: 92, SEQ ID NO: 93, SEQ ID NO: 94, SEQ ID NO: 95, SEQ ID NO: 96, SEQ ID NO: 97, SEQ ID NO: 98, SEQ ID NO: 99, SEQ ID NO: 100, SEQ ID NO: 101, SEQ ID NO: 102, SEQ ID NO: 103, SEQ ID NO: 104, SEQ ID NO: 105, SEQ ID NO: 106, SEQ ID NO: 107, SEQ ID NO: 108, SEQ ID NO: 109, SEQ ID NO: 110, SEQ ID NO: 111, SEQ ID NO: 112, SEQ ID NO: 113, SEQ ID NO: 114, SEQ ID NO: 115, SEQ ID NO: 116, SEQ ID NO: 117, SEQ ID NO: 118, SEQ ID NO: 119, SEQ ID NO: 120, SEQ ID NO: 121, SEQ ID NO: 145, or SEQ ID NO: 146, or a peptide that is 95% identical thereto.
   241. The conjugate of embodiment 240, wherein the GLP-1R agonist is SEQ ID NO: 145 or SEQ ID NO: 146, or a peptide that is 95% identical thereto.
   242. The conjugate of any one of embodiments 233-241, wherein the anti-GIP antibody, or antigen-binding fragment thereof, comprises a CL, CH1, CH2, or CH3 region, or a combination thereof.
   243. The conjugate of embodiment 242, wherein the biologically active peptide is conjugated to the anti-GIP antibody, or antigen-binding fragment thereof, within the CL, CH1, CH2, or CH3 region of the anti-GIP antibody, or antigen-binding fragment thereof.
   244. The conjugate of any one of embodiments 233-243, wherein the anti-GIP antibody, or antigen-binding fragment thereof, comprises an Fc region.
   245. The conjugate of embodiment 244, wherein the Fc region is an IgG Fc.
   246. The conjugate of embodiment 245, wherein the IgG Fc is an IgG1, IgG2, IgG3, or IgG4 Fc.
   247. The conjugate of embodiment 245 or embodiment 246, wherein IgG Fc comprises one or more substitutions of a canonical amino acid to a cysteine.
   248. The conjugate of embodiment 247, wherein the biologically active peptide is conjugated to the anti-GIP antibody, or antigen-binding fragment thereof, through the substituted cysteine residue on the anti-GIP antibody.
   249. The conjugate of any one of embodiments 245-248, wherein the IgG Fc is an IgG1 Fc.
   250. The conjugate of embodiment 249, wherein the IgG1 Fc comprises one or more substitutions of a canonical amino acid to a cysteine at position 70, 89, 109, 111, 118, 124, 140, 152, 239, 265, 272, 290, 300, 345, 359, 390, or 442.
   251. The conjugate of embodiment 250, wherein the substitution of a canonical amino acid to a cysteine comprises a D70C, E89C, V109C, A111C, A118C, S124C, A140C, E152C, S239C, V265C, E272C, K290C, Y300C, E345C, T359C, N390C, or S442C mutation.
   252. The conjugate of embodiment 251, wherein the substitution is a D70C substitution.
   253. The conjugate of embodiment 251, wherein the substitution is a E89C substitution.
   254. The conjugate of embodiment 251, wherein the substitution is a V109C substitution.
   255. The conjugate of embodiment 251, wherein the substitution is a A111C substitution.
   256. The conjugate of embodiment 251, wherein the substitution is a A118C substitution.
   257. The conjugate of embodiment 251, wherein the substitution is a S124C substitution.
   258. The conjugate of embodiment 251, wherein the substitution is a A140C substitution.
   259. The conjugate of embodiment 251, wherein the substitution is a E152C substitution.
   260. The conjugate of embodiment 251, wherein the substitution is a S239C substitution.
   261. The conjugate of embodiment 251, wherein the substitution is a V265C substitution.
   262. The conjugate of embodiment 251, wherein the substitution is a E272C substitution.
   263. The conjugate of embodiment 251, wherein the substitution is a K290C substitution.
   264. The conjugate of embodiment 251, wherein the substitution is a Y300C substitution.
   265. The conjugate of embodiment 251, wherein the substitution is a E345C substitution.
   266. The conjugate of embodiment 251, wherein the substitution is a T359C substitution.
   267. The conjugate of embodiment 251, wherein the substitution is a N390C substitution.
   268. The conjugate of embodiment 251, wherein the substitution is a S442C substitution.
   269. The conjugate of any one of embodiments 243-268, wherein the CL domain comprises one or more substitutions of a canonical amino acid to a cysteine at position 149, 205, or any combination thereof.
   270. The conjugate of embodiment 269, wherein the substitution of a canonical amino acid to a cysteine comprises a K149C or V205C substitution.
   271. The conjugate of embodiment 270, wherein the substitution is a K149C substitution.
   272. The conjugate of embodiment 270, wherein the substitution is a V205C substitution.
   273. A method of treating a disease, comprising administering the conjugate of any one of embodiments 233-272.
   274. A pharmaceutical composition comprising the conjugate of any one of embodiments 233-272.
   275. A composition comprising an anti-GIP antibody, or antigen binding fragment thereof, linked to a biologically active peptide, wherein the biologically active peptide is linked to the anti-GIP antibody, or antigen binding fragment thereof, at a residue on the antibody which reduces the potency of the biologically active peptide.
   276. The composition of embodiment 275, wherein the biologically active peptide linked to the anti-GIP antibody, or antigen binding fragment thereof, has a reduced potency as compared to the biologically active peptide that is not linked to the anti-GIP antibody, or antigen binding fragment thereof.
   277. The composition of embodiment 275, wherein the biologically active peptide linked to the anti-GIP antibody, or antigen binding fragment thereof, has a reduced potency as compared to the biologically active peptide that is linked to the anti-GIP antibody, or antigen binding fragment thereof, at another residue of the antibody.
   278. The composition of any one of embodiments 275-277, wherein the anti-GIP antibody, or antigen-binding fragment thereof, comprises a CL, CH1, CH2, or CH3 region, or a combination thereof.
   279. The composition of embodiment 278, wherein the biologically active peptide is linked to the anti-GIP antibody, or antigen-binding fragment thereof, within the CL, CH1, CH2, or CH3 region of the anti-GIP antibody, or antigen-binding fragment thereof.
   280. The composition of any one of embodiments 275-279, wherein the anti-GIP antibody, or antigen-binding fragment thereof, comprises an Fc region.
   281. The composition of embodiment 280, wherein the Fc region is an IgG Fc.
   282. The composition of embodiment 281, wherein the IgG Fc is an IgG1, IgG2, IgG3, or IgG4 Fc.
   283. The composition of embodiment 281 or embodiment 282, wherein IgG Fc comprises one or more substitutions of a canonical amino acid to a cysteine.
   284. The composition of embodiment 283, wherein the biologically active peptide is linked to the anti-GIP antibody, or antigen-binding fragment thereof, through the substituted cysteine residue on the anti-GIP antibody.
   285. The composition of any one of embodiments 281-284, wherein the IgG Fc is an IgG1 Fc.
   286. The composition of embodiment 285, wherein the IgG1 Fc comprises one or more substitutions of a canonical amino acid to a cysteine at position 70, 89, 109, 111, 118, 124, 140, 152, 239, 265, 272, 290, 300, 345, 359, 390, or 442.
   287. The composition of embodiment 286, wherein the substitution of a canonical amino acid to a cysteine comprises a D70C, E89C, V109C, A111C, A118C, S124C, A140C, E152C, S239C, V265C, E272C, K290C, Y300C, E345C, T359C, N390C, or S442C mutation.
   288. The composition of embodiment 287, wherein the substitution is a D70C substitution.
   289. The composition of embodiment 287, wherein the substitution is a E89C substitution.
   290. The composition of embodiment 287, wherein the substitution is a V109C substitution.
   291. The composition of embodiment 287, wherein the substitution is a A111C substitution.
   292. The composition of embodiment 287, wherein the substitution is a A118C substitution.
   293. The composition of embodiment 287, wherein the substitution is a S124C substitution.
   294. The composition of embodiment 287, wherein the substitution is a A140C substitution.
   295. The composition of embodiment 287, wherein the substitution is a E152C substitution.
   296. The composition of embodiment 287, wherein the substitution is a S239C substitution.
   297. The composition of embodiment 287, wherein the substitution is a V265C substitution.
   298. The composition of embodiment 287, wherein the substitution is a E272C substitution.
   299. The composition of embodiment 287, wherein the substitution is a K290C substitution.
   300. The composition of embodiment 287, wherein the substitution is a Y300C substitution.
   301. The composition of embodiment 287, wherein the substitution is a E345C substitution.
   302. The composition of embodiment 287, wherein the substitution is a T359C substitution.
   303. The composition of embodiment 287, wherein the substitution is a N390C substitution.
   304. The composition of embodiment 287, wherein the substitution is a S442C substitution.
   305. The composition of any one of embodiments 275-304, wherein the CL domain comprises one or more substitutions of a canonical amino acid to a cysteine at position 149, 205, or any combination thereof.
   306. The composition of embodiment 305, wherein the substitution of a canonical amino acid to a cysteine comprises a K149C or V205C substitution.
   307. The composition of embodiment 306, wherein the substitution is a K149C substitution.
   308. The composition of embodiment 306, wherein the substitution is a V205C substitution.
   309. The composition of any one of embodiments 275-308, wherein the anti-GIP antibody, or antigen-binding fragment thereof comprises a heavy chain (HC) comprising a variable heavy chain region (VH) and a light chain (LC) comprising a variable light chain region (VL), wherein the VH comprises a heavy chain complementarity domain 1 (HCDR1), a HCDR2, and a HCDR3 comprising amino acid sequences selected from:
a. SEQ ID NO: 15, SEQ ID NO: 26, and SEQ ID NO: 17,
b. SEQ ID NO: 1, SEQ ID NO: 2, and SEQ ID NO: 3,
c. SEQ ID NO: 15, SEQ ID NO: 16, and SEQ ID NO: 17, or
   and wherein the VL comprises a light chain complementarity domain 1 (LCDR1), a LCDR2, and a LCDR3 comprising amino acid sequences selected from:
d. SEQ ID NO: 27, SEQ ID NO: 28, and SEQ ID NO: 19,
c. SEQ ID NO: 4, SEQ ID NO: 5, and SEQ ID NO: 6,
f. SEQ ID NO: 18, SEQ ID NO: 5, and SEQ ID NO: 19,
g. SEQ ID NO: 27, SEQ ID NO: 31, and SEQ ID NO: 19, or
h. SEQ ID NO: 27, SEQ ID NO: 34, and SEQ ID NO: 19.
   310. The composition of embodiment 309, wherein the VH comprises an amino acid sequence having at least 90% identity to an amino acid sequence selected from SEQ ID NO: 29, SEQ ID NO: 13, SEQ ID NO: 24, SEQ ID NO: 32, or SEQ ID NO: 35.
   311. The composition of embodiment 309 or 310, wherein the VL comprises an amino acid sequence having at least 90% identity to an amino acid sequence selected from SEQ ID NO: 30, SEQ ID NO: 14, SEQ ID NO: 25, SEQ ID NO: 33, or SEQ ID NO: 36.
   312. The composition of any one of embodiments 309-311, wherein the VH comprises an amino acid sequence having at least 90% identity to the amino acid sequence of SEQ ID NO: 29 and the VL comprises an amino acid sequence having at least 90% identity to the amino acid sequence of SEQ ID NO: 30, wherein the VH comprises a HCDR1 comprising an amino acid sequence of SEQ ID NO: 15, a HCDR2 comprising an amino acid sequence of SEQ ID NO: 26, and a HCDR3 comprising an amino acid sequence of SEQ ID NO: 17, and the VL comprises a LCDR1 comprising an amino acid sequence of SEQ ID NO: 27, a LCDR2 comprising an amino acid sequence of SEQ ID NO: 28, and a LCDR3 comprising an amino acid sequence of SEQ ID NO: 19.
   313. The composition of any one of embodiments 309-312, wherein the HC comprises an amino acid sequence having at least 90% identity to the amino acid sequence of SEQ ID NO: 37 and the LC comprises an amino acid sequence having at least 90% identity to the amino acid sequence of SEQ ID NO: 144.
   314. The composition of any one of embodiments 275-313, wherein the GLP-1R agonist is liraglutide, albiglutide, taspoglutide, semaglutide, LY2428757, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91, SEQ ID NO: 92, SEQ ID NO: 93, SEQ ID NO: 94, SEQ ID NO: 95, SEQ ID NO: 96, SEQ ID NO: 97, SEQ ID NO: 98, SEQ ID NO: 99, SEQ ID NO: 100, SEQ ID NO: 101, SEQ ID NO: 102, SEQ ID NO: 103, SEQ ID NO: 104, SEQ ID NO: 105, SEQ ID NO: 106, SEQ ID NO: 107, SEQ ID NO: 108, SEQ ID NO: 109, SEQ ID NO: 110, SEQ ID NO: 111, SEQ ID NO: 112, SEQ ID NO: 113, SEQ ID NO: 114, SEQ ID NO: 115, SEQ ID NO: 116, SEQ ID NO: 117, SEQ ID NO: 118, SEQ ID NO: 119, SEQ ID NO: 120, SEQ ID NO: 121, SEQ ID NO: 145, or SEQ ID NO: 146, or a peptide that is 95% identical thereto.
   315. The composition of embodiment 314, wherein the GLP-1R agonist is SEQ ID NO: 145 or SEQ ID NO: 146, or a peptide that is 95% identical thereto.
   316. A method of treating a disease or disorder in a diabetic patient in need thereof, the method comprising administering to the patient the pharmaceutical composition of any one of embodiments 113-219 or 274, the conjugate of any one of embodiments 233-272, or the composition of any one of embodiments 275-315 to the diabetic patient, thereby treating the disease or disorder in the diabetic patient,
wherein the disease or disorder is selected from coronary heart disease, lipodystrophy, hyperlipidemia, hypercholesterolemia, hypertriglyceridemia, fatty liver disease, hyperphagia, metabolic syndrome, hyperthyroidism, ischemic heart disease, hypertension, stable angina pectoris, unstable angina, acute coronary syndrome, ST elevation myocardial infarction, non-ST elevation myocardial infarction, or any combination thereof.
   317. The method of embodiment 316, wherein the disease or disorder is treated and the patient simultaneously experiences significant weight loss.
   318. The method of embodiment 316, wherein the disease or disorder is treated and the patient does not experience significant weight loss.
   319. A method for blunting or reducing cravings in a subject in need thereof, the method comprising administering to the patient the pharmaceutical composition of any one of embodiments 113-219 or 274, the conjugate of any one of embodiments 233-272, or the composition of any one of embodiments 275-315 to the patient, thereby blunting or reducing the cravings.
   320. The method of embodiment 319, wherein the craving is a food craving.
   321. The method of embodiment 319, wherein the craving is related to an addictive behavior.
   322. The method of any one of embodiments 1-112, wherein the disease is obesity.
   323. The method of embodiment 322, wherein administration of the anti-GIP antibody, or an antigen-binding fragment thereof, and a biologically active peptide induces weight loss, thereby treating the obesity.
   324. A method of treating obesity or inducing weight loss in a patient in need thereof, the method comprising administering to the patient the pharmaceutical composition of any one of embodiments 113-219 or 274, the conjugate of any one of embodiments 233-272, or the composition of any one of embodiments 275-315 to the patient, thereby treating the disease or disorder.

### Examples

The following examples are illustrative, but not limiting, of the compounds, compositions, and methods described herein. Other suitable modifications and adaptations known to those skilled in the art are within the scope of the following embodiments.

### Example 1: Binding Assessment of Anti-GIP Antibody to Various GIP Peptides

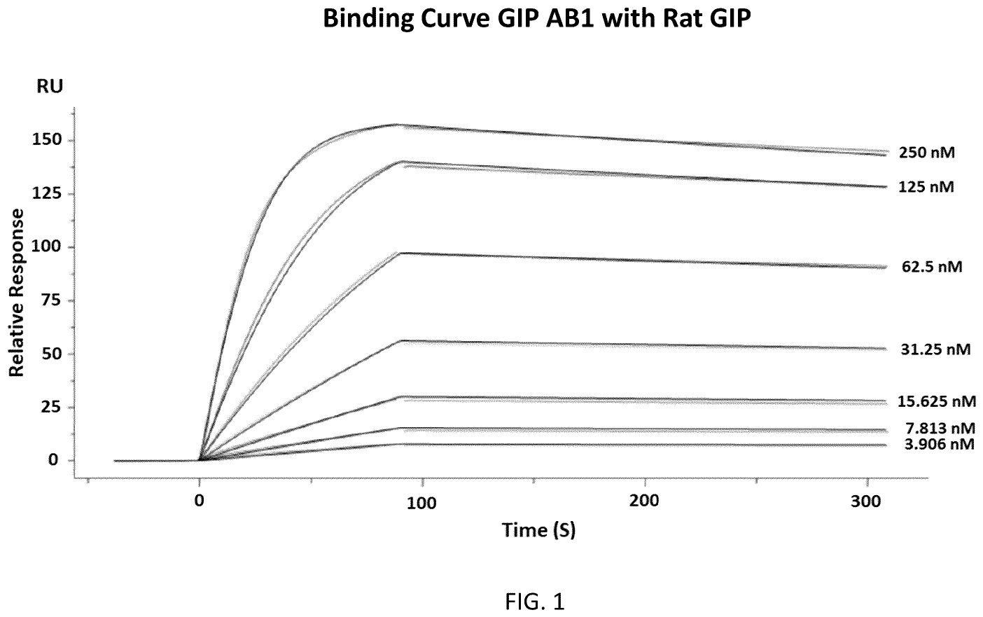

The ability of anti-GIP antibodies as provided for herein to bind to different GIP peptides was assessed. Assessment of binding affinity was carried out using a Biacore™ 8K instrument. First, a CM5 sensor ship was activated for 420 seconds with a mixture of 400 mM EDC and 100 mM NHS at a flow rate of 10 μL/min. Then, 50 μg/mL of an anti-GIP antibody comprising a VH of SEQ ID NO: 29 and a VL of SEQ ID NO: 30 (GIP AB1) in immobilization buffer (10 mM Sodium Acetate, Ph 4.5) was injected at a flow rate of 10 μL/min, which results in an amount of antibody coupled onto the sensor chip of about 3,000 RU. Then, 13 concentrations of each analyte peptide diluted in running buffer were injected for affinity and kinetics measurement. Briefly, each GIP peptide was injected at a flow rate of 30 μL/min for an associate phase of 90 seconds, which was immediately followed by a 210 second dissociation phase. After each cycle of interaction analysis, the sensor ship surface was regenerated with 10 mM glycine-HCl, pH 3.0 at a flow rate of 30 μL/min for 60 seconds to remove any bound analyte. A 1:1 binding model was used to measure the binding affinity. The results for the various GIP peptides are provided in Table 8 below and in FIGS. 1-4.

TABLE 8
Results of GIP peptide affinity analysis

| GIP Peptide | Affinity Constant |
|---|---|
| Rat GIP (SEQ ID NO: 125) | 1.77 × 10⁻⁹ M |
| Human GIP (SEQ ID NO: 123) | 0.8 × 10⁻⁹ M |
| Mouse GIP (SEQ ID NO: 124) | 0.765 × 10⁻⁹ M |
| Monkey GIP (SEQ ID NO: 126) | 1.53 × 10⁻⁹ M |

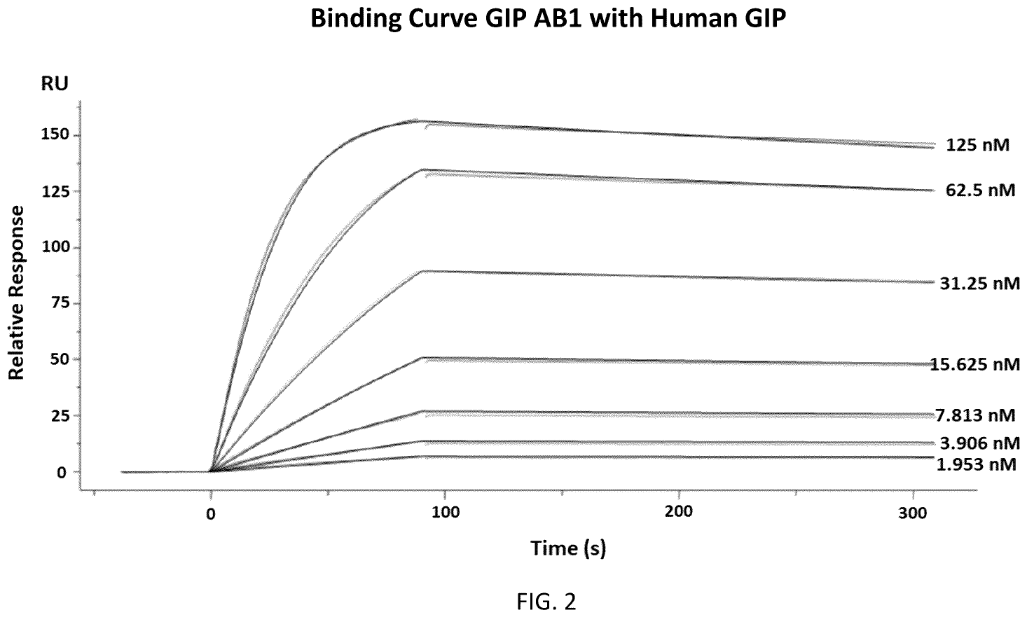

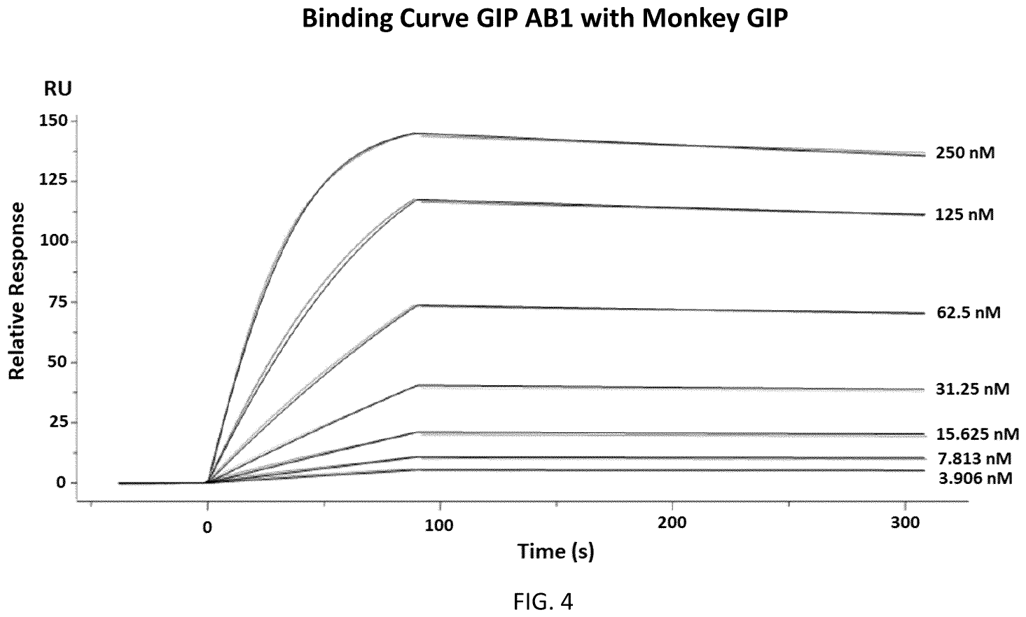

These results show that GIP AB1 is able to bind each of rat (FIG. 1), human (FIG. 2), mouse (FIG. 3), and monkey (FIG. 4) GIP with high affinity.

### Example 2: Optimization of Anti-GIP Antibody: Biologically Active Peptide Conjugates

Anti-GIP antibodies as provided for herein are engineered for conjugation to biologically active peptides as provided for herein. The surface-exposed residues of the antibodies are assessed for their amenability to be mutated to a cysteine residue. In general, residues for mutation are located with calculated accessible surface areas of greater than 20 Å, and exclude the antibody CDRs and effector binding domains as well as glycine and proline residues which can potentially alter protein structures.

Residues adhering to the above criteria are identified and tested to ensure minimal loss of antibody activity. Biologically active peptides are conjugated to the antibody and the activity of the biologically active peptide is assessed to ensure minimal loss of peptide activity. Antibodies will be retested post peptide conjugation to ensure inclusion of the biologically active peptide does not substantially effect antibody activity.

Example 3: In Vivo Comparison of Anti-GIP Antibodies in Combination with Biologically Active Peptides

Anti-GIP antibodies as provided for herein are dosed to mice in combination with biologically active peptides as provided for herein. As a comparison, commercially available anti-GIP antibodies which bind to a separate epitope on GIP are tested in a similar manner. Antibodies that bind to the C-terminus of GIP (i.e., anti-GIP antibodies as provided for herein) are found to exhibit a greater effect on mouse weight when combined with biologically active peptides as provided for herein in comparison to the commercially available anti-GIP antibodies which bind to the N-terminus of GIP.

### Example 4: In Vivo Comparison of Anti-GIP Antibodies Conjugated to Biologically Active Peptides

Anti-GIP antibody: biologically active peptide conjugates as provided for herein are dosed to mice. As a comparison, anti-GIP antibodies not conjugated to the respective biologically active peptides and biologically active peptides in the absence of anti-GIP antibody are tested in a similar manner. Antibody: peptide conjugates are found to exhibit a greater effect on mouse weight in comparison to the antibody alone or biologically active peptide alone conditions.

### Example 5: Generation of Anti-GIP Antibody Conjugates

### Generation of HCR-032

HCR-032 is an antibody-peptide conjugate comprising GIP-AB1 comprising an E272C mutation as provided for herein conjugated to SEQ ID NO: 116 as provided for herein. The method for generating HCR-032 is as follows.

### Procedure for mAb Deblocking.

For antibody reduction, a 200 mM EDTA stock solution was added to a solution containing the antibody to a final EDTA concentration of 2 mM. Proper equivalents of 10 mM TCEP stock solution was then added to the antibody solution. The reaction was incubated at 22° C. and shook at 60 rpm for 2-20 hours. Reduction efficiency was assessed by LC-MS to ensure blocker percentage was lower than 2%. The reduced antibody was purified via buffer exchange to remove excess reducing agent before re-oxidation. Alternatively, the reduced antibody may proceed directly to re-oxidation.

For re-oxidation, buffer was added to the reduced antibody solution to adjust antibody concentration for the re-oxidation reaction. A 200 mM EDTA stock solution was added to the reduced antibody solution to a final EDTA concentration of 2 mM. Proper equivalents of 100 mM DHAA stock solution was then added to the antibody solution. The reaction was incubated at 22° C. and shook at 60 rpm for 3-4 hours. The efficiency of re-oxidation was assessed via LC-MS.

Table 9 below provides the variable conditions tested during optimization of the above recited procedures:

TABLE 9
Reduction and Reoxidation Reaction
Variable Conditions - HCR-032

| | | | | Variable Reoxidation | |
|---|---|---|---|---|---|
| | Variable Reduction Conditions | | | Conditions | |
| | | Reducing | | Reoxidation | |
| | | agent/ | | agent/ | |
| | Reducing | antibody | Time/ | antibody | Time/ |
| Trial | Agent | ratio | Temp | ratio | Temp |
| 1-1 | TCEP | 20 | 22° C./20 h | 15 | 22° C./2 h |
| 1-2 | DTT | 50 | | | |
| 2 | TCEP | 4 | 22° C./20 h | 8 | 22° C./2 h |
| 3 | TCEP | 4 | 22° C./20 h | 8 | 22° C./2 h |
| 4-1 | TCEP | 2.4 | 22° C./2 h | 8 | 22° C./2.5 h |
| 4-2 | | 4 | | | |
| 4-3 | | 4 | | | |
| 5 | TCEP | 2.4 | 22° C./2 h | 8 | 22° C./4.5 h |

### Procedure for Peptide Conjugation.

The pH of the re-oxidized antibody solution was adjusted to approximately pH=7.4 by adding approximately 2.7% volume of 1M Tris, pH 8.5 buffer. A 10 mM stock solution of peptide was prepared by dissolving solid peptide with ddH₂O. Four equivalents of 10 mM peptide stock solution was added to the antibody solution, and the reaction was incubated at 22° C. and shook at 60 rpm for 14 hours. Conjugation efficiency was monitored via TOF Mass Spec until the peptide to antibody ratio reached the desired target. Crude conjugation was purified by HIC column, and proper fractions were pooled and then subject to buffer exchange into 20 mM Histidine/Histidine-HCl, 150 mM L-Arginine, pH 6.3 by ultrafiltration and diafiltration. The sample was then filtered with a 0.22 μm membrane and the product was characterized.

Table 10 below provides the variable conditions tested during optimization of the above recited procedures:

TABLE 10
Conjugation Reaction Variable Conditions - HCR-032

| Trial | Peptide/antibody ratio | Time/temp. |
|---|---|---|
| 1-1 | 8 | 22° C./18 h |
| 1-2 | 4 | 22° C./21 h |
| 2 | 8 | 22° C./18 h |
| 3 | 4 | 22° C./14 h |
| 4-1 | 4 | 22° C./16 h |
| 4-2 | 8 | 22° C./16 h |

### Procedure for 1 Gram Scale of Material Generation.

The procedure for generating HCR-032 starting from 1 gram of antibody generally follows the procedure as outlined above. Table 11 below provides the variable conditions of the 1 gram scale material generation:

TABLE 11
1 gram scale material generation - HCR-032

| | TCEP/ | | DHAA/ | |
|---|---|---|---|---|
| | antibody | Time/ | antibody | Time/ |
| Process | ratio | temp. | ratio | temp |
| Deblocking | 2.4 | 22° C./2 h | 8 | 22° C./4 h |

| Process | Peptide/antibody ratio | Time/temp. |
|---|---|---|
| Conjugation | 4.0 | 22° C./14 h |

### Generation of HCR-068

HCR-068 is an antibody-peptide conjugate comprising the GIP-AB1 isotype control comprising an E272C mutation conjugated to SEQ ID NO: 116 as provided for herein. The method for generating HCR-068 was largely the same as outlined above for HCD-032. Differences as compared to HCR-032 are outlined in Tables 12 and 13 below.

TABLE 12
Reduction and Reoxidation Reaction Variable Conditions -
HCR-068 different conditions as compared to HCR-032

| | | | | Variable Reoxidation | |
|---|---|---|---|---|---|
| | Variable Reduction Conditions | | | Conditions | |
| | | Reducing | | Reducing | |
| | | agent/ | | agent/ | |
| | Reducing | antibody | Time/ | antibody | Time/ |
| Trial | Agent | ratio | Temp | ratio | Temp |
| 4 | TCEP | 4 | 22° C./2 h | 8 | 22° C./2.5 h |
| 5 | TCEP | 4 | 22° C./2 h | 8 | 22° C./4.5 h |

TABLE 13
1 gram scale material generation - HCR-068
different conditions as compared to HCR-032

| | TCEP/ | | DHAA/ | |
|---|---|---|---|---|
| | antibody | Time/ | antibody | Time/ |
| Process | ratio | temp. | ratio | temp |
| Deblocking | 4.0 | 22° C./2 h | 8 | 22° C./6 h |

| Process | Peptide/antibody ratio | Time/temp. |
|---|---|---|
| Conjugation | 4.0 | 22° C./14 h |

TABLE 14
Further Optimization

| | TCEP/ | | DHAA/ | |
|---|---|---|---|---|
| | antibody | Time/ | antibody | Time/ |
| Process | ratio | temp. | ratio | temp |
| Deblocking | 4.0 | 22° C./2 h | 4.0 or 6.0 | 22° C./4 h |

| Process | Peptide/antibody ratio | Time/temp. |
|---|---|---|
| Conjugation | 4.0 | 22° C./16 h |

### Example 6: Assessment of Conjugation on Antibody and Peptide Activity

In vitro assessment of the antibody conjugates provided for herein was performed to determine whether conjugation affected the antibodies ability to bind GIP, the peptides ability to activate their native receptor, or both.

First, it was assessed whether conjugation of SEQ ID NO: 145 (HXEGTFTSDYSSYLEEQAAKEFIAWLVKGGG (GGGGS) 3K(SEQ ID NO: 145, X=2-Aminoisobutyric acid)) to the anti-GIP antibodies provided for herein would affect the antibodies ability to bind GIP. A brief description of the antibody conditions tested may be found in Table 15 below.

TABLE 15
Antibody Group Details

| Group | Details |
|---|---|
| HCR-188 | GIP-AB1 as provided herein. |
| HCR-032 | GIP-AB1 comprising a E272C mutation and conjugated to |
| | peptide SEQ ID NO: 145 at position 272. |
| HCR-068 | GIP-AB1 isotype control comprising a E272C mutation and |
| | conjugated to peptide SEQ ID NO: 145 at position 272. |

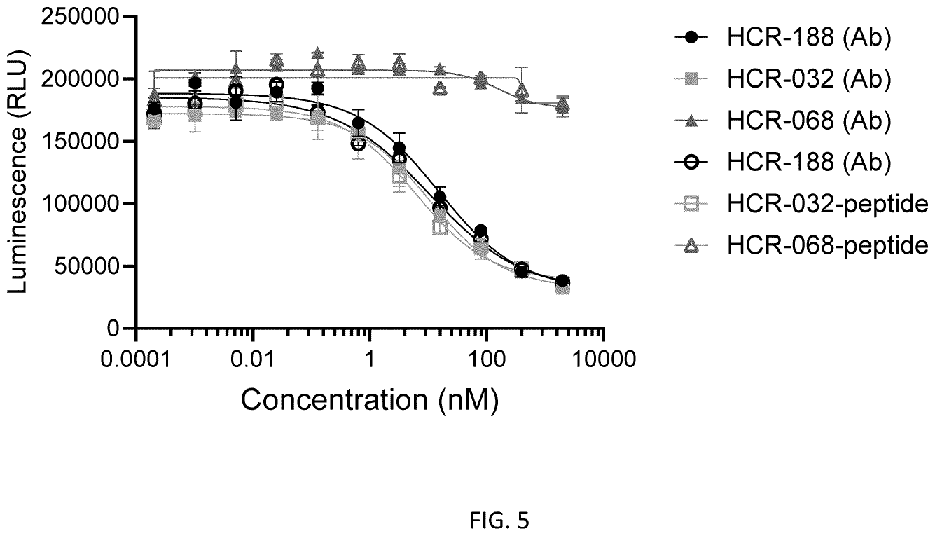

Briefly, HEK293 cells were engineered to express firefly luciferase under the control of CAMP response element (CRE) and human GIPR, allowing luciferase detection to serve as a proxy for cAMP production. Antibody samples were serially diluted and added to cells in a 96-well plate. A fixed concentration of 3.5 nM GIP was then added to each well to assess the ability of the antibodies to neutralize GIP and prevent its binding to GIPR and subsequent receptor activation. Cells were incubated at 37° C. in a CO₂ incubator for 5-6 hours followed by the addition of luciferase detection buffer and measurement of luminescence in a plate reader. A reduced level of luciferase is indicative of sequestration of GIP ligand and neutralization of GIP receptor signaling. The results are shown in FIG. 5 and Table 16 below.

TABLE 16
GIP Ligand Neutralization Results

| | Effector | Conjuga- | Conjuga- | GIPR |
|---|---|---|---|---|
| | Null | tion | tion | Antagonism |
| Group | Subst. | Site | Linker | IC50 (nM) |
| HCR-188 (Ab) n1 | — | — | — | 15.21 |
| HCR-032 (Ab) | LALA-PA | HC E272C | Ab alone | 10.87 |
| HCR-068 (Ab) | LALA-PA | HC E272C | Ab alone | ND |
| HCR-188 (Ab) n2 | — | — | — | 10.65 |
| HCR-032-peptide | LALA-PA | HC E272C | BrAc | 6.18 |
| HCR-068-peptide | LALA-PA | HC E272C | BrAc | ND |

The control conditions of WT antibody alone (HCR-188(Ab)) and isotype control (HCR-068 (Ab)) behaved as expected, with the WT antibody sequestering GIP and preventing receptor activation and the isotype control having no effect on GIP signaling. Similarly, the isotype control conjugated to SEQ ID NO: 145 (HCR-068-peptide) also had no effect on GIP signaling, demonstrating that the peptide SEQ ID NO: 145 itself was not in some way interfering with GIP signaling. The mutated antibody without peptide conjugated (HCR-032 (Ab)) was able to successfully sequester GIP to a similar degree as the WT control, indicating that the E272C mutation did not have a deleterious effect on antibody function. Finally, the functional antibody-peptide conjugate (HCR-032-peptide) was also able to successfully sequester GIP to a similar degree as the WT control, indicating that the conjugation of the biologically active peptide to the anti-GIP antibody did not affect the antibody's ability to bind GIP.

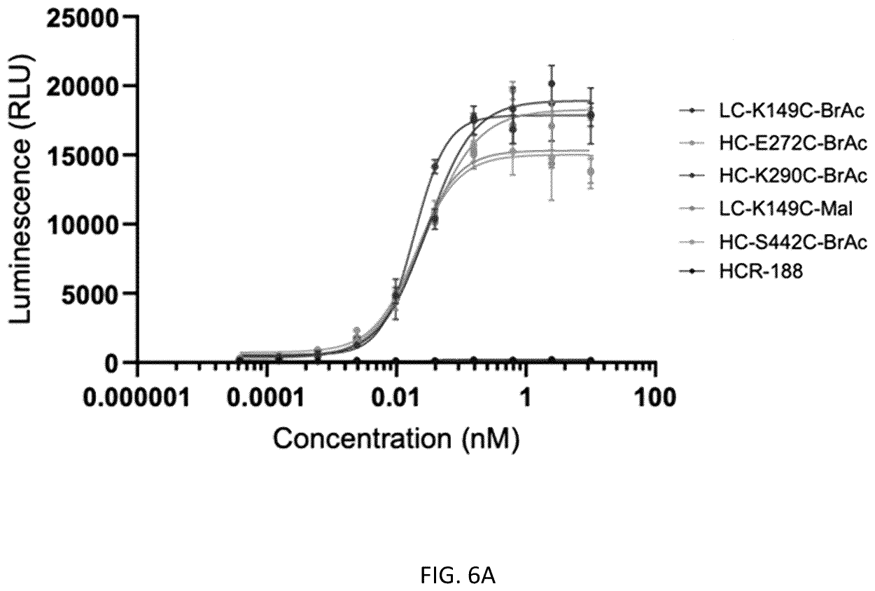

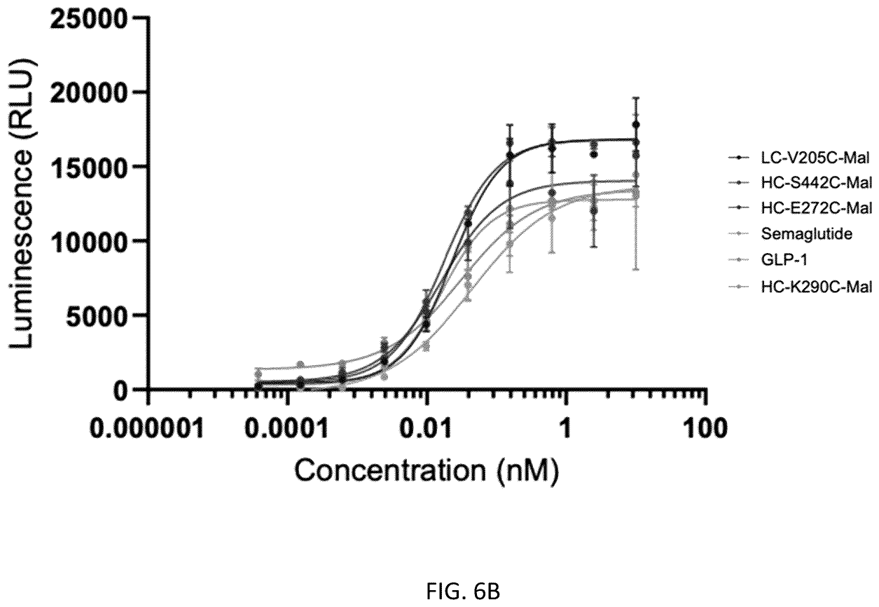

Next, it was assessed whether the peptide SEQ ID NO: 145 retained its agonist functionality at the GLP-1 receptor when conjugated to an anti-GIP antibody as provided for herein. For this assessment, different conjugation sites were tested as well as different linkers linking SEQ ID NO: 145 to the antibodies. Briefly, HEK293 cells were engineered to express firefly luciferase under the control of cAMP response element (CRE) and human GLP-1R, allowing luciferase detection to serve as a proxy for GLP-1R activation. Samples (antibody or peptide ligand) were serially diluted and incubated with cells in a 96-well plate at 37° C. in a CO₂ incubator for 5-6 hours and then assessed for dose-dependent increases in luminescence signal using a plate reader upon addition of detection buffer. The results are shown in FIGS. 6A and 6B and tabulated in Table 17 below:

TABLE 17
GLP-1R mediated cAMP activity assay results

| Treatment | mAb Cys Site | Linker | EC₅₀ (nM) |
|---|---|---|---|
| HCR-188 | None (parental ab) | N/A | Not Determined* |
| Semaglutide | — | N/A | 0.045 |
| GLP-1 | — | N/A | 0.031 |
| HCR-188 | LC-K149C | BrAc | 0.030 |
| HCR-188 | LC-K149C | Mal | 0.020 |
| HCR-188 | LC-V205C | Mal | 0.023 |
| HCR-188 | HC-E272C | BrAc | 0.029 |
| HCR-188 | HC-E272C | Mal | 0.016 |
| HCR-188 | HC-K290C | BrAc | 0.019 |
| HCR-188 | HC-K290C | Mal | 0.016 |
| HCR-188 | HC-S442C | BrAc | 0.020 |
| HCR-188 | HC-S442C | Mal | 0.018 |

*Not Determined does not mean not tested. As shown in FIG. 6A, the parental antibody returned no signal as expected.

As shown in FIGS. 6A and 6B and as tabulated in Table 17 above, conjugation of SEQ ID NO: 145 to the HCR-188 antibody did not diminish the peptides ability to activate the GLP-1 receptor. This was true for each conjugation site tested (LC-K149C, HC-E272C, HC-K290C, and HC-S442C). Additionally, conjugation method (C-terminal bromoacetyl group (BrAc) or maleimide (Mal)) did not have an effect on peptide activity.

Having demonstrated that SEQ ID NO: 145 maintained activity when conjugated to HCR-188, it was next assessed whether the conjugates would maintain their ability to bind GIP. The experimental design is the same as described above for the results shown in FIG. 5. Here, the conjugation sites tested were expanded as compared to the data in FIG. 5. Results are shown in Table 18 below.

TABLE 18
GIP Binding Assessment of Antibody Conjugates

| mAb Cys Site | Linker | K_{D} (M) | Relative K_{D}* |
|---|---|---|---|
| HCR-188 (parent n1) | N/A | 1.90E−09 | 0.94 |
| HCR-188 (parent n2) | N/A | 2.13E−09 | 1.06 |
| LC K149C | Ab alone | 1.72E−09 | 0.85 |
| LC K149C | BrAc | 1.30E−09 | 0.64 |
| LC K149C | Mal | 1.34E−09 | 0.67 |
| LC V205C | Ab alone | 1.75E−09 | 0.87 |
| LC V205C | Mal | 1.34E−09 | 0.66 |
| HC E272C | Ab alone | 1.39E−09 | 0.69 |
| HC E272C | BrAc | 9.01E−10 | 0.45 |
| HC E272C | Mal | 1.18E−09 | 0.59 |
| HC K290C | Ab alone | 1.71E−09 | 0.85 |
| HC K290C | BrAc | 1.36E−09 | 0.68 |
| HC K290C | Mal | 1.37E−09 | 0.68 |
| HC S442C | Ab alone | 1.70E−09 | 0.84 |
| HC S442C | BrAc | 1.25E−09 | 0.62 |
| HC S442C | Mal | 1.24E−09 | 0.61 |

*Relative K_{D} calculated by dividing sample K_{D}/average of parent K_{D} (n1 and n2).

As shown in Table 18 above, the parent antibody shows good binding to human GIP peptide (K_{D} approx. 2 nM), which was within the previous measurements obtained for this antibody. All cysteine variants of the parent antibody show equivalent binding (K_{D} within 2-fold) to GIP compared to the parent antibody. All conjugate molecules tested also show equivalent binding (K_{D}) within 2-fold) to GIP as compared to the parent antibody and as compared to the respective cysteine mutant antibody without peptide conjugation. These data show that conjugation of the peptide at various conjugation sites does not affect the antibody's ability to bind GIP.

### Example 7: Anti-GIP Antibody Conjugates and Combinations Promote Weight Loss in Diet Induced Obesity (DIO) Mouse Study

C57BL/6JRj male mice were fed a 60% high fat diet (HFD, D12492, Ssniff®) ad libitum for approximately 20 weeks before start of treatment. The HFD was provided ad libitum throughout the treatment phase of the study. Mice were randomized to treatment groups based on body weight and body composition as determined by EchoMRI measured on study day-7 before treatment. Treatment groups were as follows (n=8 per group): vehicle, HCR-188 (1.9, 4.75 and 9.5 mg/kg, BIW), HCR-032 (1, 2, 5 and 10 mg/kg, BIW), HCR-068 (1, 2, 5 and 10 mg/kg, BIW), combination of HCR-188 and HCR-068 (1.9 mg/kg+2 mg/kg, respectively, and 4.75 mg/kg+5 mg/kg, respectively), semaglutide (3 nmol/kg), and combination of HCR-188+semaglutide (1.9 mg/kg+3 nmol/kg, respectively). Treatment was administered for 7 weeks. Body weight and food intake were monitored daily. Additional details regarding treatment groups are provided in Table 19 below.

TABLE 19
Treatment Group Details

| Group | Details |
|---|---|
| HCR-188 | GIP-AB1 as provided herein. |
| HCR-032 | GIP-AB1 comprising a E272C mutation and conjugated to |
| | peptide SEQ ID NO: 116 at position 272. |
| HCR-068 | GIP-AB1 isotype control comprising a E272C mutation and |
| | conjugated to peptide SEQ ID NO: 116 at position 272. |

Peptide SEQ ID NO: 116 is conjugated to HCR-032 and HCR-068 through a C-terminal linker of SEQ ID NO: 131 (n=3), wherein the linker has an additional lysine at the C-terminus. Thus, the entire peptide-linker sequence is as follows: HXEGTFTSDYSSYLEEQAAKEFIAWLVKGGG(GGGGS)₃K(SEQ ID NO: 145, X=2-Aminoisobutyric acid). The peptide-linker may be further modified to comprise a C-terminal bromoacetyl group (BrAc), resulting in a peptide-liner sequence of HXEGTFTSDYSSYLEEQAAKEFIAWLVKGGG(GGGGS)₃K—[BrAc] (SEQ ID NO: 146, X=2-Aminoisobutyric acid).

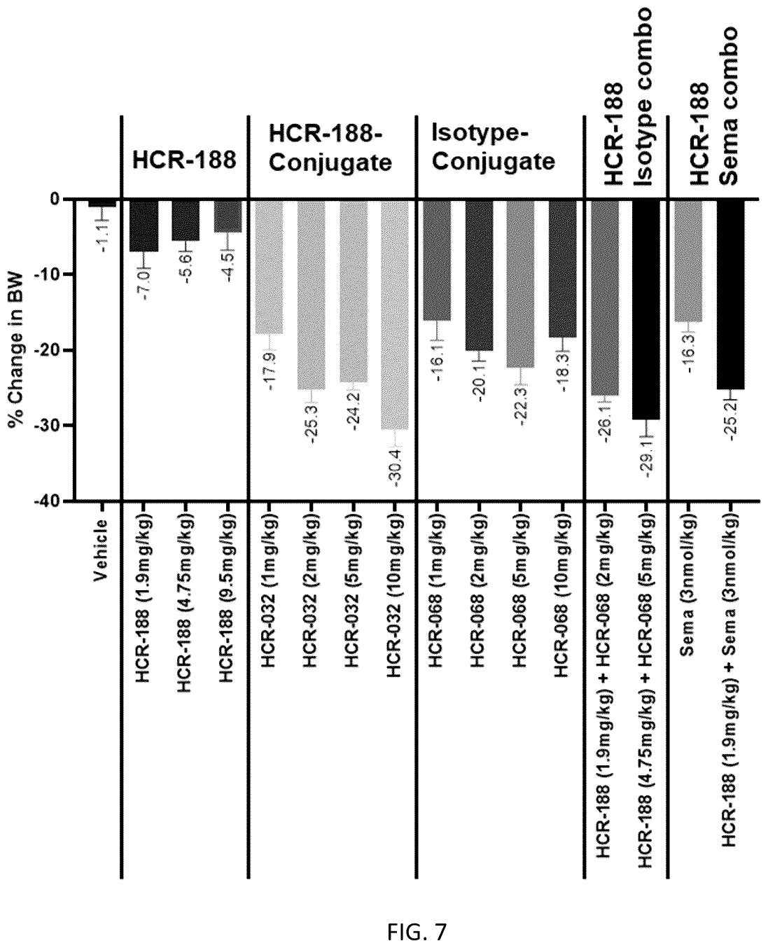

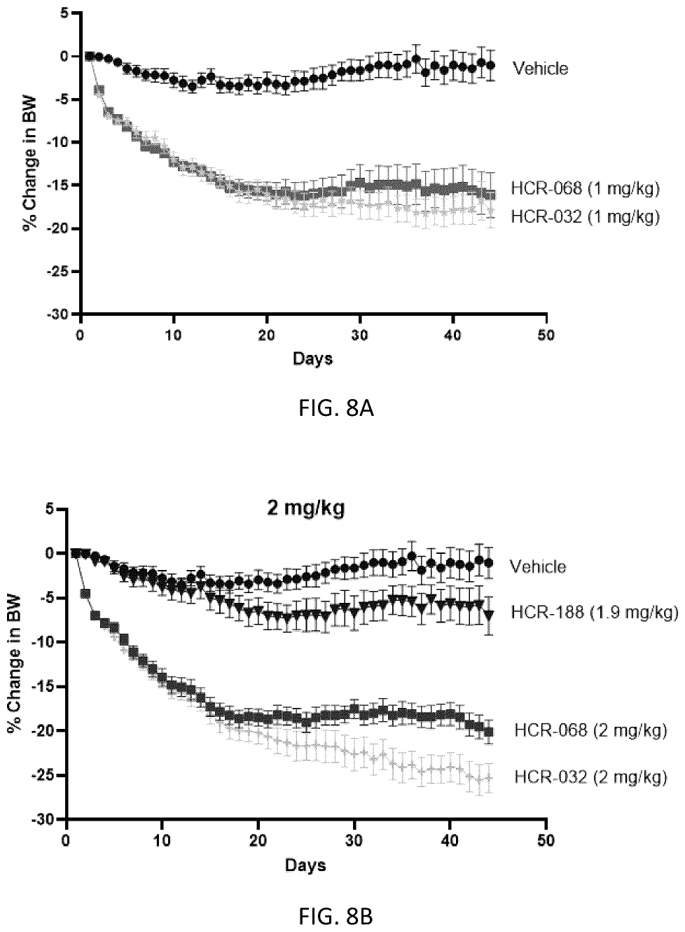

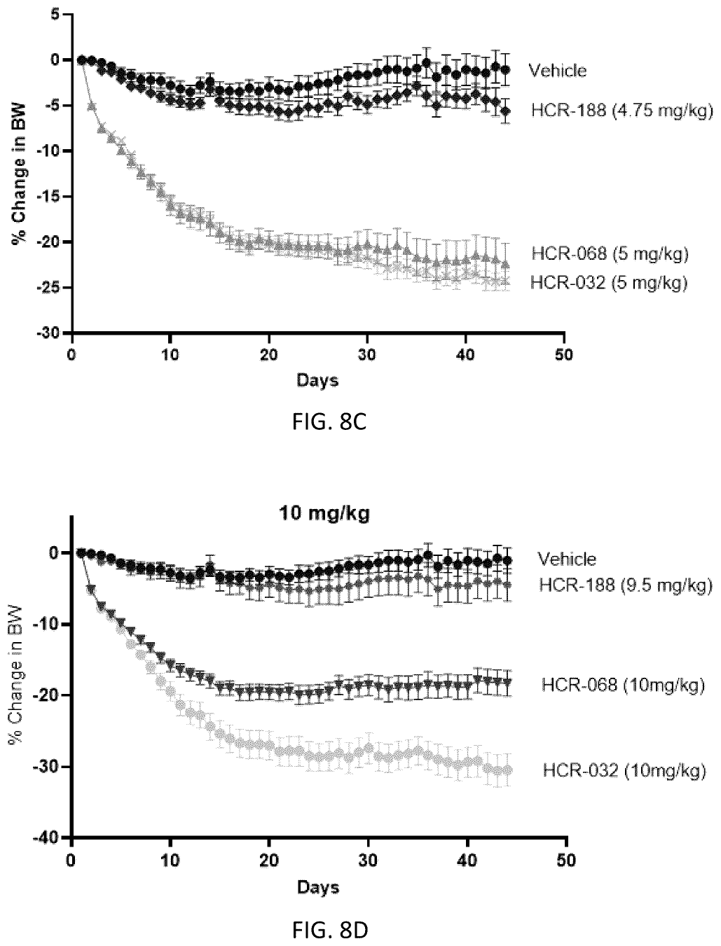

Results for the study are shown in FIGS. 7 and 8 and in Table 20 below.

TABLE 20
DIO Mouse Study Results

| | Dose | BW % | BW change vehicle |
|---|---|---|---|
| Condition | (mg/kg) | change | adjusted % |
| Vehicle | — | −1.1 | — |
| Monotherapy | 1.9 | −7 | −5.9 |
| HCR-188 | 4.75 | −5.6 | −4.5 |
| | 9.5 | −4.5 | −3.4 |
| HCR-032 | 1 | −17.9 | −16.8 |
| | 2 | −25.3 | −24.2 |
| | 5 | −24.2 | −23.1 |
| | 10 | −30.4 | −29.3 |
| Isotype control | 1 | −16.1 | −15 |
| (HCR-068) | 2 | −20.1 | −19 |
| | 5 | −22.3 | −21.2 |
| | 10 | −18.3 | −17.2 |
| HCR-188 + HCR- | 1.9 + 2 | −26.1 | −25 |
| 068 | 4.75 + 5 | −29.1 | −28 |
| Semaglutide | 3 nmol/kg | −16.3 | −15.2 |
| HCR-188 + | 1.9 mg/kg + 3 | −25.2 | −24.1 |
| semaglutide | nmol/kg | | |

As shown in FIG. 7 and FIGS. 8A-8D, treatment with HCR-188 alone resulted in a modest decrease in body weight over the course of the study. Treatment with the antibody conjugate HCR-032 resulted in a much more pronounced decrease in body weight. Treatment with the isotype control conjugate HCR-068 shows that the peptide SEQ ID NO: 116 does drive weight loss even when conjugated to a control antibody, albeit not to the same degree as when conjugated to the anti-GIP antibody HCR-188. This is most readily demonstrated at dosing concentrations of 2 mg/kg and 10 mg/kg (FIGS. 8B and 8D, respectively), where the conjugate HCR-032 clearly produces greater weight loss than the isotype control conjugate. At the highest dose, the effect appeared to be synergistic, as the conjugate HCD-032 produced a 30.4% drop in body weight, where the antibody alone and the isotype control conjugate produced a 4.5% drop and 18.3% drop in body weight, respectively.

It was also assessed whether combinations of the antibody and peptide would result in weight loss when the peptide is not conjugated to the anti-GIP antibody. As shown in FIG. 7 and Table 20 above, a combination of HCR-188 (anti-GIP antibody, no peptide conjugate) and HCR-068 (isotype control, conjugated to SEQ ID NO: 116) produce pronounced weight loss in the DIO mouse model. At both concentration pairs tested, the combination produced a more substantial weight loss that either component individually. Furthermore, at the higher concentration test, the effect appeared to be synergistic, as combination of 4.75 mg/kg HCR-188 with 5 mg/kg HCR-068 produced in a 29.1% decrease in body weight, where the antibody alone and the isotype control alone produced a 5.6% drop and a 22.3% drop in body weight, respectively, at the same concentrations.

Lastly, it was assessed whether the enhanced weight loss effects observed in combination treatments would be maintained with a different GLP-1R agonist peptide. As shown in FIG. 7, mice treated with semaglutide demonstrated a 16.3% drop in body weight over the course of the study. In contrast, the combination of semaglutide with HCR-188 resulted in a 25.2% drop in body weight over the course of the study.

The results from this example show that the antibodies provided for herein result in weight loss in a DIO mouse study, and that the combination of said antibodies with GLP-1R agonists, either via conjugation or combination treatment, result in an enhanced weight loss effect.

Example 8: Co-Treatment with Anti-GIP Antibody and Semaglutide Promotes Weight Loss in Diet Induced Obesity (DIO) NHP Study

Non-naïve, cynomolgus monkeys (Macaca fascicularis, male, >7.5 kgs, BMI>35 kg/m2, >8 years old of age) were obtained from Kunming Biomed International (KBI)'s stock colony. The animals were individually housed in stainless steel cages for the duration of the study under a controlled environment that is set to maintain a temperature of 18-29° C., relative humidity of 30-90%, and a minimum of 10 air changes/hour. A time-controlled lighting system was used (light 7:00 AM-7:00 PM to provide a regular 12-hour light/12-hour dark diurnal cycle. Cages were cleaned at regular intervals to maintain hygiene.

The animals were provided 3 meals per day consisting of 50 g of KBI proprietary standard animal formula feed (extruded pellets) in the morning 9:00-10:00 AM, one regular apple (150 g) in the afternoon 14:00-15:00 PM, and 100 g of KBI proprietary high-fat diet (HFD) in the evening 16:00-17:00 PM. Water was provided ad libitum. Food was provided ad libitum 10 days (day-10) before the dosing phase. Additional 100 g of high fat diet was provided in 20 min intervals during evening meal if food were almost consumed. All of the food was withdrawn at 17:00. After each feeding time, all the remaining food was withdrawn from the animal and intake was determined by weighing the left-over food. The animals were provided with at least 2 items of manipulanda for each cage for environmental enrichment to ensure adequate welfare and psychological well-being. During the acclimation/training period, animals were given food treats as a reward after each training activity.

At the end of the training period, 40 animals were subjected to stratified randomization based on various parameters including BW/BMI, TEI, and relevant metabolic parameters and assigned to 4 dose groups (with 10 animals each). Ten monkeys were subcutaneously dosed with HCR-188 vehicle (once a week) and semaglutide vehicle (twice a week), 10 monkeys were dosed subcutaneously with 20 mg/kg HCR-188 (one a week) and semaglutide vehicle (twice a week), 10 monkeys were dosed subcutaneously with 20 μg/kg semaglutide (twice a week) and HCR-188 vehicle (once a week), and 10 monkeys were dosed subcutaneously with 20 mg/kg HCR-188 (one a week) and 20 μg/kg semaglutide (twice a week). Semaglutide was titrated to the target dose over a period of 14 days. The dosing duration was 7 weeks with follow up assessment for an additional four weeks. During the dosing period, body weight, food intake, and blood/serum chemistries were monitored on various schedules throughout the study.

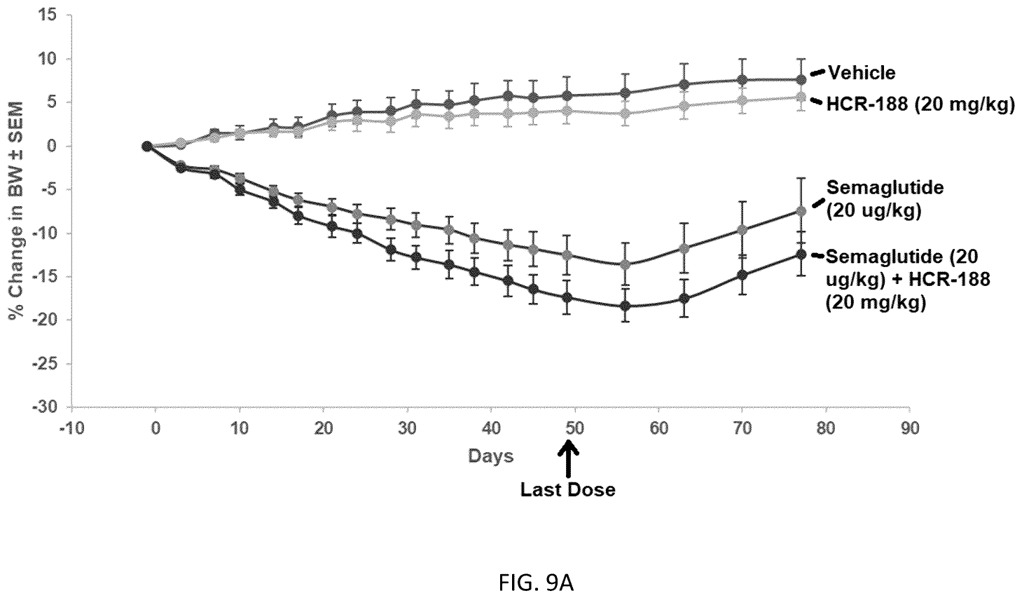

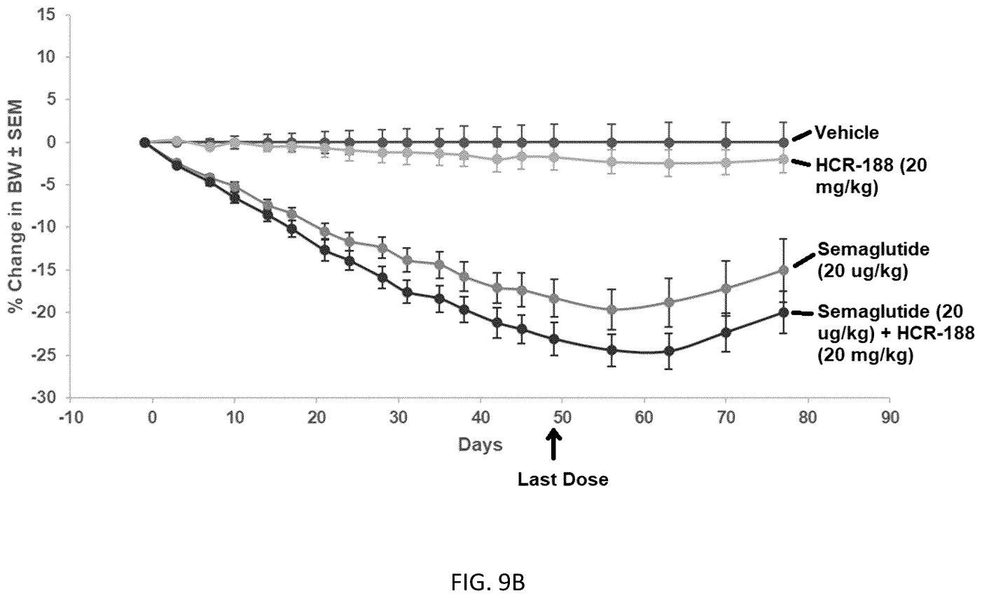

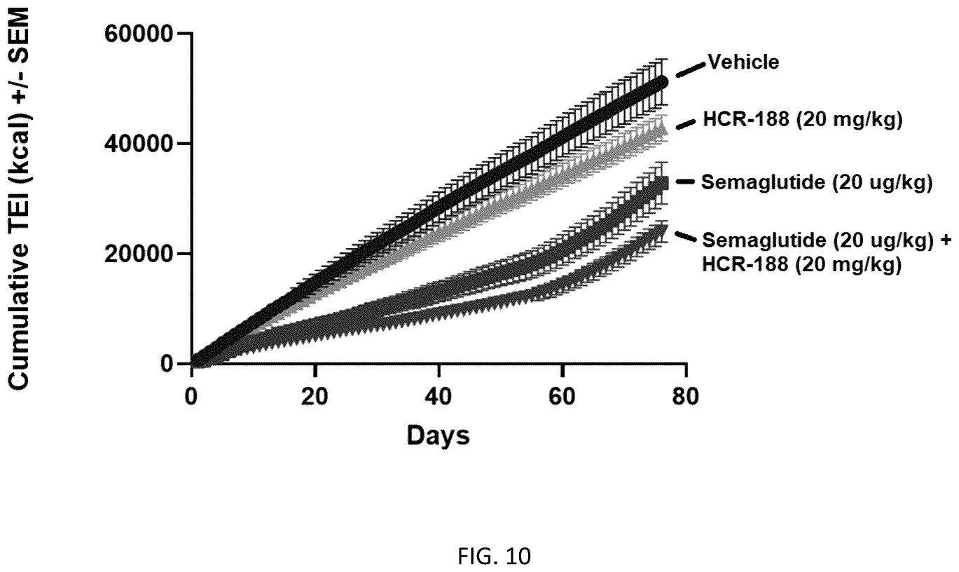

Results for the study described above are shown in FIGS. 9 and 10 and in Tables 21 and 22 below:

TABLE 21
DIO NHP Study - Change in body weight

| | Day 49 Results (last dose) | | Day 77 results (Washout) | |
|---|---|---|---|---|
| | | Vehicle | | Vehicle |
| | % Change | adjusted % | % Change | adjusted % |
| Group | in BW | change in BW | in BW | change in BW |
| Vehicle | 5.8 | — | 7.6 | — |
| HCR-188 | 4.0 | −1.8 | 5.6 | −2.0 |
| Semaglutide | −12.5 | −18.3 | −7.4 | −15.0 |
| HCR-188 + | −17.3 | −23.1 | −12.4 | −20.0 |
| Semaglutide | | | | |

TABLE 22
DIO NHP Study - Mean Total Energy Intake (TEI)

| Group | Mean TEI (kcal) - day 38 | Mean TEI (kcal) - day 76 |
|---|---|---|
| Vehicle | 732 | 652 |
| HCR-188 | 561 | 555 |
| Semaglutide | 308 | 905 |
| HCR-188 + | 181 | 781 |
| Semaglutide | | |

As shown in FIGS. 9A and 9B and in Table 21 above, monkeys treated with HCR-188 alone exhibited a minor decrease in body weight as compared to vehicle. Monkeys treated with semaglutide alone exhibited a 12.5% decrease in body weight (18.3% decrease when vehicle adjusted) by day 49 (last dose). Monkeys treated with the combination of HCR-188 and semaglutide exhibited a 17.3% decrease in body weight (23.1% decrease when vehicle adjusted) by day 49 (last dose). The results for the combination therapy appear to be synergistic, as the percent decrease in body weight is greater in the combination than the sum of the individual treatments. Both the semaglutide alone and the combination treatment exhibited a continued decrease in weight immediately after stopping treatment, which then trended toward a slight rebound in weight during days 60-77 of the study. However, even after washout the semaglutide alone group exhibited a 7.4% decrease in body weight (15.0% decrease when vehicle adjusted) and the combination group of HCR-188 and semaglutide exhibited a 12.4% decrease in body weight (20.0% decrease when vehicle adjusted) at study day 77, four weeks after treatment stoppage.

Without wishing to be bound to any particular theory, the decrease in body weight appears to be related to an overall reduction in caloric intake by the animals. Table 21 above shows the mean TEI for each group at day 38 (still receiving treatment) and day 76 (washout) of the study. As shown in the table at Day 38, the vehicle group has a mean TEI of 732 kcal, while each treatment group has a reduced TEI compared to vehicle. The group ranking for TEI mirrors the rank order for percent body weight loss. As shown in FIG. 10, the results for Table 22 day 38 are not merely the result of a select snapshot. FIG. 10 shows the cumulative TEI over the course of the study thus far. Here again, the rank order for cumulative TEI mirrors the rank order for percent body weight loss. At study day 76, both the vehicle and HCR-188 alone groups exhibit a slight decrease in mean TEI as compared to study day 38. By comparison, both the semaglutide and combination HCR-188 plus semaglutide groups show a dramatic increase in TEI at study day 76. This data shows that the treatments have been effectively cleared by four weeks after the last dose. However, as shown in FIG. 10, even with the increased TEI in the treatment groups at day 76 the cumulative TEI is still significantly lower for the treatment groups as compared to vehicle.

These results unexpectedly demonstrated that anti-GIP antibodies as provided for herein in combination with an exemplary GLP-1R agonist (semaglutide) produce a marked and synergistic weight loss in NHPs.

### Example 9: HCR-032 Single Dose Pharmacokinetic (SDPK) Study in Diet Induced Obesity NHPs

Non-naïve, cynomolgus monkeys (Macaca fascicularis, male, <8.5 kgs, BMI>35 kg/m2, >8 years old of age) were obtained from KBI's stock colony. The animals were individually housed in stainless steel cages for the duration of the study under a controlled environment that is set to maintain a temperature of 18-29° C., relative humidity of 30-90%, and a minimum of 10 air changes/hour. A time-controlled lighting system was used (light 7:00 AM-7:00 PM) to provide a regular 12-hour light/12-hour dark diurnal cycle. During the study, the cages were cleaned at regular intervals to maintain hygiene.

The animals were provided 3 meals per day consisting of 50 g of KBI proprietary standard animal formula feed (extruded pellets) in the morning 9:00-10:00 AM, one regular apple (150 g) in the afternoon 14:00-15:00 PM, and 100 g of KBI proprietary high-fat diet (HFD) in the evening 16:00-17:00 PM. Water was provided ad libitum. Food was provided ad libitum at least 1 week before the dosing phase: Additional 100 g of high fat diet was provided in 20 min intervals during evening meal if food were almost consumed. All of the food was withdrawn at 17:00. After each feeding time, all the remaining food was withdrawn from the animal and intake is determined by weighing the left-over food. The animals were provided with at least 2 items of manipulanda for each cage for environmental enrichment to ensure adequate welfare and psychological well-being. During the acclimation/training period, animals were given food treats as a reward after each training activity.

At the end of the training period, four animals were randomized into two groups of two animals each. One group received a single subcutaneous dose of the HCR-188-GLP-1 conjugate (HCR-032) at 1 mg/kg, while the second group received a single subcutaneous dose of the same conjugate at 5 mg/kg. Following administration, the monkeys were monitored for four weeks. During this follow-up period, body weight, food intake, and blood/serum chemistry parameters were assessed at various intervals throughout the study.

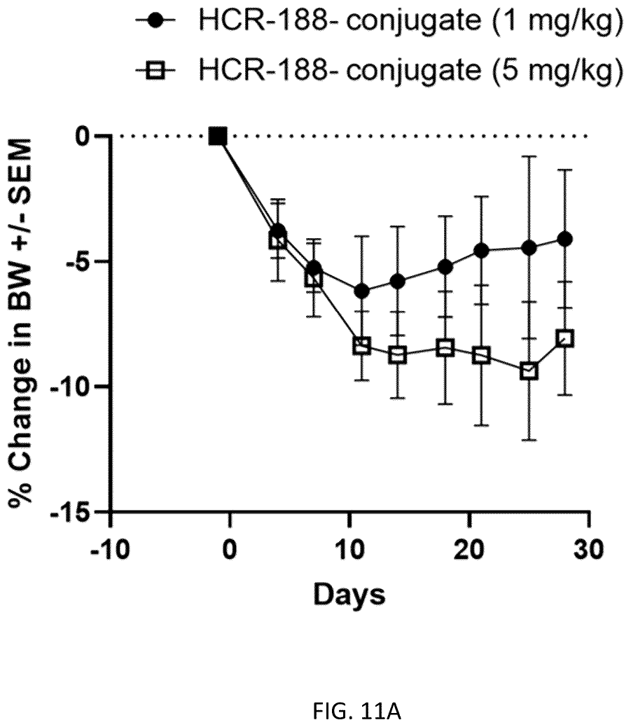

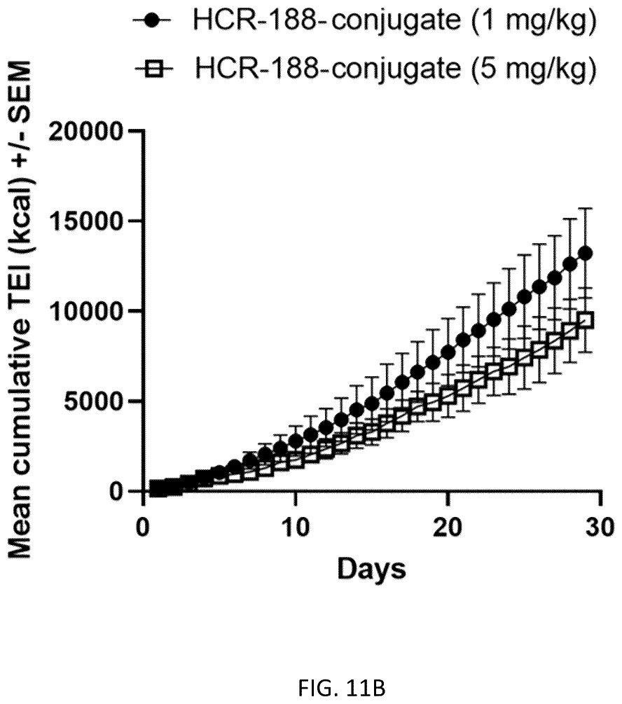

Results for the study described above are shown in FIGS. 11A and 11B. As shown in FIG. 11A, a single dose of HCR-032 resulted in a significant and sustained decrease in body weight compared to baseline. Additionally, the weight loss was dose dependent, as the animals receiving the larger 5 mg/kg single dose had a greater average decrease in body weight as compared to the animals receiving the 1 mg/kg dose. Similarly, as shown in FIG. 11B, the animals receiving the 5 mg/kg dose had a decrease in total energy intake as compared to those receiving the 1 mg/kg dose.

The data from this example show that a single dose of HCR-032 is sufficient to cause a marked and sustained reduction in body weight in a DIO NHP model.

Example 10: Treatment of Obesity Using Anti-GIP Antibodies in Combination with Biologically Active Peptides

A human subject having obesity is treated with an anti-GIP antibodies as provided for herein in combination with biologically active peptides as provided for herein and the subject's weight is reduced after treatment.

### Example 11: Treatment of Obesity Using Anti-GIP Antibodies Conjugated to Biologically Active Peptides

A human subject having obesity is treated with an anti-GIP antibody: biologically active peptide conjugate as provided for herein and the subject's weight is reduced after treatment.

Accordingly, the embodiments and examples provided herein demonstrate unexpected and surprising results with the molecules and compositions provided for herein.

1. A conjugate, comprising an anti-glucose-dependent insulinotropic polypeptide (GIP) antibody, or an antigen-binding fragment thereof, conjugated to a biologically active peptide, wherein the biologically active peptide comprises a glucagon like peptide 1 receptor (GLP-1R) agonist.

2. The conjugate of claim 1, wherein the anti-GIP antibody, or antigen-binding fragment thereof comprises a heavy chain (HC) comprising a variable heavy chain region (VH) and a light chain (LC) comprising a variable light chain region (VL), wherein the VH comprises a heavy chain complementarity domain 1 (HCDR1), a HCDR2, and a HCDR3 comprising amino acid sequences selected from:
a. SEQ ID NO: 15, SEQ ID NO: 26, and SEQ ID NO: 17,
b. SEQ ID NO: 1, SEQ ID NO: 2, and SEQ ID NO: 3,
c. SEQ ID NO: 15, SEQ ID NO: 16, and SEQ ID NO: 17, or
and wherein the VL comprises a light chain complementarity domain 1 (LCDR1), a LCDR2, and a LCDR3 comprising amino acid sequences selected from:
d. SEQ ID NO: 27, SEQ ID NO: 28, and SEQ ID NO: 19,
e. SEQ ID NO: 4, SEQ ID NO: 5, and SEQ ID NO: 6,
f. SEQ ID NO: 18, SEQ ID NO: 5, and SEQ ID NO: 19,
g. SEQ ID NO: 27, SEQ ID NO: 31, and SEQ ID NO: 19, or
h. SEQ ID NO: 27, SEQ ID NO: 34, and SEQ ID NO: 19.

3. The conjugate of claim 2, wherein the VH comprises an amino acid sequence having at least 90% identity to an amino acid sequence selected from SEQ ID NO: 29, SEQ ID NO: 13, SEQ ID NO: 24, SEQ ID NO: 32, or SEQ ID NO: 35.

4. The conjugate of claim 2, wherein the VL comprises an amino acid sequence having at least 90% identity to an amino acid sequence selected from SEQ ID NO: 30, SEQ ID NO: 14, SEQ ID NO: 25, SEQ ID NO: 33, or SEQ ID NO: 36.

5. The conjugate of claim 2, wherein the VH comprises an amino acid sequence having at least 90% identity to the amino acid sequence of SEQ ID NO: 29 and the VL comprises an amino acid sequence having at least 90% identity to the amino acid sequence of SEQ ID NO: 30, wherein the VH comprises a HCDR1 comprising an amino acid sequence of SEQ ID NO: 15, a HCDR2 comprising an amino acid sequence of SEQ ID NO: 26, and a HCDR3 comprising an amino acid sequence of SEQ ID NO: 17, and the VL comprises a LCDR1 comprising an amino acid sequence of SEQ ID NO: 27, a LCDR2 comprising an amino acid sequence of SEQ ID NO: 28, and a LCDR3 comprising an amino acid sequence of SEQ ID NO: 19.

6. The conjugate of claim 2, wherein the HC comprises an amino acid sequence having at least 90% identity to the amino acid sequence of SEQ ID NO: 37 and the LC comprises an amino acid sequence having at least 90% identity to the amino acid sequence of SEQ ID NO: 144.

7. The conjugate of claim 1, wherein the biologically active peptide is conjugated to the anti-GIP antibody, or antigen-binding fragment thereof, through the substituted cysteine residue on the anti-GIP antibody.

8. The conjugate of claim 1, wherein the GLP-1R agonist is liraglutide, albiglutide, taspoglutide, semaglutide, LY2428757, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91, SEQ ID NO: 92, SEQ ID NO: 93, SEQ ID NO: 94, SEQ ID NO: 95, SEQ ID NO: 96, SEQ ID NO: 97, SEQ ID NO: 98, SEQ ID NO: 99, SEQ ID NO: 100, SEQ ID NO: 101, SEQ ID NO: 102, SEQ ID NO: 103, SEQ ID NO: 104, SEQ ID NO: 105, SEQ ID NO: 106, SEQ ID NO: 107, SEQ ID NO: 108, SEQ ID NO: 109, SEQ ID NO: 110, SEQ ID NO: 111, SEQ ID NO: 112, SEQ ID NO: 113, SEQ ID NO: 114, SEQ ID NO: 115, SEQ ID NO: 116, SEQ ID NO: 117, SEQ ID NO: 118, SEQ ID NO: 119, SEQ ID NO: 120, SEQ ID NO: 121, SEQ ID NO: 145, or SEQ ID NO: 146, or a peptide that is 95% identical thereto.

9. (canceled)

10. The conjugate of claim 1, wherein the biologically active peptide is conjugated to the anti-GIP antibody, or antigen-binding fragment thereof, within a CL, CH1, CH2, or CH3 region of the anti-GIP antibody, or antigen-binding fragment thereof.

11. The conjugate of claim 1, wherein the anti-GIP antibody, or antigen-binding fragment thereof, comprises an Fc region.

12. The conjugate of claim 11, wherein the Fc region is an IgG Fc.

13.-14. (canceled)

15. The conjugate of claim 12, wherein the IgG Fc is an IgG1 Fc, and wherein the IgG1 Fc comprises one or more substitutions of a canonical amino acid to a cysteine at position 70, 89, 109, 111, 118, 124, 140, 152, 239, 265, 272, 290, 300, 345, 359, 390, or 442.

16. The conjugate of claim 15, wherein the substitution of a canonical amino acid to a cysteine comprises a D70C, E89C, V109C, A111C, A118C, S124C, A140C, E152C, S239C, V265C, E272C, K290C, Y300C, E345C, T359C, N390C, or S442C mutation.

17. The conjugate of claim 10, wherein the CL domain comprises one or more substitutions of a canonical amino acid to a cysteine at position 149, 205, or any combination thereof.

18. The conjugate of claim 17, wherein the substitution of a canonical amino acid to a cysteine comprises a K149C or V205C substitution.

19. (canceled)

20. A conjugate comprising an anti-glucose-dependent insulinotropic polypeptide (GIP) antibody, or an antigen-binding fragment thereof, conjugated to a biologically active peptide,
wherein the biologically active peptide is conjugated to the anti-GIP antibody, or antigen-binding fragment thereof, through one or more substitutions of a canonical amino acid to a cysteine,
wherein the one or more substitution of a canonical amino acid to a cysteine comprises an E272C, K290C, or S442C mutation in a heavy chain of the antibody, and/or a K149C or V205C substitution in a light chain of the antibody, and
wherein the biologically active peptide comprises a glucagon like peptide 1 receptor (GLP-1R) agonist.

21. (canceled)

22. A pharmaceutical composition comprising the conjugate of claim 1.

23.-40. (canceled)

41. A method of treating a disease or disorder in a patient in need thereof, the method comprising administering the conjugate of claim 1, or a pharmaceutical composition comprising the same, to the patient, thereby treating the disease or disorder.

42.-43. (canceled)

44. A method of treating a disease or disorder in a diabetic patient in need thereof, the method comprising administering to the diabetic patient the conjugate of claim 1, or a pharmaceutical composition comprising the same, to the patient, thereby treating the disease or disorder,
wherein the disease or disorder is selected from coronary heart disease, lipodystrophy, hyperlipidemia, hypercholesterolemia, hypertriglyceridemia, fatty liver disease, hyperphagia, metabolic syndrome, hyperthyroidism, ischemic heart disease, hypertension, stable angina pectoris, unstable angina, acute coronary syndrome, ST elevation myocardial infarction, non-ST elevation myocardial infarction, or any combination thereof.

45.-47. (canceled)

48. A method for blunting or reducing cravings in a subject in need thereof, the method comprising administering to the patient the conjugate of claim 1, or a pharmaceutical composition comprising the same, to the patient, thereby blunting or reducing the cravings.

49. (canceled)


SEQUENCE LISTING (146 sequences)

SEQ ID NO: 1 - protein, 5 aa, synthetic construct
DYYFH

SEQ ID NO: 2 - protein, 17 aa, synthetic construct
WIDPENGDTEYAPNFQV

SEQ ID NO: 3 - protein, 8 aa, synthetic construct
YGNNYFDY

SEQ ID NO: 4 - protein, 12 aa, synthetic construct
SASSSVSSSYLH

SEQ ID NO: 5 - protein, 7 aa, synthetic construct
RTSNLAS

SEQ ID NO: 6 - protein, 9 aa, synthetic construct
LQWSGFPFT

SEQ ID NO: 7 - protein, 7 aa, synthetic construct
GFNIKDY

SEQ ID NO: 8 - protein, 6 aa, synthetic construct
DPENGD

SEQ ID NO: 9 - protein, 8 aa, synthetic construct
GFNIKDYY

SEQ ID NO: 10 - protein, 8 aa, synthetic construct
IDPENGDT

SEQ ID NO: 11 - protein, 10 aa, synthetic construct
NSYGNNYFDY

SEQ ID NO: 12 - protein, 7 aa, synthetic construct
SSVSSSY

SEQ ID NO: 13 - protein, 117 aa, synthetic construct
QVQLQESGAALVRSGASVKLSCTASGFNIKDYYFHWVKQRPDQGLEWIGWIDPENGDTEYAPNFQVKATMTADTYSNTAYLHLSSLTSEDTAVYYCNSYGNNYFDYWGQGTTVTVSS

SEQ ID NO: 14 - protein, 109 aa, synthetic construct
DIQLTQSPAIMAASLGQKVTMTCSASSSVSSSYLHWYQQKSGASPKSLIHRTSNLASGVPARFSGSGSGTSYSLTISSVEAEDDATYYCLQWSGFPFTFGSGTKLEIKR

SEQ ID NO: 15 - protein, 5 aa, synthetic construct
DYYLH

SEQ ID NO: 16 - protein, 17 aa, synthetic construct
WIDPENGDTEYAPKFQD

SEQ ID NO: 17 - protein, 8 aa, synthetic construct
YGIYFMDY

SEQ ID NO: 18 - protein, 12 aa, synthetic construct
SASSSISSNSLH

SEQ ID NO: 19 - protein, 11 aa, synthetic construct
QQGSSFPRMLT

SEQ ID NO: 20 - protein, 7 aa, synthetic construct
GFNIRDY

SEQ ID NO: 21 - protein, 8 aa, synthetic construct
GFNIRDYY

SEQ ID NO: 22 - protein, 10 aa, synthetic construct
NVYGIYFMDY

SEQ ID NO: 23 - protein, 7 aa, synthetic construct
SSISSNS

SEQ ID NO: 24 - protein, 117 aa, synthetic construct
QVQLQESGAELVRSGASVKLSCTASGFNIRDYYLHWIKQRPEQGLEWIGWIDPENGDTEYAPKFQDKATVTADTSSNTAYLQLNSLTSEDTAVYYCNVYGIYFMDYWGQGTTVTVSS

SEQ ID NO: 25 - protein, 111 aa, synthetic construct
DIQLTQSPSTMAASPGEKITITCSASSSISSNSLHWYQQKPGFSPKLLIYRTSNLASGVPGRFSGSGSGTSYSLTIGTMETEDVATYYCQQGSSFPRMLTFGTGTKLEIKR

SEQ ID NO: 26 - protein, 17 aa, synthetic construct
WIDPENGDTEYAPKFQG

SEQ ID NO: 27 - protein, 12 aa, synthetic construct
RASSSISSNSLH

SEQ ID NO: 28 - protein, 7 aa, synthetic construct
RTSNLQS

SEQ ID NO: 29 - protein, 117 aa, synthetic construct
QVQLVQSGAEVKKPGATVKISCKASGFNIRDYYLHWVQQAPGKGLEWMGWIDPENGDTEYAPKFQGRVTITADTSTNTAYMELSSLRSEDTAVYYCNVYGIYFMDYWGQGTMVTVSS

SEQ ID NO: 30 - protein, 111 aa, synthetic construct
DIQLTQSPSSVSASVGDRVTITCRASSSISSNSLHWYQQKPGKAPKLLIYRTSNLQSGVPSRFSGSGSGTDYTLTISSLQPEDFATYYCQQGSSFPRMLTFGQGTKLEIKR

SEQ ID NO: 31 - protein, 7 aa, synthetic construct
RTSSLQS

SEQ ID NO: 32 - protein, 117 aa, synthetic construct
QVQLVQSGAEVKKPGATVKISCKVSGFNIRDYYLHWVQQAPGKGLEWMGWIDPENGDTEYAPKFQGRVTITADTSTDTAYMELSSLRSEDTAVYYCNVYGIYFMDYWGQGTMVTVSS

SEQ ID NO: 33 - protein, 111 aa, synthetic construct
DIQMTQSPSSVSASVGDRVTITCRASSSISSNSLHWYQQKPGKAPKLLIYRTSSLQSGVPSRFSGSGSGTDYTLTISSLQPEDFATYYCQQGSSFPRMLTFGQGTKLEIKR

SEQ ID NO: 34 - protein, 7 aa, synthetic construct
RTSNRAT

SEQ ID NO: 35 - protein, 117 aa, synthetic construct
QVQLQESGAEVKKPGASVKVSCKASGFNIRDYYLHWVRQAPGQGLEWMGWIDPENGDTEYAPKFQGRVTTTADTSISTAYMELSRLRSDDTAVYYCNVYGIYFMDYWGQGTLVTVSS

SEQ ID NO: 36 - protein, 111 aa, synthetic construct
DIQMTQSPGTLSLSPGERATLSCRASSSISSNSLHWYQQKPGQAPRLLIYRTSNRATGIPARFSGSGSGTDYTLTISSLEPEDFAVYYCQQGSSFPRMLTFGGGTKVEIKR

SEQ ID NO: 37 - protein, 447 aa, synthetic construct
QVQLVQSGAEVKKPGATVKISCKASGFNIRDYYLHWVRQAPGKGLEWMGWIDPENGDTEYAPKFQGRVTITADTSTNTAYMELSSLRSEDTAVYYCNVYGIYFMDYWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 38 - protein, 39 aa, synthetic construct
HGEGTFTSDLSKQMEEEAVRLFIEWLKNGGPSSGAPPPS

SEQ ID NO: 39 - protein, 39 aa, synthetic construct
HSDGTFTSDLSKQMEEEAVRLFIEWLKNGGPSSGAPPPS

SEQ ID NO: 40 - protein, 39 aa, synthetic construct
HGEGTFTSDLSKQLEEEAVRLFIEWLKNGGPSSGAPPPS

SEQ ID NO: 41 - protein, 39 aa, synthetic construct
HGEGTFTSDLSKQLEEEAVRLFIEFLKNGGPSSGAPPPS

SEQ ID NO: 42 - protein, 39 aa, synthetic construct
HGEGTFTSDLSKQLEEEAARLFIEFLKNGGPSSGAPPPS

SEQ ID NO: 43 - protein, 30 aa, synthetic construct
HGEGTFTSDLSKQMEEEAVRLFIEWLKNGG

SEQ ID NO: 44 - protein, 30 aa, synthetic construct
HGEGTFTSDLSKQLEEEAVRLFIEWLKNGG

SEQ ID NO: 45 - protein, 30 aa, synthetic construct
HGEGTFTSDLSKQLEEEAVRLFIEFLKNGG

SEQ ID NO: 46 - protein, 30 aa, synthetic construct
HGEGTFTSDLSKQLEEEAARLFIEFLKNGG

SEQ ID NO: 47 - protein, 28 aa, synthetic construct
HGEGTFTSDLSKQMEEEAVRLFIEWLKN

SEQ ID NO: 48 - protein, 28 aa, synthetic construct
HGEGTFTSDLSKQLEEEAVRLFIEWLKN

SEQ ID NO: 49 - protein, 28 aa, synthetic construct
HGEGTFTSDLSKQLEEEAVRLFIEFLKN

SEQ ID NO: 50 - protein, 28 aa, synthetic construct
HGEGTFTSDLSKQLEEEAARLFIEFLKN

SEQ ID NO: 51 - protein, 39 aa, synthetic construct
HGEGTFTSDLSKQLEEKAAKEFIEFLKQGGPSSGAPPPS

SEQ ID NO: 52 - protein, 39 aa, synthetic construct
HGEGTFTSDLSKQLEEKAAKEFIEWLKQGGPSSGAPPPS

SEQ ID NO: 53 - protein, 39 aa, synthetic construct
  SITE 14: note = Octyl glycine
HGEGTFTSDLSKQGEEEAVRLFIEWLKQGGPSSGAPPPS

SEQ ID NO: 54 - protein, 39 aa, synthetic construct
  SITE 34: note = Octyl Glycine
HGEGTFTSDLSKQLEEEAVRLFIEWLKQGGPSSGAPPPS

SEQ ID NO: 55 - protein, 37 aa, synthetic construct
HGEFTFTSDLSKQLEEEAVRLFIEWLKQGGPSKEIIS

SEQ ID NO: 56 - protein, 39 aa, synthetic construct
HGEFTFTSDLSKQLEEKAAKEFIEWLKQGGPSSGAPPPS

SEQ ID NO: 57 - protein, 39 aa, synthetic construct
HGEGTFTSDLVKILEAEAVRKFIEFLKNGGPSSGAPPPS

SEQ ID NO: 58 - protein, 40 aa, synthetic construct
HGEGTFTSDLSKQMEEEAVRLFIEWLKNGGPSSGAPPPSK

SEQ ID NO: 59 - protein, 31 aa, synthetic construct
HAEGTFTSDVSSYLEGQAAKEFIAWLVKGRG

SEQ ID NO: 60 - protein, 30 aa, synthetic construct
HAEGTFTSDVSSYLEGQAAKEFIAWLVKGR

SEQ ID NO: 61 - protein, 30 aa, synthetic construct
  SITE 2: note = X is 2-Aminoisobutyric acid
  SITE 29: note = X is 2-Aminoisobutyric acid
HXEGTFTSDVSSYLEGQAAREFIAFLVRXR

SEQ ID NO: 62 - protein, 31 aa, synthetic construct
  SITE 2: note = X is Ala, Val, or Gly
  SITE 16: note = X is Gly, Lys, or Glu
  SITE 17: note = X is Gln or Lys
  SITE 24: note = X is Ala or Glu
  SITE 27: note = X is Val or Lys
  SITE 28: note = X is Lys, Asn, or Arg
  SITE 30: note = X is Arg or Gly
  SITE 31: note = X is Gly, His, Pro, or not present
HXEGTFTSDVSSYLEXXAAKEFIXWLXXGXX

SEQ ID NO: 63 - protein, 31 aa, synthetic construct
HAEGTFTSDVSSYLEGQAAKEFIAWLVRGRG

SEQ ID NO: 64 - protein, 31 aa, synthetic construct
HAEGTFTSDVSSYLEGQAAKEFIEWLVKGRG

SEQ ID NO: 65 - protein, 31 aa, synthetic construct
HAEGTFTSDVSSYLEKQAAKEFIAWLVKGRG

SEQ ID NO: 66 - protein, 31 aa, synthetic construct
HGEGTFTSDVSSYLEEQAAKEFIAWLVKGGG

SEQ ID NO: 67 - protein, 31 aa, synthetic construct
HVEGTFTSDVSSYLEEQAAKEFIAWLVKGGG

SEQ ID NO: 68 - protein, 31 aa, synthetic construct
HGEGTFTSDVSSYLEEQAAKEFIAWLKNGGG

SEQ ID NO: 69 - protein, 31 aa, synthetic construct
HVEGTFTSDVSSYLEEQAAKEFIAWLKNGGG

SEQ ID NO: 70 - protein, 31 aa, synthetic construct
HGEGTFTSDVSSYLEEQAAKEFIAWLVKGGP

SEQ ID NO: 71 - protein, 31 aa, synthetic construct
HVEGTFTSDVSSYLEEQAAKEFIAWLVKGGP

SEQ ID NO: 72 - protein, 31 aa, synthetic construct
HGEGTFTSDVSSYLEEQAAKEFIAWLKNGGP

SEQ ID NO: 73 - protein, 31 aa, synthetic construct
HVEGTFTSDVSSYLEEQAAKEFIAWLKNGGP

SEQ ID NO: 74 - protein, 30 aa, synthetic construct
HGEGTFTSDVSSYLEEQAAKEFIAWLVKGG

SEQ ID NO: 75 - protein, 30 aa, synthetic construct
HVEGTFTSDVSSYLEEQAAKEFIAWLVKGG

SEQ ID NO: 76 - protein, 30 aa, synthetic construct
HVEGTFTSDVSSYLEEQAAKEFIAWLVNGG

SEQ ID NO: 77 - protein, 30 aa, synthetic construct
HGEGTFTSDVSSYLEEQAAKEFIAWLVNGG

SEQ ID NO: 78 - protein, 40 aa, synthetic construct
  SITE 2: note = X is D-Ala, Gly, Val, Leu, Ile, Ser, or Thr
  SITE 16: note = X is Gly, Glu, Asp, or Lys
  SITE 27: note = X is Val or Ile
HXEGTFTSDVSSYLEXQAAKEFIAWLXKGGPSSGAPPPCC

SEQ ID NO: 79 - protein, 40 aa, synthetic construct
HVEGTFTSDVSSYLEEQAAKEFIAWLIKGGPSSGAPPPCC

SEQ ID NO: 80 - protein, 39 aa, synthetic construct
  SITE 20: note = Lysine at position 20 is modified with 17-carboxyheptadecanoic acid
HGEGTFTSDLSKQMEEEAVKLFIEWLKNGGPSSGAPPPS

SEQ ID NO: 81 - protein, 39 aa, synthetic construct
  SITE 32: note = Lysine at position 32 is modified with a 17-carboxyheptadecanoyl moiety
HGEGTFTSDLSKQMEEEAVRLFIEWLKNGGPKSGAPPPS

SEQ ID NO: 82 - protein, 38 aa, synthetic construct
  SITE 19: note = Lysine at position 19 is modified with a 17-carboxyheptadeconyl moiety
GEGTFTSDLSKQMEEEAVKLFIEWLKNGGPSSGAPPPS

SEQ ID NO: 83 - protein, 39 aa, synthetic construct
  SITE 14: note = X is Norleucine
  SITE 32: note = Lysine at position 32 is modified with a 17-carboxyheptadecanoyl moiety
HGEGTFTSDLSRQXEEEAVRLFIEWLRNGGPKSGAPPPS

SEQ ID NO: 84 - protein, 39 aa, synthetic construct; identical to SEQ ID NO: 80
  SITE 20: note = Lysine at position 20 modified with 19-carboxy-nonadecanoylamino moiety

SEQ ID NO: 85 - protein, 39 aa, synthetic construct; identical to SEQ ID NO: 80
  SITE 20: note = Lysine at position 20 modified with 15-carboxypentadecanoylamino moiety

SEQ ID NO: 86 - protein, 39 aa, synthetic construct; identical to SEQ ID NO: 80
  SITE 20: note = Lysine at position 20 modified with 13-carboxytridecanoylamino moiety

SEQ ID NO: 87 - protein, 39 aa, synthetic construct; identical to SEQ ID NO: 80
  SITE 20: note = Lysine at position 20 modified with 11-carboxy-undecanoyl-amino moiety

SEQ ID NO: 88 - protein, 40 aa, synthetic construct; identical to SEQ ID NO: 58

SEQ ID NO: 89 - protein, 40 aa, synthetic construct; identical to SEQ ID NO: 58

SEQ ID NO: 90 - protein, 40 aa, synthetic construct; identical to SEQ ID NO: 58

SEQ ID NO: 91 - protein, 40 aa, synthetic construct; identical to SEQ ID NO: 58

SEQ ID NO: 92 - protein, 40 aa, synthetic construct; identical to SEQ ID NO: 58

SEQ ID NO: 93 - protein, 40 aa, synthetic construct; identical to SEQ ID NO: 58

SEQ ID NO: 94 - protein, 30 aa, synthetic construct
AEGTFTSDVSSYLEGQAAREFIAWLVKGRG

SEQ ID NO: 95 - protein, 30 aa, synthetic construct; identical to SEQ ID NO: 94

SEQ ID NO: 96 - protein, 32 aa, synthetic construct
HAEGTFTSDVSSYLEGQAAREFIAWLVRGRGK

SEQ ID NO: 97 - protein, 32 aa, synthetic construct; identical to SEQ ID NO: 96

SEQ ID NO: 98 - protein, 31 aa, synthetic construct
  SITE 2: note = X is 2-Aminoisobutyric acid
HXEGTFTSDVSSYLEGQAAKEFIAWLVKGRK

SEQ ID NO: 99 - protein, 31 aa, synthetic construct
  SITE 2: note = X is 2-Aminoisobutyric acid
HXEGTFTSDVSSYLEGQAAKEFIAWLVKGKG

SEQ ID NO: 100 - protein, 30 aa, synthetic construct
  SITE 2: note = X is 2-Aminoisobutyric acid
HXEGTFTSDVSSYLEXQAAKEFIAWLVKGK

SEQ ID NO: 101 - protein, 30 aa, synthetic construct
  SITE 2: note = X is 2-Aminoisobutyric acid
  SITE 16: note = X is 2-Aminoisobutyric acid
  SITE 26: note = X is beta leucine
HXEGTFTSDVSSYLEXQAAKEFIAWXVKGK

SEQ ID NO: 102 - protein, 31 aa, synthetic construct
  SITE 2: note = X is 2-Aminoisobutyric acid
  SITE 16: note = X is 2-Aminoisobutyric acid
HXEGTFTSDVSSYLEXQAAKEFIAWLVKGRK

SEQ ID NO: 103 - protein, 31 aa, synthetic construct
  SITE 2: note = X is 2-Aminoisobutyric acid
  SITE 16: note = X is 2-Aminoisobutyric acid
  SITE 26: note = X is beta leucine
HXEGTFTSDVSSYLEXQAAKEFIAWXVKGRK

SEQ ID NO: 104 - protein, 31 aa, synthetic construct; identical to SEQ ID NO: 103
  SITE 2: note = X is 2-Aminoisobutyric acid
  SITE 16: note = X is 2-Aminoisobutyric acid
  SITE 26: note = X is alpha methyl leucine

SEQ ID NO: 105 - protein, 31 aa, synthetic construct
  SITE 2: note = X is 2-Aminoisobutyric acid
  SITE 6: note = X is AMEF
  SITE 16: note = X is 2-Aminoisobutyric acid
HXEGTXTSDVSSYLEXQAAKEFIAWLVKGRK

SEQ ID NO: 106 - protein, 31 aa, synthetic construct
  SITE 2: note = X is 2-Aminoisobutyric acid
  SITE 10: note = X is beta leucine
  SITE 16: note = X is 2-Aminoisobutyric acid
HXEGTFTSDXSSYLEXQAAKEFIAWLVKGRK

SEQ ID NO: 107 - protein, 31 aa, synthetic construct
  SITE 2: note = X is 2-Aminoisobutyric acid
  SITE 16: note = X is 2-Aminoisobutyric acid
HXEGTFTSDVSSYLEXQAAKEFIAWLVKGGK

SEQ ID NO: 108 - protein, 31 aa, synthetic construct
  SITE 2: note = X is 2-Aminoisobutyric acid
  SITE 16: note = X is 2-Aminoisobutyric acid
HXEGTFTSDVSSYLEXQAAKEFIAWLKNGGK

SEQ ID NO: 109 - protein, 31 aa, synthetic construct
  SITE 2: note = X is 2-Aminosiobutyric acid
  SITE 16: note = X is 2-Aminosiobutyric acid
  SITE 31: note = X is Aminoethylethanolamine (Aeea)
HXEGTFTSDVSSYLEXQAAKEFIAWLKNGGX

SEQ ID NO: 110 - protein, 31 aa, synthetic construct
  SITE 2: note = X is 2-Aminoisobutyric acid
  SITE 16: note = X is 2-Aminoisobutyric acid
HXEGTFTSDVSSYLEXQAAKEFIAWLVKGGG

SEQ ID NO: 111 - protein, 31 aa, synthetic construct
  SITE 2: note = X is 2-Aminoisobutyric acid
HXEGTFTSDVSSYLEGQAAKEFIAWLVKGGG

SEQ ID NO: 112 - protein, 31 aa, synthetic construct
  SITE 2: note = X is 2-Aminoisobutyric acid
HXEGTFTSDVSSYLEGQAAKEFIAWLVKGGK

SEQ ID NO: 113 - protein, 39 aa, synthetic construct
  SITE 2: note = X is 2-Aminoisobutyric acid
  SITE 16: note = X is 2-Aminoisobutyric acid
HXEGTFTSDVSSYLEXEAVRLFIEWLKNGGPSSGAPPPS

SEQ ID NO: 114 - protein, 31 aa, synthetic construct; identical to SEQ ID NO: 66

SEQ ID NO: 115 - protein, 31 aa, synthetic construct
  SITE 2: note = X is 2-Aminoisobutyric acid
HXEGTFTSDVSSYLEEQAAKEFIAWLVKGGG

SEQ ID NO: 116 - protein, 31 aa, synthetic construct
  SITE 2: note = X is 2-Aminoisobutyric acid
HXEGTFTSDYSSYLEEQAAKEFIAWLVKGGG

SEQ ID NO: 117 - protein, 31 aa, synthetic construct
  SITE 2: note = X is 2-Aminoisobutyric acid
HXEGTFTSDVSKYLEEEAVRLFIEWLKNGGG

SEQ ID NO: 118 - protein, 31 aa, synthetic construct
  SITE 2: note = X is 2-Aminoisobutyric acid
HXEGTFTSDVSKYLEEEAAKLFIEWLKNGGG

SEQ ID NO: 119 - protein, 31 aa, synthetic construct
  SITE 2: note = X is 2-Aminoisobutyric acid
HXEGTFTSDVSKYLEEEAAKLFIEWLVKGGG

SEQ ID NO: 120 - protein, 30 aa, synthetic construct
  SITE 2: note = X is 2-Aminoisobutyric acid
  SITE 16: note = X is 2-Aminoisobutyric acid
HXIGTFTSDVSSYLEXQAAKEFIAWLVKGG

SEQ ID NO: 121 - protein, 30 aa, synthetic construct
  SITE 2: note = X is 2-Aminoisobutyric acid
  SITE 16: note = X is 2-Aminoisobutyric acid
HXEGTFTSEVSSYLEXQAAKEFIAWLVKGG

SEQ ID NO: 122 - protein, 153 aa, Homo sapiens
MVATKTFALLLLSLFLAVGLGEKKEGHFSALPSLPVGSHAKVSSPQPRGPRYAEGTFISDYSIAMDKIHQQDFVNWLLAQKGKKNDWKHNITQREARALELASQANRKEEEAVEPQSSPAKNPSDEDLLRDLLIQELLACLLDQTNLCRLRSR

SEQ ID NO: 123 - protein, 42 aa, Homo sapiens
YAEGTFISDYSIAMDKIHQQDFVNWLLAQKGKKNDWKHNITQ

SEQ ID NO: 124 - protein, 42 aa, Mus musculus
YAEGTFISDYSIAMDKIRQQDFVNWLLAQRGKKSDWKHNITQ

SEQ ID NO: 125 - protein, 42 aa, Rattus rattus
YAEGTFISDYSIAMDKIRQQDFVNWLLAQKGKKNDWKHNLTQ

SEQ ID NO: 126 - protein, 42 aa, Macaca mulatta; identical to SEQ ID NO: 123

SEQ ID NO: 127 - protein, 330 aa, synthetic construct [sequence omitted]

SEQ ID NO: 128 - protein, 325 aa, synthetic construct
STKGPSVFPLAPCSRSTSESTAALGCLVKDYFPEPVTVSWNSGALTSGVHTFPAVLQSSGLYSLSSVVTVPSSSLGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 129 - protein, 326 aa, synthetic construct
STKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 130 - protein, 5 aa, synthetic construct
  REPEAT 1..5: note = Sequence is present n times, wherein n is 1, 2, 3, 4, or 5
GGGGA

SEQ ID NO: 131 - protein, 5 aa, synthetic construct
  REPEAT 1..5: note = Sequenceis present n times, wherein n is 1, 2, 3, 4, or 5
GGGGS

SEQ ID NO: 132 - protein, 8 aa, synthetic construct
GGGGGGGG

SEQ ID NO: 133 - protein, 6 aa, synthetic construct
GGGGGG

SEQ ID NO: 134 - protein, 5 aa, synthetic construct
  REPEAT 1..5: note = Sequence is present n times, wherein n is 1, 2, 3, 4, or 5
EAAAK

SEQ ID NO: 135 - protein, 7 aa, synthetic construct
  REPEAT 2..6: note = Sequence is present n times, wherein n is 1, 2, 3, 4, or 5
AEAAAKA

SEQ ID NO: 136 - protein, 12 aa, synthetic construct
AEAAAKEAAAKA

SEQ ID NO: 137 - protein, 45 aa, synthetic construct
AEAAAKEAAAKEAAAKEAAAKALEAEAAAKEAAAKEAAAKEAAAK

SEQ ID NO: 138 - protein, 5 aa, synthetic construct
PAPAP

SEQ ID NO: 139 - protein, 10 aa, synthetic construct
  REPEAT 1..2: note = Sequence may be present n times, wherein n is 1, 2, 3, 4, or 5. SEQ ID NO: 139 represents the embodiment where the sequence is present n=5 times.
  SITE 2: note = X is A, K, or E
  SITE 4: note = X is A, K, or E; identity of X is equal to the identity at position 2
  SITE 6: note = X is A, K, or E; identity of X is equal to the identity at position 2
  SITE 8: note = X is A, K, or E; identity of X is equal to the identity at position 2
  SITE 10: note = X is A, K, or E; identity of X is equal to the identity at position 2
XPXPXPXPXP

SEQ ID NO: 140 - protein, 12 aa, synthetic construct
GSAGSAAGSGEF

SEQ ID NO: 141 - protein, 18 aa, synthetic construct
KESGSVSSEQLAQFRSLD

SEQ ID NO: 142 - protein, 14 aa, synthetic construct
EGKSSGSGSESKST

SEQ ID NO: 143 - protein, 12 aa, synthetic construct
AEAAAKEAAAKA

SEQ ID NO: 144 - protein, 217 aa, synthetic construct
DIQLTQSPSSVSASVGDRVTITCRASSSISSNSLHWYQQKPGKAPKLLIYRTSNLQSGVPSRFSGSGSGTDYTLTISSLQPEDFATYYCQQGSSFPRMLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 145 - protein, 47 aa, synthetic construct
  SITE 2: note = X is 2-Aminoisobutyric acid
HXEGTFTSDYSSYLEEQAAKEFIAWLVKGGGGGGGSGGGGSGGGGSK

SEQ ID NO: 146 - protein, 47 aa, synthetic construct; identical to SEQ ID NO: 145
  SITE 2: note = X is 2-aminoisobutyric acid
  SITE 47: note = Terminal lysine is modified to be linked to a bromoacetyl group (BrAc)